(19) United States
(12) Patent Application Publication
(10) Pub. No.: US 2026/0364186 A1
(43) Pub. Date: Nov. 19, 2026

(54) Chemical Conjugates, Compositions, and Methods of Preparation and Use Thereof

(71) Applicant: Transfection Holdings Limited, Auckland (NZ)

(72) Inventors: Ricardo Lopez-Gonzalez, Auckland (NZ); Sean Niall O'Byrne, Auckland (NZ); Stephen Sowerby, Auckland (NZ)

(21) Appl. No.: 19/107,269

(22) PCT Filed: Aug. 31, 2023
(86) PCT No.: PCT/NZ2023/050089

§ 371 Date: Feb. 27, 2025

Related U.S. Application Data
(60) Provisional application No. 63/402,935, filed on Aug. 31, 2022.

(51) Int. Cl. A61K 47/55; A61K 31/69; A61K 49/00; A61P 35/00

(52) U.S. Cl. CPC A61K 47/551; A61K 31/69; A61K 49/0052; A61P 35/00

(57) ABSTRACT

This disclosure provides compounds, including chemical conjugates, and compositions comprising these, as well as kits, which are useful in therapeutic and diagnostic procedures, amongst others. This disclosure also provides preparative methods and uses for these compounds, compositions, and kits.

### RELATED APPLICATION

This application is a U.S. National Stage application under 35 U.S.C. § 371 from International Patent Application No. PCT/NZ2023/050089 filed Aug. 31, 2023, which claims the benefit of U.S. provisional patent application 63/402,935, filed Aug. 31, 2022.

### FIELD

The present disclosure relates to various compounds, including chemical conjugates, and compositions comprising these. Also related are methods of preparing and utilising these compounds or compositions, as well as kits comprising these compounds or compositions.

### BACKGROUND

Animals, including humans, are comprised of many different types of cells which, under normal biological control, contribute to the growth, health, and reproduction. Cells that inherit and/or incorporate changes to their control mechanisms may become malignant and show changed cellular metabolism, unregulated cell growth, and lack programmed cell death. Such cells can lead to the development of cancers and other proliferative conditions, which can cause severe debilitation and, ultimately, death.

The incidence of malignancies in the human populations is very high. Approximately one third of human deaths can be attributed to malignant disease. The early identification of malignant cells generally results in better prognosis for the treatment of malignant disease because the scale of the disease is less, and the management of few malignant cells is more efficient than the management of many malignant cells. Inaccurate, late, and missed detection of malignant cells can have detrimental consequences for a subject and can create significant healthcare costs due to the additional treatment burden of advanced disease.

Yet, the identification of malignant cells is often difficult because these cells can lack readily identifiable features when viewed against high background levels of number of normal cells. In addition to this, most cancer treatments utilise the unselective distribution of the drugs throughout the body leading to deleterious events. Recently research has shifted to identifying gene mutations and the associated perturbations of signal transduction pathways in melanoma, in the hope that specific and targeted therapies can be developed. However, both conventional therapeutic agents and the newer inhibitors are still capable of damaging normal cells as their molecular targets are not unique to melanoma cells. Moreover, many malignancies (including melanoma) show high levels of resistance to conventional chemotherapeutic agents and radiation.

Malignant cells are often used in cell cultures and manipulated for experimental, industrial, and manufacturing purposes. Many current methods for modifying malignant cells rely on the administration of polynucleotides or other agents by disruption of the cell wall. These methods are highly inefficient, time consuming, and damaging to the cells.

As such, there remains an urgent a need for compounds and compositions that are effective for targeting malignant cells, for providing improvements in identification, treatment, and modification methods for malignant cells. This disclosure seeks to address these needs, or to at least provide the public with a useful alternative.

### SUMMARY

Unexpectedly and surprisingly, the present inventors have found that pantothenic acid groups, pantothenic acid derivative groups, or pantothenic acid analogues (e.g., 138 group) may be conjugated to chemical agents to provide delivery of such agent(s) to malignant cells. These compounds exhibit marked specificity and selectivity for malignant cells, and provide many valuable applications, including, for example, detection methods, therapeutic methods, and cell modification methods.

In one aspect, the present disclosure provides a compound for use in targeting at least one chemical agent to a malignant cell.

In particular aspects, the compound is a chemical conjugate comprising a pantothenic acid group, a pantothenic acid derivative group, or pantothenic acid analogue, and at least one chemical agent.

In specific aspects:

The compound is of formula (I):

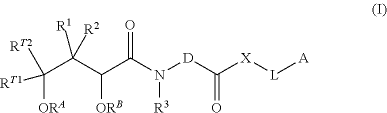

wherein:
—R^{A} and —R^{B} are each independently selected from hydrogen, alkyl, alkenyl, alkynyl, aralkyl, cycloalkylalkyl, alkanoyl and aralkanoyl;
or —R^{A} and —R^{B} are together —C(R^{C1})(R^{C2})—, forming a 6-membered ring, where —R^{C1} is independently selected from hydrogen, alkyl, alkenyl, alkynyl, aralkyl and cycloalkylalkyl, and —R^{C2} is independently selected from hydrogen, alkyl, alkenyl, alkynyl, aralkyl, cycloalkylalkyl, alkoxy, alkenoxy, alkynoxy, aralkoxy and cycloalkylalkoxy, or —R^{C1} and —R^{C2} are together oxo (═O);
—R^{T1} and —R^{T2} are each independently hydrogen or alkyl;
—R¹ and —R² are each independently selected from hydrogen, alkyl, alkenyl, alkynyl, aralkyl and cycloalkylalkyl;
—R³ is hydrogen or alkyl;
-D- is C₂₋₄ alkenylene or C₁₋₄ alkylene, where the alkenylene or alkylene is optionally substituted with alkyl or halo;
—X— is a covalent bond, —N(R⁴)—, —O—, —S—, or —Se—, where —R⁴ is hydrogen or alkyl;
-L- is a linker or a covalent bond; and
-A comprises at least one chemical agent;
and salts, solvates and protected forms thereof.

The linker is selected from the group consisting of bivalent linkers; multivalent linkers; cleavable linkers; enzyme cleavable linkers; protease cleavable linkers; esterase cleavable linkers.

The linker is selected from the group consisting of a triazole linker; glycol linker; polyethylene glycol (PEG) linker, alkyl linker; heteroalkyl linker; and alkylene linker.

The at least one chemical agent is at least one detection agent; at least one therapeutic agent; and/or at least one cell modifying agent.

The at least one detection agent is selected from the group consisting of: dyes; fluorophores; fluorescent proteins; labelled chelating agents; unlabelled chelating agents; optical contrast agents; X-ray contrast agents; magnetic resonance imaging contrast agents; and positron emission tomography contrast agents.

The at least one detection agent is selected from the group consisting of BODIPY; BODIPY derivatives; DOTA chelator; indocyanine green; fluorine-18 [¹⁸F] radioisotopes.

The at least one therapeutic agent is selected from the group consisting of: antibodies; antimetabolites; biologic response modifiers; chain terminators; growth factor inhibitors; hormonal agents; nucleic acid intercalators; pathway inhibitors; proteasome inhibitors; enzyme inhibitors; radionuclides; and radionucleotides.

The at least one therapeutic agent is selected from the group consisting of auranofin; vemurafenib; dabrafenib; and trametinib.

The at least one cell modifying agent is selected from the group consisting of: polypeptides; peptides; enzymes; enzyme fragments; antibodies; antibody fragments; antibody epitopes; polynucleotides; RNAs; DNAs; genes; and gene fragments.

The malignant cell is a THL positive cell.

The malignant cell is selected from the group consisting of: bladder cancer cells, bone cancer cells, breast cancer cells, brain cancer cells, colorectal cancer cells, endometrial cancer cells, head and neck cancer cells, kidney cancer cells, leukaemia cells, liver cancer cells, lung cancer cells, lymphoma cells, melanoma cells, ovarian cancer cells, pancreatic cancer cells, prostate cancer cells, and thyroid cancer cells.

The malignant cell is selected from the group consisting of: melanoma cells; colon cancer cells; rectal cancer cells; and leukaemia cells.

The malignant cell is selected from the group comprising: malignant melanocytes; melanoma tissue cells; melanoma lesion cells; and melanoma tumour cells.

The malignant cell is selected from the group consisting of: melanoma metastasis cells; cutaneous melanoma cells; ocular melanoma cells; superficial spreading melanoma cells; nodular melanoma cells; lentigo maligna melanoma cells; amelanotic melanoma cells; acral lentiginous melanoma cells; mucosal melanoma cells; desmoplastic melanoma cells; and uveal melanoma cells.

The malignant cell is selected from the group consisting of: melanoma cells having mutation of one or more of CDK2NA, MDm2, CDK4EGF, RB1, MC1R, TYR, TYRP1, and ASIP genes; or having mutation of one or more of BRAF, EGF, Fas, and PTEN genes.

The malignant cell is selected from the group consisting of: colorectal adenocarcinoma cells; colorectal mucinous adenocarcinoma cells; colorectal signet ring adenocarcinoma cells; colorectal lymphoma cells; gastrointestinal stromal tumour cells; colorectal leiomyosarcoma cells; colorectal melanoma cells; colorectal squamous cell carcinoma cells; hereditary nonpolyposis colorectal cancer cells; metastatic colorectal cancer cells; familial adenomatous polyposis cells; juvenile polyposis coli cells; Turcot Syndrome cancer cells; and Peutz-Jeghers Syndrome cancer cells.

The malignant cell is selected from the group consisting of: lymphocytic leukaemia cells; acute lymphocytic leukaemia cells; chronic lymphocytic leukaemia cells; myeloid leukaemia cells; acute myeloid leukaemia cells; chronic myeloid leukaemia cells; prolymphocytic leukaemia cells; large granular lymphocytic (LGL) leukaemia cells; hairy cell leukaemia cells; and myelodysplastic syndrome cells.

The malignant cell is selected from the group consisting of: tumour node and metastasis (TNM) staged cancer cells; Stage 0 cancer cells; Stage I cancer cells; Stage II cancer cells; Stage III cancer cells; Stage IIIA cancer cells; Stage IIIB cancer cells; Stage IIIC cancer cells; Stage IIID cancer cells; Stage IV cancer cells.

The targeting is detected by one or more of: illumination; visualisation; imaging; optical imaging; fluorescence imaging; infra-red imaging; radio imaging; microscopy imaging; machine imaging; extended depth of field imaging; image recording; image capturing; image analysis; image filtering; image reconstruction; computer-aided image analysis; image analysis using artificial intelligence (AI); deep learning; image storage; image storage by cloud computing; image retrieval; image transmission; image recognition; image reconstruction; tomography; computed tomography (CT or CAT); positron emission tomography (PET) or PET-CT; and magnetic resonance imaging (MRI).

In further aspects:

The compound is selected from the group consisting of formulae:

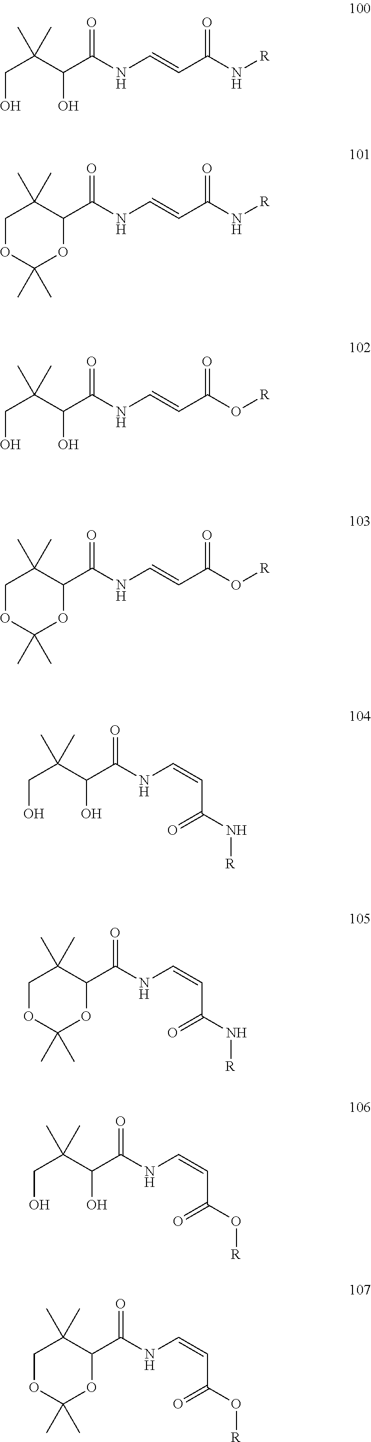

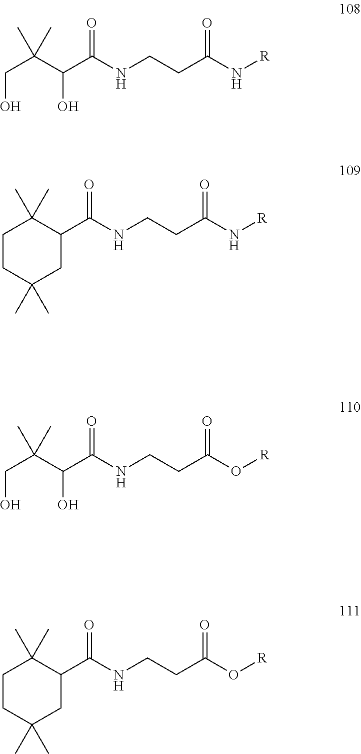

wherein —R is -L-A;
wherein -L- is a linker or a covalent bond; and -A is at least one chemical agent,
and salts, solvates, and protected forms thereof.

In still further aspects:

The compound is selected from the group consisting of formulae:

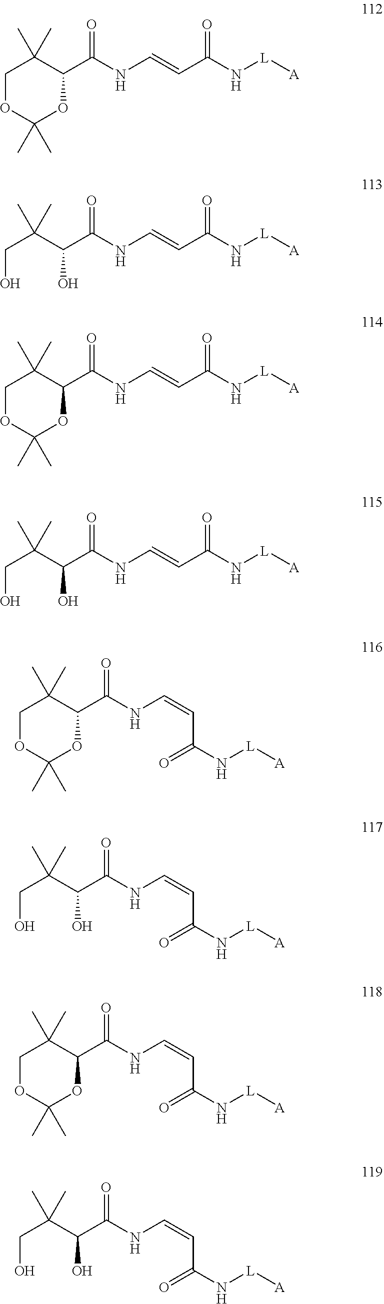

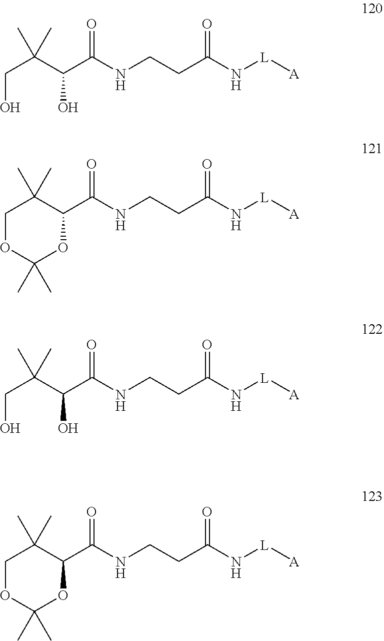

wherein -L- is a linker or a covalent bond; and -A is at least one chemical agent, and salts, solvates, and protected forms thereof.

In even further aspects:

The compound is selected from the group consisting of

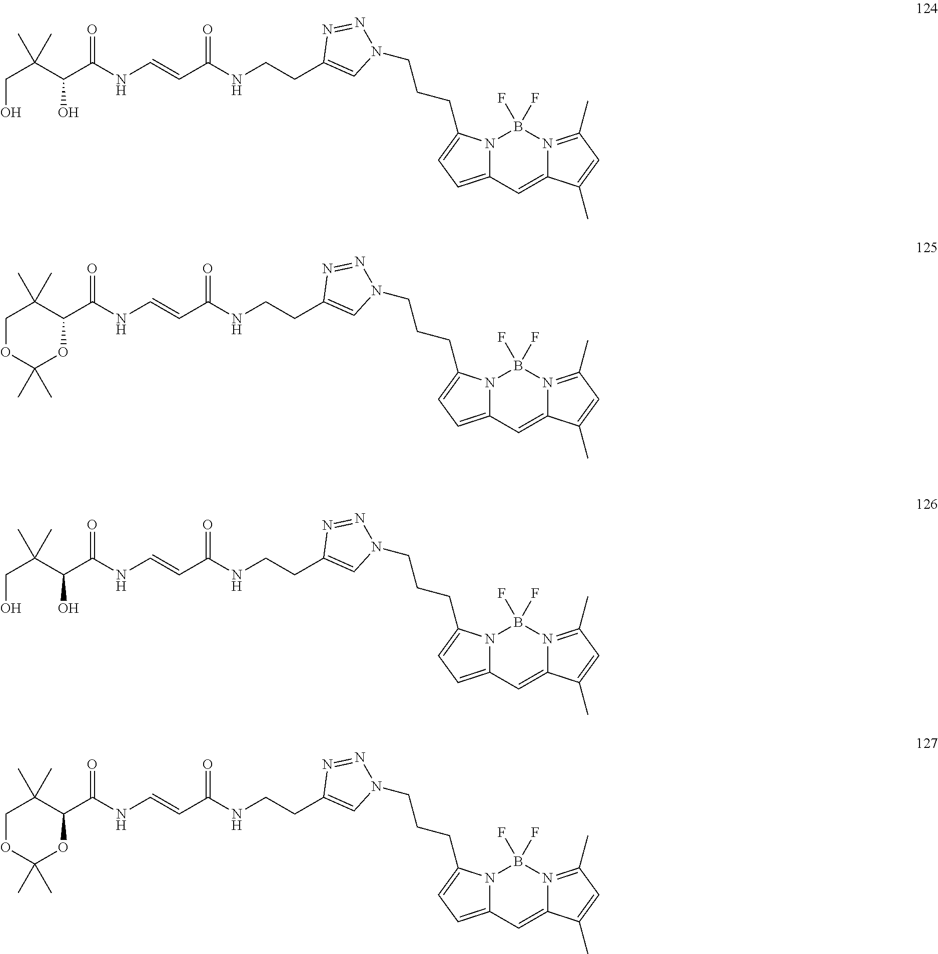

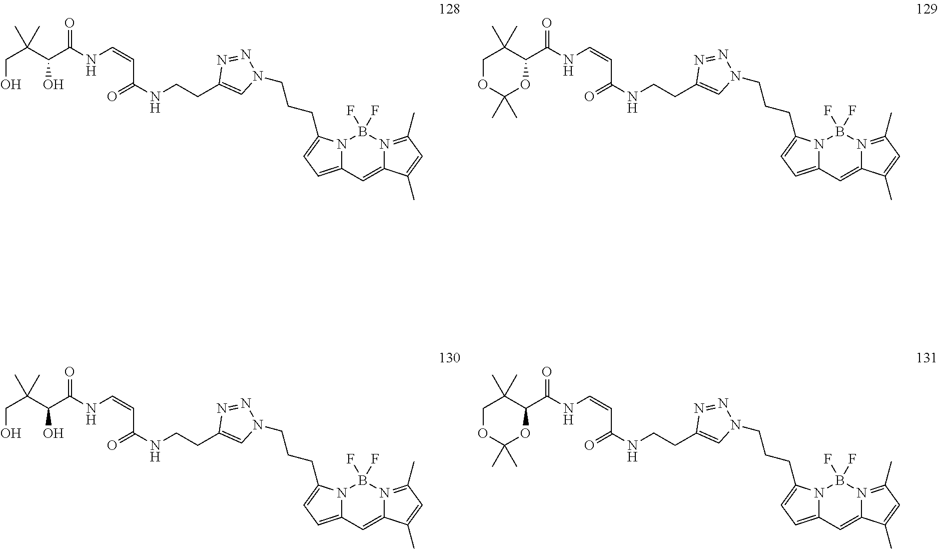

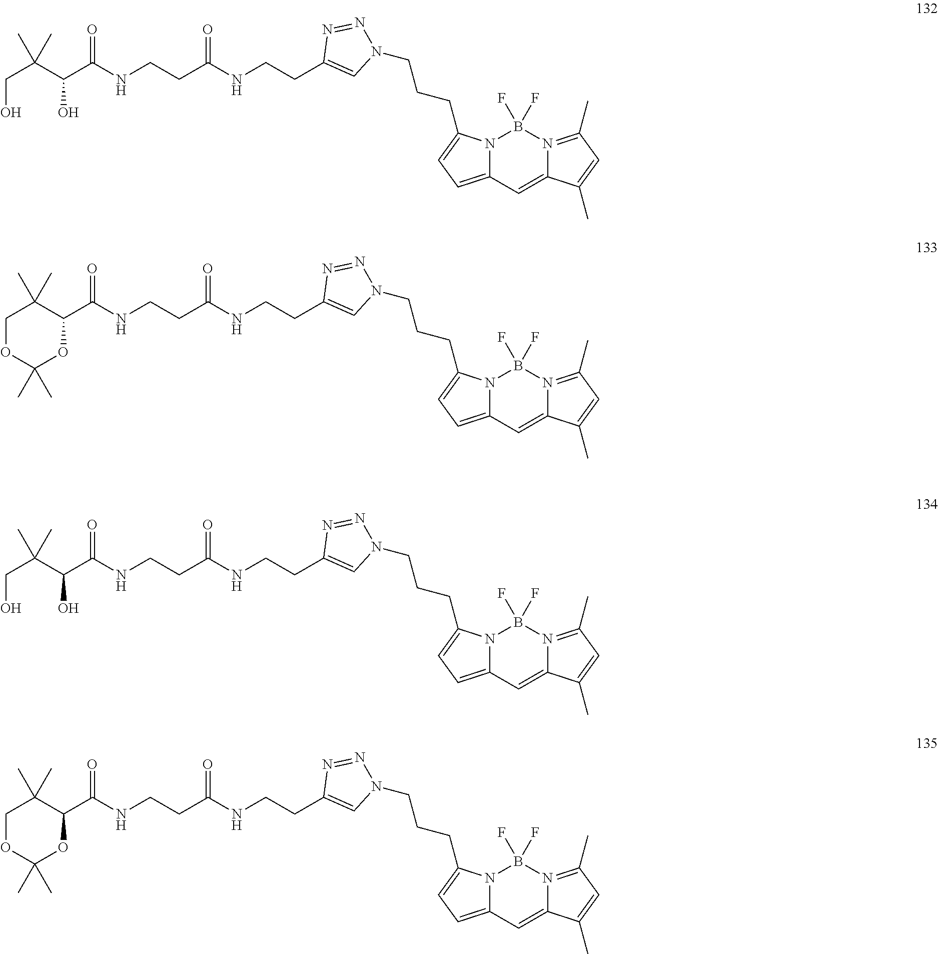

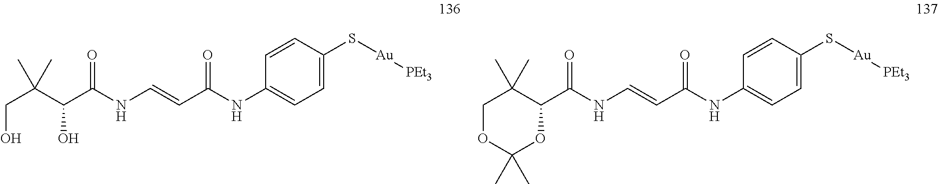

and salts, solvates, and protected forms thereof.

In even further aspects:

The compound is selected from the group consisting of:

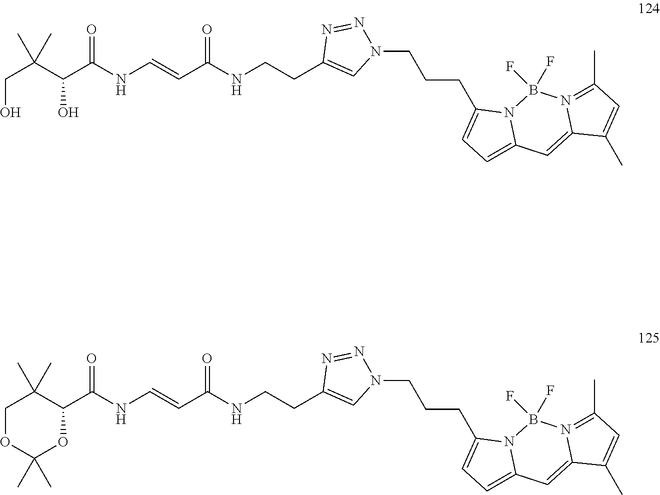

and salts, solvates, and protected forms thereof.

The compound is:

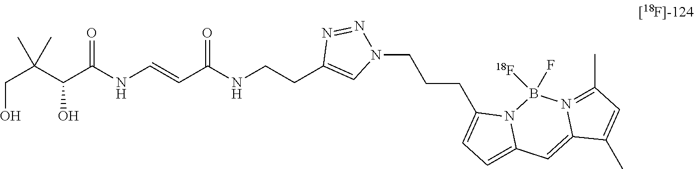

or salts, solvates, or protected forms thereof.

In one other aspect, the present disclosure provides a compound for use in detecting a malignant cell.

In particular aspects, the compound is a chemical conjugate comprising a pantothenic acid group, pantothenic acid derivative group, or pantothenic acid analogue, and at least one detection agent.

In specific aspects:

The at least one detection agent is defined in accordance with a preceding aspect.

The malignant cell is defined in accordance with a preceding aspect.

The compound is defined in accordance with a preceding aspect.

In one other aspect, the present disclosure provides a compound for use in treating a malignant cell.

In particular aspects, the compound is a chemical conjugate comprising a pantothenic acid group, a pantothenic acid derivative group, or pantothenic acid analogue, and at least one therapeutic agent.

In specific aspects:

The at least one therapeutic agent is defined in accordance with a preceding aspect.

The malignant cell is defined in accordance with a preceding aspect.

The compound is defined in accordance with a preceding aspect.

In one further aspect, the present disclosure provides a compound for use in modifying a malignant cell.

In particular aspects, the compound is a chemical conjugate comprising a pantothenic acid group, a pantothenic acid derivative group, or pantothenic acid analogue, and at least one cell modifying agent.

In specific aspects:

The at least one cell modifying agent is defined in accordance with a preceding aspect.

The malignant cell is defined in accordance with a preceding aspect.

The compound is defined in accordance with a preceding aspect.

In one other aspect, the present disclosure provides a method of treating a malignant cell comprising contacting the malignant cell with a compound, the compound being a chemical conjugate comprising a pantothenic acid group, a pantothenic acid derivative group, or a pantothenic acid analogue, and at least one therapeutic agent, thereby treating the malignant cell.

In specific aspects:

The at least one therapeutic agent is defined in accordance with a preceding aspect.

The malignant cell is defined in accordance with a preceding aspect.

The compound is defined in accordance with a preceding aspect.

In one other aspect, the present disclosure provides a method of preventing an activity a malignant cell comprising contacting the malignant cell with a compound, the compound being a chemical conjugate comprising a pantothenic acid group, a pantothenic acid derivative group, or a pantothenic acid analogue, and at least one therapeutic agent, thereby preventing the activity of the malignant cell.

In specific aspects:

The at least one therapeutic agent is defined in accordance with a preceding aspect.

The malignant cell is defined in accordance with a preceding aspect.

The compound is defined in accordance with a preceding aspect.

In one further aspect, the present disclosure provides a method of modifying a malignant cell comprising contacting the malignant cell with a compound, the compound being a chemical conjugate comprising a pantothenic acid group, a pantothenic acid derivative group, or a pantothenic acid analogue, and at least one cell modifying agent, thereby modifying the malignant cell.

In specific aspects:

The at least one cell modifying agent is defined in accordance with a preceding aspect.

The malignant cell is defined in accordance with a preceding aspect.

The compound is defined in accordance with a preceding aspect.

In one other aspect, the present disclosure provides a method of treating a malignant cell or preventing an activity a malignant cell, comprising administering a compound to a subject, the compound being a chemical conjugate comprising a pantothenic acid group, a pantothenic acid derivative group, or a pantothenic acid analogue, and at least one therapeutic agent, thereby treating the malignant cell or preventing the activity of the malignant cell.

In specific aspects:

The at least one therapeutic agent is defined in accordance with a preceding aspect.

The malignant cell is defined in accordance with a preceding aspect.

The administering is by local or systemic means.

The administering is by topical administration or injection.

The compound is defined in accordance with a preceding aspect.

In one other aspect, the present disclosure provides a kit for use in detecting a malignant cell, the kit comprising a compound, the compound being a chemical conjugate comprising a pantothenic acid group, a pantothenic acid derivative group, or a pantothenic acid analogue, and at least one detection agent.

In specific aspects:

The at least one detection agent is defined in accordance with a preceding aspect.

The malignant cell is defined in accordance with a preceding aspect.

The compound is defined in accordance with a preceding aspect.

In one other aspect, the present disclosure provides a kit for use in treating a malignant cell, the kit comprising a compound, the compound being a chemical conjugate comprising a pantothenic acid group, a pantothenic acid derivative group, or a pantothenic acid analogue and at least one therapeutic agent.

In specific aspects:

The at least one therapeutic agent is defined in accordance with a preceding aspect.

The malignant cell is defined in accordance with a preceding aspect.

The compound is defined in accordance with a preceding aspect.

In one other aspect, the present disclosure provides a kit for use in preventing an activity of a malignant cell, the kit comprising a compound, the compound being a chemical conjugate comprising a pantothenic acid group, a pantothenic acid derivative group, or a pantothenic acid analogue, and at least one therapeutic agent.

In specific aspects:

The at least one therapeutic agent is defined in accordance with a preceding aspect.

The malignant cell is defined in accordance with a preceding aspect.

The compound is defined in accordance with a preceding aspect.

In one further aspect, the present disclosure provides a kit for use in modifying a malignant cell, the kit comprising a compound, the compound being a chemical conjugate comprising a pantothenic acid group, a pantothenic acid derivative group, or a pantothenic acid analogue, and at least one cell modifying agent.

In specific aspects:

The at least one cell modifying agent is defined in accordance with a preceding aspect.

The malignant cell is defined in accordance with a preceding aspect.

The compound is defined in accordance with a preceding aspect.

In one other aspect, the present disclosure provides use of a compound for preparing a medicament for detecting a malignant cell in a subject, the compound being a chemical conjugate comprising a pantothenic acid group, a pantothenic acid derivative group, or a pantothenic acid analogue, and at least one detection agent.

In specific aspects:

The at least one detection agent is defined in accordance with a preceding aspect.

The malignant cell is defined in accordance with a preceding aspect.

The compound is defined in accordance with a preceding aspect.

In one other aspect, the present disclosure provides use of a compound for preparing a medicament for treating a malignant cell in a subject, the compound being a chemical conjugate comprising a pantothenic acid group, a pantothenic acid derivative group, or a pantothenic acid analogue, and at least one therapeutic agent.

In specific aspects:

The at least one therapeutic agent is defined in accordance with a preceding aspect.

The malignant cell is defined in accordance with a preceding aspect.

The compound is defined in accordance with a preceding aspect.

In one other aspect, the present disclosure provides use of a compound for preparing a medicament for preventing an activity of a malignant cell in a subject, the compound being a chemical conjugate comprising a pantothenic acid group, a pantothenic acid derivative group, or a pantothenic acid analogue, and at least one therapeutic agent.

In specific aspects:

The at least one therapeutic agent is defined in accordance with a preceding aspect.

The malignant cell is defined in accordance with a preceding aspect.

The compound is defined in accordance with a preceding aspect.

In one further aspect, the present disclosure provides use of a compound for preparing a medicament for modifying a malignant cell in a subject, the compound being a chemical conjugate comprising a pantothenic acid group, a pantothenic acid derivative group, or a pantothenic acid analogue, and at least one cell modifying agent.

In specific aspects:

The at least one cell modifying agent is defined in accordance with a preceding aspect.

The malignant cell is defined in accordance with a preceding aspect.

The compound is defined in accordance with a preceding aspect.

In one other aspect, the present disclosure provides a method of detection comprising: (i) contacting a plurality of cells with a compound, the compound being a chemical conjugate comprising a pantothenic acid group, a pantothenic acid derivative group, or a pantothenic acid analogue, and at least one detection agent, and (ii) determining targeting of the compound to at least one of the plurality of cells or an absence of such targeting, wherein the targeting indicates presence of at least one malignant cell in the plurality of cells, while the absence of targeting indicates absence of at least one malignant cell in the plurality of cells.

In specific aspects:

The at least one detection agent is defined in accordance with a preceding aspect.

The malignant cell is defined in accordance with a preceding aspect.

The method is performed ex vivo.

The method is performed in vivo.

The determining includes measuring levels of the compound in the malignant cell.

The determining includes localisation of the compound to the malignant cell.

The level of the compound is compared to one or more standards.

The localisation of the compound is compared to one or more standards.

The targeting is determined in accordance with a preceding aspect.

The compound is defined in accordance with a preceding aspect.

In one other aspect, the present disclosure provides a method of detection comprising: (i) administering a compound to a subject, the compound being a chemical conjugate comprising a pantothenic acid group, a pantothenic acid derivative group, or a pantothenic acid analogue, and at least one detection agent, and (ii) determining targeting of the compound to at least one cell in the subject or an absence of such targeting, wherein the targeting indicates presence of at least one malignant cell in the subject, while the absence of targeting indicates absence of at least one malignant cell in the subject.

The at least one detection agent is defined in accordance with a preceding aspect.

The malignant cell is defined in accordance with a preceding aspect.

The determining includes measuring levels of the compound in the subject.

The determining includes localisation of the compound in the subject.

The level of the compound is compared to one or more standards.

The localisation of the compound is compared to one or more standards.

The targeting is determined in accordance with a preceding aspect.

The compound is defined in accordance with a preceding aspect.

In one other aspect, the present disclosure provides a method of detection comprising: (i) contacting a biological sample with a compound, the compound being a chemical conjugate comprising a pantothenic acid group or pantothenic acid derivative group and at least one detection agent, and (ii) determining targeting of the compound to at least one cell in the biological sample or an absence of such targeting, wherein the targeting indicates presence of at least one malignant cell in the subject, while the absence of targeting indicates absence of at least one malignant cell in the subject.

The at least one detection agent is defined in accordance with a preceding aspect.

The malignant cell is defined in accordance with a preceding aspect.

The determining includes measuring a level of the compound in the biological sample.

The determining includes localisation of the compound in the biological sample.

The level of the compound is compared to one or more standards.

The localisation of the compound is compared to one or more standards.

The targeting is determined in accordance with a preceding aspect.

The compound is defined in accordance with a preceding aspect.

The foregoing brief summary broadly describes the features and technical advantages of certain aspects of this disclosure. Further technical advantages will be described in the detailed description and examples that follows.

Novel features that are believed to be characteristic will be better understood from the detailed description when considered in connection with any accompanying figures and examples. However, the figures and examples provided herein are intended to help illustrate what is disclosed or assist with developing an understanding what is disclosed, and are not intended to limit the scope of this disclosure.

### BRIEF DESCRIPTION OF THE DRAWINGS

FIG. 1. Conjugate compounds coupled to BODIPY FL were tested for uptake using 50 nM each conjugate and 5 minute incubation with HEM (human epidermal melanocytes) and melanoma cell line NZM11 (BRAF V600E mutant metastatic melanoma). Mean fluorescent intensities (MFI). For these tests, n=300-500, mean±SD. Results were from a single experiment done in triplicate. Cells were sorted by FACS.

FIG. 2. Conjugate compounds coupled to BODIPY FL were tested for uptake using 200 nM each conjugate and 5 minute incubation with melanoma cell line NZM11 (BRAF V600E mutant metastatic melanoma), melanoma cell line NZM40 (NRAS Q16H mutant metastatic melanoma) and melanoma cell line NZM9 (BRAF and NRAS wildtype metastatic melanoma). Mean fluorescent intensities (MFI). 2,000 cells (NZM11), 1,000 cells (NZM40) and 500-1,000 cells (NZM9). Cells were sorted by FACS. Bars represent mean±SD. Results were from a single experiment done in triplicate.

FIG. 3A. Conjugate compounds coupled to BODIPY FL were tested for uptake using 135 nM each conjugate and 4.5 minute incubation with human lymphocytes, monocytes and neutrophils (from a healthy donor). Mean fluorescent intensities (MFI). 1,000 cells (lymphocytes), 500 cells (monocytes) and 250 cells (neutrophils) sorted by FACS. Bars represent mean±SD. Results represent a single experiment done in duplicate.

FIG. 3B: Conjugate compounds coupled to BODIPY FL were tested for uptake using 200 nM each conjugate and 5 minute incubation with immortalized T lymphocyte cell line, Jurkat cells. Mean fluorescent intensities (MFI). 1,000 cells sorted by FACS. Bars represent mean±SD. Results represent a single experiment done in duplicate.

FIG. 4A: Conjugate compounds coupled to BODIPY FL were tested for uptake using 200 nM each conjugate and 5 minute incubation with colorectal cancer cell line HT29. Mean fluorescent intensities (MFI). 1,000 cells were sorted by FACS. These results represent a single experiment done in duplicate.

FIG. 4B: Conjugate compounds coupled to BODIPY FL were tested for uptake using 200 nM each conjugate and 5 minute incubation with mouse embryonic fibroblasts MEF. Mean fluorescent intensities (MFI). 1,000 cells were sorted by FACS. These results represent a single experiment done in duplicate.

FIG. 5A: Time course of uptake for 124, 125, 131, 132 conjugate compounds. Tests were performed using 200 nM each conjugate and 5, 30, or 60 minute incubation with melanoma cell line NZM11 (BRAF V600E mutant metastatic melanoma). Mean fluorescent intensities (MFI), n=300 mean±SD. The cells were sorted by FACS. These results represent a single experiment done in duplicate.

FIG. 5B: Time course of uptake for 124 and 3-azido BODIPY FL. Tests were performed using 200 nM each compound and 1 or 3 hour incubation with melanoma cell line NZM11 (BRAF V600E mutant metastatic melanoma). Mean fluorescent intensities (MFI). n=2000-3000 mean±SD. The cells were sorted by FACS. These results represent a single experiment done in duplicate.

FIG. 6: Time course of uptake for 124, 126, and 132 conjugate compounds. Tests were performed using 200 nM each conjugate and 5, 30 and 60 min incubation with Jurkat cells (immortalized T lymphocytes). Mean fluorescent intensities (MFI). 1,200 cells sorted by FACS. Bars represent mean±SD. Results represents a single experiment done in triplicate.

FIG. 7: The 124 conjugate was tested for toxicity using 1 μM final concentration and 72 hour incubation with melanoma cell line NZM40 (NRAS Q16H mutant metastatic melanoma). Cell densities were calculated according with sulforhodamine B assay (SRB assay), absorbance at 515 nm. Cells images of DMSO control and 124 were taken at 24, 48 and 72 h through brightfield using conventional microscope. Results represents a single experiment done in a single sample.

FIG. 8: Effect of 136 and 137 (auranofin conjugates) on viability of NZM11 melanoma cells. Cells were incubated with 136 and 137 at 10 μM final concentration and kept at 37 °C for 48 hours. Pantothenic acid (PA), CJ-15,801, and biotin were tested at 200 μM final concentration. The auranofin conjugates were also tested at 200 μM final concentration. Two batches of auranofin were tested at 10 μM final concentration.

FIG. 9: The viability data from FIG. 8 was normalised to the treatment plate and DMSO control and plotted as a percentage of cell growth. Individual data points represent technical replicates. Curve indicates non-linear regression slope, which allows determination of IC₅₀ values. 136 IC₅₀=1.936 μM; 137 IC₅₀=2.940 μM; auranofin IC₅₀=0.4905 μM.

FIGS. 10A-10B: Protein content of glioblastoma cells after 48 hour incubation with compound 138 or PA at 5, 25 and 100 μM final concentration. To indicates protein levels at the beginning of the test. Control indicates protein levels in cells which were incubated for 48 hours without exposure to 138 or PA.

FIG. 11: Uptake for compounds 124, 126, and 132 at 200 nM and 30 minute incubation with melanoma cell lines: NZM12 (NZM012) (BRAF V600E mutant metastatic melanoma, homozygous deletion CDKN2A and low invasive cell line), NZM77 (NZM077) (BRAF V600E mutant metastatic melanoma and missense NF1), NZM22 (NZM022) (BRAF G466A mutant metastatic melanoma, P53 241S/T mutant, NF1 non-frameshift insertion and PTEN epigenetic silencing). NZM9 (NZM009) (P53 179C/T mutant and homozygous silencing CDKN2A). NZM11 (NZM011) (BRAF V600E mutant metastatic melanoma and homozygous deletion CDKN2A), as positive control. Mean fluorescent intensities (MFI). 1,500-2,000 cells were obtained. Bars represent mean±SD. Results are from a single experiment done in triplicate. Cells were analysed by FACS.

FIG. 12: Time course of uptake for compounds 124, 126, and 132 at 200 nM after 5, 30 or 60 minute incubation with Jurkat cells. Mean fluorescent intensities (MFI). 1,000 cells were sorted by FACS. Bars represent mean±SD. Results represents a single experiment done in triplicate.

FIG. 13A: Cryoimaging photos showing the control mouse with no tumour implanted and no administration of compound 124 prior to euthanizing. The gastrointestinal tract showed autofluorescence, probably due to the presence of chow. Autofluorescence was also observed in the bladder, which was expected.

FIG. 13B: Cryoimaging photos showing the tumour bearing mouse (right flank) which was administered compound 124 at a dose of 0.7 mg/kg 1 hour prior to euthanising. A strong 124-specific fluorescence was seen in the bladder. 124-specific fluorescence was also seen in the region of the implanted tumour.

FIG. 13C: Cryoimaging photos showing the tumour bearing mouse administered compound 124 at a dose of 0.7 mg/kg 1 hour prior to euthanising. Specific 124-fluorescence was observed in the region of the tumour and in the bladder.

FIG. 13D: Cryoimaging photos showing the tumour bearing mouse administered compound 124 at a dose of 0.7 mg/kg 1 hour prior to euthanising. 124-specific fluorescence was observed in the kidney medulla. Together with the strong fluorescence observed in the bladder this indicates the renal clearance of the compound.

### DETAILED DESCRIPTION

The following description sets forth numerous exemplary configurations, parameters, and the like. It should be recognised, however, that such description is not intended as a limitation on the scope of the present disclosure, but is instead provided as a description of exemplary aspects and embodiments.

All references, including patents and patent applications, cited in this specification are hereby incorporated by reference. No admission is made that any reference constitutes prior art. Nor does discussion of any reference constitute an admission that such reference forms part of the common general knowledge in the art, in New Zealand or in any other country.

### DEFINITIONS

Throughout the specification and claims, the following terms take the meanings explicitly associated herein, unless the context clearly dictates otherwise.

The various examples, embodiments, and aspects as set out herein may be readily combined, without departing from the scope or spirit of this disclosure. Thus, the phrase “for example”, “in one example”, “in one embodiment”, or “in one aspect” is not necessarily exclusive of other examples, embodiments, or aspects that are also described. In the same way, the phrase “in a further example”, “in another example”, “in a further embodiment”, “in another embodiment”, “in a further aspect”, or “in another aspect” is not necessarily exclusive of other examples, embodiments, or aspects that are described. Similarly, when certain exemplifications are referred to, this is not exclusive of other exemplifications.

In each instance herein, in descriptions, embodiments, aspects, and examples of the present disclosure, the terms “comprising”, “including”, etc, are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

In the present description, the articles “a” and “an” are used to refer to one or to more than one (i.e., to at least one) of the grammatical object of the article. By way of example, “an element” can be taken to mean one element or more than one element.

Throughout this description, the term “about” is used to indicate that a value includes the standard deviation of error for the method being employed to determine the value, for example, levels of compounds or dosage levels, as described in detail herein. In particular, the term “about” encompasses up to a 10% deviation (positive and negative) in the stated value or range.

As used herein, an “isolated” component refers to a component that has been purified from other components. An isolated component may be: about 70% pure or greater, about 80% pure or greater, about 90% pure or greater; or, in preferred aspects, about 91%, about 92%, about 93%, about 94%, about 95%, about 96%, about 97%, about 98%, or about 99% pure or greater.

The term “biological sample” is used herein in its broadest sense to include any sample, specimen, or culture obtained from a subject or other biological source. The sample may be obtained from a combination of subjects, or a combination of biological sources. Biological samples may include any fluid, cellular matter, or extracellular matter, or any combination thereof. The biological sample may include a plurality of cells, and in particular, a plurality of malignant cells or potentially malignant cells, as described herein. A “healthy” sample, as used herein, refers to a biological sample obtained from a subject with no evidence of a condition, e.g., cancer, syndrome, or other proliferative disorder. “Healthy” and “normal” are used interchangeably herein.

As used herein, the term “detection” can be used to mean identification of the presence or absence of a malignant cell, or the presence or absence of the corresponding condition, e.g., tumour, cancer, syndrome, proliferative disorder, etc, or the localisation of a malignant cell, or the localisation of a tumour, cancer, proliferative formation, etc. “Detecting” in this instance is synonymous with “determining”, “identifying”, “testing”, “assessing”, and “establishing”. Specifically included herein is the identification of the presence or absence of a malignant cell. For example, this may include one or more malignant cells selected from melanoma cells, colorectal cancer cells, leukaemia cells, and others. Accordingly, “detected” may mean to determine the presence of at least one malignant cell and/or the localisation of at least one malignant cell. For example, this may include at least one melanoma cell, at least one colorectal cancer cell, or at least one leukaemia cell. Suitable detection methods are described herein. Cells suitable for detection are described herein.

As used herein, the term “level” refer to the amount of the compound that is being measured. The levels of such can be measured by any means available in the field. For example, the terms “measuring”, “detecting”, and “determining” in regard to compound levels, relates to any means and methods useful for assessing and quantifying the level of the compound.

The term “threshold level” refers to a detected level of a compound that is sufficient to distinguish the presence of the compound above the background, i.e., above noise. Detection may be carried out by fluorescence measurement; positron emission scanning; radioimaging; or other means.

The term “THL positive” refers to any cell (e.g., any malignant cell) which shows a measurable increase in binding and/or uptake of one of more pantothenic acid (PA) conjugates or CJ-15,801 (138) conjugates, as compared to a control cell (e.g., a normal cell). Such increase may be evident at any given concentration of conjugate over any given time period. The detection method may utilise, for example, fluorescence (e.g., FACS if used with a suitable fluorophore), scintillation, (e.g., where a suitable isotope is used), PET, SPECT, CT (e.g., where a suitable radiotracer is used). Other detection methods are described in detail herein.

The term “malignant cell” refers to cell that grows in an uncontrolled way and can invade nearby tissues or regions. Specifically included are cancer cells, such as melanoma cells, colorectal cancer cells, and leukaemia cells. A melanoma cell includes, for example, cutaneous melanoma cells, mucosal melanoma cells, ocular melanoma cells, and metastatic melanoma cells. A colorectal cancer cell includes, for example, cancer cells that develop in one or more of the colon, rectum, and other parts of the gastrointestinal tract, e.g., the stomach or duodenum, as well as metastatic colorectal cancer cells. A leukaemia cell includes, for example, myeloid leukaemia cells and lymphocytic leukaemia cells. Other examples are provided herein.

As used herein, the term “targeting” refers to the directing and localising of a compound to a cell. A cell may be targeted, for example, by attachment of the compound to the cell (e.g., specific binding) and/or uptake of the compound into the cell (e.g., specific transport). The targeting of malignant cells is described in detail herein.

The term “pharmaceutically acceptable”, as used herein, pertains to compounds, ingredients, materials, compositions, dosage forms, etc., which are, within the scope of sound medical judgment, suitable for use by the subject in question (e.g., human). Each carrier, diluent, excipient, etc. must also be “acceptable” in the sense of being compatible with the other ingredients of the formulation.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly noted are ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Salts can be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, ethanesulfonic, mucic, nitric, pamoic, pantothenic, phosphoric, succinic, sulfuric, tartaric, p-toluenesulfonic acid, and the like. Particularly noted are citric, hydrobromic, hydrochloric, maleic, phosphoric, sulfuric, fumaric, and tartaric acids.

The term “solvate” refers to an aggregate that consists of a solute ion or molecule with one or more solvent molecules. Solvates include hydrates, that is, aggregates of a compound of interest with water.

The term “chemically protected form” as used herein, refers to a compound in which one or more reactive functional groups are protected from undesirable chemical reactions, that is, are in the form of a protected or protecting group, i.e., a masked or masking group or a blocked or blocking group.

As used herein, the term “subject” refers to any animal. This includes all mammals and specifically includes humans. The terms “subject” and “patient” are used interchangeably. A “healthy subject” refers to a subject who is not inflicted with a condition, e.g., cancer, syndrome, proliferative disorder, etc.

As used herein, “simultaneous” administration means that a compound of this disclosure and an additional active compound are administered to a subject together or essentially together. This may be, for example, administration by the same route, and may be, for example, administration in a single dose.

As used herein “separate” administration means that a compound of this disclosure and an additional active compound are administered to a subject by different means. This may be, for example, administration by two different routes. As an exemplification, this may occur where one compound is administered by infusion and the other is given orally during the infusion or before or after the infusion.

As used herein “sequential” administration means that a compound of this disclosure and an additional active compound are administered at different points in time. As an exemplification, this may occur where one compound is administered by infusion and the other is given orally before or after the infusion.

“Treating” as used herein is meant as reducing the viability, replication, and/or tumour formation of a malignant cell. Treating may reduce at least one symptom caused by the presence of malignant cells in a subject. Treatment may result in the reduction in number of malignant cells in a subject, or the elimination of malignant cells in a subject. A treatment may reduce or halt the progression of a malignancy in a subject. Treatment methods for tumours, cancers, syndromes, proliferative disorders are specifically noted.

“Preventing” as used herein is meant as impeding the growth or spread of a malignant cell. Prevention of metastasis is specifically noted. Preventing may hinder onset of at least one symptom caused by the presence of malignant cells in a subject. For example, a preventative measure may halt or delay a symptom from occurring, or may reduce the severity of a symptom should such arise. It should be understood that the term “treating or preventing” does not exclude the possibility of obtaining both treatment and prevention (e.g., at the same time or at different times) in any given subject. Preventative measures for tumours, cancers, syndromes, proliferative disorders are specifically noted.

As used herein, a “linker” may couple, link, bond, attach, or otherwise associate two or more chemical entities.

An “alkyl” group refers to a monovalent hydrocarbon group. The alkyl group is fully saturated. It may be linear or branched. An alkyl group may be C₁₋₁₀ alkyl, C₁₋₆ alkyl, C₁₋₄ alkyl, C₁₋₂ alkyl or C₁ alkyl (methyl).

An “alkylene” group refers to a bivalent hydrocarbon group. The alkylene group is fully saturated. It may be linear or branched. An alkylene group may be C₁₋₁₀ alkylene, C₁₋₆ alkylene, C₁₋₄ alkylene, C₁₋₃ alkylene, C₂₋₃ alkylene, C₁ alkylene, C₂ alkylene (ethylene) or C₁ alkylene (methylene).

An “alkenyl” group refers to a monovalent hydrocarbon group having one or more carbon-carbon double bonds, such as one double bond. The alkenyl group may be fully or partially unsaturated, such as partially unsaturated. It may be linear or branched. An alkenyl group may be C₂₋₁₀ alkenyl, C₂₋₆ alkenyl, C₂₋₄ alkenyl, C₂₋₃ alkenyl, C₃ alkenyl (allyl) or C₂ alkenyl (vinyl).

An “alkenylene” group refers to a bivalent hydrocarbon group having one or more carbon-carbon double bonds, such as one double bond. The alkenyl group may be fully or partially unsaturated, such as partially unsaturated. It may be linear or branched. An alkylene group may be C₂₋₁₂ alkenylene, C₂₋₁₀ alkenylene, C₂₋₆ alkenylene, C₄₋₆ alkylene, C₂₋₄ alkenylene, C₂₋₃ alkenylene, C₂ alkenylene, or C₃ alkenylene.

An “alknyl” group refers to a monovalent hydrocarbon group having one or more carbon-carbon triple bonds, such as one triple bond. The alkynyl group may be fully or partially unsaturated, such as partially unsaturated. It may be linear or branched. An alkynyl group may be C₂₋₁₀ alkynyl, C₂₋₆ alkynyl. C₂₋₄ alkynyl, C₂₋₃ alkynyl, C₃ alkynyl (propargyl) or C₂ alkynyl.

A “cycloalkyl” group refers to a monovalent cyclic hydrocarbon group. The cycloalkyl group is fully saturated. A cycloalkyl group may have one ring, or two or more fused rings. The cycloalkyl group may be C₃₋₁₀ cycloalkyl, such as C₃₋₆ cycloalkyl, such C₄₋₆ cycloalkyl, such as C₆ cycloalkyl (cyclohexyl).

A “cycloalkylene” group refers to a bivalent cyclic hydrocarbon group. The cycloalkylene group is fully saturated. A cycloalkylene group may have one ring, or two or more fused rings. The cycloalkyl group may be C₃₋₁₀ cycloalkylene, such as C₃₋₆ cycloalkylene, such C₄₋₆ cycloalkylene, such as C₆ cycloalkylene (cyclohexylene).

A “heteroalkylene group” refers to a bivalent hydrocarbon group where one or more carbon atoms is replaced with a heteroatom. The heteroalkylene group is fully saturated. It may be linear or branched. A heteroalkylene group may be a C₂₋₁₂ heteroalkylene group, such as a C₃₋₁₂ heteroalkylene, such as C₃₋₁₂ heteroalkylene. A heteroalkylene group may be an alkylene glycol group, such as a polyalkylene glycol group. Examples include an ethylene glycol group, such as a polyethylene glycol group. The heteroalkylene group may include one or two heteroatoms, particularly, one heteroatom. The heteroatoms may be —O—, —S— and/or —NH—.

An “aryl” group refers to a monovalent aromatic group. The aryl group may be a carboaryl group or a heteroaryl group. A carboaryl group may be C₆₋₁₄ carboaryl, C₆₋₁₀ carboaryl, such as C₆ carboaryl (phenyl) or C₁₀ carboaryl (naphthyl). A heteroaryl group may be C₅₋₁₀ heteroaryl, such as C₅₋₆ heteroaryl, such as C₅ heteroaryl or C₆ heteroaryl. A heteroaryl group has one or more aromatic ring atoms which may be N, S and/or O. Examples of C₅ heteroaryl groups include pyrrolyl and oxazolyl. Examples of C₆ heteroaryl groups include pyridyl and pyrimidinyl. An aryl group may have one ring, or two or more fused rings. Where a heteroaryl group has two or more rings, each ring may have from 5 to 7 ring atoms, of which 0 to 4 are heteroatoms (with the proviso that at least one ring has one heteroatom).

An “arylene” group refers to a bivalent aromatic group. The arylene group may be a carboarylene group or a heteroarylene group. A carboarylene group may be C₆₋₁₄ carboarylene, C₆₋₁₀ carboarylene, such as C₆ carboarylene (phenylene) or C₁₀ carboarylene (naphthylene). A heteroarylene group may be C₅₋₁₀ heteroarylene, such as C₅₋₆ heteroarylene, such as C₅ heteroaryl or CG heteroarylene. A heteroaryl group has one or more aromatic ring atoms selected from N, S and O. Examples of C₅ heteroarylene groups include triazolylene, pyrrolylene and oxazolylene. Examples of C₆ heteroarylene groups include pyridylene and pyrimidylene. An arylene group may have one ring, or two or more fused rings. Where a heteroarylene group has two or more rings, each ring may have from 5 to 7 ring atoms, of which 0 to 4 are heteroatoms (with the proviso that at least one ring has one heteroatom).

A “heterocyclene” group refers to a bivalent heterocycle. The heterocyclene group is fully saturated. A heterocyclene may be C₅₋₁₂ heterocyclene, such as C₅₋₇ heterocyclene, such as C5-6 heterocyclene, such as CG heterocyclene. A heterocyclene may have one or two fused rings. Where two rings are present, one or both rings may have a heteroatom. The heterocyclene may have one or more ring heteroatoms selected from O, S and N (such as NH). The sulfur atom may be oxides, such as SO and SO₂. A carbon ring atom in a heterocyclene group may have an oxo substituent (═O). The oxo substituent may be provided on a carbon ring atom having a neighbouring nitrogen ring atom, thereby to provide an amido-like group in the heterocycle. Where the heterocyclene has a nitrogen ring atom, the heterocyclene may be connected via the nitrogen ring atom.

An “aralkyl” group refers to an alkyl group having one or more, such as one, aryl substituents. The aralkyl is connected via the alkyl group. An example of an aralkyl group is benzyl. The alkyl and aryl groups may each be as defined herein.

A “cycloalkylalkyl” group refers to an alkyl group having a cycloalkyl substituent. The cycloalkylalkyl group is connected via the alkyl group. The alkyl and cycloalkyl groups may each be as defined herein.

An “alkanoyl” group refers to an alkyl group where the carbon of the alkyl group that forms the connection is substituted with oxo (═O). An example of an alkanoyl group is acyl (C₂ alkanoyl). The alkanoyl group may be based on an alkyl group as described herein.

An “aralkanoyl” group refers to an aralkyl group where the carbon of the alkyl group that forms the connection is substituted with oxo (═O). An example of an aralkanoyl group is benzoyl. The aralkanoyl group may be based on an aralkyl group as described herein.

An “alkoxy” group refers to an alkyl ether, which is connected via the ether oxygen atom. The alkyl group is as defined herein.

An “alkenoxy” group refers to an alkenyl ether, which is connected via the ether oxygen atom. The alkenyl group is as defined herein. In one aspect, the ether oxygen is not provided at a carbon atom that also participates in carbon-carbon double bond.

An “alkynoxy” group refers to an alkynyl ether, which is connected via the ether oxygen atom. The alkynyl group is as defined herein. In one aspect, the ether oxygen is not provided at a carbon atom that also participates in carbon-carbon triple bond.

An “aralkoxy” group refers to an aralkyl ether, which is connected via the ether oxygen atom provided on the alkyl of the aralkyl. The aralkyl group is as defined herein.

A “cycloalkylalkoxy” group refers to a cycloalkylalkyl ether, which is connected via the ether oxygen atom provided on the alkyl of the cycloalkylalkyl. The cycloalkylalkyl group is as defined herein.

### Compounds

The present inventors have found that pantothenic acid groups and pantothenic acid derivative groups may be conjugated to chemical agents to provide delivery of such agent(s) to malignant cells. The delivery of these compounds to malignant cells has been shown to be selective and specific. The findings provided herein are entirely unexpected, and provide many advantageous applications.

Thus, in specific aspects, the disclosure provides compounds which are chemical conjugates comprising a pantothenic acid group, a pantothenic acid derivative group, or a pantothenic acid analogue, that is covalently linked to a chemical agent, either directly or via a linker group. In certain aspects, the chemical agent may be one or more of a detection agent, a therapeutic agent, or a cell modifying agent, as described herein.

In specific aspects, the pantothenic acid derivative group may be a CJ-15,801 group (termed here as a 138 group) Structurally, CJ-15,801 differs from pantothenic acid in the fact that it has a double bond in the β-alanine moiety (see below).

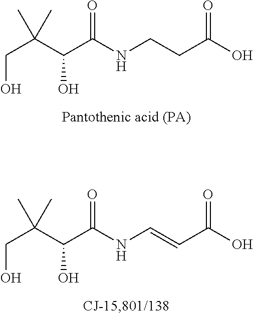

The present disclosure demonstrates that a pantothenic acid group, a pantothenic acid derivative group, or a pantothenic acid analogue, for example, a 138 group or analogue thereof, can be conjugated to at least one chemical agent to allow targeting of the chemical agent(s) to a malignant cell. This can provide delivery of the chemical agent(s) into cancer cells, and thereby allow identification, treatment and/or modification of the cancer cells.

In one aspect, this disclosure provides a compound comprising a pantothenic acid group, a pantothenic acid derivative group, or a pantothenic acid analogue, for example, a 138 group or analogue thereof, conjugated together with at least one agent.

In one particular aspect, the compound is of formula (I):

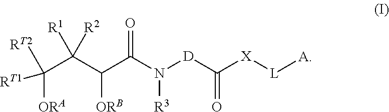

Included are salts, solvates, and protected forms thereof.

In particular aspects, the substituents —R^{A} and —R^{B} for formula (I) may be provided as follows.

The groups —R^{A} and —R^{B} may be each independently selected from hydrogen, alkyl, alkenyl, alkynyl, aralkyl, cycloalkylalkyl and alkanoyl, or —R^{A} and —R^{B} may be together as —C(R^{C1})(R^{C2}), forming a 6-membered ring.

Where -R^{A} and —RB are together a group —C(R^{C1})(R^{C2}), this six-membered ring may be formed together with the oxy gen to which each of —R^{A} and —R^{B} are attached, and the carbon atoms that are a and b to those oxygen atoms, within the pantoyl moiety:

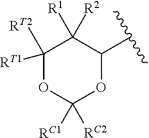

The six-membered ring may be a 1,3-dioxane group. The compound may be referred to as an acetal, for example where —R^{C1} and —R^{C2} are alkyl or hydrogen. In another example, —R^{C1} and —R^{C2} may together form an oxo (═O) group. Here, the compound may be referred to a cyclic carbonate.

The —R^{A} and —R^{B} may be each independently selected from hydrogen and alkyl, or —R^{A} and —R^{B} may be together as —C(R^{C1})(R^{C2}), forming a 6-membered ring. As one example, —R^{A} and —R^{B} may be each independently selected from hydrogen and alkyl, such as hydrogen.

A compound where —R^{A} and —R^{B} are both hydrogen may be formed from a compound where —R^{A} and —R^{B} are together —C(R^{C1})(R^{C2})—. Similarly, compounds where —R^{A} and —R^{B} are not both hydrogen may be formed from compounds where —R^{A} and —R^{B} are both hydrogen.

An alkanoyl group may be a C₁₋₆ alkanoyl group, such as C₁₋₄, such as C₂ alkanoyl (an acyl group). A compound having an alkanoyl group may be formed by reaction of the alcohol with an appropriate acid chloride or anhydride, for example.

Typically, both —R^{A} and —R^{B} may be hydrogen or —R^{A} and —R^{B} may be together C(R^{C1})(R^{C2})—, such as —C(Me)₂-. In one aspect, —R^{A} is hydrogen. In one aspect. —R^{B} is hydrogen.

In one aspect, the substituents for formula (I) may be provided as follows.
—R^{A} and —R^{B} may each independently be selected from hydrogen, alkyl, alkenyl, alkynyl, aralkyl, cycloalkylalkyl, alkanoyl and aralkanoyl;
or —R^{A} and —R^{B} may be together —C(R^{C1})(R^{C2})—, forming a 6-membered ring, where —R^{C1} is independently selected from hydrogen, alkyl, alkenyl, alkynyl, aralkyl and cycloalkylalkyl, and —R^{C2} is independently selected from hydrogen, alkyl, alkenyl, alkynyl, aralkyl, cycloalkylalkyl, alkoxy, alkenoxy, alkynoxy, aralkoxy and cycloalkylalkoxy, or —R^{C1} and —R^{C2} are together oxo (═O);
—R^{T1} and —R^{T2} may each independently be hydrogen or alkyl;
—R¹ and —R² may each independently be selected from hydrogen, alkyl, alkenyl, alkynyl, aralkyl and cycloalkylalkyl;
R³ may be hydrogen or alkyl;
-D- may be C₂₋₄ alkenylene or C₁₋₄ alkylene, where the alkenylene or alkylene is optionally substituted with alkyl or halo;
—X— may be a covalent bond, —N(R⁴)—, —O—, —S—, or —Se—, where —R⁴ is hydrogen or alkyl;
-L- may be a linker or a covalent bond; and
A- may comprise at least one chemical agent,
and also salts, solvates, and protected forms thereof.

In certain aspects, -D- is C₂₋₄ alkenylene.

In certain aspects, -D- is not C₁₋₄ alkylene.

In one aspect, the compound of formula (I) may be of formula (Ib):

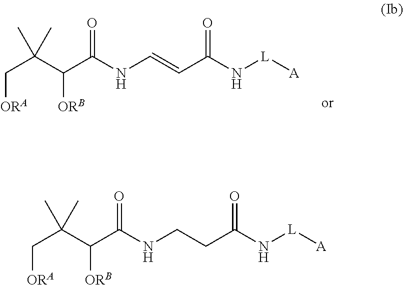

where —R^{A}, —R^{B}, -L- and -A are as defined for the compounds of formula (I), and also salts, solvates, and protected forms thereof.

In one aspect, the compound of formula (I) may be of formula (Ic):

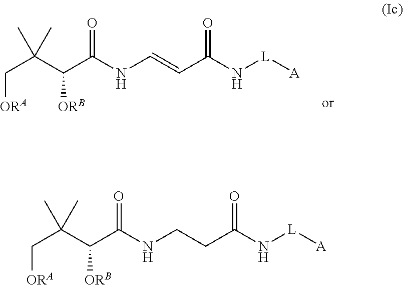

where —R^{A}, —R^{B}, -L- and -A are as defined for the compounds of formula (I), and also salts, solvates, and protected forms thereof.

In one aspect, the compound of formula (I) may be of formula (Id):

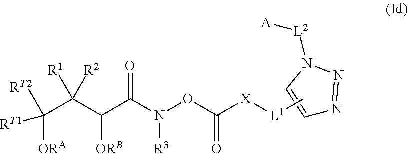

where —R^{T1}, —R^{T2}, —R^{A}, —R^{B}, —R¹, —R², —R³, -D-, and —X— are as defined for the compounds of formula (I);
-L¹- is alkylene or heteroalkylene;
-L²- is alkylene or heteroalkylene;
and also salts, solvates and protected forms thereof.

In certain aspects, the compound of formula (I) is selected from one of the following formulae:

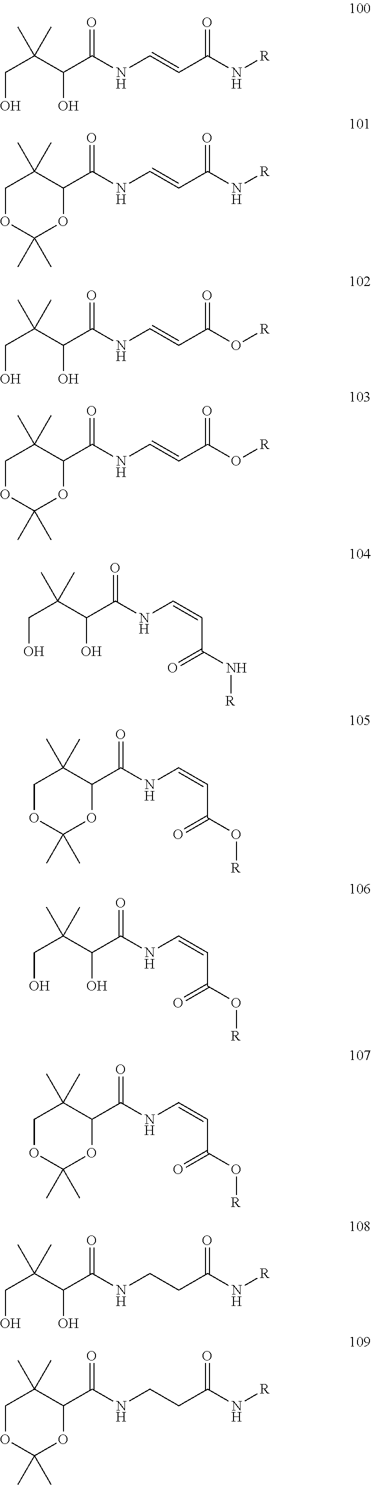

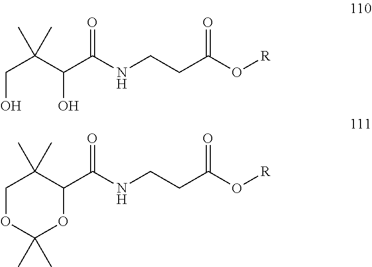

wherein —R is -L-A;
wherein -L- is a linker or a covalent bond; and -A is at least one chemical agent,
and also salts, solvates, and protected forms thereof.

In certain aspects, the compound of formula (I) is selected from one of the following formulae:

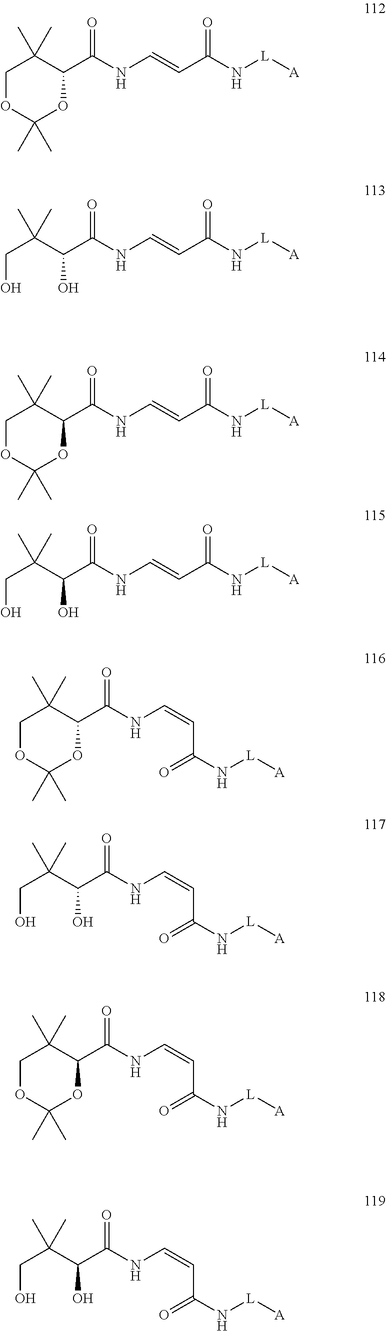

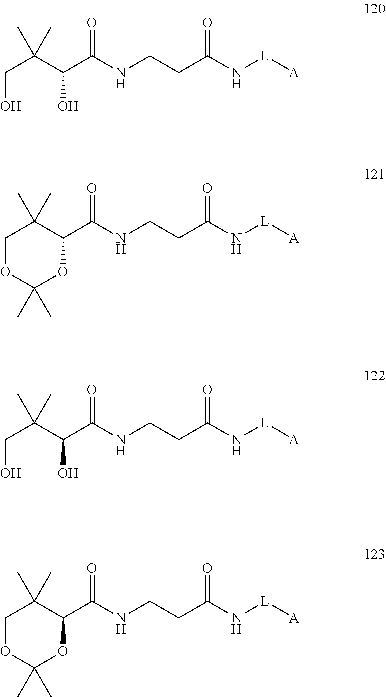

wherein -L- is a linker or a covalent bond; and -A is at least one chemical agent,
and also salts, solvates, and protected forms thereof.

In certain aspects, the group -D- may be C₁₋₄ alkylene or C₂₋₄ alkenylene, where the alkylene or alkenylene is optionally substituted with alkyl or halo, such as optionally substituted mono- or di-substituted with alkyl or halo, such as optionally substituted mono- or di-substituted with alkyl.

As exemplifications, -D- may be C₂₋₄ alkenylene where the alkenylene is optionally substituted with alkyl or halo. D- may be C₁₋₃ alkylene or C₂₋₃ alkenylene where the alkylene or alkenylene is optionally substituted with alkyl or halo. -D- may be C₂₋₃ alkenylene where the alkenylene is optionally substituted with alkyl or halo. -D- may be C₂ alkylene or C₂ alkenylene, where the alkylene or alkenylene is optionally substituted with alkyl or halo. -D- may be C₂ alkenylene, where the alkenylene is optionally substituted with alkyl or halo. The alkylene or alkenylene may be a linear alkylene or linear alkenylene.

In certain aspects, -D- may be —C(R⁵)₂—C(R⁶)₂— or —C(R⁵)═C(R⁶)—, where each —R⁵ is hydrogen or alkyl and each —R⁶ is hydrogen or alkyl. -D- may be C₂ alkylene or C₂ alkenylene, optionally substituted with alkyl. -D- may be C₂ alkenylene, optionally substituted with alkyl. Where -D- is C₄ alkenylene, the alkenylene may be a diene. In particular aspects, -D- may be a C₂₋₃ alkenylene; or -D- may be a C2 alkenylene.

In certain aspects, a double bond is present in the conjugate of formula (I). The double bond may have a trans or cis arrangement. As an exemplification, the double bond may have a trans arrangement. Here, trans refers to the arrangement of the amido groups across the double bond. For example, the compound of formula (Ia-1) has a trans arrangement:

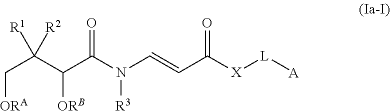

As an exemplification, the double bond may have a cis arrangement. Here, cis refers to the arrangement of the amido groups across the double bond. For example, the compound of formula (Ia-11) has a cis arrangement:

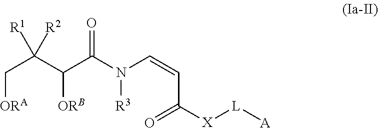

The geometry of the double bond may influence the selectivity of conjugate to deliver the agent into a particular cell or a particular tissue. Where the group is a diene, the double bonds may both have trans or cis geometry, or one may be trans and the other may be cis.

Where the pantothenic acid group has a double bond, one group —R may be present. Where the pantothenic acid group does not have a double bond, two groups —R⁵ may be present. Here, the groups —R⁵ may be the same or different. For example, each —R⁵ may be hydrogen. Each —R⁵ may be alkyl such as —R⁵ methyl.

In certain aspects, the substituent -L- for each of the formulae as set out herein may be a linker as defined for the compounds of formula (Id), above. For example, -L¹- may be a C₂ alkylene, and -L²- may a C3 alkylene or a C5 alkylene.

In certain aspects, the compound of this disclosure may contain a triazole group, particularly a 1,2,3-triazole, which may be present in the linker -L-, or in a precursor group to the linker -L-. Where the 1,2,3-triazole is present, it is 1,4- or 1,5-substituted. In one aspect, the 1,2,3-triazole is 1,4-substituted.

As exemplifications, the linker may include one or more bivalent linkers; multivalent linkers; cleavable linkers; enzyme cleavable linkers; protease cleavable linkers; and esterase cleavable linkers. For example, multivalent linkers such as bivalent linkers may be used to conjugate the pantothenic acid group or pantothenic acid derivative group to two or more chemical agents. This may be used to obtain various conjugates, for example, for conjugation of two or more diagnostic agents, two or more therapeutic agents, and/or two or more cell modifying agents as described herein.

In particular aspects, the linker may be selected from: a triazole linker; glycol linker; polyethylene glycol (PEG) linker, alkyl linker; heteroalkyl linker; and alkylene linker. Other suitable linkers are set out herein. Any combination of linkers may be utilised.

In one aspect, the conjugates molecules of this disclosure may possess the stereochemical configuration of pantothenic acid. This is the (2R)-configuration. Thus, a compound of this disclosure may have (2R)-stereochemistry. In particular, a compound of formula (I) may be:

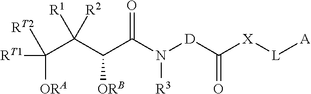

As an alternative, a compound this disclosure may have (2S)-stereochemistry. In particular, a compound of formula (I) may be:

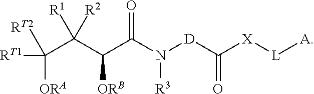

In certain aspects the compound of formula (I) is selected from the group consisting of:

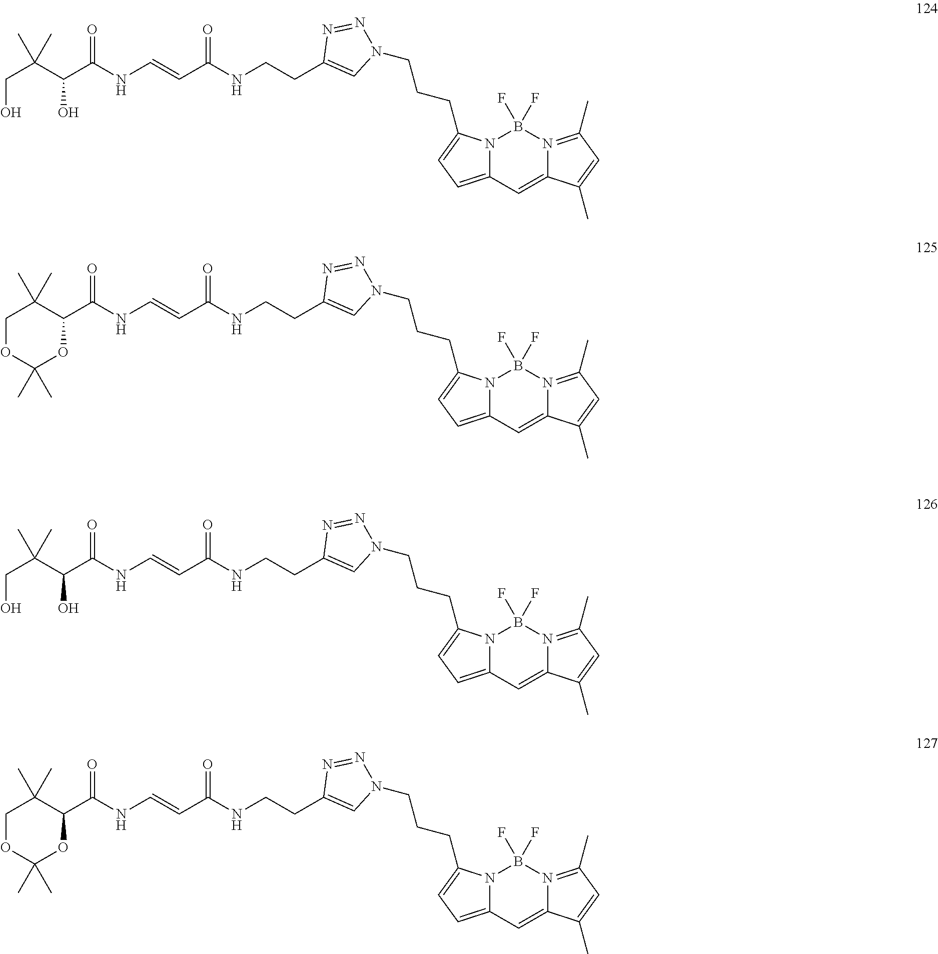

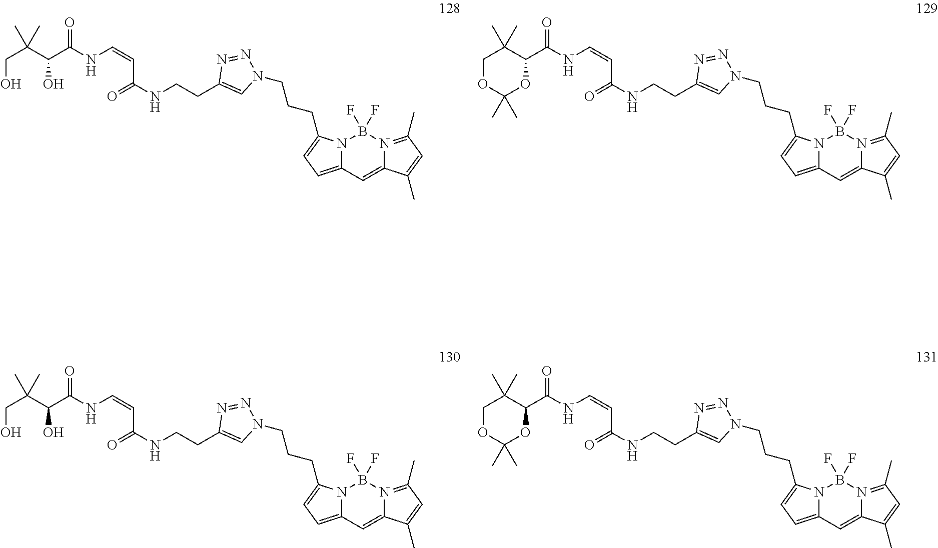

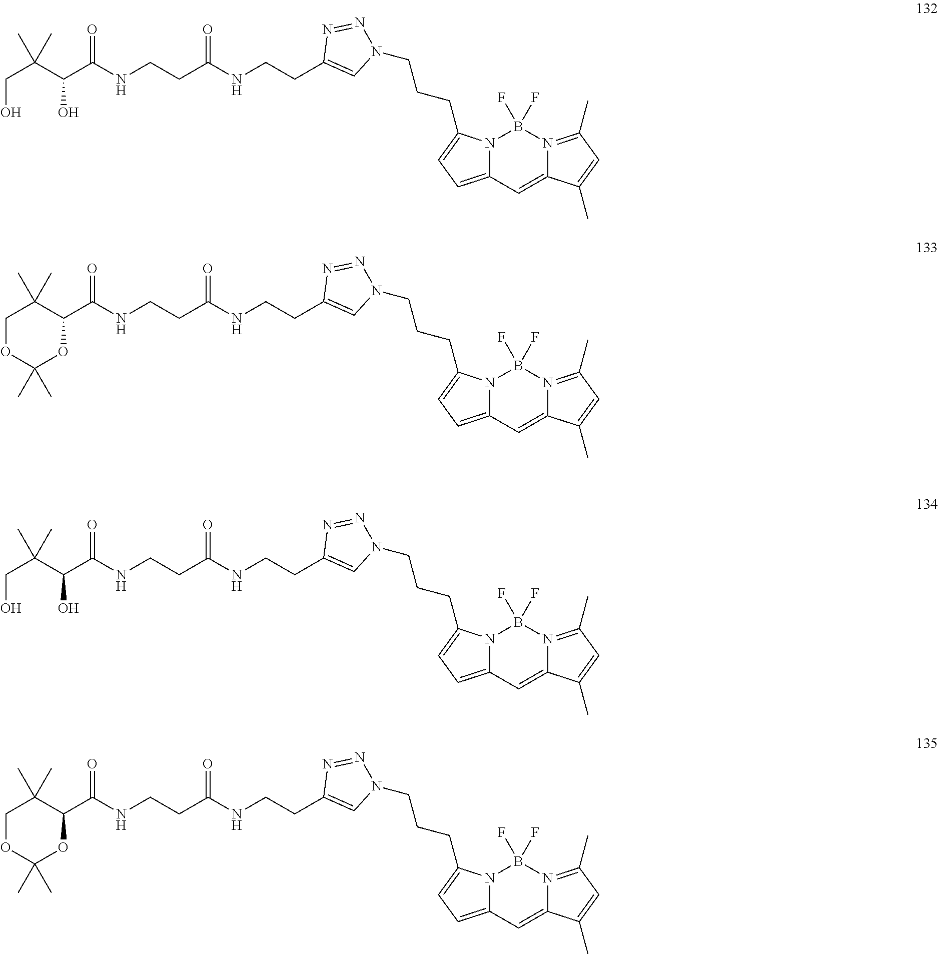

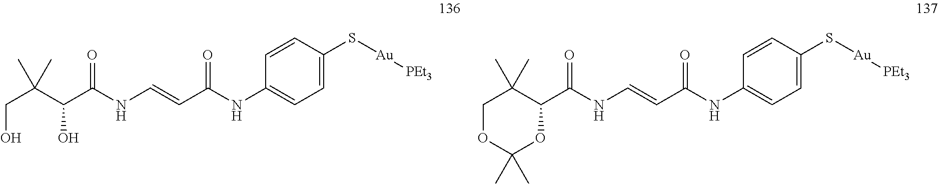

and salts solvates, and protected forms thereof.

In certain aspects, the compound of formula (I) is selected from the group consisting of:

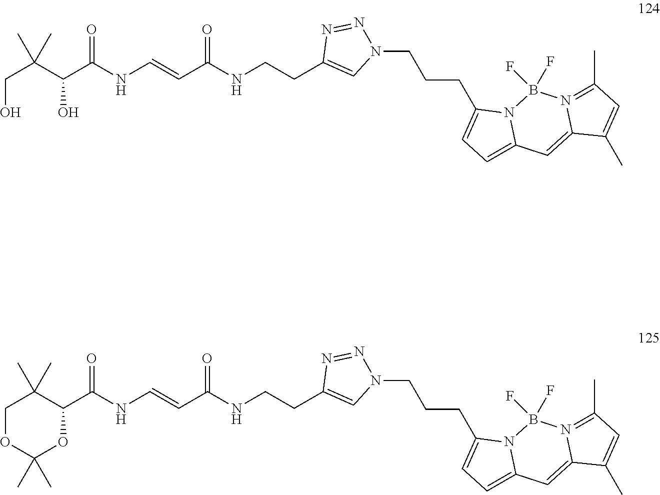

and salts, solvates, and protected forms thereof.

In particular aspects, the compound is:

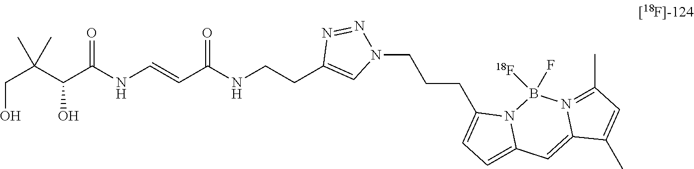

or salts, solvates, or protected forms thereof.

In this disclosure, the compound 124 corresponds to compound 5 from GB 1820626.8 and PCT/EP2019/086120, while the compound 125 corresponds to compound 1 from GB 1820626.8 and PCT/EP2019/086120, these being hereby incorporated by reference in their entirety.

It will be understood that the 124 compound comprises a 138 moiety covalently bonded via a triazole linker to a fluorescent BODIPY dye, and may also be referred to as (R,E)-N-((((3-[4,4-difluoro-5,7-dimethyl-4-bora-3a,4a-diaza-s-indacene-3-yl]propyl)-1H-1,2,3-tri-azol-4-yl)ethylamino)-3-oxoprop-1-en)yl)-2,4-dihydroxy-3,3-dimethylbutanamide.

Similarly, the 125 compound comprises a structural analogue of the 138 moiety covalently bonded via a triazole linker to a fluorescent BODIPY dye, and may also be referred to as (R,E)-N-((((3-[4,4-difluoro-5,7-dimethyl-4-bora-3a,4a-diaza-s-indacene-3-yl]propyl)-1H-1,2,3-triazol-4-yl)eth-ylamino)-3-oxoprop-1-enyl)-2,2,5,5-tetramethyl-1,3-dioxane-4-carboxamide.

Of course, it will be recognised that the specific compounds 124 and 125 represent examples only, and are in no way intended to limit the scope of conjugates encompassed by this disclosure. General synthesis methods for the compounds of this disclosure, including 124 and 125, are set out in detail herein and also in GB 1820626.8 and PCT/EP2019/086120.

### Chemical Agents

A key finding of this disclosure is that pantothenic acid groups and pantothenic acid derivative groups may be conjugated to one or more chemical agents to provide delivery of such agent(s) to malignant cells. Thus, this disclosure provides compounds, these being chemical conjugates comprising a pantothenic acid group, a pantothenic acid derivative group, or a pantothenic acid analogue, that is covalently linked to a chemical agent, either directly or via a linker group. In certain aspects, the chemical agent may be one or more of a detection agent, a therapeutic agent, and/or a cell modifying agent, as described herein.

In certain aspects, the group -A of formula (I) may comprise a radical of the chemical agent, which may be formally (and not necessarily in practice) derived by removal of a hydrogen radical from the chemical agent. Generally, the chemical agent will have a functional group for forming a covalent connection with the linker. Thus, the chemical agent may have one or more groups selected from —OH, —SH, —NH₂, —NHR^{a}, —COOH, —COH, —COOR^{c}, —NS, —C═CH₂, —C° CH and maleimidyl. For example, thiol-containing (—SH) chemical agents, such as cysteine-containing polypeptides, may form a connection to a maleimide group on a linker precursor, such as a compound (III) or (IV). The chemical agent may be modified to incorporate a particular functional group for forming a covalent connection to the pantothenic acid group or the pantothenic acid derivative group.

The chemical agent is not particularly limited, and may be any agent(s) whose presence at a particular location is considered desirable, for example, for the purposes of therapy, detection, and/or cell modification. In various aspect, the at least one chemical agent for conjugation may be selected from various detection agents, therapeutic agents, a cell modifying agents, and any combination of these. The chemical agent may be a biologically active agent, such as an agent for use in therapy or cell modification, or a visualisation agent, such as an agent for use in detection. The chemical agent may be biologically active even when not attached to the structure shown below:

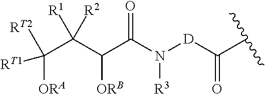

where —R^{A}, —R^{B}, —R^{T1}, —R^{T2}, —R¹—R², —R³, and -D- are as defined above (that is, a chemical agent not in conjugation with a pantothenic acid group). The chemical agent may be a small organic molecule (e.g., small drug).

As exemplifications, the chemical agent may have a molecular weight of 1000 Da or less, 900 Da or less, 800 Da or less. 700 Da or less, 600 Da or less, 500 Da or less, 400 Da or less, 300 Da or less, 200 Da or less, or 100 Da or less; or the chemical agent may have a molecular weight of 100 Da or more, 125 Da or more, 150 Da or more, 175 Da or more, 200 Da or more, 250 Da or more, 300 Da or more, 350 Da or more, 400 Da or more, 450 Da or more; or the chemical agent may have a molecular weight of about 100 to about 1000 Da, or about 100 to about 800 Da, or about 100 to about 500 Da, about 125 to about 1000 Da, or about 125 to about 800 Da, or about 125 to about 500 Da, or about 150 to about 1000 Da, or about 150 to about 800 Da, or about 150 to about 500 Da, or about 175 to about 1000 Da, or about 175 to about 800 Da, or about 175 to about 500 Da.

In certain aspects, at least one detection agent may be conjugated to a compound of this disclosure. As detection agents, various dyes may be utilised, for example, fluorophores, fluoresceins, fluorescent proteins, coumarins, cyanines, phenoxazines, and rhodamines. Fluorescent dyes may include, for example, Oregon Green (e.g., Oregon Green 488, Oregon Green 514, etc), AlexaFluor (e.g., AlexaFluor 488, AlexaFluor 647, etc), fluorescein and related analogues, BODIPY fluorescent agents (e.g., BODIPY FI, BODIPY 505, etc), rhodamine fluorescent agents (e.g., tetramethylrhodamine, etc), DyLight fluorescent agents (e.g., DyLight 680, DyLight 800, etc); cyan dyes (e.g., Indocyanine Green, Heptamethine IR-780, Heptamethine IR-808, IRDye® 800CW, DY-675, DY-676, DY-677, DY-678, Cy5, Cy7, etc). Also useful are organic fluorophores.

Specifically noted herein is dipyrrometheneboron difluoride 1 (4,4-difluoro-4-boron-3a,4a-diaza-s-indacene), i.e., BODIPY, as well as various BODIPY derivatives. The incorporation of specific substituents at different positions of the BODIPY structure makes derivatives to be readily obtained with desired chemical and photophysical properties. Various BODIPY derivatives span most of the visible and NIR wavelength range (see, for example, below).

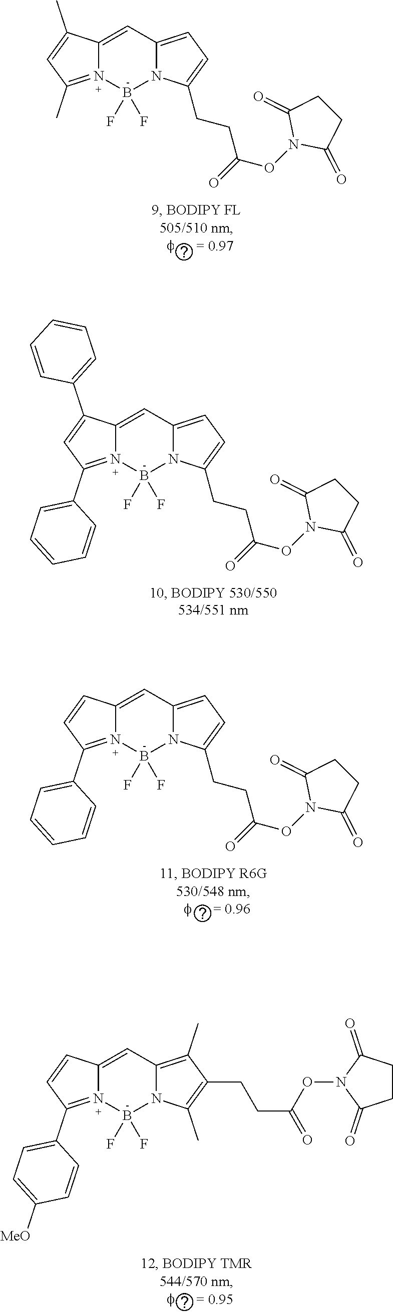

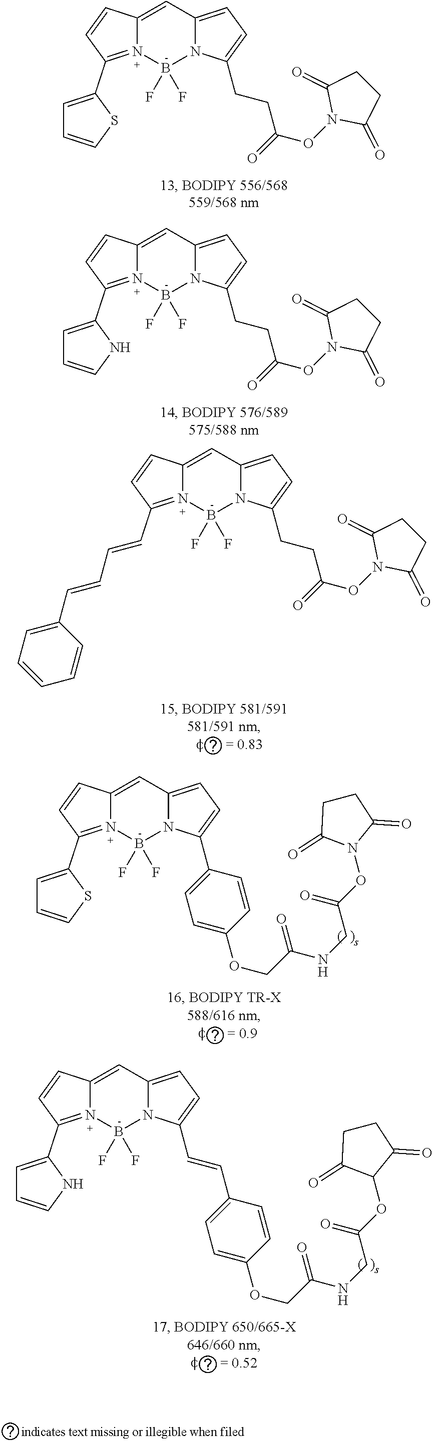

Most fluorophores derived from BODIPY produce stable fluorescent labels. Moreover, fluorescent BODIPY derivatives have been obtained with optical properties that change upon illumination with light of a specific wavelength (phototransformation) or in the presence of certain analytes. Noted derivatives include, for example, water-soluble derivatives of BODIPY containing hydrophilic substituents, BODIPY derivatives with carbohydrate and PEG substituents, mono-styryl and distyryl-BODIPY substituted subphthalocyanines, mono-styryl and distyryl-BODIPY substituted cyclophosphazenes, distyryl-BODIPY substituted cyclophosphazenes, aza-BODIPY derivatives, and hexa-borondipyrromethene cyclotriphosphazene platforms (HBTC). See, e.g., Martynov and Pakhomov, 2021, Russ. Chem. Rev. 90 1213; Cetindere, 2020, Photophysics of BODIPY Dyes, DOI. 10.5772/intechopen.92609; Saha et al., 2021, Photophysics, Photochemical, and Substitution Reactions 10.5772/intechopen.77916.

Other detection agents include radioactive substances, for example, a radioisotope. These include, for example, ¹⁸F, ³²P, ³³P, ⁴⁵Ti, ⁴⁷Sc, ⁷⁵Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Sr, ⁸⁹Sr, ⁸⁹Zr, ⁹⁴Tc, ¹²⁴I, ¹²⁵I, ¹³¹I, ¹⁴²Pr, ¹⁴³Pr, ¹⁴⁹Pr, ¹⁴⁹Pm, ¹⁵³Sm, ¹⁵⁴⁻¹⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions may be used as detection agents. These include, for example, ions of transition and lanthanide metals (e.g., metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Other detection agents include enzymes. These include, for example, horseradish peroxidase, alkaline phosphatase, acid phosphatase, glucose oxidase, β-galactosidase, β-glucuronidase, and β-lactamase. Such enzymes may be used in combination with one or more chromogen, fluorogenic compound, or luminogenic compound to generate a detectable signal. Noted also as detection agents are chelating agents, including labelled and unlabelled chelating agents. DOTA chelator is specifically noted.

Other detection agents include contrast agents. These include, for example, contrast agents for radionuclide imaging. Specifically noted are radiopharmaceuticals labelled with positron-emitters such as ¹¹C, ¹³N, ¹⁵O, ¹⁸F, ⁸²Rb, ⁶²Cu, or ⁶⁸Ga. Also included are contrast agents for single photon emission computed tomography (SPECT). Specifically noted are radiopharmaceuticals labelled with positron emitters such as ⁹⁴mTc, ²⁰¹Tl, or ⁶⁷Ga. For positron emission tomography (PET), the substitution of one of the stable fluorine atoms [¹⁹F] in the BODIPY with a positron emitting radionuclide fluorine atom [¹⁸F], permits conjugates of this disclosure to be used as a PET contrast agents for detecting malignant cells and their location in the body. For example, a compound of this disclosure may be substituted with a positron emitting radionuclide [¹⁸F] and may be formulated into a pharmaceutical composition suitable administration to a subject (e.g., local or systemic administration).

Contrast agents for CT imaging include, for example, iodinated or brominated contrast media. Specifically noted are iothalamate, isohexyl, diatrizoate, iopamidol, ethiodol, and iopanoate. Gadolinium agents may also be utilised as CT contrast agents, for example, gadopentate agents may be used. Contrast agents for optical imaging include, for example, fluorescein, a fluorescein derivative, BODIPY, a BODIPY derivative, indocyanine green, Oregon green, a derivative of Oregon green, rhodamine green, a derivative of rhodamine green, an eosin, an erythrosin, Texas red, a derivative of Texas red, malachite green, nanogold sulfosuccinimidyl ester, cascade blue, a coumarin derivative, a naphthalene, a pyridyloxazole derivative, cascade yellow dye, and dapoxyl dye.

Contrast agents for MRI include gadolinium chelates, manganese chelates, chromium chelates, and iron particles. Specifically noted is ¹⁹F. Other MRI contrast agents include complexes of metals selected from the group consisting of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (ILL) and erbium (III). Specifically noted are gadolinium-based compounds. These include, for example, gadolinium, gadolinium pentate, and gadodiamide.

In other aspects, at least one therapeutic agent may be conjugated to a compound of this disclosure. In various aspects, the at least one therapeutic agent may be one or more cancer therapeutic selected from the group consisting of: antibodies; antimetabolites; biologic response modifiers; chain terminators; growth factor inhibitors; hormonal agents; nucleic acid intercalators; pathway inhibitors; proteasome inhibitors; enzyme inhibitors; radionuclides; and radionucleotides. For example, the one or more cancer therapeutic may be a melanoma therapeutic, a leukaemia therapeutic, or a colorectal cancer therapeutic. Any combination of therapeutic agents may be utilised.

As examples, the cancer therapeutic may be selected from: cytotoxic compounds; cytostatic compounds; alkylating compounds (e.g., dacarbazine, fotemustine, temozolomide, etc); non-selective BRAF inhibitors (e.g., sorafenib); selective BRAF inhibitors (e.g., dabrafenib, encorafenib, vemurafenib, etc); Ral GTPase inhibitors; MEK inhibitors (e.g., binimetinib, cobimetinib, pimasertib, selumetinib, trametinib, etc); ERK inhibitors; CDK inhibitors (e.g., abemaciclib, flavopiridol, palbociclib, ribociclib roscovitine, etc); PI3K/AKT inhibitors (e.g., alpelisib, buparlisib, copanlisib, pictilisib, etc); c-Kit inhibitors (e.g., dasatinib, imatinib, nilotinib, sunitinib, etc); VEGF inhibitors (e.g., axitinib, pazopanib, etc); ERRB4 inhibitors (e.g., lapatinib); glutamate release inhibitors (e.g., riluzole); target-specific inhibitors (e.g., nelfinavir). Specific exemplifications include, for example, auranofin; vemurafenib; dabrafenib; and trametinib.

In particular aspects, one or more compounds of this disclosure may be used to achieve a therapeutic effect (e.g., treatment for a malignant cell and/or preventative action for a malignant cell) without conjugation to a therapeutic agent.

In other aspects, at least one cell modifying agent may be conjugated to a compound of this disclosure. In various aspects, the at least one cell modifying agent may be selected from the group consisting of: polypeptides; peptides; enzymes; enzyme fragments; antibodies; antibody fragments (e.g., Fab fragments, scFv fragments, scAb fragments, single domain antibodies (dAbs), etc); antibody epitopes; polynucleotides; genes; and gene fragments; RNAs; and DNAs.

In some cases, more than one chemical agent may be conjugated to a compound of this disclosure. For example, two or more chemical agents may be conjugated to a compound via separate linkages, or two or more chemical agents may be conjugated to a compound via the same linkage (e.g., via bivalent or multivalent linkers). Where two or more chemical agents are to be conjugated to the compound, this may be carried out via conjugation to a substrate, such as a bead or other particle. For example, multiple chemical agents (e.g., more than one type of agent) or multiple copies of a chemical agent (e.g., more than one of same agent) may be conjugated to a bead, and this, in turn, may be conjugated to a compound of this disclosure. It will be understood that in certain circumstances, it may be beneficial to use conjugate compounds that include multiple chemical agents, for example, one or more diagnostic agents along with one or more therapeutic agents; one or more diagnostic agents with one or more cell modifying agents; one ore more therapeutic agents along with one or more cell modifying agents.

### Formulations and Other Preparations

The compounds of this disclosure, including the chemical conjugates of formula (I), may be prepared as any pharmaceutically acceptable salts. Examples include acid addition salts of strong mineral acids such as HCl and HBr salts and addition salts of strong organic acids such as a methanesulfonic acid salt. Further examples of salts include sulphates and acetates such as trifluoroacetate or trichloroacetate. Other examples are provided herein. Salts a solid form may exist in more than one crystal structure and may also be in the form of hydrates.

The compounds of this disclosure may be formulated as prodrugs. For example, amino groups may be protected with a group which can be cleaved in vivo, to liberate the biologically active compound. In particular, the prodrug may be an amine prodrug. Examples of amine prodrugs include sulphamoyl, HSO₃-FMOC, and salts thereof, as well as others. See, e.g., Bergen et al., Antimicrob Agents and Chemotherapy, 2006, 50, 1953; Schechter et al., J. Med Chem 2002, 45(19) 4264; Krise and Oiiyai in Biotechnology: Pharmaceutical Aspects, 2007, 5(2), 101-131. The compounds of this disclosure may be formulated as solvates. Examples of solvates include hydrates.

The compounds of this disclosure may be prepared such that one or more atom is replaced by a naturally occurring or non-naturally occurring isotope. In one aspect, the isotope may be a stable isotope. For example, a compound may include deuterium. As exemplifications, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

The compounds of this disclosure may be provided as one or more particular forms, for example, geometric, optical, enantiomeric, diasteriomeric, epimeric, atropic, stereoisomeric, tautomeric, conformational, or anomeric forms, including but not limited to, cis- and trans-forms; E- and Z-forms; c-, I-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; a- and b-forms; axial and equatorial forms; boat-, chair-, twist-, envelope-, and half chair-forms; and any combination thereof.

Specifically noted are isomers or isomeric forms, which include all of the above forms, except tautomeric forms. Distinguished from these isomeric forms are structural (or constitutional) isomers, i.e., isomers which differ in the connections between atoms rather than merely by the position of atoms in space. For example, a reference to a methoxy group, —OCH₃, is not to be construed as a reference to its structural isomer, a hydroxymethyl group, —CH₂OH Similarly, a reference to ortho-chlorophenyl is not to be construed as a reference to its structural isomer, meta-chlorophenyl. However, a reference to a class of structures may well include structurally isomeric forms falling within that class (e.g., C₁₋₆ alkyl includes n-propyl and iso-propyl; butyl includes n-, iso-, sec-, and tert-butyl; methoxyphenyl includes ortho-, meta-, and para-methoxyphenyl).

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are readily obtained by utilising standard methods, or by adapting the methods taught herein.

The compounds disclosed herein may be provided in protected forms. Thus, one or more functional groups within compound may be provided with a protecting group to prevent their unintended reaction, for example during synthesis or storage. For example, it may be convenient or desirable to prepare, purify, and/or handle a compound in a chemically protected form. By protecting a reactive functional group, reactions involving other unprotected reactive functional groups can be performed, without affecting the protected group; the protecting group may be removed, usually in a subsequent step, without substantially affecting the remainder of the molecule. See, e.g., Green and Wuts, Protective Groups in Organic Synthesis, 3rd Edition, John Wiley and Sons, 1999.

As an example, an amine group may be protected as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)2C6H₄C6H5, —NH-Bpoc), as a 9-fluorenylmethoxy amide (—NH—Fmoc), as a 6-nitroveratryloxy amide (—NH—Nvoc), as a 2-trimethylsilylethyloxy amide (—NH—Teoc), as a 2,2,2-trichloroethyloxy amide (—NH-Troc), as an allyloxy amide (—NH—Alloc), as a 2(-phenylsulphonyl)ethyloxy amide (—NH—Psec); or, in suitable cases, as an N-oxide (>NO·).

As a further example, a carboxylic acid group may be protected as an ester for example, as: a C₁₋₇ alkyl ester (e.g. a methyl ester; a f-butyl ester); a C₁₋₇ haloalkyl ester (e.g., a C₁₋₇ trihaloalkyl ester); a triC₁₋₇ alkylsilyl-C₁₋₇ alkyl ester; or a C₅₋₂₀ aryl-C₁₋₇ alkyl ester (e.g., a benzyl ester; a nitrobenzyl ester); or as an amide, for example, as a methyl amide. As a further example, a hydroxyl group may be protected as an ether (—OR) or an ester (—OC(═O)R), for example, as: a f-butyl ether; a benzyl, benzhydryl (diphenylmethyl), or trityl (triphenylmethyl) ether; a trimethylsilyl or f-butyldimethylsilyl ether; or an acetyl ester (—OC(═O)CH₃, —OAc).

The disclosed compounds may have 1,3-diol functionality, where each of —R^{A} and —R^{B} is hydrogen. Each of the hydroxyl groups may be independently protected as ether or ester forms. In the present disclosure, the diol may also be protected as an acetal. Thus, —R^{A} and —R^{B} may be together as —C(R^{C1})(R^{C2})— forming a 6-membered ring, where each —R^{C1} and —R^{C2} is each independently selected from hydrogen, alkyl, alkenyl, alkynyl, aralkyl and cycloalkylalkyl.

As still further examples, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

The compounds of this disclosure may be produced in a substantially purified form and/or in a form substantially free from contaminants. For example, the substantially purified form may be at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to a compound in any stereoisomeric or enantiomeric form. For example, the substantially purified form may refer to a mixture of stereoisomers, i.e., purified with respect to other compounds. The substantially purified form may refer to one stereoisomer, e.g., optically pure stereoisomer. The substantially purified form may refer to a mixture of enantiomers. The substantially purified form may refer to an equimolar mixture of enantiomers (i.e., a racemic mixture, a racemate). The substantially purified form refers to one enantiomer, e.g., optically pure enantiomer.

In certain aspects, the contaminants represent no more than 50% by weight, e.g., no more than 40% by weight, e.g., no more than 30% by weight, e.g., no more than 20% by weight. e.g., no more than 10% by weight, e.g., no more than 5% by weight, e.g., no more than 3% by weight, e.g., no more than 2% by weight, e.g., no more than 1% by weight. Unless specified, the contaminants refer to other compounds, that is, other than stereoisomers or enantiomers. In one aspect, the contaminants refer to other compounds and other stereoisomers. In one aspect, the contaminants refer to other compounds and the other enantiomer.

In specific aspects, the substantially purified form is at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

In various aspects, one or more compounds of this disclosure may be included in a pharmaceutical composition, together with a pharmaceutically acceptable excipient. This may comprise one or more carriers, which may be used together with one or more other pharmaceutically acceptable ingredients according to standard methods. Such include but are not limited to, pharmaceutically acceptable carriers, diluents, excipients, adjuvants, fillers, buffers, preservatives, antioxidants, lubricants, stabilisers, solubilisers, surfactants (e.g., wetting agents), masking agents, colouring agents, flavouring agents, and sweetening agents. Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

Thus, the present disclosure provides pharmaceutical compositions, and methods of making pharmaceutical compositions, which may be provided with one or more other pharmaceutically acceptable ingredients as are utilised in standard methods, e.g., carriers, diluents, excipients, etc. If formulated as discrete units (e.g., tablets, etc.), each unit may include a predetermined amount (dosage) of the compound(s). Administration may include bringing one or more compounds of this disclosure, or a pharmaceutical composition comprising such, into physical contact with the cell, the tissue, or the subject by any convenient route of administration, e.g., systemically, peripherally, or topically.

The pharmaceutical compositions may be prepared by any methods that are standard in the field of pharmacy. The compositions may be prepared to provide for rapid or slow release; immediate, delayed, timed, or sustained release; or a combination thereof. Compositions may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, lozenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

In certain aspects, one or more compounds of this disclosure may be administered in conjunction with an additional active compound. These include other detection compounds and/or therapeutic or prophylactic compounds. Administration may be simultaneous, separate or sequential. The methods and manner of administration can be varied based on the pharmacokinetics of the compound and/or the additional active compound. The composition may therefore include one or more additional active compound in a predetermined amount.

As exemplifications, a suitable dose of a compound of this disclosure may be in the range of about 10 pg to about 50 mg per kilogram body weight of the subject per day, or about 100 pg to about 75 mg, or about 1 mg to about 65 mg, or about 1 mg to about 55 mg, or about 1 mg to about 50 mg, or about 1 mg to about 45 mg, or about 1 mg to about 40 mg, or about 1 mg to about 35 mg, or about 1 mg to about 30 mg, or about 1 mg to about 25 mg, per kilogram body weight of the subject per day. Where the compound is a salt, a prodrug, or the like, the amount administered is calculated on the basis of the parent compound and so the actual weight to be used is increased proportionately. It will be appreciated that appropriate dosages can vary from subject to subject. The selected dosage level will depend on a variety of factors including, for example, the route of administration, the time of administration, the rate of excretion of the compound, the duration of the treatment, other drugs, compounds, and/or materials used in conjunction, the severity of the condition, and the species, sex, age, weight, condition, general health, and prior medical history of the subject. The amount of the compound and the route of administration can be determined by the clinician to achieve the desired effect.

### Detection Methods

The present disclosure provides methods for delivering compounds to a malignant cell, for example, to a tumour site. As demonstrated herein, the delivery of the disclosed compounds to malignant cells has been shown to be selective and specific. The compounds of this disclosure, which include chemical conjugates comprising at least one detection agent, can thereby provide effective detection methods. The detection agents may be selected from dyes; optical contrast agents; X-ray contrast agents; magnetic resonance imaging contrast agents; and positron emission tomography contrast agents; amongst others.

The compounds that are delivered to malignant cells can be used to identify the presence of the malignant cells, and thereby confirm the malignancy. In addition, the compounds that are delivered to malignant cells can be used to localise the malignant cells, and thereby identify at least one site for treatment, e.g., site(s) for drug delivery, radiation, and/or surgery.

The disclosed compounds can be targeted to specific locations, including primary tumour sites, secondary tumour sites, and/or metastatic sites. Therefore, the compounds of this disclosure, and pharmaceutical compositions comprising one or more of these compounds, are suitable for use in methods of detection, particularly in relation to malignant cells and malignancies.

Accordingly, in certain aspects, one or more of the compounds as described herein, or a composition comprising such, may be used in detection methods for malignant cells. The compounds and compositions of this disclosure may be utilised in detection methods for the human body, or the animal body.

In certain aspects, one or more compound of this disclosure, or a composition comprising such, may be administered to a mammalian subject, such as a human, to provide detection methods for malignant cells. The compounds and compositions of this disclosure may be administered to the human body, or the animal body.

In certain aspects, one or more compound of this disclosure, or a composition comprising such, may be manufactured as a medicament for detection methods for malignant cells. The compounds and compositions of this disclosure may be prepared as medicaments for malignancy detection for the human body, or the animal body.

In certain aspects, one or more compound of this disclosure, or a composition comprising such, may be used in in vitro detection methods. The compounds and compositions of this disclosure may be applied to malignant cells that are located outside of the human body, or the animal body.

As an example, a method for detecting the presence or absence of a malignant cell may comprise: (i) contacting a plurality of cells with one or more compounds of this disclosure, these one or more compounds comprising at least one detection agent, and (ii) determining targeting of the one or more compounds to at least one of the plurality of cells or an absence of such targeting, wherein the targeting indicates presence of at least one malignant cell in the plurality of cells, while the absence of targeting indicates absence of at least one malignant cell in the plurality of cells.

As a further example, a method for detecting the presence or absence of a malignant cell in a subject may comprise administering to the subject one or more of the compounds of this disclosure, these one or more compounds comprising at least one detection agent, and determining targeting of the one or more compounds to at least one cell in the subject or an absence of such targeting, wherein the targeting indicates presence of at least one malignant cell in the subject, while the absence of targeting indicates absence of at least one malignant cell in the subject.

As a further example, a method for detecting the presence or absence of a malignant cell in a biological sample may comprise contacting one or more of the compounds of this disclosure, these one or more compounds comprising at least one detection agent, or a composition comprising such compounds, with a biological sample obtained from a subject, and determining the targeting of the one or more compounds to at least one cell in the biological sample or an absence of such targeting, wherein the targeting indicates presence of at least one malignant cell in the biological sample, while the absence of targeting indicates absence of at least one malignant cell in the biological sample.

In further aspects, the detection method may comprise (i) administering to a subject at least one compound of this disclosure comprising at least one detection agent, (ii) allowing the at least one compound to enter into cells in the subject, (iii) applying an imaging technique to detect accumulation of the at least one compound in the cells in the subject or a lack of such accumulation, wherein the accumulation indicates presence of malignant cells in the subject, while the absence of accumulation indicates absence of malignant cells in the subject.

In further aspects, the detection method may comprise (i) contacting a biological sample with at least one compound of this disclosure comprising at least one detection agent, (ii) allowing the at least one compound to enter into cells in the biological sample, (iii) applying an imaging technique to detect accumulation of the at least one compound in the cells in the biological sample or a lack of such accumulation, wherein the accumulation indicates presence of malignant cells in the biological sample, while the absence of accumulation indicates absence of malignant cells in the biological sample.

In further aspects, the detection method may comprise administering to a subject at least one compound of this disclosure comprising at least one detection agent, determining the level of the at least one compound in the subject, and comparing this determined level to a threshold level, wherein malignant cells are present if the level of the at least one compound in the subject is above the threshold level, and malignant cells are absent if the level of the at least one compound in the subject is below the threshold level.

In further aspects, the detection method may comprise contacting to a biological sample at least one compound of this disclosure comprising at least one detection agent, determining the level of the at least one compound in the biological sample, and comparing this determined level to a threshold level, wherein malignant cells are present if the level of the at least one compound in the biological sample is above the threshold level, and malignant cells are absent if the level of the at least one compound in the biological sample is below the threshold level.

In further aspects, the detection method may comprise administering to a subject at least one compound of this disclosure comprising at least one detection agent, and visually detecting the presence or absence of the at least one compound in the subject, wherein malignant cells are present if the at least one compound is visually detected in the subject above a threshold level, and malignant cells are absent if the at least one compound is not visually detected in the subject above the threshold level.

In further aspects, the detection method may comprise contacting a biological sample with at least one compound of this disclosure comprising at least one detection agent, and visually detecting the presence or absence of the at least one compound in the biological sample, wherein malignant cells are present if the at least one compound is visually detected in the sample above a threshold level, and malignant cells are absent if the at least one compound is not visually detected in the sample above the threshold level.

In further aspects, the detection method may comprise administering to a subject at least one compound of this disclosure comprising at least one detection agent, and imaging the subject to detect the presence or absence of the at least one compound in the subject, wherein malignant cells are present if the at least one compound is detected in the imaging above a threshold level, and malignant cells are absent if the at least one compound is not detected in the imaging above the threshold level.

In further aspects, the detection method may comprise contacting a biological sample with at least one compound of this disclosure comprising at least one detection agent, and imaging the biological sample to detect the presence or absence of the at least one compound in the biological sample, wherein malignant cells are present if the at least one compound is detected in the imaging above a threshold level, and malignant cells are absent if the at least one compound is not detected in the imaging above the threshold level.

The detection methods can be carried out in vitro or in vivo. The detection method may involve determining levels of the one or more compounds in the malignant cell(s) and/or determining the localisation of the one or more compounds to the malignant cell(s). Where levels are determined, these may be compared to one or more standards, for example, expected levels in malignant cell and/or expected levels in normal cells. One or more suitable positive and/or negative controls may be utilised. In addition, a threshold level may be established to indicate the presence of malignancy. Where localisation is determined, this may be compared to one or more standards, for example, expected signal for malignant cells and/or expected signal for normal cells. One or more suitable positive and/or negative controls may be utilised. In addition, a threshold signal may be established to indicate malignancy.

As noted, the compounds of this disclosure show marked selectivity and specificity in targeting malignant cells. In certain aspects, the one or more compounds of this disclosure (e.g., compound(s) conjugated to at least one detection agent) may target a malignant cell such that the signal to noise ratio (e.g., test level to background level ratio; test localisation to background localisation ratio) is at least 2 to 1, at least 2.1 to 1, at least 2.2 to 1, at least 2.3 to 1, at least 2.4 to 1, at least 2.5 to 1, at least 2.6 to 1, at least 2.7 to 1, at least 2.8 to 1, at least 2.9 to 1, or at least 3 to 1.

In one aspect, a compound for detection methods has the structure Ie or II.

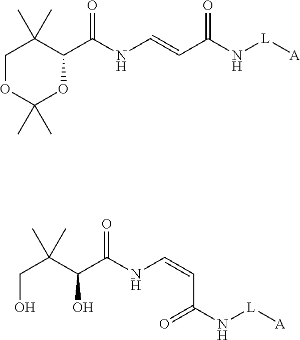

wherein -L- is a linker or a covalent bond; and -A is at least one chemical agent;
and also salts, solvates, and protected forms thereof.

In one aspect, L is a linker as defined for the compounds of formula (Id). In one aspect, -L²- is a C₃ alkylene. In a particular aspect, -L¹- is a C₂ alkylene and -L²- is a C₃ alkylene.

In one aspect, the linker -L- is a group *-L³-G-L^{A}-,
wherein the asterisk indicates the point of attachment to —X—;
-L³- is an alkylene; and
-G- is a maleimide-derived group; and
-L^{A}- is a covalent bond.

In a particular aspect, -L³- is a C₃ alkylene.

In one aspect, a compound for detection methods has the structure Ie or Ii.

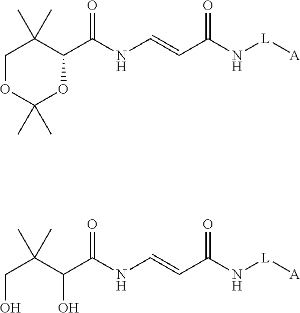

wherein -L- is a linker or a covalent bond; and -A is at least one chemical agent; and also salts, solvates, and protected forms thereof.

In one aspect, L is a linker as defined for the compounds of formula (Id). In one aspect, -L²- is a C₃ alkylene. In a particular aspect, -L- is a C₂ alkylene and -L²- is a C₃ alkylene.

In one aspect, the linker -L- is a group-L³G--L^{A}-,
wherein the asterisk indicates the point of attachment to —X—;
-L³- is an alkylene; and
-G- is a maleimide-derived group; and
-L^{A}- is a covalent bond.

In a particular aspect, -L³- is a C₅ alkylene.

In one aspect, a compound for detection methods has the structure If.

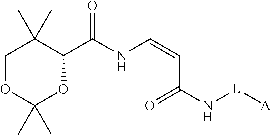

wherein -L- is a linker or a covalent bond; and -A is at least one chemical agent;
and also salts, solvates, and protected forms thereof.

In one aspect, L is a linker as defined for the compounds of formula (Id). In one aspect, -L²- is a C₃ alkylene. In a particular aspect, -L¹- is a C₂ alkylene and -L²- is a C₃ alkylene.

In one aspect, the linker -L- is a group *-L³-G-L^{A}-,
wherein the asterisk indicates the point of attachment to —X—;
-L³- is an alkylene; and
-G- is a maleimide-derived group; and
-L^{A}- is a covalent bond.

In a particular aspect, -L³- is a C₅ alkylene.

In certain aspects, a compound for detection methods is selected from one of the following formulae:

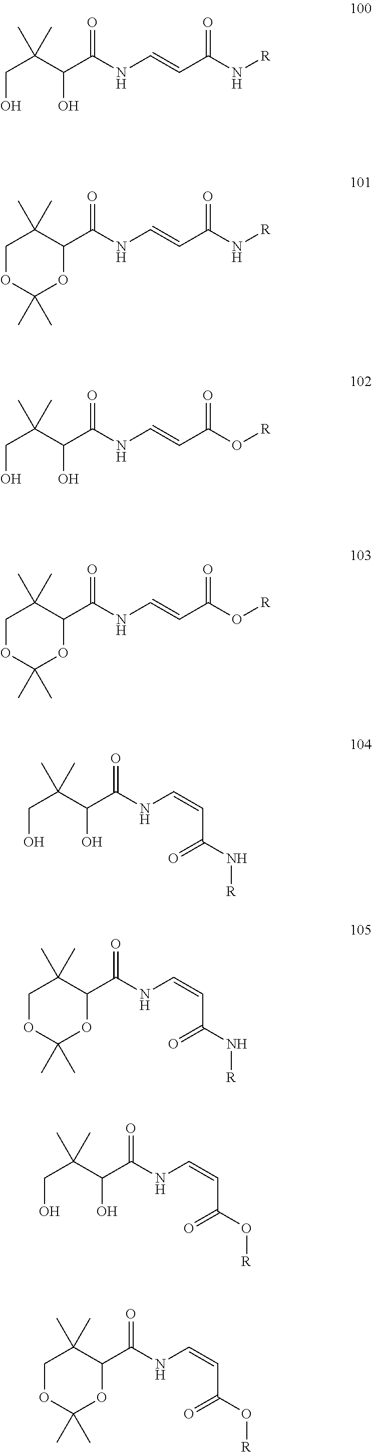

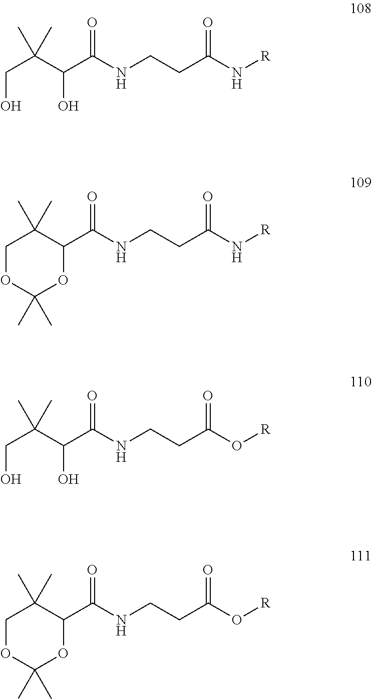

wherein —R is -L-A;
wherein -L- is a linker or a covalent bond; and -A is at least one chemical agent,
and also salts, solvates, and protected forms thereof.

In certain aspects, a compound for detection methods is selected from one of the following formulae:

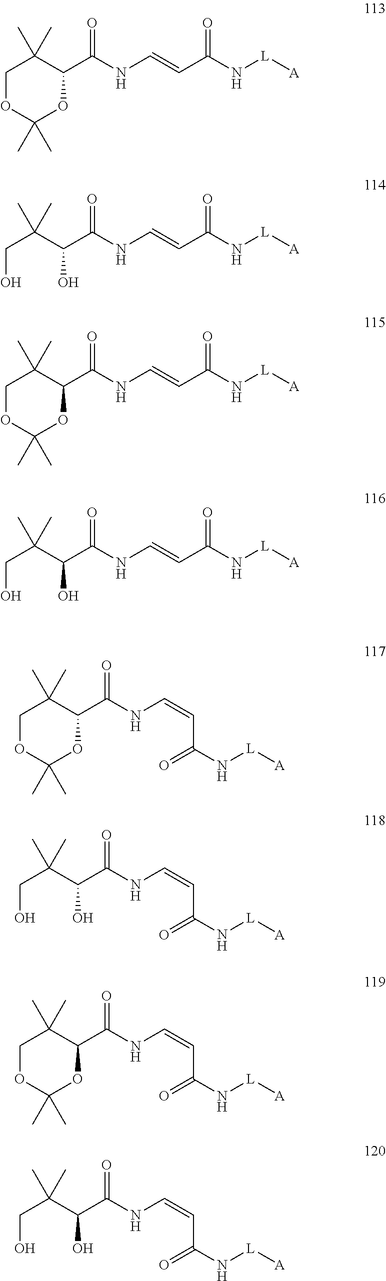

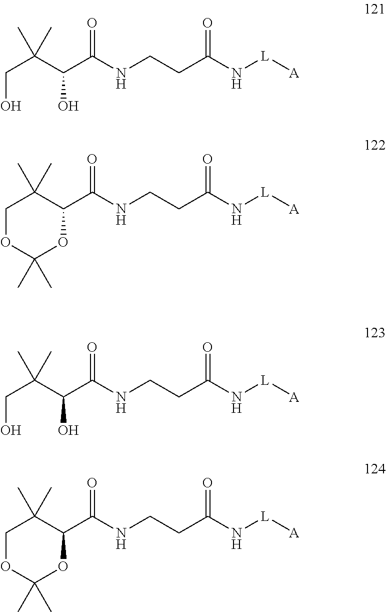

wherein -L- is a linker or a covalent bond; and -A is at least one chemical agent,
and also salts, solvates, and protected forms thereof.

In certain aspects, the substituent -L- for each of the formulae as set out herein may be a linker as defined for the compounds of formula (Id), above. For example, -L¹- may be a C₂ alkylene, and -L²- may a C₃ alkylene or a C₅ alkylene.

In certain aspects, a compound of this disclosure may contain a triazole group, particularly a 1,2,3-triazole, which may be present in the linker -L-, or in a precursor group to the linker -L-. Where the 1,2,3-triazole is present, it is 1,4- or 1,5-substituted. In one aspect, the 1,2,3-triazole is 1,4-substituted. Other suitable linkers are set out herein.

The compounds of this disclosure are described in detail herein. Various chemical agents for conjugation are also described herein. For example, to facilitate detection and imaging of tumour sites (e.g., primary and/or secondary sites) conjugates comprising detection agents can be delivered to malignant cells as described herein. Detection agents may include, for example, radioactive substances (e.g., radioisotopes, radionuclides, radiolabels or radiotracers), dyes, contrast agents, fluorescent compounds or molecules, bioluminescent compounds or molecules, enzymes, and enhancing agents (e.g., paramagnetic or superparamagnetic ions). Nanoparticles, for example quantum dots and metal nanoparticles (described below) may also be suitable for use as a detection agent. Exemplary detection agents are described in detail herein.

As set out in this document, one or more of the disclosed compounds may be included in a composition suitable for administration to a subject, e.g., a pharmaceutical composition. Exemplary routes of administration include: oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously and intramuscularly.

Broadly speaking, the detection methods of this disclosure may be applied to any THL positive cell. This includes any cell, for example, any abnormal cell, any afflicted cell, any diseased cell, any malignant cell, etc., which shows a measurable increase in binding and/or uptake of one of more pantothenic acid (PA) conjugates or CJ-15,801 (138) conjugates, as compared to a control cell (e.g., normal cell, non-afflicted cell, non-diseased cell, non-malignant cell, etc). Such increase may be evident at any given concentration of conjugate over any given time period. This increase in uptake may be assessed in accordance with any detection method set out herein. As an example, standard IC₅₀ calculations may be utilised.

In various aspects, the malignant cells to be targeted may be selected from the group consisting of bladder cancer cells, bone cancer cells, breast cancer cells, brain cancer cells, colorectal cancer cells, endometrial cancer cells, head and neck cancer cells, kidney cancer cells, leukaemia cells, liver cancer cells, lung cancer cells, lymphoma cells, melanoma cells, ovarian cancer cells, pancreatic cancer cells, prostate cancer cells, and thyroid cancer cells.

As exemplifications, the malignant cell may be a cell of: acute lymphoblastic leukaemia (ALL), acute myeloid leukaemia (AML), adrenocortical carcinoma, AIDS-related cancer (including AIDS-related lymphoma, Kaposi sarcoma, CNS lymphoma, primary CNS lymphoma, etc), astrocytoma (or other childhood brain cancers), atypical teratoid/rhabdoid tumour, basal cell carcinoma, bile duct cancer, bladder cancer, bone cancer (including Ewing sarcoma, osteosarcoma, malignant fibrous histiocytoma, etc), brain cancer, breast cancer, bronchial tumour (or other lung cancers), or Burkitt lymphoma.

As exemplifications, the malignant cell may be a cell of: a carcinoid tumour (or other gastrointestinal tumours), or carcinoma (including unknown primary carcinomas), cardiac tumour, central nervous system cancer (including various brain tumours, medulloblastoma, etc), cervical cancer, cholangiocarcinoma (or other bile duct cancers), chordoma (or other childhood bone cancers), chronic lymphocytic leukaemia (CLL), chronic myelogenous leukaemia (CML), chronic myeloproliferative neoplasms, colorectal cancer, craniopharyngioma, cutaneous T cell lymphomas (including mycosis fungoides, Sezary syndrome, etc), ductal carcinoma (or other types of breast cancer), embryonal tumours, endometrial cancer, ependymoma, oesophageal cancer, esthesioneuroblastoma (or other forms of head and neck cancer), extracranial germ cell tumour, extragonadal germ cell tumour, eye cancer (including intraocular melanoma, retinoblastoma, etc).

As exemplifications, the malignant cell may be a cell of: fallopian tube cancer, gallbladder cancer, gastric cancer, gastrointestinal carcinoid tumour, gastrointestinal stromal tumour, germ cell tumour, ovarian germ cell tumour, testicular cancer, gestational trophoblastic disease, hairy cell leukaemia, hepatocellular cancer, histiocytosis, Langerhans cell cancer, Hodgkin lymphoma, non-Hodgkin lymphoma, hypopharyngeal cancer, intraocular melanoma, Islet cell tumour, pancreatic neuroendocrine tumour, kidney cancer, Langerhans cell histiocytosis, laryngeal cancer, leukaemia, lip or oral cavity cancer, liver cancer, lung cancer (including non-small cell lung cancer, small cell lung cancer, pleuropulmonary blastoma, pulmonary inflammatory myofibroblastic tumour, tracheobronchial tumour, etc), or lymphoma.

As exemplifications, the malignant cell may be a cell of: male breast cancer, melanoma, Merkel cell carcinoma (or other skin cancers), mesothelioma, metastatic cancer, midline tract carcinoma, mouth cancer, multiple endocrine neoplasia syndrome, multiple myeloma neoplasm, plasma cell neoplasm, myelodysplastic syndrome, myelodysplastic/myeloproliferative neoplasm, or myeloproliferative neoplasm, nasal cavity cancer, paranasal sinus cancer, nasopharyngeal cancer, neuroblastoma, oral cancer, oropharyngeal cancer, osteosarcoma, pleomorphic sarcoma, ovarian cancer, pancreatic cancer, pancreatic neuroendocrine tumour (including Islet cell tumours), papillomatosis (or other childhood laryngeal cancers), paraganglioma, parathyroid cancer, penile cancer, pharyngeal cancer, pheochromocytoma, pituitary tumour, multiple myeloma, pleuropulmonary blastoma (or other forms of lung cancer), peritoneal cancer, prostate cancer, or pulmonary inflammatory myofibroblastic tumour.

As exemplifications, the malignant cell may be a cell of: rectal cancer, renal cell cancer, rhabdomyosarcoma, salivary gland cancer, sarcoma (including vascular tumour sarcoma, soft tissue sarcoma, Ewing sarcoma, osteosarcoma, uterine sarcoma, etc), skin cancer, small cell lung cancer, small intestine cancer, squamous cell carcinoma, squamous neck cancer, or stomach cancer, T cell lymphoma (including cutaneous T cell lymphoma), testicular cancer, throat cancer, nasopharyngeal cancer, oropharyngeal cancer, hypopharyngeal cancer, thymoma, thymic carcinoma, thyroid cancer, tracheobronchial tumour, transitional cell cancer of the renal pelvis, transitional cell cancer of the ureter, urethral cancer, uterine cancer, uterine sarcoma, vaginal cancer, vulvar cancer, Wilms tumour (or other childhood kidney tumours). See, e.g., information at the National Cancer Institute of the National Institutes of Health; www.cancer.gov.

In particular aspects, the malignant cell to be targeted may be selected from the group consisting of: melanoma cells; colon cancer cells; rectal cancer cells; and leukaemia cells.

In particular aspects, the malignant cell may be selected from the group consisting of: malignant melanocytes; melanoma tissue cells; melanoma lesion cells; and melanoma tumour cells; as well as melanoma metastasis cells; cutaneous melanoma cells; ocular melanoma cells; superficial spreading melanoma cells; nodular melanoma cells; lentigo maligna melanoma cells; amelanotic melanoma cells; acral lentiginous melanoma cells; mucosal melanoma cells; desmoplastic melanoma cells; and uveal melanoma cells. As further examples, the malignant cell may be selected from: melanoma cells having mutation of one or more of CDK2NA, MDm2, CDK4EGF, RBT, MC1R, TYR, TYRP1, and ASIP genes; or having mutation of one or more of BRAF, EGF, Fas, and PTEN genes.

In particular aspects, the malignant cell may be selected from the group consisting of: colorectal adenocarcinoma cells; colorectal mucinous adenocarcinoma cells; colorectal signet ring adenocarcinoma cells; colorectal lymphoma cells; gastrointestinal stromal tumour cells; colorectal leiomyosarcoma cells; colorectal melanoma cells; colorectal squamous cell carcinoma cells; hereditary nonpolyposis colorectal cancer cells; metastatic colorectal cancer cells; familial adenomatous polyposis cells; juvenile polyposis coli cells; Turcot Syndrome cancer cells; and Peutz-Jeghers Syndrome cancer cells.

In particular aspects, the malignant cell may be selected from the group consisting of: lymphocytic leukaemia cells; acute lymphocytic leukaemia cells; chronic lymphocytic leukaemia cells; myeloid leukaemia cells; acute myeloid leukaemia cells; chronic myeloid leukaemia cells; prolymphocytic leukaemia cells; large granular lymphocytic (LGL) leukaemia cells; hairy cell leukaemia cells; and myelodysplastic syndrome cells.

The detection method may utilise real-time viewing of the region (for example by direct visualization including under magnification); recording an image of the region (for example by digital photography, macrophotography or microphotography); transmitting an image of the region (for example by email or other form of electronic communication); storing an image of the region (for example in cloud-based storage); retrieving an image of the region (for example by email or other forms of electronic communication); and/or analysis of an image of the region (for example by an expert or machine).

As exemplifications, the subject in need of detection methods may be selected from: a vertebrate, a mammal, a placental mammal, a human, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a caprine (e.g. goat), a primate, simian (e.g., a monkey or ape), a monkey (e.g., marmoset, baboon), and an ape (e.g., gorilla, chimpanzee, orang-outan, gibbon). Other suitable subjects are described herein.

As exemplifications, the biological sample subjected to the detection method may be selected from the group consisting of: at least one malignant cell; at least one melanoma cell; at least one leukaemia cell; at least one colorectal cancer cell; at least one normal cell; malignant tissue; normal tissue; melanoma tissue; non-melanoma tissue; colorectal cancer tissue; organ tissue; skin tissue (epidermis (outer layer), dermis (inner layer) and hypodermis (subcutaneous tissue)); eye tissue (iris, ciliary body, choroid); bones; brain; liver; lung; lymph; and mucosal tissue (anus, vagina, penis, vulva, mouth, stomach, colon, rectum, etc). Particular biological samples include, for example, samples of blood, cell culture cells, cell culture media, urine, faeces, and faecal occult samples, as well as ascites fluid, bile, and saliva. Blood samples may include plasma, serum, and/or whole blood. Specifically included as biological samples are secretions, fluids, tissues, cells such as cells from biopsy, cellular matter, and extracellular matter. Other biological samples are described herein. Any combination of samples may be utilised.

In various aspects, the detection method of this disclosure (e.g., determining the targeting by a compound, determining levels of a compound, determining localisation of a compound) may utilise one or more of: illumination; visualisation; imaging; optical imaging; fluorescence imaging; infra-red imaging; microscopy imaging; machine imaging; extended depth of field imaging; image recording; image capturing; image analysis; image filtering; image reconstruction; computer-aided image analysis; image analysis using artificial intelligence (AI); deep learning; image storage; image storage by cloud computing; image retrieval; image transmission; image recognition; image reconstruction; tomography; Computed tomography (CT or CAT); positron emission tomography (PET) or PET-CT; magnetic resonance imaging (MRI). Any combination of detection methods may be utilised.

As exemplifications, the detection methods may be utilised for the purpose of screening, localising, or otherwise diagnosing malignancies. In particular aspects, one or more compounds of this disclosure may be administered locally or systemically to a subject. For example, for local administration, the one or more compounds may be applied topically to the skin or may be injected into the skin, e.g., as in dermal administration. The site of administration may then be visualised, for example, by illumination of the site and/or imaging of the site to detect the presence of the compound(s). As one example, PET imaging may be utilised. In particular aspects, PET imaging (or other visualisation means) may be performed in conjunction with surgical methods. In particular, visualisation of the site may be used to identify the perimeter of the skin lesion or tumour, and this information can be utilised to allow accurate and complete surgical excision.

In the methods set out herein, detection may include a determination of the presence, absence, levels (e.g., amount or concentration), localisation, or specific distribution of a compound of this disclosure. This determination may be carried out in vitro or in vivo. For in vitro detection various cell screening methods may be utilised. These include, for example, magnetic activated cell sorters (MACS), microfluorometry, cytophotometry, image cytometry, mass cytometry, flow cytometry, and fluorescence activated cell sorting (FACS). For in vivo detection various imaging techniques may be utilised. These include, for example, magnetic resonance imaging (MRI), positron emission tomography (PET), microPET, computed tomography (CT), PET/CT combination imager, cooled charged coupled device (CCD), camera optical imaging, optical imaging and single photon emission computed tomography (SPECT), amongst others. In certain aspects, mPET/mCT or PET/CT imaging may be used. Multimodal imaging methods may be used. In addition, it will be possible to illuminate a site of administration (e.g., local injection or topical administration) with light of an appropriate wavelength to excite and cause fluorescence of a compound of this disclosure, and to thereby identify one or more malignant cells.

Specifically noted are radionuclide imaging modalities (e.g., positron emission tomography, (PET); single photon emission computed tomography (SPECT)). These are diagnostic cross-sectional imaging techniques that map the location and concentration of radionuclide-labelled radiotracers. PET provides anon-invasive whole-body imaging modality that detects pairs of gamma rays, a secondary product of the positron annihilation. Also noted is computerized tomography (CT) as an imaging modality. CT makes it possible to assemble three-dimensional images of any part of the body, and to display two-dimensional slices from any angle and at any depth. In CT, a radiopaque contrast agent can assist in the identification and delineation of soft tissue masses. Additionally noted is magnetic resonance imaging (MRI). MRI scanners obtain three-dimensional images using magnetic field gradients and radio waves, and can be assisted by paramagnetic and superparamagnetic contrast agents.

For in vivo detection methods, detection (e.g., imaging) may be performed at any time during or after administration of the conjugate. For example, the detection may be assessed during administration of the compound, i.e., to aid in guiding the delivery to a specific location, or at any time thereafter. Detection may be repeated at different time points. For example, additional images may be obtained at different time points after administration. As exemplifications, detection may be carried out from about 15 minutes to about 96 hours after administration, or about 30 minutes to about 48 hours after administration, or at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 35 hours, at least 40 hours, at least 45 hours, at least 48 hours, at least 54 hours, at least 60 hours, at least 66 hours, at least 72 hours, at least 78 hours, at least 84 hours, at least 90 hours, at least 96 hours after administration. In a specific aspect, the detection may be carried out at a time selected from about 15 minutes, about 30 minutes, about 1 hour, about 4 hours, about 24 hours, and about 48 hours after administration.

The detection methods of this disclosure may be carried out in conjunction with other investigative procedures. For example, physical examination may be performed including inspection of any potential malignancies (e.g., suspicious skin growths). Surgical biopsies, including sentinel lymph node biopsies, and pathological examination may also be used. Also noted are ultrasound and other imaging technologies (e.g., CAT, MRI, PET, PET-CT, etc), as well as sentinel lymph node mapping. Where multiple methods for assessment are being utilised, these may be applied concurrently or sequentially. The detection methods may also be utilised in conjunction with one or more treatment or preventative methods. For example, if malignant cells are detected in a subject, the subject may be treated by one or more procedures, e.g., chemotherapy, targeted therapy, immunotherapy (e.g., immune checkpoint inhibitors, cancer vaccines, monoclonal antibodies, etc), adoptive cell transfer, gene therapy, hormone therapy, radiotherapy (e.g., external beam radiation, internal radiation, etc), intervention radiology, photodynamic therapy, hyperthermia, stem cell transplant, bone marrow transplant, surgery (e.g., open surgery, minimally invasive surgery, cryosurgery, laser surgery, etc), or other methods.

In particular aspects, the detection methods can be utilised to determine whether particular therapeutic methods are suitable for a subject or a group of subjects. For example, a subject may be administered with one or more compounds of this disclosure (e.g., compound(s) comprising a detection agent) to determine targeting of the one or more compounds into a malignant cell in the subject. Where targeting is confirmed, the subject can then be administered with one or more compounds of this disclosure (e.g., compound(s) comprising a therapeutic agent), thereby treating the malignant cell in the subject. These and similar methods can be used to provide personalised medicine to subjects.

In certain aspects, detection may be carried out using a kit. In particular, a kit may be provided which includes one or more compounds of this disclosure. The one or more compounds used in the kit may be provide in the form of a composition, for example, a pharmaceutical composition as described herein. The one or more compounds (e.g., formulated as composition(s)) may be provided in one or more containers in the kit. Additional components may also be provided with the kit, for example, one or more excipients, or one or more additional detection agents, intended for use with the one or more compounds. Optionally, instructions may be provided with the kit, as well as any other item, such as any number of containers, labels, or medical tools, including bottles, pads, etc. The instructions for the administration of the pharmaceutical composition may include information as to dosage, dosing schedule, routes of administration, amongst other information. The kit may comprise a description of selecting an individual suitable for detection methods based on identifying whether that individual has a malignancy or a symptom of a malignancy or is at risk of having such. The kit may include one or more reagents determining levels of the compound(s) in the subject, and/or localising the compound(s) in the subject. In this way, the individual may be assessed for the presence of malignant cells, and may also be assessed for response to any therapies being utilised.

### Therapeutic and Modification Methods

The present disclosure provides methods for delivering compounds to a malignant cell, for example, to a tumour site. As demonstrated herein, the delivery of the disclosed compounds to malignant cells has been shown to be selective and specific. The compounds of this disclosure, which include chemical conjugates comprising at least one therapeutic agent, can thereby provide effective treatment and preventative methods. The therapeutic agents may be selected from antimetabolites; chain terminators; nucleic acid intercalators; enzyme inhibitors; growth factor inhibitors; pathway inhibitors; proteasome inhibitors; and radionucleotides; amongst others.

The compounds that are delivered to a site of malignancy can reduce the number of malignant cells or eliminate the malignant cells entirely, and thereby treat the malignancy. In addition, the compounds that are delivered to a malignancy can reduce or stop the dispersal of malignant cells, and thereby prevent metastasis.

The disclosed compounds can be targeted to specific locations, including primary tumour sites, secondary tumour sites, and/or metastatic sites. Therefore, the compounds of this disclosure, and pharmaceutical compositions comprising one or more of these compounds, are suitable for use in methods of treatment (e.g., for use in treating cancer) and methods of prevention (e.g., for use in preventing cancer metastasis), particularly in relation to malignant cells and malignancies.

Accordingly, in certain aspects, one or more of the compounds as described herein, or a composition comprising such, may be used in treatment methods and preventative methods for malignant cells. The compounds and compositions of this disclosure may be utilised in therapeutic and prophylactic methods for the human body, or the animal body.

In certain aspects, one or more compound of this disclosure, or a composition comprising such, may be administered to a mammalian subject, such as a human, to provide treatment methods and preventative methods for malignant cells. The compounds and compositions of this disclosure may be administered to the human body, or the animal body.

In certain aspects, one or more compound of this disclosure, or a composition comprising such, may be manufactured as a medicament for treatment methods and preventative methods for malignant cells. The compounds and compositions of this disclosure may be prepared as therapeutic and prophylactic compositions for the human body, or the animal body.

In certain aspects, one or more compound of this disclosure, or a composition comprising such, may be used in in vitro treatment methods and in in vitro preventative methods. The compounds and compositions of this disclosure may be applied to malignant cells that are located outside a human body, or outside an animal body, to modify these cells and reduce one or more of the malignant characteristics of these cells.

As an example, a method of treating a malignant cell or tumour may comprise contacting the malignant cell or tumour with one or more compounds of this disclosure, which may comprise at least one therapeutic agent, thereby treating the malignant cell or the tumour. The treatment may reduce the viability, replication, and/or tumour formation of the malignant cell. The treatment may reduce the vascularity, the growth, and/or the size of the tumour. The treatment may reduce at least one symptom caused by the presence of the malignant cell(s)/tumour(s) in the subject. The treatment may result in the reduction in number of malignant cells in a subject, or the reduction of the number of tumours in a subject. The treatment may eliminate malignant cells from the subject, or may eliminate tumours from the subject. The treatment may reduce or halt the progression of a malignancy in a subject.

As an example, a method of preventing an activity of a malignant cell or tumour may comprise contacting the malignant cell or tumour with one or more compounds of this disclosure, which may comprise at least one therapeutic agent, thereby preventing the activity of the malignant cell or the tumour. The preventative method may impede the growth or spread of the malignant cell. The preventative method may impede the growth or spread of the tumour. The preventative method may halt, decrease, or delay metastasis. The preventative method may hinder an appearance of at least one symptom caused by the presence of the malignant cell(s)/tumour(s) in the subject. For example, the preventative method may halt or delay at least one symptom from occurring, or may reduce the severity of at least one symptom should such arise.

As an example, a method of modifying a malignant cell or tumour may comprise contacting the malignant cell or the tumour with a compound of this disclosure, such comprising at least one cell modifying agent, thereby modifying the malignant cell or tumour. As examples, the malignant cell may be from a subject or a tissue: the tumour may be from a subject or from a tissue. This includes, for example, malignant cells from a cell culture: a cell line; an immortalised cell line; a malignant cell line; a leukaemia cell line; a melanoma cell line; a colorectal cell line; amongst others.

In one other aspect, the present disclosure provides a method of treating a malignant cell or preventing an activity a malignant cell, comprising administering to a subject one or more compounds of this disclosure comprising at least one therapeutic agent, thereby treating the malignant cell or preventing the activity of the malignant cell.

As described herein, it is also possible to utilise one or more compounds of this disclosure without providing conjugation to at least one therapeutic agent. In certain aspects, a compound of this disclosure may be used to achieve a therapeutic effect (e.g., treatment for a malignant cell and/or preventative action for a malignant cell), without needing to be conjugated to at least one therapeutic agent.

In one aspect, a compound for treatment and/or preventative methods has the structure Ie or II.

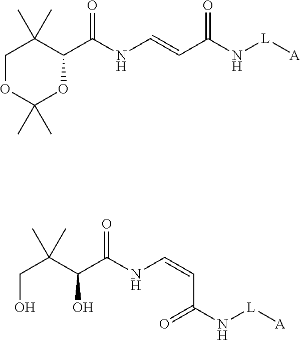

wherein -L- is a linker or a covalent bond; and -A is at least one chemical agent;
and also salts, solvates, and protected forms thereof.

In one aspect, L is a linker as defined for the compounds of formula (Id). In one aspect, -L²- is a C₃ alkylene. In a particular aspect, -L¹- is a C₂ alkylene and -L²- is a C₃ alkylene.

In one aspect, the linker -L- is a group *L³G -L^{A}-,
wherein the asterisk indicates the point of attachment to —X—;
-L³- is an alkylene; and
-G- is a maleimide-derived group; and
-L^{A}- is a covalent bond.

In a particular aspect, -L- is a C₃ alkylene.

In one aspect a compound for treatment and/or preventative methods has the structure Ie or Ii.

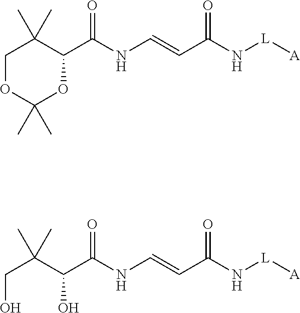

wherein -L- is a linker or a covalent bond; and -A is at least one chemical agent;
and also salts, solvates, and protected forms thereof.

In one aspect, L is a linker as defined for the compounds of formula (Id). In one aspect, -L²- is a C₃ alkylene. In a particular aspect, -L¹- is a C₂ alkylene and -L²- is a (alkylene.

In one aspect, the linker -L- is a group *-3-G-L^{A}-,
wherein the asterisk indicates the point of attachment to —X—;
-L³- is an alkylene; and
-G- is a maleimide-derived group; and
-L^{A}- is a covalent bond.

In a particular aspect, -L³- is a C₅ alkylene.

In one aspect, a compound for treatment and/or preventative methods has the structure If.

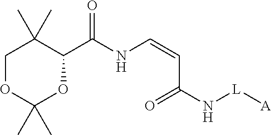

wherein -L- is a linker or a covalent bond; and -A is at least one chemical agent;
and also salts, solvates, and protected forms thereof.

In one aspect, L is a linker as defined for the compounds of formula (Id). In one aspect, -L²- is a C₃ alkylene. In a particular aspect, -L¹- is a C₂ alkylene and -L²- is a C₃ alkylene.

In one aspect, the linker -L- is a group *-L³-G-L^{A}-,
wherein the asterisk indicates the point of attachment to —X—;
-L³- is an alkylene; and
-G- is a maleimide-derived group; and
-L^{A}- is a covalent bond.

In a particular aspect, -L³- is a C₅ alkylene.

In certain aspects, a compound for treatment and/or preventative methods is selected from one of the following formulae:

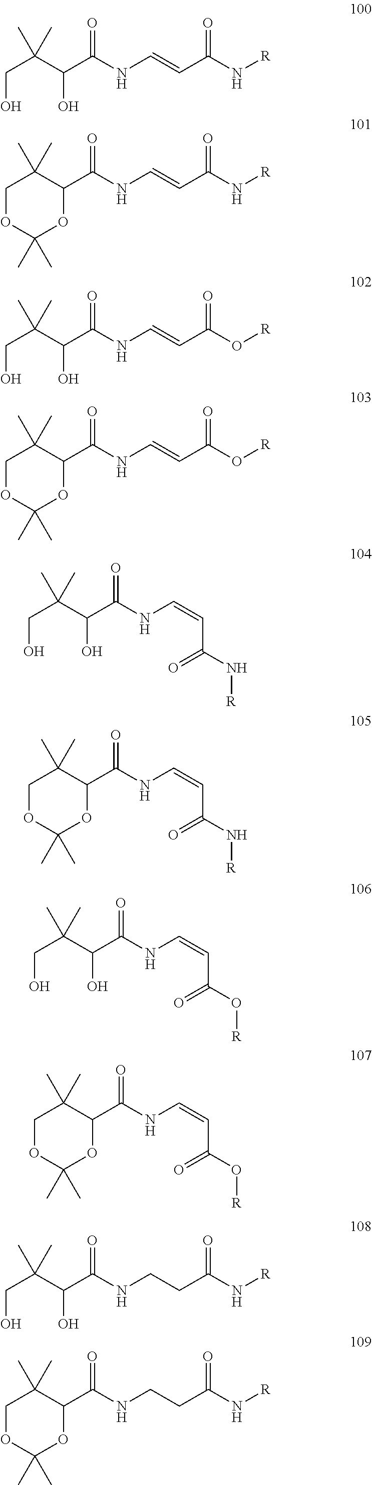

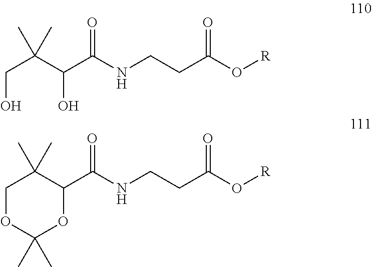

wherein —R is -L-A;
wherein -L- is a linker or a covalent bond; and -A is at least one chemical agent,
and also salts, solvates, and protected forms thereof.

In certain aspects, a compound for treatment and/or preventative methods is selected from one of the following formulae:

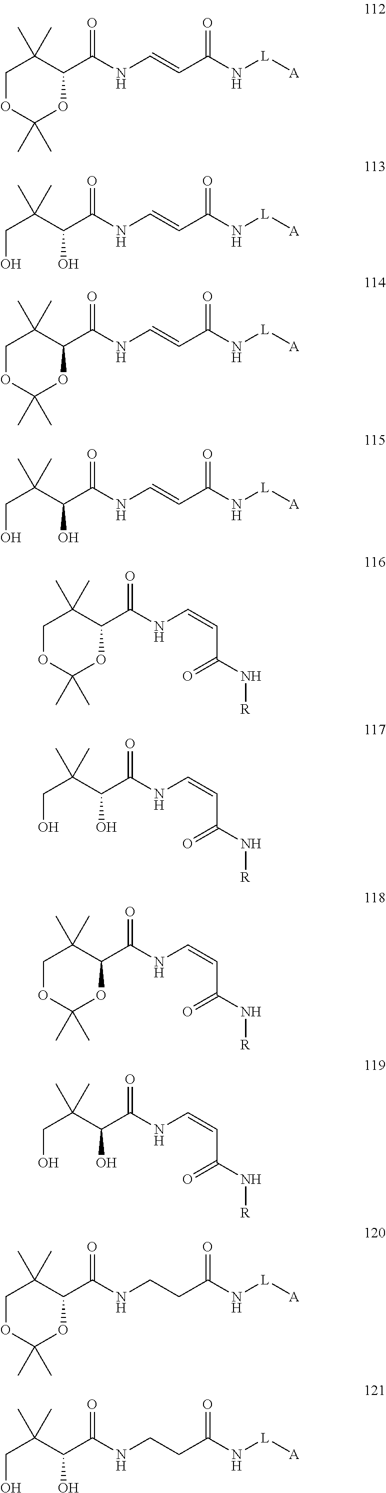

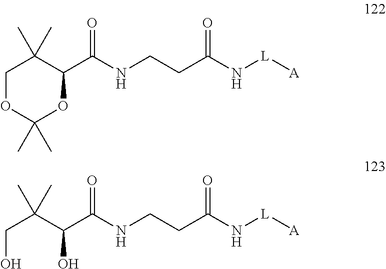

wherein -L- is a linker or a covalent bond; and -A is at least one chemical agent,
and also salts, solvates, and protected forms thereof.

In certain aspects, the substituent -L- for each of the formulae as set out herein may be a linker as defined for the compounds of formula (Id), above. For example, -L- may be a C2 alkylene, and -L² may a C3 alkylene or a C5 alkylene.

In certain aspects, a compound of this disclosure may contain a triazole group, particularly a 1,2,3-triazole, which may be present in the linker -L-, or in a precursor group to the linker -L-. Where the 1,2,3-triazole is present, it is 1,4- or 1,5-substituted. In one aspect, the 1,2,3-triazole is 1,4-substituted. Other suitable linkers are set out herein.

The compounds of this disclosure are described in detail herein. Various chemical agents for conjugation are also described herein. For example, to facilitate therapeutic or preventative methods the chemical conjugates comprising one or more therapeutic agents can be delivered to malignant cells (e.g., at primary and/or secondary sites). Therapeutic agents, include various cancer therapeutics, for example, chemotherapy compounds, targeted therapy compounds, immunotherapy compounds, gene therapy compounds, and hormone therapy compounds.

As set out in this document, one or more of the disclosed compounds may be included in a pharmaceutical composition. Exemplary routes of administration include: oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

Administration can be effected in one dose, continuously, or intermittently (e.g., in divided doses at appropriate intervals) throughout the course of therapy. The means and dosage of administration may be varied, for example in relation to the compound used for therapy, the formulation used for therapy, the treatment goal(s), the target cell(s) being treated, and/or the subject being treated. Single or multiple administrations can be carried out with the dose level and pattern being selected by the clinician. Different formulations may be used in conjunction. Different modes of administration may be used in conjunction. Such may be utilised concurrently or sequentially.

Broadly speaking, the therapeutic and modification methods of this disclosure may be applied to any THL positive cell. This includes any cell, for example, any abnormal cell, any afflicted cell, any diseased cell, any malignant cell, etc., which shows a measurable increase in binding and/or uptake of one of more pantothenic acid (PA) conjugates or CJ-15,801 (138) conjugates, as compared to a control cell (e.g., normal cell, non-afflicted cell, non-diseased cell, non-malignant cell, etc). Such increase may be evident at any given concentration of conjugate over any given time period. This increase in uptake may be assessed in accordance with any detection method set out herein.

In various aspects, the malignant cells to be targeted may be selected from the group consisting of: bladder cancer cells, bone cancer cells, breast cancer cells, brain cancer cells, colorectal cancer cells, endometrial cancer cells, head and neck cancer cells, kidney cancer cells, leukaemia cells, liver cancer cells, lung cancer cells, lymphoma cells, melanoma cells, ovarian cancer cells, pancreatic cancer cells, prostate cancer cells, and thyroid cancer cells.

In particular aspects, the malignant cell to be targeted may be selected from the group consisting of: melanoma cells; colon cancer cells; rectal cancer cells; and leukaemia cells. Specifically, the malignant cell may be selected from the group consisting of: malignant melanocytes; melanoma tissue cells; melanoma lesion cells; and melanoma tumour cells; as well as melanoma metastasis cells; cutaneous melanoma cells; ocular melanoma cells; superficial spreading melanoma cells; nodular melanoma cells; lentigo maligna melanoma cells; amelanotic melanoma cells; acral lentiginous melanoma cells; mucosal melanoma cells; desmoplastic melanoma cells; and uveal melanoma cells. As further examples, the malignant cell may be selected from: melanoma cells having mutation of one or more of CDK2NA, MDm2, CDK4EGF, RB1, MC1R, TYR, TYRP1, and ASIP genes; or having mutation of one or more of BRAF, EGF, Fas, and PTEN genes.

In particular aspects, the malignant cell to be targeted may be selected from the group consisting of: colorectal adenocarcinoma cells; colorectal mucinous adenocarcinoma cells; colorectal signet ring adenocarcinoma cells; colorectal lymphoma cells; gastrointestinal stromal tumour cells; colorectal leiomyosarcoma cells; colorectal melanoma cells; colorectal squamous cell carcinoma cells; hereditary nonpolyposis colorectal cancer cells; metastatic colorectal cancer cells; familial adenomatous polyposis cells; juvenile polyposis coli cells; Turcot Syndrome cancer cells; and Peutz-Jeghers Syndrome cancer cells.

In particular aspects, the malignant cell to be targeted may be selected from the group consisting of: lymphocytic leukaemia cells; acute lymphocytic leukaemia cells; chronic lymphocytic leukaemia cells; myeloid leukaemia cells; acute myeloid leukaemia cells; chronic myeloid leukaemia cells; prolymphocytic leukaemia cells; large granular lymphocytic (LGL) leukaemia cells; hairy cell leukaemia cells; and myelodysplastic syndrome cells. Other exemplifications for malignant cells are provided herein.

In various aspects, the subject in need of treatment methods and/or preventative methods may be a vertebrate, a mammal, a placental mammal, a human, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a caprine (e.g. goat), a primate, simian (e.g., a monkey or ape), a monkey (e.g., marmoset, baboon), an ape (e.g., gorilla, chimpanzee, orang-outan, gibbon).

It has already been noted that the compounds of this disclosure demonstrate marked selectivity and specificity in targeting malignant cells. In certain aspects, the one or more compounds of this disclosure (e.g., compound(s) conjugated to at least one therapeutic agent) may target a malignant cell such that the signal to noise ratio (e.g., test level to background level ratio; test localisation to background localisation ratio) is at least 1.5 to 1, at least 1.6 to 1, at least 1.7 to 1, at least 1.8 to 1, at least 1.9 to 1, at least 2 to 1, at least 2.1 to 1, at least 2.2 to 1, at least 2.3 to 1, at least 2.4 to 1, at least 2.5 to 1, at least 2.6 to 1, at least 2.7 to 1, at least 2.8 to 1, at least 2.9 to 1, or at least 3 to 1.

The treatment and preventative methods of this disclosure may be carried out in conjunction with one or more detection methods as described herein or other investigative procedures. For example, physical examination may be performed including inspection of any potential malignancies (e.g., suspicious skin growths). Surgical biopsies, including sentinel lymph node biopsies, and pathological examination may also be used. Also noted are ultrasound and other imaging technologies (e.g., CAT, MRI, PET, PET-CT, etc), as well as sentinel lymph node mapping. The treatment and preventative methods may also be utilised in conjunction with other therapeutic procedures. For example, in conjunction with the disclosed methods, the subject may be treated by one or more of chemotherapy, targeted therapy, immunotherapy (e.g., immune checkpoint inhibitors, cancer vaccines, monoclonal antibodies, etc), adoptive cell transfer, gene therapy, hormone therapy, radiotherapy (e.g., external beam radiation, internal radiation, etc), intervention radiology, photodynamic therapy, hyperthermia, stem cell transplant, bone marrow transplant, surgery (e.g., open surgery, minimally invasive surgery, cryosurgery, laser surgery, etc), or other methods. Where multiple medicinal methods are being utilised, these may be applied concurrently or sequentially.

In certain aspects, treatment may be carried out using a kit. In particular, a kit may be provided which includes one or more compounds of this disclosure. The one or more compounds used in the kit may be provide in the form of a composition, for example, a pharmaceutical composition as described herein. The one or more compounds (e.g., formulated as composition(s)) may be provided in one or more containers in the kit. Additional components may also be provided with the kit, for example, one or more excipients, or one or more additional cancer therapeutics (or one or more other therapeutics), intended for use with the one or more compounds. Optionally, instructions may be provided with the kit, as well as any other item, such as any number of containers, labels, or medical tools, including bottles, pads, etc. The instructions for the administration of the pharmaceutical composition may include information as to dosage, dosing schedule, routes of administration, amongst other information. The kit may comprise a description of selecting an individual suitable for treatment or preventative methods based on identifying whether that individual has a malignancy or a symptom of a malignancy or is at risk of having such. The kit may include one or more reagents determining levels of the compound(s) in the subject, and/or localising the compound(s) in the subject. In this way, the individual may be assessed for the presence of the disorder, and may also be assessed for expected response to administration of the composition.

### Synthesis Methods

Various methods of synthesis may be utilised for the disclosed compounds. Exemplary methods are provided herein. Additional methods may be obtained from GB 1820626.8 and PCT/EP2019/086120, these being hereby incorporated by reference in their entirety.

General materials and methods: Reactions involving air sensitive reagents and anhydrous solvents were performed in glassware that had been dried in an oven (130 °C). These reactions were carried out with the exclusion of air using an argon atmosphere. Acetonitrile, dichloromethane, diethyl ether, tetrahydrofuran and toluene were purified through a Pure Solv 400-5 MD solvent purification system (Innovative Technology, Inc). Microwave reactions were performed in a Biotage Initiator system.

All reagents were used as received, unless otherwise stated. Solvents were evaporated under reduced pressure at 40 °C using a Buchi Rotavapor. Column chromatography was performed under pressure using silica gel (Fluorochem Silica LC 60 A) as the stationary phase and HPLC grade solvents as eluent. Reactions were monitored by thin layer chromatography. TLC was performed on aluminium sheets pre-coated with silica gel (Merck or Fluorochem Silica Gel 60 F254). The plates were visualised by the quenching of UV fluorescence (λmax 254 nm) and/or by staining with a KMnO₄ solution dip.

Proton magnetic resonance spectra (¹H NMR) and carbon magnetic resonance spectra (¹³C NMR) were recorded at 400 MHz and 101 MHz or at 500 MHz and 126 MHz using either a Bruker DPX Avance400 instrument or a Bruker AvanceIII500 instrument. Chemical shifts (δ) are reported in parts per million (ppm) and are referenced to the residual solvent peak. NMR signals are described by multiplicity as singlet (s), doublet (d), triplet (t), quartet (q), quintet (quin) or multiplet (m) and broad (b) or by a combination of these terms. Coupling constant (J) are quoted in Hertz to the nearest 0.1 Hz.

IR spectra were obtained employing a Golden Gate™ attachment that uses a Type IIa diamond as a single reflection element so that the IR spectrum of the compound (solid or liquid) could be detected directly (thin layer) without any sample preparation (Shimadzu FTIR-8400). Only significant absorptions are reported in wavenumbers with the following terms to describe intensity: w (weak), m (medium) or s (strong).

UV-Vis absorption spectra were recorded using a Shimadzu UV-3600 UV-Vis-NIR spectrophotometer. A Brand® UV-Cuvette UV-Transparent spectrophotometry plastic cuvette with a 10 mm pathlength and 3 mL volume was used. Fluorescent emission spectra were recorded using a Shimadzu RF-5301 PC spectrofluorophotometer and Panorama fluorescence 1.1 software.

High resolution mass spectra were recorded by the analytical group of the chemistry department at the University of Glasgow on a JEOL JMS-700 mass spectrometer by electrospray and electron ionisation operating at a resolution of 15000 full widths at half height.

Experimental details for compounds:

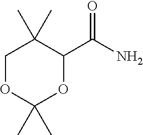

### 2,2,5,5-Tetramethyl-1,3-dioxane-4-carboxamide (R-140, S-140)

2-Methoxypropene (7 mL, 81.5 mmol) and pTsOH (400 mg, 0.47 mmol) were dissolved in acetone (55 mL) at 0 °C giving a red solution. It was further diluted with acetone (55 mL) and CH₂Cl₂ (75 mL). 2,4-dihydroxy-3,3-dimethylbutanamide (R-139, S-139) (5.5 g, 37.4 mmol) was added via a funnel in one portion. The mixture was stirred at 0 °C for 1 h, then allowed to warm to rt and stirred for a further 2 h. The reaction was quenched by addition of Et₃N dropwise until a pale yellow solution was obtained. The solution was dried with Na₂SO₄, filtered, and solvent removed in vacuo to give the crude product as a yellow/orange solid. The crude was triturated with petroleum ether affording a light yellow solid which was purified by column chromatography (3% MeOH/CH₂Cl₂) to afford the acetonide protected diol as an off-white solid (4.66 g, 67%). The NMR data obtained for this compound matches the literature data (see, e.g., Sewell et al., 2011).

¹H NMR (CDCl₃, 400 MHz) δ: 6.47 (1H, br s, NH₂), 5.77 (1H, br s, NH₂), 4.10 (1H, s, CH), 3.72 (1H, d, ²J_{H-H}=11.7, CH₂O), 3.31 (1H, d, ²J_{H-H}=11.7, CH₂O), 1.46 (3H, s, OC(CH₃)₂), 1.44 (3H, s, OC(CH₃)₂), 1.06 (3H, s, C(CH₃)₂), 1.04 (3H, s, C(CH₃)₂); ¹³C NMR (CDCl₃, 101 MHz) δ: 172.5 (C═O), 99.0 (OC(CH₃)₂), 77.3 (OCH), 71.5 (OCH₂), 32.9 (OCH₂C), 29.4 (OC(CH₃)₂), 22.1 (OC(CH₃)₂), 18.9 (CH₂C(CH₃)₂), 18.6 (CH₂C(CH₃)₂).
(R-140); [α]_{D}+88.3 (c=1.1, CHCl₃, T=22.0 °C).
(S-140); [α]_{D}−91.7 (c=1.4, CHCl₃, T=22.1 °C).

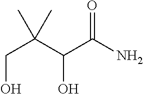

### 2,4-Dihydroxy-3,3-dimethylbutanamide (R-139, S-139)

Pantolactone (D-(−)-Pantolactone or (S)-(+)-Pantolactone) (5.2 g, 40 mmol) was added to a 2-neck round-bottomed flask with a stirrer bar. An oil bubbler was connected to one neck and a cold finger condenser, with solid carbon dioxide and acetone, to the other. At −78 °C, ~60 mL of liquid ammonia was collected from a cylinder. The oil bubbler was replaced with a glass stopper and the ammonia inlet was then replaced with the oil bubbler. The flask was then placed into a solid carbon dioxide/acetone bath and left to stir vigorously for 16 h while the ammonia evaporated. After 16 h, a solid white lump was obtained. The solid was dissolved in a mixture of CH₂Cl₂ (50 mL) and MeOH (50 mL) before removing the solvent and residual NH₃ in vacuo to give the diol as a white solid (5.8 g, 98%). The NMR data obtained for this compound matches the literature data (see, e.g., Sewell et al., 2011).

¹H NMR (DMSO-d⁶, 400 MHz) δ: 7.10 (2H, s, NH₂), 5.25 (1H, d, ³J_{H-H}=5.8, CHOH), 4.49 (1H, t, ³J_{H-H}=5.5, CH₂OH), 3.67 (1H, d, ³J_{H-H}=5.8, CHOH), 3.41 (1H, dd, ²J_{H-H}=10.44, ³J_{H-H}=5.5, CH₂OH), 3.18 (1H, d, ²J_{H-H}=10.44, CH₂OH), 0.83 (3H, s, C(CH₃)₂), 0.81 (3H, s, C(CH₃)₂); ¹³C NMR (DMSO-d⁶, 101 MHz) δ: 174.8 (C═O), 76.1 (CHOH), 69.3 (CH₂OH), 39.1 (CCH₂OH), 20.9 (C(CH₃)₂), 20.5 (C(CH₃)₂).
(R-82); [α]_{D}+45.6 (c=0.9, MeOH, T=25.5 °C).
(S-372); [α]_{D}−51.5 (c=1.2, CHCl₃, T=22.0 °C).

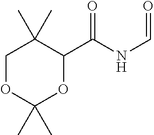

### N-Formyl-2,2,5,5-tetramethyl-1,3-dioxane-4-carboxamide (R-141, S-141)

2,2,5,5-Tetramethyl-1,3-dioxane-4-carboxamide (R-140, S-140) (3.68 g, 19.7 mmol) was dissolved in THF (100 mL) then cooled to 0 °C. nBuLi (13.6 mL of a 1.6 M solution in hexanes, 21.7 mmol) was added dropwise via syringe and left at 0 °C to stir for 0.5 h. A solution of N-formylbenzotriazole (3.47 g, 23.6 mmol) in THF (40 mL) was then added slowly via syringe. The reaction mixture was allowed to warm up to rt and left to stir for 20 h. The solution was diluted with iPrOH (65 mL) and washed with sat. NaHCO₃ (100 mL). The aqueous phase was separated and extracted with Et₂O (3×75 mL). The combined organics were washed with brine (150 mL), dried (Na₂SO₄) and filtered before removal of the solvent in vacuo to give the crude product as a pale yellow solid. The crude was purified by column chromatography (20% EtOAc/petroleum ether) to give the N-formylimide product as a white solid (3.16 g, 77%). The NMR data obtained for this compound matches the literature data (see, e.g., Sewell et al., 2011).

¹H NMR (CDCl₃, 400 MHz) δ: 9.16 (1H, d, ³J_{H-H}=10.4, HCO), 8.89 (1H, br s, NH), 4.20 (1H, s, CH), 3.72 (1H, d, ²J_{H-H}=11.8, CH₂), 3.34 (1H, d, ²J_{H-H}=11.8, CH₂), 1.50 (3H, s, OC(CH₃)₂), 1.46 (3H, s, OC(CH₃)₂), 1.07 (3H, s, C(CH₃)₂), 1.05 (3H, s, C(CH₃)₂); ¹³C NMR (CDCl₃, 101 MHz) δ: 170.6 (CC═O), 161.4 (NC═O), 99.7 (OCO), 77.1 (OCH), 71.2 (OCH₂), 33.4 (OCH₂C), 29.3 (OC(CH₃)₂), 21.7 (OC(CH₃)₂), 18.9 (C(CH₃)₂), 18.6 (C(CH₃)₂).
(R-141); [α]_{D}+88.3 ((c=1.1, CHCl₃, T=22.0 °C).
(S-141); [α]_{D}−91.7 (c=1.4, CHCl₃, T=22.0 °C).

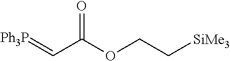

### 2-(Trimethylsilyl)ethyl 2-(triphenylphosphoranylidene)acetate (142)

2-(Trimethylsilyl)ethyl 2-bromoacetate (2.52 g, 11.3 mmol) was dissolved in toluene (50 mL) then triphenylphosphine (2.91 g, 11.3 mmol) was added in one portion. The solution was left to stir at rt for 4 days. A white precipitate formed and was collected via filtration, washed with toluene (2×50 mL) and dried under vacuum to give the phosphonium salt. The salt was dissolved in CH₂Cl₂ (50 mL) and stirred with sat. aqueous NaHCO₃ (15 mL) for 2 h. The layers were separated and the aqueous washed with CH₂Cl₂ (3×15 mL). The combined organics were washed with brine (30 mL), dried (Na₂SO₄) filtered and the solvent removed in vacuo to give the phosphonium ylide as a viscous pale yellow oil (3.3 g, 70%). The NMR data obtained for this compound matches the literature data (see, e.g., Sewell et al., 2011).

¹H NMR (CDCl₃, 400 MHz) δ: 7.71-7.64 (6H, m, ArH), 7.57-7.53 (3H, m, ArH), 7.49-7.46 (6H, m, ArH), 4.02 (2H, br s, OCH₂), 2.88 (1H, br s, Ph₃P═CH), 0.83 (1H, br s, CH₂Si(CH₃)₃), −0.02 (9H, s, 3×Si(CH₃)₃); ¹³C NMR (CDCl₃, 126 MHz) δ: 132.1 (6×ArC), 132.0 (6×ArC), 131.9 (3×ArC), 131.9 (3×ArC_{q}), 62.5 (OCH₂), 21.6 (d, ¹J_{C-P}=121.2, C═P), 17.2 (CH₂Si(CH₃)₃), −1.5 (3×Si(CH₃)₃).

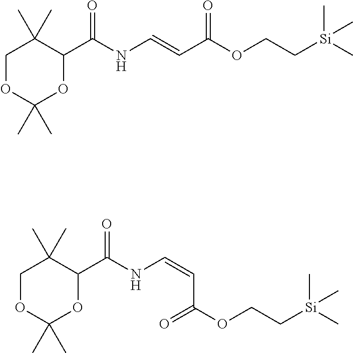 [SMILES: CC1(C)OCC(C)(C)C(C(=O)N/C=C/C(=O)OCC[Si](C)(C)C)O1 ; CC1(C)OCC(C)(C)C(C(=O)N/C=C\C(=O)OCC[Si](C)(C)C)O1]

### (E)-2-(Trimethylsilyl)ethyl-3-(2,2,5,5-tetramethyl-1,3-dioxane-4-carboxamido)acrylate (R-143, S-143) & (Z)-2-(Trimethylsilyl)ethyl-3-(2,2,5,5-tetramethyl-1,3-dioxane-4-carboxamido)acrylate (R-144, S-144)

2-(Trimethylsilyl)ethyl-2-(triphenylphosphoranylidene)acetate 142 (0.84 g, 3.9 mmol) was dissolved in benzene (60 mL) and N-formyl-2,2,5,5-tetramethyl-1,3-dioxane-4-carboxamide (R-141, S-141) (0.84 g, 3.9 mmol) was added. The solution was heated to 80 °C for 16 h then let cool to rt. The solvent was removed in vacuo to give the crude as a yellow oil. The crude was purified using column chromatography (10-20% EtOAc/petroleum ether) to give the enamide products as a separable mixture of cis (0.46 g, 33%) and trans (0.75 g, 54%) isomers as a yellow oil and white solid respectively. The NMR data obtained for this compound matches the literature data (see, e.g., Sewell et al., 2011).

### (E)-2-(Trimethylsilyl)ethyl-3-(2,2,5,5-tetramethyl-1,3-dioxane-4-carboxamido)acrylate (R-143, S-143)

¹H NMR (CDCl₃, 400 MHz) δ: 8.37 (1H, d, ³J_{H-H}=11.8, NH), 7.97 (1H, dd, ³J_{H-H}=14.2, 11.8, NCH), 5.58 (1H, d, ³J_{H-H}=14.2, CH═CHCO₂), 4.24-4.22 (2H, m, CO₂CH₂), 4.20 (1H, s, OCH), 3.72 (1H, d, ²J_{H-H}=11.8, CH₂C(CH₃)₂), 3.33 (1H, d, ²J_{H-H}=11.8, CH₂C(CH₃)₂), 1.52 (3H, s, OC(CH₃)₂), 1.46 (3H, s, OC(CH₃)₂), 1.06 (3H, s, C(CH₃)₂), 1.01 (3H, s, C(CH₃)₂), 0.05 (9H, s, 3×Si(CH₃)₃); ¹³C NMR (CDCl₃, 101 MHz) δ: 169.4 (NC═O), 168.7 (C═O), 137.6 (NCH═CH), 104.7 (CH═CHCO₂), 101.0 (OC(CH₃)₂), 78.7 (OCH), 72.7 (OCH₂), 63.8 (OCH₂CH₂), 34.9 (OCH₂C), 30.9 (OC(CH₃)₂), 23.3 (OC(CH₃)₂), 20.9 (C(CH₃)₂), 20.1 (C(CH₃)₂), 18.8 (CH₂Si(CH₃)₃), 0.00 (3×Si(CH₃)₃).
(R-143); [α]_{D}+58.9 (c=1.1, CHCl₃, T=22.5 °C).
(S-143); [α]_{D}−56.7 (c=0.6, CHCl₃, T=21.6 °C).

### (Z)-2-(Trimethylsilyl)ethyl 3-(2,2,5,5-tetramethyl-1,3-dioxane-4-carboxamido)acrylate (R-144, S-144)

¹H NMR (CDCl₃, 400 MHz) δ: 11.09 (1H, d, ³J_{H-H}=11.4, NH), 7.41 (1H, dd, ³J_{H-H}=11.4, 8.9, NHCH═CH), 5.14 (1H, d, ³J_{H-H}=8.9, CH═CHCO₂), 4.26-4.22 (2H, m, OCH₂CH₂), 4.21 (1H, s, CH), 3.72 (1H, d, ²J_{H-H}=11.7, OCH₂C), 3.32 (1H, d, ²J_{H-H}=11.7, OCH₂C), 1.59 (3H, s, OC(CH₃)₂), 1.47 (3H, s, OC(CH₃)₂), 1.05 (3H, s, C(CH₃)₂), 1.04 (3H, s, OC(CH₃)₂), 1.01 (2H, m, CH₂Si(CH₃)₃), 0.05 (9H, s, 3×Si(CH₃)₃); ¹³C NMR (CDCl₃, 101 MHz) δ: 170.2 (NC═O), 169.8 (C═O), 137.3 (NCH═CH), 100.8 (CH═CHCO₂), 99.8 (OC(CH₃)₂), 78.7 (OCH), 72.8 (OCH₂), 63.7 (OCH₂CH₂), 34.8 (OCH₂C), 30.8 (OC(CH₃)₂), 23.3 (OC(CH₃)₂), 20.5 (C(CH₃)₂), 20.1 (C(CH₃)₂), 18.9 (CH₂Si(CH₃)₃), 0.0 (3×Si(CH₃)₃).
(R-144); [α]_{D}+40.5 (c=0.7, CHCl₃, T=22.5 °C).
(S-144); [α]_{D}−38.0 (c=0.8, CHCl₃, T=22.5 °C).

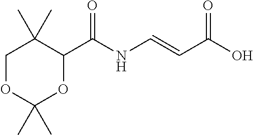 [SMILES: CC1(C)OCC(C)(C)C(C(=O)N/C=C/C(=O)O)O1]

### (E)-3-(2,2,5,5-Tetramethyl-1,3-dioxane-4-carboxamido)acrylic acid (R-145, S-145)

(E)-2-(Trimethylsilyl)ethyl-3-(2,2,5,5-tetramethyl-1,3-dioxane-4-carboxamido)acrylate (R-143, S-143) (378 mg, 1.1 mmol) was dissolved in DMF (4 mL) and cooled to 0 °C. A solution of TAS-F (453 mg, 1.6 mmol) in DMF (2 mL) was added dropwise giving a yellow solution. The solution was stirred for 16 h while warming to rt giving a small amount of precipitate. The solution was diluted with 5 mL of EtOAc before 10% citric acid was added until pH 4/5 was reached. 5 mL of 10% LiCl solution was added and stirred before the layers were separated. The organic layer was washed with 10% LiCl solution (4×5 mL) then, dried (Na₂SO₄), filtered and the solvent removed in vacuo to give the acid, CJ-15,801, as an off-white solid (219 mg, 80%) The NMR data obtained for this compound matches the literature data (see, e.g., Sewell et al., 2011).

¹H NMR (CDCl₃, 400 MHz) δ: 8.46 (1H, d, ³J_{H-H}=11.9, NH), 8.07 (1H, dd, ³J_{H-H}=14.1, 11.9, NHCH), 5.60 (1H, d, ³J_{H-H}=14.1, NCH═CH), 4.22 (1H, s, CH), 3.72 (1H, d, ²J_{H-H}=11.8, OCH₂), 3.33 (1H, d, ²J_{H-H}=11.8, OCH₂), 1.53 (3H, s, OC(CH₃)₂), 1.47 (3H, s, OC(CH₃)₂), 1.06 (3H, s, C(CH₃)₂), 1.02 (3H, s, C(CH₃)₂); ¹³C NMR (CDCl₃, 101 MHz) δ: 172.0 (NC═O), 167.9 (C═O), 137.9 (NC═C), 101.9 (C═CHCO₂), 99.6 (OC(CH₃)₂), 77.6 (OCH), 71.2 (OCH₂), 33.5 (OCH₂C), 29.4 (OC(CH₃)₂), 21.9 (OC(CH₃)₂), 18.8 (C(CH₃)₂), 18.7 (C(CH₃)₂).
(R-145); [α]_{D}+81.3 (c=0.5, CHCl₃, T=22.7 °C).
(S-145); [α]_{D}−77.6 (c=1.2, CHCl₃, T=22.8 °C).

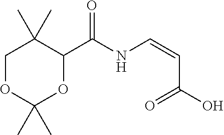 [SMILES: CC1(C)OCC(C)(C)C(C(=O)N/C=C\C(=O)O)O1]

### (Z)-3-(2,2,5,5)-Tetramethyl-1,3-dioxane-4-carboxamido)acrylic Acid (R-146, S-146)

(Z)-2-(Trimethylsilyl)ethyl-3-(2,2,5,5-tetramethyl-1,3-dioxane-4-carboxamido)acrylate (R-144, S-144) (100 mg, 0.28 mmol) was dissolved in THF (10 mL) and water (0.1 mL). The solution was cooled to 0 °C and TBAF (0.39 mL of a 1 M solution, 0.39 mmol) was added dropwise via syringe. The reaction was allowed to warm to rt while stirring overnight. The solvent was then removed in vacuo to give the crude acid as a pale yellow oil. The crude was purified by flash column chromatography (2% MeOH/CH₂Cl₂) to give the cis enamide product as a colourless oil (55 mg, 76%). The NMR data obtained for this compound matches the literature data (see, e.g., Sewell et al., 2011).

¹H NMR (CDCl₃, 500 MHz) δ: 11.19 (1H, d, ³J_{H-H}=11.4, NH), 7.49 (1H, dd, ³J_{H-H}=11.4, 8.9, NHCH═CH), 5.18 (1H, d, ³J_{H-H}=8.9, NHCH═CH), 4.23 (1H, s, CH), 3.73 (1H, d, ²J_{H-H}=11.7), 3.32 (1H, d, ²J_{H-H}=11.7), 1.56 (3H, s, OC(CH₃)₂), 1.47 (3H, s, OC(CH₃)-2), 1.04 (3H, s, C(CH₃)₂), 1.01 (3H, s, C(CH₃)₂); ¹³C NMR (CDCl₃, 126 MHz) δ: 172.9 (NC═O), 168.4 (C═O), 138.1 (NC═C), 98.9 (OC(CH₃)₂), 96.5 (CH═CHCO₂), 76.8 (OCH), 71.1 (OCH₂), 33.1 (OCH₂C), 29.0 (OC(CH₃)₂), 21.3 (OC(CH₃)₂), 18.6 (C(CH₃)₂), 18.4 (C(CH₃)₂).
(R-146); [α]_{D}+51.0 (c=0.5, CHCl₃, T=22.7 °C).
(S-146); [α]_{D}−47.1 (c=0.8, CHCl₃, T=21.7 °C).

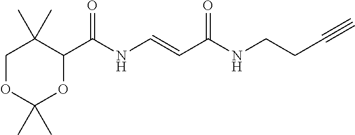 [SMILES: C#CCCNC(=O)/C=C/NC(=O)C1OC(C)(C)OCC1(C)C]

### (E)-N-(3-(But-3-yn-1-ylamino)-3-oxoprop-1-en-1-yl)-2,2,5,5-tetramethyl-1,3-dioxane-4-carboxamide (R-147, S-147)

(E)-3-(2,2,5,5-Tetramethyl-1,3-dioxane-4-carboxamido)acrylic acid (R-145, S-145) (100 mg, 0.37 mmol) was dissolved in CH₂Cl₂ (1 mL) in a 0.5-2 mL microwave veil with a magnetic stirring bar. BTFFH (184 mg, 0.58 mmol) was added then DIPEA (102 μL, 0.58 mmol) and 1-amino-3-butyne (46.0 μL, 0.58 mmol) via micropipette. The vial was capped and heated to 80 °C under microwave conditions for 2.5 h. The solvent was then removed in vacuo to give the crude as a pale yellow oil. This was purified by flash column chromatography (50% EtOAc/petroleum ether) to give the alkyne product as a white solid (86 mg, 68%).

¹H NMR (CDCl₃, 400 MHz) δ: 8.29 (1H, d, ³J_{H-H}=10.6, NHCH), 7.81 (1H, dd, ³J_{H-H}=13.8, 10.6, NHCH═CH), 5.84 (1H, d, ³J_{H-H}=13.8, NHCH═CH), 5.72 (1H, br s, NHCH₂), 4.19 (1H, s, OCH), 3.71 (1H, d, ²J_{H-H}=11.8, OCH₂), 3.52-3.47 (2H, m, NHCH₂), 3.31 (1H, d, ²J_{H-H}=11.8, OCH₂), 2.44 (2H, td, ³J_{H-H}=6.3, ⁴J_{H-H}=2.6, CH₂CCH), 2.01 (1H, t, ⁴J_{H-H}=2.6, CCH), 1.50 (3H, s, OC(CH₃)₂), 1.45 (3H, s, OC(CH₃)₂), 1.05 (3H, s, C(CH₃)₂), 1.00 (3H, s, C(CH₃)₂); ¹³C NMR (CDCl₃, 126 MHz) δ: 168.1 (NC═O), 166.1 (NC═O), 133.2 (NC═C), 105.9 (CH═CHCO), 99.5 (OC(CH₃)₂), 81.7 (CH₂CCH), 77.6 (OCH), 71.3 (OCH₂), 70.0 (CH₂CCH), 38.0 (NHCH₂), 33.4 (OCH₂C), 29.5 (OC(CH₃)₂), 21.9 (OC(CH₃)₂), 19.6 (CH₂CCH), 18.8 (C(CH₃)₂), 18.7 (C(CH₃)₂); IR vₘₐₓ(film)/cm⁻¹ (neat): 3305 (w), 3290 (m), 2958 (w), 2947 (w), 2888 (w); HRMS (EI) calcd for C₁₆H₂₄O₄N₂BF₂ [M]⁺: m/z 308.1736, found m/z 308.1739; MP Range: 153-154 °C.
(R-147); [α]_{D}+49.9 (c=0.7, (CH₃)₂CO, T=24.4 °C).
(S-147); [α]_{D}−65.5 (c=1.1, (CH₃)₂CO, T=24.4 °C).

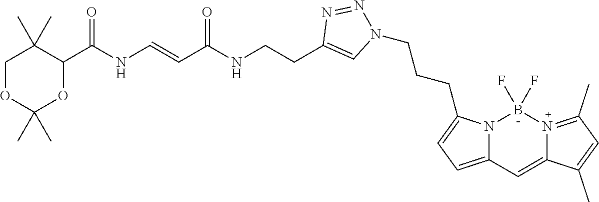

### (E)-N-((((3-[4,4-Difluoro-5,7-dimethyl-4-bora-3a,4a-diaza-s-indacene-3-yl]propyl)-1H-1,2,3-triazol-4-yl)ethylamino)-3-oxoprop-1-enyl)-2,2,5,5-tetramethyl-1,3-dioxane-4-carboxamide (125, 127)

(E)-N-(3-(But-3-yn-1-ylamino)-3-oxoprop-1-en-1-yl)-2,2,5,5-tetramethyl-1,3-dioxane-4-carboxamide (R-147, S-147) (86 mg, 0.27 mmol) was dissolved in THF (4 mL) before 3-azido[4,4-difluoro-5,7-dimethyl-4-bora-3a,4a-diaza-s-indacene-3-yl]propane 148 (114 mg, 0.37 mmol) was added in one portion followed by DIPEA (1 drop) and CuI (5 mg, 10 mol %). The mixture was heated to 70 °C for 18 h then concentrated in vacuo to give the crude product as a brown/reddish oil. The crude was purified by flash column chromatography (5% MeOH/CH₂Cl₂) to give the triazole product as a green/red solid (134 mg, 79%).

¹H NMR (CDCl₃, 400 MHz) δ: 8.33 (1H, d, ³J_{H-H}=11.6, NHCH), 7.84 (1H, dd, ³J_{H-H}=13.9, 11.6, NHCH═CH), 7.40 (1H, s, ArCH_{triazole}), 7.09 (1H, s, ArCH_{BODIPY-C8}), 6.86 (1H, d, ³J_{H-H}=4.0, ArCH_{BODIPY-C1}), 6.42 (1H, t, ³J_{H-H}=5.4, NHCH₂), 6.22 (1H, d, ³J_{H-H}=4.0, ArCH_{BODIPY-C2}), 6.11 (1H, s, ArCH_{BODIPY-C6}), 5.73 (1H, d, ³J_{H-H}=13.9, NHCH═CH), 4.39 (2H, t, ³J_{H-H}=7.0, ArNCH₂), 4.15 (1H, s, OCH), 3.68 (1H, d, ²J_{H-H}=11.8, OCH₂), 3.65-3.60 (2H, m, NHCH₂), 3.27 (1H, d, ²J_{H-H}=11.8, OCH₂), 2.96 (2H, t, ³J_{H-H}=7.5, NHCH₂CH₂), 2.92 (2H, t, ³J_{H-H}=6.2, ArC_{BODIPY-C3}CH₂), 2.53 (3H, s, ArC_{BODIPY-C5}CH₃), 2.34 (2H, quin, ³J_{H-H}=7.2, ArNCH₂CH₂), 2.24 (3H, s, ArC_{BODIPY-C7}H₃), 1.45 (3H, s, OC(CH₃)₂), 1.41 (3H, s, OC(CH₃)₂), 1.01 (3H, s, C(CH₃)₂), 0.96 (3H, s, C(CH₃)₂); ¹³C NMR (CDCl₃, 126 MHz) δ: 167.9 (NC═O), 166.2 (NC═O), 160.6 (ArC_{q}), 156.5 (ArC_{q}), 144.2 (ArC_{q}), 135.3 (ArC_{q}), 133.2 (NC═C), 132.9 (ArC_{q}), 128.1 (ArCH), 126.0 (ArCH_{triazole}), 123.9 (ArCH), 120.7 (ArCH), 116.6 (ArCH), 106.2 (CH═CHCO), 99.5 (OC(CH₃)₂), 77.2 (OCH), 71.3 (OCH₂), 49.9 (ArNCH₂), 38.6 (NHCH₂), 33.4 (OCH₂C), 29.7 (ArNHCH₂CH₂), 29.4 (OC(CH₃)₂), 25.6 (C_{BODIPY-C3}CH₂), 25.3 (NHCH₂CH₂), 21.9 (OC(CH₃)₂), 18.8 (CH₂C(CH₃)₂), 18.7 (CH₂C(CH₃)₂), 14.9 (C_{BODIPY-C5} CH₃), 11.3 (C_{BODIPY-C7}CH₃). IR vₘₐₓ(film)/cm⁻¹ (neat): 2989 (w) 2826 (w), 1660 (m), 1592 (s), 1390 (s); HRMS (ESI) calcd for C₃₀H₃₉N₇O₄BF₂ (M−H)⁻: m/z 609.3166, found m/z 609.3141
(R-125); [α]_{D}+29.6 (c=0.6, (CH₃)₂CO, T=24.4 °C).
(S-127); [α]_{D}−21.8 (c=1.2, (CH₃)₂CO, T=24.4 °C).

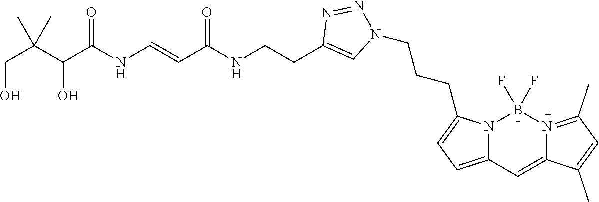

### (E)-N-((((3-[4,4-Difluoro-5,7-dimethyl-4-bora-3a,4a-diaza-s-indacene-3-yl]propyl)-1H-1,2,3-triazol-4-yl)ethylamino)-3-oxoprop-1-enyl)-2,4-dihydroxy-3,3-dimethylbutanamide (124, 126)

(E)-N-((((3-[4,4-Difluoro-5,7-dimethyl-4-bora-3a,4a-diaza-s-indacene-3-yl]propyl) -1H-1,2,3-triazol-4-yl)ethylamino)-3-oxoprop-1-enyl)-2,2,5,5-tetramethyl-1,3-dioxane-4-carboxamide (125, 127) (134 mg, 0.22 mmol) was dissolved in MeCN (7 mL) and H₂O (0.3 mL) before BiCl₃ (7 mg, 0.02 mmol) was added. The mixture was stirred vigorously for 16 h before filtering through celite and washing through with MeCN (40 mL) before removal of solvent in vacuo to give the crude diol as a brown oil. The crude was purified by flash column chromatography (5% MeOH/CH₂Cl₂) to give the diol as a red oil that solidified upon cooling (63 mg, 54%) together with 30 mg (22%) of starting material.

¹H NMR (CDCl₃, 500 MHz) δ: 9.02 (1H, d, ³J_{H-H}=11.6, NHCH), 7.85 (1H, dd, ³J_{H-H}=13.8, 10.9, NHCH═CH), 7.43 (1H, s, ArCH_{triazole}), 7.10 (1H, s, ArCH_{BODIPY-C8}), 6.88 (1H, d, ³J_{H-H}=4.0, ArCH_{BODIPY-C1}), 6.55 (1H, br s, NHCH₂), 6.24 (1H, d, ³J_{H-H}=4.0, ArCH_{BODIPY-C2}), 6.13 (1H, s, ArCH_{BODIPY-C6}), 5.69 (1H, d, ³J_{H-H}=13.8, NHCH═CH), 4.74 (1H, br s, CHOH), 4.40 (2H, t, ³J_{H-H}=6.9, ArNCH₂), 4.15 (1H, s, OCH), 3.63-3.58 (4H, m, NHCH₂, OCH₂), 3.50 (1H, br s, CH₂OH), 2.96 (2H, t, ³J_{H-H}=7.6, NHCH₂CH₂), 2.91 (2H, t, ³J_{H-H}=5.76, ArC_{BODIPY-C3}CH₂), 2.54 (3H, s, ArC_{BODIPY-C5}CH₃), 2.34 (2H, quin, ³J_{H-H}=7.2, ArNCH₂CH₂), 2.26 (3H, s, ArC_{BODIPY-C7}CH₃), 1.00 (3H, s, C(CH₃)₂), 0.95 (3H, s, C(CH₃)₂); ¹³C NMR (CDCl₃, 126 MHz) δ: 171.5 (NC═O), 167.6 (NC═O), 160.1 (ArC_{q}), 156.4 (ArC_{q}), 145.7 (ArC_{q}), 144.6 (ArC_{q}), 135.4 (ArC_{q}), 133.3 (NC═C), 132.3 (ArC_{q}), 128.3 (ArCH), 126.0 (ArCH_{triazole}), 123.6 (ArCH), 120.7 (ArCH), 116.6 (ArCH), 105.9 (CH═CHCO), 77.6 (OCH), 71.2 (OCH₂), 50.0 (ArNCH₂), 39.5 (OCH₂C), 38.5 (NHCH₂), 29.7 (ArNHCH₂CH₂), 25.3 (NHCH₂CH₂), 25.0 (C_{BODIPY-C3}CH₂), 21.5 (C(CH₃)₂), 20.3 (C(CH₃)₂), 15.3 (C_{BODIPY-C5}CH₃), 9.9 (C_{BODIPY-C7}CH₃). IR vₘₐₓ(film)/cm⁻¹ (neat): 2975 (w) 2824 (w), 1665 (m), 1596 (s), 1385 (s); HRMS (ESI) calcd for C₃₀H₃₉N₇O₄BF₂ (M−H)⁻: m/z 569.2853, found m/z 569.2831; MP Range: 104-106 °C.
(124); [α]_{D}+28.1 (c=0.5, (CH₃)₂CO, T=24.4 °C).
(126); [α]_{D}−24.0 (c=1.0, (CH₃)₂CO, T=24.4 °C).

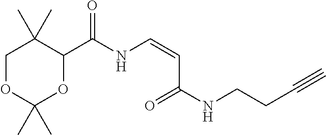 [SMILES: C#CCCNC(=O)/C=C\NC(=O)C1OC(C)(C)OCC1(C)C]

### (Z)—N-(3-(But-3-yn-1-ylamino)-3-oxoprop-1-en-1-yl)-2,2,5,5-tetramethyl-1,3-dioxane-4-carboxamide (R-149, S-149)

(Z)-3-(2,2,5,5-Tetramethyl-1,3-dioxane-4-carboxamido)acrylic acid (R-146, S-146) (125 mg, 0.49 mmol) was dissolved in CH₂Cl₂ (1 mL) in a 0.5-2 mL microwave veil with a magnetic stirring bar. BTFFH (233 mg, 0.74 mmol) was added then DIPEA (150 μL, 0.74 mmol) and 1-amino-3-butyne (61 μL, 0.74 mmol) via micropipette. The vial was capped and heated to 80 °C under microwave conditions for 2.5 h. The solvent was then removed in vacuo to give the crude as a pale yellow oil. This was purified by flash column chromatography (10-25% EtOAc/petroleum ether) to give the alkyne product as a white solid (110 mg, 73%).

¹H NMR (CDCl₃, 400 MHz) δ: 11.66 (1H, d, ³J_{H-H}=10.9, NHCH), 7.31 (1H, dd, ³J_{H-H}=10.9, 8.9, NHCH), 5.74 (1H, br s, NHCH₂), 5.03 (1H, d, ³J_{H-H}=8.9, NHCHCH), 4.20 (1H, s, CH), 3.72 (1H, d, ²J_{H-H}=11.6, OCH₂), 3.48 (2H, m, NHCH₂), 3.32 (1H, d, ²J_{H-H}=11.6, OCH₂), 2.44 (2H, dt, ³J_{H-H}=6.5, ⁴J_{H-H}=2.6, NHCH₂CH₂), 2.03 (1H, t, ⁴J_{H-H}=2.6, CCH), 1.61 (3H, s, OCCH₃), 1.47 (3H, s, OCCH₃), 1.05 (6H, s, 2×CCH₃); ¹³C NMR (CDCl₃, 101 MHz) δ: 168.9 (NC═O), 167.7 (NC═O), 133.7 (NC═C), 100.2 (CH═CHCO), 99.3 (OC(CH₃)₂), 81.5 (CH₂CCH), 77.3 (OCH), 71.4 (OCH₂), 70.2 (CH₂CCH), 37.6 (NHCH₂), 33.3 (OCH₂C), 29.4 (OC(CH₃)₂), 21.9 (C(CH₃)₂), 19.5 (CH₂CCH), 19.1 (OC(CH₃)₂), 18.7 (C(CH₃)₂); IR vₘₐₓ(film)/cm⁻¹ (neat): (3294 (w), 2955 (w), 1656 (s), 1467 (s), 1249 (s); HRMS (ESI) calcd for C₁₆H₂₃N₂O₄ (M−H)⁻: m/z 307.1663 found m/z 307.1658; MP Range: 122-125 °C.
(R-149); [α]_{D}+20.6 (c=1.0, (CH₃)₂CO, T=24.4 °C).
(S-149); [α]_{D}−23.1 (c=1.4, (CH₃)₂CO, T=24.4 °C).

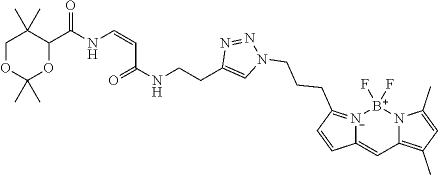

### (Z)—N-((((3-[4,4-Difluoro-5,7-dimethyl-4-bora-3a,4a-diaza-s-indacene-3-yl]propyl)-1H-1,2,3-triazol-4-yl)ethylamino)-3-oxoprop-1-enyl)-2,2,5,5-tetramethyl-1,3-dioxane-4-carboxamide (129,131)

(Z)—N-(3-(But-3-yn-1-ylamino)-3-oxoprop-1-en-1-yl)-2,2,5,5-tetramethyl-1,3-dioxane-4-carboxamide (R-149, S-149) (96 mg, 0.3 mmol) was dissolved in THF (1.5 mL) before 3-azido[4,4-difluoro-5,7-dimethyl-4-bora-3a,4a-diaza-s-indacene-3-yl]propane 148 (122 mg, 0.4 mmol) was added in one portion followed by DIPEA (1 drop) and catalytic amounts of CuI. The resulting mixture was heated to 70 °C for 16 h then concentrated in vacuo to give the crude as a dark brown oil. The crude was purified via column chromatography (5% MeOH/CH₂Cl₂) to give the triazole product (161 mg, 85%) as a red solid.

¹H NMR (CDCl₃, 500 MHz) δ: 11.68 (1H, d, ³J_{H-H}=11.1, NHCH), 7.39 (1H, s, ArCH_{triazole}), 7.23 (1H, dd, ³J_{H-H}=11.1, 8.9, NHCH═CH), 7.10 (1H, s, ArCH_{BODIPY-C8}), 6.88 (1H, d, ³J_{H-H}=3.9, ArCH_{BODIPY-C1}), 6.35 (1H, t, ³J_{H-H}=5.5, NHCH₂), 6.24 (1H, d, ³J_{H-H}=3.9, ArCH_{BODIPY-C2}), 6.12 (1H, s, ArCH_{BODIPY-C6}), 5.03 (1H, d, ³J_{H-H}=8.9, NHCH═CH), 4.42 (2H, t, ³J_{H-H}=6.9, ArNCH₂), 4.19 (1H, s, OCH), 3.71 (1H, d, ²J_{H-H}=11.7, OCH₂), 3.68-3.58 (2H, m, NHCH₂), 3.32 (1H, d, ²J_{H-H}=11.7, OCH₂), 2.97 (2H, t, ³J_{H-H}=7.5, ArC_{BODIPY-C3}CH₂), 2.92 (2H, t, ³J_{H-H}=6.2, NHCH₂CH₂), 2.54 (3H, s, ArC_{BODIPY-C5}CH₃), 2.37 (2H, quin, ³J_{H-H}=7.2, ArNCH₂CH₂), 2.26 (3H, s, ArC_{BODIPY-C7}CH₃), 1.60 (3H, s, OC(CH₃)₂), 1.46 (3H, s, OC(CH₃)₂), 1.04 (6H, s, 2×C(CH₃)₂); ¹³C NMR (CDCl₃, 126 MHz) δ: 168.7 (NC═O), 167.8 (NC═O), 160.6 (ArC_{q}), 156.5 (ArC_{q}), 145.4 (ArC_{q}), 144.2 (ArC_{q}), 135.3 (ArC_{q-triazole}), 133.1 (ArC_{q}), 133.0 (NC═C), 128.1 (ArCH), 123.8 (ArCH), 121.7 (ArCH_{triazole}), 120.6 (ArCH), 116.5 (ArCH), 100.8 (CH═CHCO), 99.2 (OC(CH₃)₂), 77.2 (OCH), 71.4 (OCH₂), 49.6 (ArNCH₂), 38.2 (NHCH₂), 33.3 (OCH₂C), 29.4 (OC(CH₃)₂), 29.2 (ArNCH₂CH₂), 25.5 (C_{BODIPY-C3}CH₂), 25.4 (NHCH₂CH₂), 22.0 (CH₂C(CH₃)₂), 19.0 (OC(CH₃)₂), 18.6 (CH₂C(CH₃)₂), 14.9 (C_{BODIPY-C5}CH₃), 11.3 (C_{BODIPY-C7}CH₃); IR vₘₐₓ(film)/cm⁻¹ (neat): 2926 (w), 2360 (w), 1660 (m), 1610 (s), 1139 (m); HRMS (ESI) calcd for C₃₀H₄₀N₇BF₂O₄Na [M+Na]⁺: m/z 633.3131, found m/z 633.3107; MP Range: 66-69 °C.
(R-129); [α]_{D}+3.8 (c=0.9, (CH₃)₂CO, T=24.4 °C).
(S-131); [α]_{D}−1.6 (c=1.0, (CH₃)₂CO, T=24.4 °C).

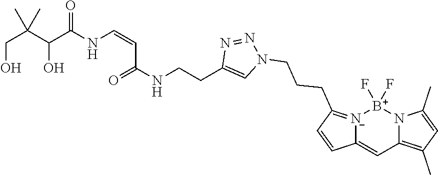

### (Z)—N-((((3-[4,4-Difluoro-5,7-dimethyl-4-bora-3a,4a-diaza-s-indacene-3-yl]propyl)-1H-1,2,3-triazol-4-yl)ethylamino)-3-oxoprop-1-enyl)-2,4-dihydroxy-3,3-dimethylbutanamide (128, 130)

(Z)—N-((((3-[4,4-Difluoro-5,7-dimethyl-4-bora-3a,4a-diaza-s-indacene-3-yl]propyl)-1H-1,2,3-triazol-4-yl)ethylamino)-3-oxoprop-1-enyl)-2,2,5,5-tetramethyl-1,3-dioxane-4-carboxamide (129,131) (60 mg, 0.1 mmol) was dissolved in MeCN (1.2 mL) and H₂O (0.1 mL) before BiCl₃ (6 mg, 0.02 mmol) was added. The mixture was stirred vigorously for 16 h. before filtering through celite and washing through with MeCN (40 mL). Removal of solvent in vacuo to give the crude diol as a dark brown oil. The crude was purified by flash column chromatography (5% MeOH/CH₂Cl₂) to give the diol as a red oil (52 mg, 92%).

¹H NMR (CDCl₃, 400 MHz) δ: 11.85 (1H, d, ³J_{H-H}=11.1, NHCH), 7.42 (1H, s, ArCH_{triazole}), 7.23 (1H, dd, ³J_{H-H}=11.1, 8.8, NHCH═CH), 7.10 (1H, s, ArCH_{BODIPY-C8}), 6.89 (1H, d, ³J_{H-H}=4.0, ArCH_{BODIPY-C1}), 6.55 (1H, t, ³J_{H-H}=5.5, NHCH₂), 6.24 (1H, d, ³J_{H-H}=4.0, ArCH_{BODIPY-C2}), 6.13 (1H, s, ArCH_{BODIPY-C6}), 5.05 (1H, d, ³J_{H-H}=8.8, NHCH═CH), 4.42 (2H, t, ³J_{H-H}=6.9, ArNCH₂), 4.16 (1H, s, OCH), 3.66-3.55 (2H, m, NHCH₂), 3.53 (1H, s, OCH₂), 2.96 (2H, t, ³J_{H-H}=7.5, ArCH_{BODIPY-C3}CH₂), 2.92 (2H, t, ³J_{H-H}=6.2, NHCH₂CH₂), 2.54 (3H, s, ArCH_{BODIPY-C5}), 2.35 (2H, quin, ³J_{H-H}=7.4, ArNCH₂CH₂), 2.26 (3H, s, ArCH_{BODIPY-C7}CH₃), 1.03 (3H, s, C(CH₃)₂), 0.98 (3H, s, C(CH₃)₂); ¹³C NMR (CDCl₃, 126 MHz) δ: 172.0 (NC═O), 168.1 (NC═O), 160.6 (ArC_{q}), 156.5 (ArC_{q}), 145.2 (ArC_{q}), 144.2 (ArC_{q}), 135.3 (ArC_{q-triazole}), 133.5 (ArC_{q}), 133.1 (NC═C), 128.2 (ArCH), 123.9 (ArCH), 121.8 (ArCH_{triazole}), 120.6 (ArCH), 116.5 (ArCH), 100.6 (CH═CHCO), 77.9 (OCH), 71.1 (OCH₂), 49.7 (ArNCH₂), 39.4 (NHCH₂), 38.4 (OCH₂C), 29.2 (ArNCH₂CH₂), 25.5 (C_{BODIPY-C3}CH₂), 25.4 (NHCH₂CH₂), 20.9 (CH₂C(CH₃)₂), 20.6 (CH₂C(CH₃)₂), 14.9 (C_{BODIPY-C5}CH₃), 11.3 (C_{BODIPY-C7}CH₃); IR vₘₐₓ(film)/cm⁻¹ (neat): 3309 (w), 2953 (w), 1654 (m), 1610 (s) 1139 (m); HRMS (ESI) calcd for C₂₇H₃₆N₇BF₂O₄Na [M+Na]⁺: m/z 593.2818, found m/z 593.2810
(128); [α]_{D}+15.3 (c=0.6, (CH₃)₂CO, T=24.4 °C).
(130); [α]_{D}−16.0 (c=1.0, (CH₃)₂CO, T=24.4 °C).

Further experimental details for compounds:

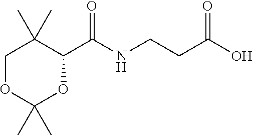 [SMILES: CC1(C)OCC(C)(C)[C@H](C(=O)NCCC(=O)O)O1]

(R)-3-(2,2,5,5-Tetramethyl-1,3-dioxane-4-carboxamido)propanoic acid, 150. A solution of D-pantothenic acid hemicalcium salt (500 mg, 1.0 mmol) in acetone (25 mL) was treated with p-TsOH·H₂O (560 mg, 3.0 mmol) and 1.0 g of 4 Å molecular sieves. The reaction was stirred at rt until completion by TLC analysis (18 h). The suspension was then filtered through a celite bed, washed with acetone (2×15 mL) and the organic phases combined. The combined organic washes were concentrated in vacuo before addition of EtOAc (30 mL) to the crude residue. The resulting solution was then washed with brine (2×30 mL), dried over Na₂SO₄ and concentrated under reduced pressure. Before total reduction of solvent, a few drops of hexane were added until crystallization was induced to afford the expected acetonide as white solid (300 mg, 55%), which required no further purification. The NMR data matches the literature data for this compound (see, e.g., Sewell et al., 2011; Gaudelli and Townsend, 2013).

¹H NMR (CDCl₃, 400 MHz) δ: 7.04 (1H, appt, NH), 4.12 (1H, s, CH), 3.71 (1H, d, J=11.6 Hz, CH₂Cq), 3.66-3.47 (2H, m, NHCH₂), 3.30 (1H, d, J=11.6 Hz, CH₂Cq), 2.65 (2H, t, J=6.0 Hz, CH₂CO₂H), 1.48 (3H, s, Cq(CH₃)₂), 1.45 (3H, s, Cq(CH₃)₂), 1.06 (3H, s, Cq(CH₃)₂), 1.00 (3H, s, Cq(CH₃)₂). ¹³C NMR (CDCl₃, 100 MHz) δ: 174.7 (CO), 170.1 (CO), 99.0 (Cq(CH₃)₂), 77.2 (OCH), 71.4 (OCH₂), 34.1 (CH₂Cq), 33.7 (HNCH₂), 32.9 (CH₂CO₂H), 29.4 (Cq(CH₃)₂), 22.0 (Cq(CH₃)₂), 18.8 (Cq(CH₃)₂), 18.7 (Cq(CH₃)₂).

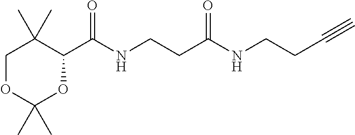 [SMILES: C#CCCNC(=O)CCNC(=O)[C@@H]1OC(C)(C)OCC1(C)C]

(R)—N-(3-(But-3-yn-1-ylamino)-3-oxopropyl)-2,2,5,5-tetramethyl-1,3-dioxane-4-carboxamide, R-151. A solution of (R)-3-(2,2,5,5-tetramethyl-1,3-dioxane-4-carboxamido)propanoic acid 150 (160 mg, 0.6 mmol) in CH₂Cl₂ (1.5 mL) was treated with HBTU (340 mg, 0.9 mmol), followed by 1-amino-3-butyne (70 μL, 0.9 mmol) and N,N-diisopropylethylamine (150 μL, 0.9 mmol). The reaction mixture was then heated to 80 °C for 3.5 h in the microwave oven. The reaction was then concentrated under reduced pressure. Purification of the crude residue by flash column chromatography (0-5% MeOH/CH₂Cl₂) gave the desired alkyne as a red oil (175 mg, 90%).

¹H NMR (CDCl₃, 400 MHz) δ: 7.05 (1H, br s, NH), 6.24 (1H, br s, NH), 4.10 (1H, s, CH), 3.71 (1H, d, J=12.0 Hz, CH₂Cq), 3.65-3.50 (2H, m, CH₂NH), 3.30 (1H, d, J=12.0 Hz, CH₂Cq), 3.24-3.17 (2H, m, CH₂CO), 2.50 (2H, t, J=8.0 Hz, NHCH₂), 2.42 (2H, td, J=6.4 Hz, 2.4 Hz, CH₂Cq), 2.03 (1H, t, J=2.4 Hz, CqCH), 1.48 (3H, s, Cq(CH₃)₂), 1.46 (3H, s, Cq(CH₃)₂), 1.06 (3H, s, Cq(CH₃)₂), 0.99 (3H, s, Cq(CH₃)₂). ¹³C NMR (CDCl₃, 100 MHz) δ: 171.4 (CO), 170.5 (CO), 99.1 (Cq(CH₃)₂), 88.1 (CqCH), 77.2 (OCH), 71.4 (OCH₂), 70.2 (CqCH), 38.1 (HNCH₂), 35.9 (CH₂CO), 34.8 (NHCH₂), 33.0 (CH₂Cq), 29.4 (Cq(CH₃)₂), 22.1 (Cq(CH₃)₂), 18.9 (Cq(CH₃)₂), 18.6 (Cq(CH₃)₂). HRMS (ESI) calculated for C₁₆H₂₆N₂O₄ [M+Na]⁺: m/z 333.1785, found m/z 333.1765. IR vₘₐₓ (film)/cm⁻¹: 3293 (m), 2989 (m), 1740 (s), 1650 (m), 1536 (m), 1368 (m), 1232 (s), 1090 (s). [α]_{D}+9.500 (c=0.5, CHCl₃, T=23.0 °C).

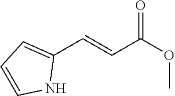

(E)-Methyl-3-(1H-pyrrol-2-yl)acrylate, 152. A suspension of 1H-pyrrole-2-carboxaldehyde (2.0 g, 21.0 mmol) in benzene (160 mL) was treated with methyl (triphenylphosphoranylidene)acetate (10.9 g, 32.5 mmol). The resulting mixture was heated to 80 °C until completion by TLC analysis (18 h). The reaction mixture was cooled down to rt and concentrated in vacuo to give a crude orange oil. Purification of the crude residue by flash column chromatography (0-20% EtOAc/PE) gave the desired vinylogous ester as a white solid (2.33 g, 73%). The NMR data matches the literature data for this compound (see, e.g., Hansen et al., 2013).

¹H NMR (CDCl₃, 400 MHz) δ: 8.93 (1H, br s, NH), 7.58 (1H, d, J=16.0 Hz, CqCH), 6.97-6.94 (1H, m, CH_{pyrrole}), 6.60-6.57 (1H, m, CH_{pyrrole}), 6.32-6.29 (1H, m, CH_{pyrrole}), 6.06 (1H, d, J=16.0 Hz, CHCO), 3.80 (3H, s, OCH₃). ¹³C NMR (CDCl₃, 100 MHz) δ: 168.0 (CO), 134.3 (CqCH), 128.3 (ArCq), 122.4 (CHCO), 114.5 (ArCH), 111.0 (ArCH), 110.8 (ArCH), 51.5 (OCH₃).

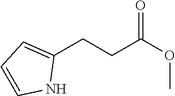

Methyl 3-(1H-pyrrol-2-yl)propanoate, 153. A solution of (E)-methyl-3-(1H-pyrrol-2-yl)acrylate 152 (2.33 g, 15.4 mmol) in anhydrous MeOH (100 mL) was stirred for 5 minutes under an argon atmosphere. Pd/C (10%, 240 mg, 14 mol %) was added before replacing the argon with a hydrogen atmosphere, and stirred at rt until completion by TLC analysis (18 h). The reaction was then filtered through a celite bed, and washed with MeOH (2×25 mL). The organic phases were combined and concentrated in vacuo to afford the desired ester as a brown oil (2.2 g, 93%), which required no further purification. The NMR data matches the literature data for this compound (see, e.g., Hansen et al., 2013).

¹H NMR (CDCl₃, 400 MHz) δ: 8.54 (1H, br s, NH), 6.71-6.69 (1H, m, ArCH_{pyrrole}), 6.14-6.11 (1H, m, ArCH_{pyrrole}), 5.96-5.93 (1H, m, ArCH_{pyrrole}), 3.72 (3H, s, OCH₃), 2.94 (2H, t, J=8.0 Hz, CqCH₂), 2.67 (2H, t, J=8.0 Hz, CH₂CO). ¹³C NMR (CDCl₃, 100 MHz) δ: 174.5 (CO), 130.9 (ArCq), 116.8 (ArCH), 108.0 (ArCH), 105.5 (ArCH), 51.8 (OCH₃), 34.3 (CqCH₂), 22.5 (CH₂CO).

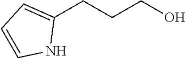

3-(1H-Pyrrol-2-yl)propan-1-ol, 154. A 0 °C solution of methyl 3-(1H-pyrrol-2-yl)propanoate 153 (2.1 g, 13.7 mmol) in Et₂O(110 mL) was stirred for 15 minutes before being treated slowly with LiAlH₄ (1.0 g, 26.3 mmol). The solution was allowed to warm up to rt until completion by TLC analysis (16 h). The reaction was quenched by the dropwise addition of 1 M NaOH solution until pH neutral. The solvent was decanted off, and the lithium/aluminium salts were washed with Et₂O (2×100 mL). The combined organic washes were dried over Na₂SO₄, and concentrated in vacuo to afford the expected alcohol as a brown oil (1.7 g, 100%), which required no further purification. The NMR data matches the literature data for this compound (see, e.g., Hansen et al., 2013).

¹H NMR (CDCl₃, 400 MHz) δ: 8.16 (1H, br s, NH), 6.72-6.68 (1H, m, ArCH_{pyrrole}), 6.18-6.13 (1H, m, ArCH_{pyrrole}), 5.98-5.94 (1H, m, ArCH_{pyrrole}), 3.74 (2H, t, J=6.0 Hz, CH₂OH), 2.76 (2H, t, J=8.0 Hz, CqCH₂), 2.00-1.80 (2H, m, CH₂CH₂CH₂). ¹³C NMR (CDCl₃, 100 MHz) δ: 130.9 (ArCq), 116.4 (ArCH), 108.4 (ArCH), 105.3 (ArCH), 63.7 (CH₂OH), 28.9 (CqCH₂), 24.0 (CH₂CH₂CH₂).

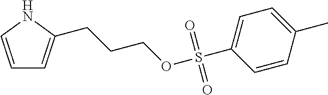 [SMILES: Cc1ccc(S(=O)(=O)OCCCc2ccc[nH]2)cc1]

3-(1H-Pyrrol-2-yl)propyl 4-methylbenzenesulfonate, 155. A 0 °C solution of 3-(1H-pyrrol-2-yl)propan-1-ol 154 (1.2 g, 10.0 mol) and p-TsCl (2.9 g, 15.0 mol) in CH₂Cl₂ (75 mL) was treated with triethylamine (2.8 mL, 20.0 mol). The resultant mixture was then stirred at rt until completion by TLC analysis (2 h). The reaction was then washed with 1 M HCl (3×75 mL), NaHCO₃ saturated solution (2×75 mL) and brine (75 mL). The organic phase was dried over Na₂SO₄, and concentrated in vacuo to afford the expected product as a brown oil (1.66 g, 60%). The crude product was used without further purification.

¹H NMR (CDCl₃, 400 MHz) δ: 8.05 (1H, br s, NH), 7.81 (2H, d, J=8.0 Hz, ArCH_{benzene}), 7.37 (2H, d, J=7.6 Hz, ArCH_{benzene}), 6.69-6.65 (1H, m, ArCH_{pyrrole}), 6.14-6.10 (1H, m, ArCH_{pyrrole}), 5.88-5.84 (1H, m, ArCH_{pyrrole}), 4.08 (2H, t, J=6.0 Hz, CH₂O), 2.71 (2H, t, J=7.0 Hz, CqCH₂), 2.48 (3H, s, CH₃), 2.01-1.92 (2H, m, CH₂). ¹³C NMR (CDCl₃, 100 MHz) δ: 144.8 (ArCq), 133.0 (ArCq), 130.3 (ArCq), 129.9 (2×ArCH), 127.9 (2×ArCH), 116.6 (ArCH), 108.4 (ArCH), 105.5 (ArCH), 69.5 (CH₂O), 29.3 (CqCH₂), 23.3 (CH₂CH₂CH₂), 21.6 (CqCH₃). HRMS (ESI) calculated for C₁₄H₁₇NO₃S [M+(CH₃CN+NH₄)⁺]⁺: m/z 338.1532, found m/z 338.3412. IR vₘₐₓ (film)/cm⁻¹: 3362 (br), 2920 (m), 2848 (m), 1645 (s), 1469 (m), 1025 (s).

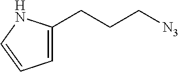 [SMILES: [N-]=[N+]=NCCCc1ccc[nH]1]

2-(3-Azidopropyl)-1H-pyrrole, 156. A solution of 3-(1H-pyrrol-2-yl)propyl 4-methylbenzenesulfonate 155 (1.0 g, 3.6 mmol) in DMF (30 mL) was treated with NaN₃ (980 mg, 15.0 mmol), and the resultant mixture was heated to 70 °C until completion by TLC analysis (16 h). The reaction was then cooled down to rt, and diluted with EtOAc (60 mL) followed by H₂O (30 mL). The phases were separated, and the aqueous layer was extracted with EtOAc (2×60 mL). The combined organic layers were washed with brine (5×100 mL), dried over Na₂SO₄ and concentrated under reduced pressure. The crude residue was purified by flash column chromatography (0-20% EtOAc/PE) to afford the desired azide as a yellow oil (370 mg, 68%). The NMR data matches the literature data for this compound (see, e.g., Hansen et al., 2013).

¹H NMR (CDCl₃, 400 MHz) δ: 7.98 (1H, br s, NH), 6.73-6.69 (1H, m, ArCH_{pyrrole}), 6.19-6.14 (1H, m, ArCH_{pyrrole}), 5.98-5.94 (1H, m, ArCH_{pyrrole}), 3.35 (2H, t, J=6.8 Hz, CH₂N₃), 2.74 (2H, t, J=7.6 Hz, CqCH₂), 1.98-1.89 (2H, m, CH₂CH₂CH₂). ¹³C NMR (CDCl₃, 100 MHz) δ: 130.2 (ArCq), 127.5 (ArCH), 108.5 (ArCH), 105.5 (ArCH), 50.6 (CH₂N₃), 28.8 (CqCH₂), 24.6 (CH₂CH₂CH₂).

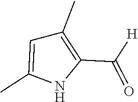

3,5-Dimethyl-1H-pyrrole-2-carbaldehyde, 157. Anhydrous DMF (20 mL) was cooled down to 0 °C and treated with POCl₃ (1.0 mL, 11.0 mmol). The solution was stirred at 0 °C for 5 min., and then at rt for 30 min. The reaction mixture was cooled back down to 0 °C, and treated with 2,4-dimethyl-1H-pyrrole (1.0 mL, 10.0 mmol). The reaction was then heated to 40 °C until completion by TLC analysis (18 h). The mixture was cooled down to rt, and diluted with EtOAc (40 mL) followed by H₂O (20 mL). The phases were separated, and the aqueous layer was extracted with EtOAc (2×40 mL). The combined organic layers were washed with H₂O (5×100 mL), brine (2×100 mL) and dried over Na₂SO₄. The resulting solution was concentrated under reduced pressure, and the crude residue was purified by flash column chromatography (0-50% Et₂O/PE) to afford the desired aldehyde as a white solid (540 mg, 45%). The NMR data matches the literature data for this compound (see, e.g., Hansen et al., 2013).

¹H NMR (CDCl₃, 400 MHz) δ: 9.93 (1H, br, NH), 9.49 (1H, s, CHO), 5.88 (1H, s, ArCH_{pyrrole}), 2.36 (3H, s, CH₃), 2.33 (3H, s, CH₃). ¹³C NMR (CDCl₃, 100 MHz) δ: 175.8 (CO), 138.3 (ArCq), 134.6 (ArCq), 128.7 (ArCq), 112.0 (ArCH), 13.1 (CH₃Cq), 10.5 (CqCH₃).

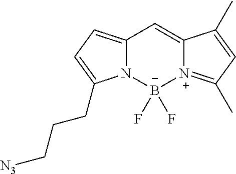 [SMILES: CC1=CC(C)=[N+]2C1=Cc1ccc(CCCN=[N+]=[N-])n1[B-]2(F)F]

3-Azido[4,4-difluoro-5,7-dimethyl-4-bora-3a,4a-diaza-s-indacene-3-yl]propane, 148. A 0 °C solution of 2-(3-azidopropyl)-1H-pyrrole 156 (270 mg, 1.8 mmol) in CH₂Cl₂ (10 mL), was treated with a solution of 3,5-dimethyl-1H-pyrrole-2-carbaldehyde 157 (203 mg, 1.6 mmol) in CH₂Cl₂ (2 mL). The solution was then treated with the dropwise addition of POCl₃ (100 μL, 1.0 mmol), and the mixture was stirred at rt for 6.5 h before being cooled back down to 0 °C. The reaction mixture was then treated sequentially with BF₃·Et₂O (0.5 mL, 4.0 mmol) followed by N,N-diisopropylethylamine (0.7 mL, 4.0 mmol). The resulting reaction mixture was stirred at rt until completion by TLC analysis (18 h). The reaction was then quenched with H₂O (15 mL), diluted with CH₂Cl₂ (5 mL) and filtered through a bed of celite. The celite was washed thoroughly with CH₂Cl₂ (2×10 mL), and the organic phases combined. The combined organic washes were dried over Na₂SO₄, and concentrated in vacuo to give the crude residue as a red oil. Purification of the crude residue by flash column chromatography (0-20% EtOAc/PE) yielded the expected azide as a red oil (290 mg, 59%). The NMR data matches the literature data for this compound (see, e.g., Hansen et al., 2013).

¹H NMR (CDCl₃, 400 MHz) δ: 7.11 (1H, s, ArCH_{BODIPY-C8}), 6.94 (1H, d, J=4.0 Hz, ArCH_{BODIPY-C1}), 6.31 (1H, d, J=4.0 Hz, ArCH_{BODIPY-C2}), 6.14 (1H, s, ArCH_{BODIPY-C6}), 3.41 (2H, t, J=8.0 Hz CH₂N₃), 3.07 (2H, t, J=8.0 Hz CqCH₂), 2.59 (3H, s, CH₃), 2.28 (3H, s, CH₃), 2.06 (2H, m, CH₂CH₂N₃).

¹³C NMR (CDCl₃, 100 MHz) δ: 160.5 (ArCq), 156.5 (ArCq), 146.7 (ArCq), 143.7 (ArCq), 134.1 (ArCq), 128.3 (ArCH), 123.8 (ArCH), 120.2 (ArCH), 116.6 (ArCH), 50.9 (CH₂N₃), 28.2 (CH₂CH₂CH₂), 25.8 (CqCH₂), 14.9 (CH₃Cq), 11.4 (CH₃Cq).

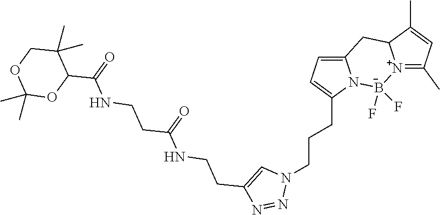

N-((((3-[4,4-Difluoro-5,7-dimethyl-4-bora-3a,4a-diaza-s-indacene-3-yl]propyl)-1H-1,2,3-triazol-4-yl)ethylamino)-3-oxopropyl)-2,2,5,5-tetramethyl-1,3-dioxane-4-carboxamide, 133, 135. A solution of 3-azido[4,4-difluoro-5,7-dimethyl-4-bora-3a,4a-diaza-s-indacene-3-yl]propane 148 (40 mg, 0.1 mmol) in THF (2.5 mL) was treated with a solution of N-(3-(but-3-yn-1-ylamino)-3-oxopropyl)-2,2,5,5-tetramethyl-1,3-dioxane-4-carboxamide (R-151, S-151) (43 mg, 0.1 mmol) in THF (2.5 mL). The resulting mixture was treated with a catalytic amount of CuI (5 mg) followed by N,N-diisopropylethylamine (0.1 mL, 0.6 mmol). The reaction was then heated to 70 °C until completion by TLC analysis (18 h). The reaction mixture was cooled down to rt, and diluted with H₂O (5 mL). The aqueous phase was extracted with EtOAc (3×10 mL) and the combined organics were washed with brine (3×30 mL), dried over MgSO₄ and concentrated under reduced pressure. Purification of the crude residue by flash column chromatography (0-5% MeOH/CH₂Cl₂) afforded the expected triazole as a red oil (74 mg, 87%).

¹H NMR (CDCl₃, 400 MHz) δ: 7.45 (1H, s, ArCH_{Triazole}), 7.13 (1H, s, ArCH_{BODIPY-C8}), 6.91 (1H, d, J=4.0 Hz, ArCH_{BODIPY-C1}), 6.61 (1H, t, J=5.0 Hz, CONH), 6.28 (1H, d, J=4.0 Hz, ArCH_{BODIPY-C2}), 6.15 (1H, s, ArCH_{BODIPY-C6}), 4.44 (2H, t, J=8.0 Hz, CH₂N), 4.07 (1H, s, CH), 3.70 (1H, d, J=12.0 Hz, CH₂Cq), 3.62-3.56 (2H, m, NHCH₂), 3.54-3.45 (2H, m, NHCH₂), 3.28 (1H, d, J=12.0 Hz, CH₂Cq), 3.01 (2H, t, J=8.0 Hz, CH₂Cq), 2.90 (2H, t, J=6.4 Hz, CH₂CO), 2.56 (3H, s, CH₃), 2.43 (2H, t, J=6.0 Hz, CH₂Cq), 2.39-2.33 (2H, m, CH₂CH₂CH₂), 2.28 (3H, s, CH₃), 1.46 (3H, s, Cq(CH₃)₂), 1.44 (3H, s, Cq(CH₃)₂), 1.03 (3H, s, Cq(CH₃)₂), 0.96 (3H, s, Cq(CH₃)₂). ¹³C NMR (CDCl₃, 100 MHz) δ: 171.2 (CO), 169.9 (CO), 160.5 (ArCq), 156.5 (ArCq), 145.2 (ArCq), 135.0 (ArCq), 133.1 (ArCq), 132.0 (ArCq), 128.8 (ArCH), 123.9 (ArCH), 121.9 (ArCH), 120.6 (ArCH), 116.5 (ArCH), 99.1 (Cq(CH₃)₂), 77.1 (OCH), 71.4 (OCH₂), 49.7 (NCH₂), 38.7 (HNCH₂), 35.9 (CH₂CO), 34.8 (NHCH₂), 32.9 (CH₂Cq), 29.4 (Cq(CH₃)₂), 29.3 (CH₂CH₂CH₂), 25.8 (CH₂ArCq), 25.4 (CH₂Cq), 22.1 (Cq(CH₃)₂), 18.9 (Cq(CH₃)₂), 18.6 (Cq(CH₃)₂), 14.9 (CH₃Cq), 11.3 (CH₃Cq). ¹⁹F NMR (CDCl₃, 376 MHz) δ: −70.1, −72.0. HRMS (ESI) calculated for C₂₉H₄₄BF₂N₇O₄ [M+H+Na]⁺: m/z 637.3252, found m/z 637.3214. IR vₘₐₓ(film)/cm⁻¹: 3357 (br), 2922 (m), 2850 (m), 1740 (s), 1650 (m).
133, (R): [α]_{D}+15.500 (c=0.1, CHCl₃, T=23.0 °C).
135, (S): [α]_{D}−21.33 (c=0.075, CHCl₃, T=23.1 °C).

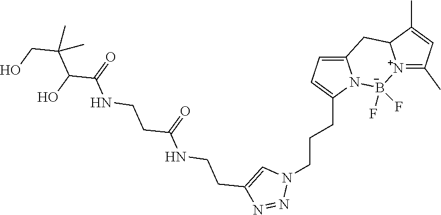 [SMILES: CC1=CC(C)=[N+]2C1Cc1ccc(CCCn3cc(CCNC(=O)CCNC(=O)C(O)C(C)(C)CO)nn3)n1[B-]2(F)F]

N-((((3-[4,4-Difluoro-5,7-dimethyl-4-bora-3a,4a-diaza-s-indacene-3-yl]propyl)-1H-1,2,3-triazol-4-yl)ethylamino)-3-oxopropyl)-2,4-dihydroxy-3,3-dimethylbutanamide, 132, 134. A solution of N-((((3-[4,4-Difluoro-5,7-dimethyl-4-bora-3a,4a-diaza-s-indacene-3-yl]propyl)-1H-1,2,3-triazol-4-yl)ethylamino)-3-oxopropyl)-2,2,5,5-tetramethyl-1,3-dioxane-4-carboxamide (133, 135) (40 mg, 60 μmol) in CH₃CN (3 mL) was treated with a catalytic amount of BiCl₃ (5 mg) followed by H₂O (0.1 mL). The reaction mixture was stirred at rt until completion by TLC analysis (17 h). The reaction was then quenched by the addition of five drops of sat. aq. NaHCO₃, and diluted with EtOAc (5 mL). The resulting suspension was filtered through a bed of celite, and the celite was washed with EtOAc (2×5 mL). The organic phases were combined, dried over Na₂SO₄, and concentrated under reduced pressure. Purification of the crude residue by flash column chromatography (0-10% 2 M NH₃ in MeOH/CH₂Cl₂) afforded the expected diol as a red oil (10 mg, 28%).

¹H NMR (CD₃OD, 400 MHz) δ: 8.57 (1H, s, NH), 7.45 (1H, s, ArCH_{Triazole}), 7.06 (1H, s, ArCH_{BODIPY-C8}), 7.00 (1H, d, J=4.0 Hz, ArCH_{BODIPY-C1}), 6.71 (1H, br s, CONH), 6.43 (1H, d, J=4.0 Hz, ArCH_{BODIPY-C2}), 6.24 (1H, s, ArCH_{BODIPY-C6}), 4.51 (2H, t, J=8.0 Hz, NCH₂), 4.09 (1H, s, CH), 3.69 (1H, d, J=12.0 Hz, CH₂Cq), 3.63-3.59 (2H, m, NHCH₂), 3.56-3.53 (2H, m, NHCH₂), 3.15 (1H, d, J=12.0 Hz, CH₂Cq), 3.00 (2H, t, J=8.0 Hz, CH₂Cq), 2.92 (2H, t, J=6.4 Hz, CH₂CO), 2.51 (3H, s, CqCH₃), 2.31 (2H, t, J=6.0 Hz, CH₂Cq), 2.20-2.12 (2H, m, CH₂CH₂CH₂), 2.07 (3H, s, CqCH₃), 1.04 (3H, s, Cq(CH₃)₂), 0.74 (3H, s, Cq(CH₃)₂). ¹³C NMR (CD₃OD, 100 MHz) δ: 179.0 (CO), 167.8 (CO), 160.3 (ArCq), 155.8 (ArCq), 145.4 (ArCq), 134.2 (ArCq), 132.4 (ArCq), 130.8 (ArCq), 129.6 (ArCH), 124.3 (ArCH), 122.5 (ArCH), 120.9 (ArCH), 116.2 (ArCH), 100.5 (Cq(CH₃)₂), 77.4 (OCH), 71.8 (OCH₂), 50.1 (NCH₂), 38.7 (HNCH₂), 35.9 (CH₂CO), 35.1 (NHCH₂), 31.6 (CH₂Cq), 29.0 (CH₂CH₂CH₂), 25.5 (CH₂Cq), 25.4 (CH₂Cq), 18.7 (Cq(CH₃)₂), 18.4 (Cq(CH₃)₂), 13.0 (CH₃Cq), 11.4 (CH₃Cq). ¹⁹F NMR (CD₃OD, 376 MHz) δ: −74.1, −76.0. HRMS (ESI) calculated for C₂₇H₃₈BF₂N₇O₄ [M]⁺: m/z 573.3046, found m/z 573.3890. IR vₘₐₓ(film)/cm⁻¹: 3491 (br), 2927 (m), 1843 (m), 1644 (m), 1557 (m), 1411 (s), 1258 (s).
132, (R): [α]_{D}+26.000 (c=0.02, CHCl₃, T=23.0 °C).
134, (S): [α]_{D}−2.670 (c=0.15, MeOH, T=23.0 °C).

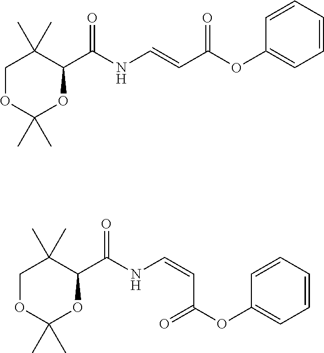 [SMILES: CC1(C)OCC(C)(C)[C@@H](C(=O)N/C=C/C(=O)Oc2ccccc2)O1 ; CC1(C)OCC(C)(C)[C@@H](C(=O)N/C=C\C(=O)Oc2ccccc2)O1]

(S,E)-Benzyl-3-(2,2,5,5-tetramethyl-1,3-dioxane-4-carboxamido)acrylate, 158 and (S,Z)-Benzyl 3-(2,2,5,5-tetramethyl-1,3-dioxane-4-carboxamido)acrylate, 159. A solution of N-Formyl-(S)-2,2,5,5-tetramethyl-[1,3]dioxane-4-carboxylic acid amide, S-141 (180 mg, 0.82 mmol) in benzene (6 mL) was treated with benzyltriphenylphosphoranylidene (800 mg, 1.95 mmol) and the resulting mixture heated to 80 °C for 18 h. Upon reaction completion as indicated by TLC analysis, the solvent was removed under vacuum. Purification of the crude residue by flash column chromatography (0-30% EtOAc/PE) afforded the desired enamides 158 (200 mg) as a white solid and 159 (70 mg) as a colorless oil, in quantitative yield (97%) (see, e.g., Sewell et al., 2011).

(S,Z)-Benzyl 3-(2,2,5,5-tetramethyl-1,3-dioxane-4-carboxamido)acrylate, 159. ¹H NMR (CDCl₃, 400 MHz) δ: 11.14 (1H, d, J=11.3 Hz, NH), 7.36 (1H, dd, J=11.6, 8.8 Hz, NCH), 7.35-7.30 (5H, m, PhH), 5.21 (1H, d, J=8.8 Hz, CHCO₂), 4.20 (1H, s, CH), 3.72 (1H, d, J=12.0 Hz, CH₂Cq), 3.32 (1H, d, J=12.0 Hz, CH₂Cq), 1.57 (3H, s, Cq(CH₃)₂), 1.45 (3H, s, Cq(CH₃)₂), 1.03 (6H, s, Cq(CH₃)₂). [α]_{D}−30.91 (c=0.44, CHCl₃, T=22.1 °C).

(S,E)-Benzyl-3-(2,2,5,5-tetramethyl-1,3-dioxane-4-carboxamido)acrylate, 158. ¹H NMR (CDCl₃, 500 MHz) δ: 8.44 (1H, d, J=12.1 Hz, NH), 8.04 (1H, dd, J=14.5, 12.1 Hz, NCH), 7.37 (5H, m, PhH), 5.68 (1H, d, J=14.5 Hz, CHCO₂), 4.20 (1H, s, OCH), 3.72 (1H, d, J=12.0 Hz, CH₂Cq), 3.32 (1H, d, J=11.5 Hz, CH₂Cq), 1.52 (3H, s, Cq(CH₃)₂), 1.46 (3H, s, Cq(CH₃)₂), 1.06 (3H, s, Cq(CH₃)₂), 1.01 (3H, s, Cq(CH₃)₂). ¹³C NMR (CDCl₃, 125 MHz) δ: 167.9 (CO), 166.9 (CO), 136.4 (NCH), 136.2 (PhH), 128.5 (PhH), 128.2 (PhH), 128.1 (PhH), 102.7 (CHCO₂), 99.6 (Cq(CH₃)₂), 77.2 (OCH), 71.3 (OCH₂), 66.0 (OCH₂), 33.4 (CH₂Cq), 29.5 (Cq(CH₃)₂), 21.9 (Cq(CH₃)₂), 18.8 (Cq(CH₃)₂), 18.7 (Cq(CH₃)₂). [α]_{D}−57.3 (c=0.75, CHCl₃, T=22.0 °C).

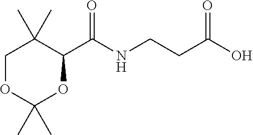 [SMILES: CC1(C)OCC(C)(C)[C@@H](C(=O)NCCC(=O)O)O1]

(S)-3-(2,2,5,5-Tetramethyl-1,3-dioxane-4-carboxamido)propanoic acid, 160. A solution of (S,E)-benzyl 3-(2,2,5,5-tetramethyl-1,3-dioxane-4-carboxamido)acrylate 158 (170 mg, 4.8 mmol) in MeOH (2 mL) and EtOH (1 mL) was treated with Pd/C (10% wt/wt). The heterogenous mixture was then stirred under a H₂ atmosphere for 24 h. Upon reaction completion as indicated by TLC analysis, the solvent was removed under vacuum. The crude colourless oil (127 mg) was found by NMR to be the clean, desired acetonide (96% yield) which required no further purification. The NMR data matches the literature data for this compound (see, e.g., Sewell et al., 2011; Gaudelli and Townsend, 2013).

¹H NMR (CDCl₃, 600 MHz) δ: 7.01 (1H, br s, NH), 4.08 (1H, s, CH), 3.68 (1H, d, J=11.4 Hz, CH₂Cq), 3.61-3.55 (2H, m, NHCH₂), 3.27 (1H, d, J=11.4 Hz, CH₂Cq), 2.60 (2H, appt, CH₂CO₂H), 1.44 (3H, s, Cq(CH₃)₂), 1.41 (3H, s, Cq(CH₃)₂), 1.02 (3H, s, Cq(CH₃)₂), 0.96 (3H, s, Cq(CH₃)₂). ¹³C NMR (CDCl₃, 150 MHz) δ: 175.6 (CO), 170.2 (CO), 99.0 (Cq(CH₃)₂), 77.1 (OCH), 71.4 (OCH₂), 34.1 (CH₂Cq), 33.8 (HNCH₂), 33.0 (CH₂CO₂H), 29.5 (Cq(CH₃)₂), 22.1 (Cq(CH₃)₂), 18.8 (Cq(CH₃)₂), 18.7 (Cq(CH₃)₂).

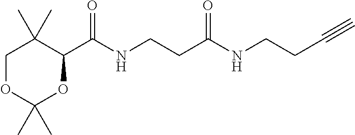 [SMILES: C#CCCNC(=O)CCNC(=O)[C@H]1OC(C)(C)OCC1(C)C]

(S)—N-(3-(But-3-yn-1-ylamino)-3-oxopropyl)-2,2,5,5-tetramethyl-1,3-dioxane-4-carboxamide, S-151. A solution of (S)-3-(2,2,5,5-tetramethyl-1,3-dioxane-4-carboxamido)propanoic acid 160 (127 mg, 0.5 mmol) in CH₂Cl₂ (1.5 mL) was treated with HBTU (200 mg, 0.5 mmol), followed by 1-amino-3-butyne (50 μL, 0.6 mmol) and N,N-diisopropylethylamine (100 μL, 0.6 mmol). The reaction mixture was then heated to 80 °C for 2.5 h in the microwave oven. The reaction was then concentrated under reduced pressure. Purification of the crude residue by flash column chromatography (0-10% MeOH/CH₂Cl₂) gave the desired alkyne as a brown oil with HBTU side product traces (144 mg, 94%).

¹H NMR (CDCl₃, 400 MHz) δ: 7.01 (1H, br s, NH), 6.40 (1H, br s, NH), 4.06 (1H, s, CH), 3.71 (1H, d, J=11.2 Hz, CH₂Cq), 3.60-3.50 (2H, m, CH₂NH), 3.25 (1H, d, J=11.6 Hz, CH₂Cq), 3.19-3.12 (2H, m, CH₂CO), 2.56-2.53 (2H, m, NHCH₂), 2.48 (2H, td, J=6.0 Hz, 2.0 Hz, CH₂Cq), 1.98 (1H, t, J=2.4 Hz, CqCH), 1.45 (3H, s, Cq(CH₃)₂), 1.40 (3H, s, Cq(CH₃)₂), 1.01 (3H, s, Cq(CH₃)₂), 0.95 (3H, s, Cq(CH₃)₂).

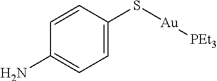

### (4-Aminobenzenethiolato)(triethylphosphine) Gold(I), 161

A 0 °C solution of 4-aminothiophenol (22 mg, 0.17 mmol) in MeOH (2 mL) was treated with 200 μL of freshly prepared NaOMe aqueous solution (0.6 M) and stirred for 10 min. Reaction mixture was then treated with dropwise addition of suspension of chloro(triphosphine)gold(I) (50 mg, 0.14 mmol) in 1 mL (CH₂Cl₂:MeOH, 1:1) and stirred for 1.5 h. After confirmation of completion by TLC analysis, the solvent was removed under reduced pressure. The crude oil was resuspended with H₂O (5 mL) and extracted with CH₂Cl₂ (5 mL, 5×). Combined organics were washed with brine (25 mL), dried over Na₂SO₄, filtered and solvent was removed under reduced pressure to afford the expected product as a white solid (22 mg, 35%). No further purification was required for the next reaction. The NMR data matches the literature data for this compound (see, e.g., Wu et al., 2019).

¹H NMR (CDCl₃, 400 MHz) δ: 7.30-7.25 (2H, m, ArH), 6.62-6.48 (2H, m, J=4.0 Hz, ArH)), 3.45 (2H, br s, NH₂), 1.81 (6H, dq, J=9.6, 6.8 Hz P(CH₂CH₃)₃), 1.18 (9H, dt, J=11.4, 7.6 Hz, P(CH₂CH₃)₃).

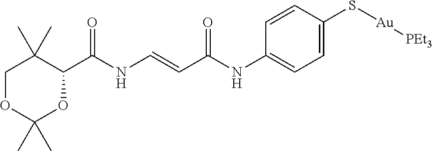

(R,E)-((4-(3-(2,2,5,5-tetramethyl-1,3-dioxane-4-carboxamido)acrylamido)phenyl)thio) (triethyl-phosphaneyl) Gold (I), 136-Aur. A suspension of (R,E)-3-(2,2,5,5-Tetramethyl-1,3-dioxane-4-carboxamido)acrylic acid, 145 (20 mg, 0.08 mmol) in dry CH₂Cl₂ (300 μL) was treated sequentially with HBTU (29 mg, 0.08 mmol) and DIPEA (15 μL, 0.08 mmol), and stirred at room temperature for 5 minutes. A solution of 4-Aminobenzenethiolato)(triethylphosphine) Gold(I), 161 (20 mg, 0.05 mmol) in dry CH₂Cl₂ (600 μL) was then added to the reaction mixture and stirred at room temperature for 72 h. Solvent was removed under reduced pressure and purification of the crude residue by flash column chromatography (0-50% Ethyl Acetate/Hexane) afforded the desired product as a yellow oil (2 mg, 7%).

¹H NMR (CDCl₃, 400 MHz) δ: 8.39 (1H, d, J=12.0 Hz, NH), 8.00 (1H, m, NHCH), 7.20-7.17 (2H, m, ArH), 6.69-6.66 (2H, m, ArH)), 6.04 (1H, d, J=14.0 Hz, CHCO₂), 4.19 (1H, s, OCH), 3.69 (1H, d, J=12.0 Hz, CH₂Cq), 3.30 (1H, d, J=12.0 Hz, CH₂Cq), 1.58-1.55 (6H, m, P(CH₂CH₃)₃), 1.50 (3H, s, Cq(CH₃)₂), 1.44 (3H, s, Cq(CH₃)₂), 1.23 (9H, m, P(CH₂CH₃)₃), 1.03 (3H, s, Cq(CH₃)₂), 0.99 (3H, s, Cq(CH₃)₂).

HRMS (ESI) calculated for C₂₄H₃₉AuN₂O₄PS [M+H]⁺: m/z 679.2028, found m z 679.2009.

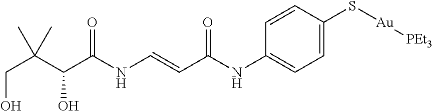

(R,E)-((4-(3-(2,4-dihydroxy-3,3-dimethylbutanamido)acrylamido)phenyl)thio)(triethyl-phosphaneyl) Gold (I), 137-Aur. A suspension of (R,E)-3-(2,4-Dihydroxy-3,3-dimethylbutanamido)acrylic acid, 138 (50 mg, 0.23 mmol) in dry DMF (2 mL) was treated sequentially with HBTU (50 mg, 0.14 mmol) and DIPEA (100 μL, 0.53 mmol), and stirred at room temperature for 5 minutes. A solution of 4-Aminobenzenethiolato)(triethylphosphine) Gold(I), 161 (36 mg, 0.09 mmol) in dry DMF (1.5 mL) was then added to the reaction mixture and stirred at room temperature for 72 h. Solvent was removed under reduced pressure and purification of the crude residue by flash column chromatography (0-10 MeOH/CH₂Cl₂) afforded the desired product as a yellow oil (1 mg, 1%).

HRMS (ESI) calculated for C₂₁H₃₄AuN₂O₄PS [M+H+CH₃CN+C₃H₇NO]⁺: m/z 754.2581, found m/z 754.1340.

### EXAMPLES

The examples described herein are provided for the purpose of illustrating specific embodiments and are not intended to limit this disclosure in any way.

### Example 1: Selective Targeting of Fluorescent Conjugates to a Melanoma Cancer Cell Line

Overview: In relation to melanoma, F-18 fluoro-2-deoxyglucose ([¹⁸F] FDG) is commonly used as a PET imaging agent. However, there are significant limitations to this compound. It is only useful in stage III and IV cancers, it has poor sensitivity for detecting brain metastases, and it yields high levels of false positives due to normal patient physiology (see, e.g., Momodu and Vangu, 2022). The following experiments evaluated the uptake and selectivity of chemical conjugates comprising a pantothenic acid group or pantothenic derivative group coupled to BODIPY FL in relation to human epithelial melanocytes (HEM) and NZM11 melanoma cancer cell line.

Chemical conjugates tested: The tested compounds included 124 (also called CJ-15,801-BODIPY); 125, 126, 127, 128, 129, 130, 131, 132 (also called pantothenic acid-BODIPY); 133, 134, 135, each of which are coupled to BODIPY FL. The control compound was BODIPY FL.

Materials and methods: NZM11 is an adherent metastatic melanoma cell derived from a 47-year-old male patient with a subcutaneous lesion on his mid back. This human metastatic melanoma is identified with code CBA-1398 from Cell bank Australia. These cancer cells possess a BRAF mutation V600E. Cells of NZM11 diluted in MEMα media and cells of HEMnDP (primary melanocyte cell line) diluted in media 254 were left to settle in 96-wells microplate for 48 hours. 10 μL of 10 mM stock solutions in DMSO from 138 and pantothenic acid/pantothenic acid derivatives coupled to BODIPY were diluted with HBSS to afford 100 nM solution. Then, 100 nM compounds and DMSO solutions were diluted 1:1 with MEMα media for NZM11 and media 254 for HEMnDP to afford 50 nM solutions. After the 48 hour settle time, media was removed from the cells and 200 μL of 50 nM media solutions were poured to each well and incubated for 5 minutes at 37 °C. After 5 minutes, the compounds and DMSO solutions were removed and cells were washed with PBS. Then, 100 μL of TrypLE trypsin were added to each well and incubated for 5 minutes at 37 °C. After this time, 100 μL of MEMα media or media 254 were added to the wells and samples were sorted through FACS biosort. The studies were completed in triplicate wells. The FACS results output represented the mean of the fluorescence intensities (in arbitrary units) measured for cells sorted in every sample

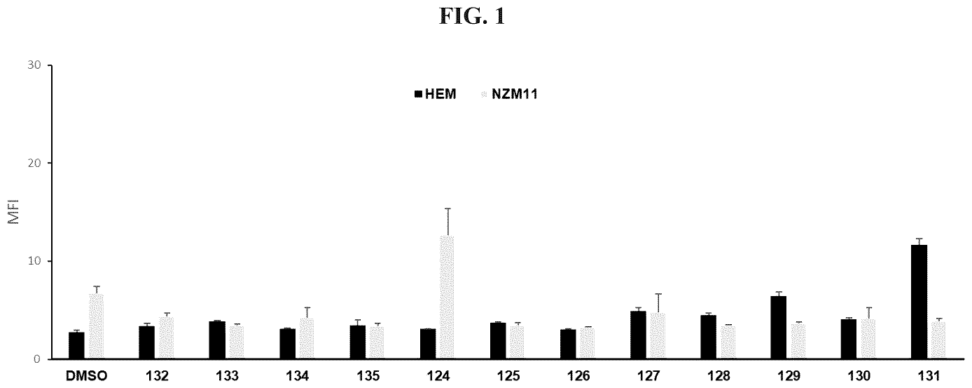

Results: The results are shown in FIG. 1. The set of conjugates coupled to BODIPY FL reveals selective uptake in both HEM and NZM11 cell types. Cells incubated with fluorescent conjugate 124 showed significant uptake in this BRAF melanoma cell type. The 124 conjugate targeted NZM11 cells with the highest intensities. By comparison, the enantiomer 126, displayed 25% of mean intensity compared to the 124 conjugate. Without wishing to be bound by theory, it is possible that the spatial arrangement of the atoms in the pantoyl region assists in recognition and uptake. Interestingly, 131 showed significant uptake into HEM cells. Thus, the 124 conjugate showed the highest uptake in NZM11 melanoma cell line, and the 124 conjugate showed no significant uptake in normal melanocytes, particularly as compared to 131.

Conclusions: The set of conjugates coupled to BODIPY FL reveal a selective uptake into melanocytes and NZM11 melanoma cells. The 124 conjugate showed the best targeting activity, with high uptake into cancer cells and no significant uptake into normal melanocytes.

### Example 2: Selective Targeting of Fluorescent Conjugates to Melanoma Cancer Cell Lines

Overview: The following experiments evaluated the uptake and selectivity of chemical conjugates comprising pantothenic acid groups or pantothenic acid analogue groups coupled to BODIPY FL in relation to metastatic melanoma cell lines NZM11 (BRAF V600E), NZM40 (NRAS Q16H) and NZM9 (BRAF and NRAS wildtype).

Chemical conjugates tested: The tested compounds included 124 (also called CJ-15,801-BODIPY); 125, 126, 127, 128, 129, 130, 131, 132 (also called pantothenic acid-BODIPY); 133, 134, 135, each of which are coupled to BODIPY FL. The control compound was BODIPY FL.

Materials and methods: NZM11 is an adherent metastatic melanoma cell which was originally derived from a 47-year-old male patient with a subcutaneous lesion on his mid back. This human metastatic melanoma is identified with code CBA-1398 from Cell Bank Australia. These cancer cells possess a BRAF mutation V600E. BRAF is a gene that makes a protein called B-Raf. The gene is involved in sending signals within cells that direct the cell's growth. Around 50% of melanomas bear activating BRAF mutations. In this family of mutations, V600E is the most common, which is a single mutation on codon 600. BRAF mutation is also most common in patients whose tumours arise on skin without chronic sun-induced damage. See Ascierto et al., 2012.

NZM40 is an adherent cell line which was originally derived from a metastatic melanoma from a 47 year old female patient, in 2004. This human metastatic melanoma is identified with code CBA-1381 from Cell bank Australia. These cancer cells possess a NRAS mutant Q61H. Ras is a superfamily of proteins implicated in cell growth, survival and differentiation. NRAS (neuroblastoma rat sarcoma) was the first oncogene recognized in melanoma and mutations in NRAS account for 20% of all melanomas. NRAS mutations primarily occur at position 61 and involve amino acid change from glutamine (Q) to arginine (R), lysine (K), leucine (L) or histidine (H). These mutations block NRAS into a GTP-bound state damaging its GTPase activity (FIG. 1). Patients are usually older (>55 years) with previous UV exposure. See Garcia-Alverez et al., 2021.

NZM9 is an adherent cell line which was originally derived from a metastatic melanoma from an 80 year old male patient (from axillary lymph node, source: Cellosaurus database). These cancer cells are BRAF and NRAS wild type. NZM9 present a mutation on gene TP53, which is the most frequent in human cancers, on 179C/T. This gene encodes the synthesis of protein p53 which acts as a tumour suppressor. See Tran et al., 2021; Levine and Owen, 2009; Duffy and Crown, 2021.

For screening, NZM11, NZM40 and NZM9 cells were suspended at 500 cells per mL in MEMα media and 100 mL dispensed into 96 well plates and left to settle for 48 hours. Then, 10 μL of 10 mM stock solutions in DMSO for the conjugates were diluted with HBSS to afford 400 nM solution. Next, 400 nM compounds and DMSO solutions were diluted 1:1 with MEMα media to afford 200 nM solutions. After 48 hours the media was removed from the 96 well plates and 200 μL of 200 nM conjugates solutions dispensed to each well (in triplicate) and incubated for 5 minutes at 37 °C. After 5 minutes, the media was removed and cells washed with PBS. Then, 100 μL of TrypLE trypsin (ThermoFisher Scientific) were added to each well and incubated for 5 minutes at 37 °C after which, 100 μL of MEMα media were added and samples were sorted through FACS biosort. The FACS results output were represented as the mean of the fluorescence intensities (in arbitrary units) measured for cells sorted in every sample.

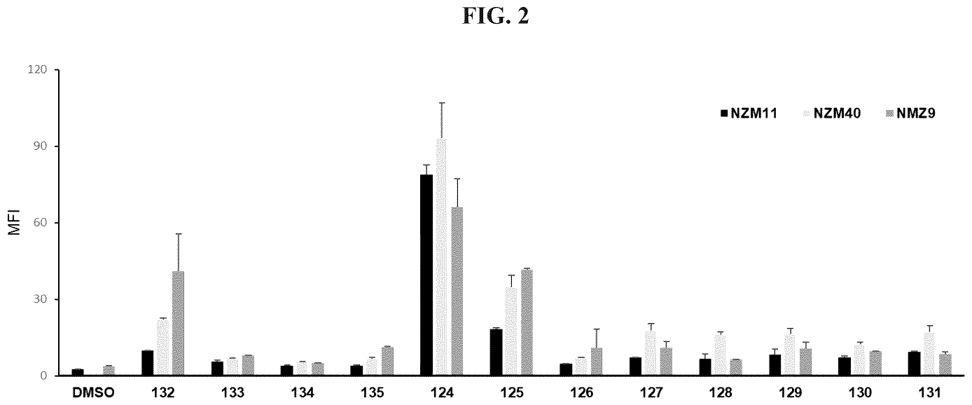

Results: The results are shown in FIG. 2. The set of conjugates coupled to BODIPY FL reveals selective uptake in NZM11, NZM40 and NZM9 cells. With remarkable uptake levels, the 124 conjugate displayed the highest intensities for all three metastatic melanoma cell lines, confirming our previous results (see Example 1). The 124 conjugate performed better than the other conjugates, showing 15 fold higher (NZM11), 13 times higher (NZM40) and 7 times higher (NZM9) fluorescence intensities when compared to enantiomer 126. The 125 conjugate also showed selective uptake in relation to all three cell lines. It was noted that cells incubated with 125 showed significant intensities. The 132 conjugate also showed uptake, particularly in relation to NZM40 and NZM9 cells. In addition, 127, 128, 129, 130, and 131 showed selectivity in relation to NZM40 cells.

Without wishing to be bound by theory, it was postulated that the steric effect of the ketal group on the chemical conjugates could reduce uptake into cells. However, the 132 conjugate also showed selective uptake in relation to the three cell lines. Cells incubated with this conjugate exhibited 8 times less (NZM11), 4 times less (NZM40) and 1.5 times less (NZM9) fluorescence intensities than the best performer 124. It was postulated that higher uptake could relate to different levels of expression of the transporter/channel and/or different affinities to the conjugate.

Conclusion: The set of conjugates coupled to BODIPY FL reveals a selective uptake into the three melanoma cell lines, NZM11, NZM40, and NZM9. The 124 conjugate showed the best targeting activity for the three genotypes. 125, its ketal version, also showed significant uptake in the three genotypes, NZM11, NZM40, NMZ9. Other conjugates showed targeting, as well. Considerations for targeting may possibly include channels/transporters (expression levels), activity, and availability.

### Example 3: Selective Targeting of Fluorescent Conjugates to Leukaemia Cell Line

The following experiments evaluated the uptake and selectivity of chemical conjugates coupled to BODIPY FL in relation to human lymphocytes, monocytes and neutrophils and Jurkat cells.

Chemical conjugates tested: The tested compounds included 124 (also called CJ-15,801-BODIPY); 125, 126, 127, 128, 129, 130, 131, 132 (also called pantothenic acid-BODIPY); 133, 134, 135, each of which are coupled to BODIPY FL. The control compound was BODIPY FL.

Materials and methods: Jurkat cells have been previously shown to be defective in the expression of two lipid phosphatases, PTEN (phosphatase and tensin homologue) and SHIP (SH2-domain-containing inositol polyphosphate 5′ phosphatase). The biological implications of SHIP deficiency in T cells remain under evaluation, but the loss of PTEN leads to activation of the phosphatidylinositol 3-kinase (PI3K)-signalling pathway. See Abraham and Weiss, 2004. This activation of PI3K protein is also on NZM40 melanoma cell line, and represents a key mutation for its sustained proliferation. See Silva et al., 2017. For these experiments, Jurkat cells were diluted on RPMI media (supplemented with 10% FBS+1% penicillin-streptomycin) to afford 400 cells/μL. 2 μL of 100 μM stock solutions in DMSO from 138 and pantothenic acid/pantothenic acid derivatives coupled to BODIPY were diluted with 998 μL of RPMI media (supplemented with 10% FBS+1% penicillin-streptomycin) to afford 200 nM solution. 100,000 cells were dispensed to Eppendorf tubes, centrifuged for 1.5 min at 5000 rcf and the supernatant was removed. The cells then were incubated with 500 μL of solutions at 200 nM for 4.5 min and 37 °C. After the incubation time, cells were centrifuged for 1.5 minutes at 5000 rcf. Supernatant was removed and cell pellet re-suspended in 200 μL fresh RPMI media, transferred to 96 well-plate and run on FACS machine, approximately 1,000 cells analysed.

In addition to this, 25 mL of full blood was collected from a healthy donor in lithium-heparin vacutainer. Peripheral blood mononuclear cells (PBMCs) were collected after ficoll separation. PBMCs then were centrifuge at 400 g for 5 minutes. Supernatant was removed from the cells and resuspended with 20 mL of HBSS with a final concentration of 417,500 cells/mL. Then, 479 μL of cells suspension were poured into Eppendorf tube and 1000 μL of 200 nM drug solution in HBSS were added to afford 135 nM final drug concentration, incubated at 37 °C for 5 minutes. After incubation time, PBMCs were centrifuged at 400 g for 5 minutes. Supernatant was removed from the cells and pellet resuspended with 200 μL of HBSS. Suspension was transferred to 96 well-plate and run on FACS machine, approximately 1,000-250 cells analysed. The FACS results output represented the mean of the fluorescence intensities (in arbitrary units) measured for cells sorted in every sample.

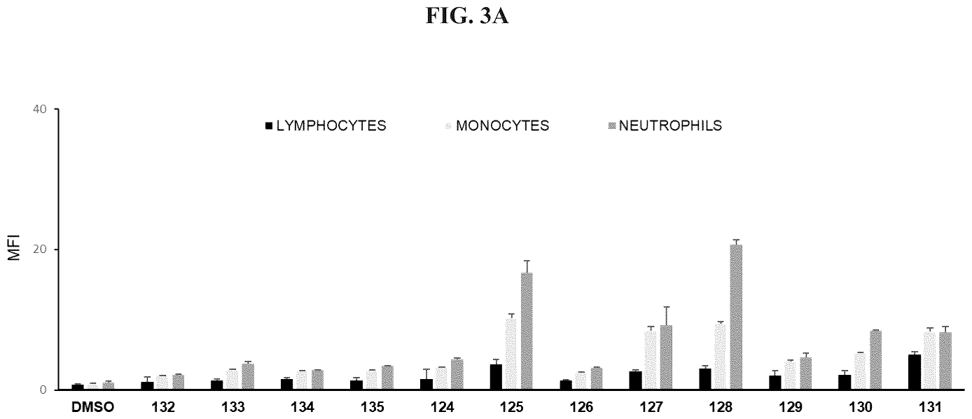

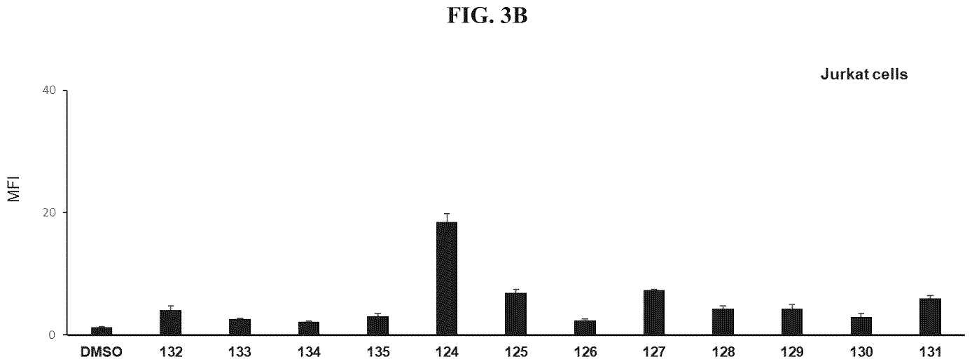

Results: The results are shown in FIG. 3A and FIG. 3B. The conjugates coupled to BODIPY FL reveal selective uptake in both PBMCs and Jurkat cells. Lymphocytes, which represent the majority in the PBMC sample (70-90%), did not show important fluorescence intensities with the conjugates. Monocytes and neutrophils, on the other hand, showed targeting by the 125 conjugate and its enantiomer 127, both derivatives bear a ketal group on the pantoyl core. As well as these ketal derivatives, the 128 conjugate provided significant intensities. This derivative displayed a cis geometry and a free diol group on the pantoyl core. It is possible that these features may increase uptake, but it is also possible that the R enantiomer (128) may be taken up better than the S enantiomer (130).

Remarkable selectivity was observed for Jurkat cells. The 124 conjugate displayed the highest uptake from the full set of conjugates, having two times higher intensities than 125 (the CJ ketal analogue) and 127 (125 enantiomer). Other conjugates that showed targeting of Jurkat cells include 125, 127, 128, 129, 131, and 132. It was concluded that the set of conjugates coupled to BODIPY FL revealed a selective uptake into PBMCs and Jurkat cells. The 124 conjugate showed the best targeting activity, with higher uptake into cancer cells and no significant uptake into normal blood cells.

### Example 4: Selective Targeting of Fluorescent Conjugates to Colorectal and Embryonic Cell Lines

Overview: Experiments were carried out to evaluate the uptake and selectivity of the chemical conjugates on HT29 (colorectal cancer cell line) and mouse embryonic fibroblast (MEFs) non-cancerous cell line.

Chemical conjugates tested: The tested compounds included 124 (also called CJ-15,801-BODIPY); 125, 126, 127, 128, 129, 130, 131, 132 (also called pantothenic acid-BODIPY); 133, 134, 135, each of which are coupled to BODIPY FL. The control compound was BODIPY FL.

Materials and methods: The HT-29 line was originally isolated from a primary tumour in 1964 from a 44-years-old female. This cell line is positive for expression of N-ras and overexpression of p53 antigen. The human colon adenocarcinoma cell line HT29, is not only used to study the biology of human colon cancers, but it is receiving special interest in studies focused on food digestion and bioavailability due to the ability to express characteristics of mature intestinal cells. The application of this cell line to transport studies of drugs and food compounds is illustrated, especially when the effect of the mucus layer is considered or used as co-culture in combination with Caco-2 cells. These cells have shown a high rate of glucose consumption and therefore, they require high glucose concentration in the medium. Verhoeck et al., 2015.

Fibroblasts are ubiquitous mesenchymal cells that are thought to play important roles in wound repair and healing, and serve as reservoirs of multipotent progenitors capable of repopulating depleted cell compartments. Primary mouse embryonic fibroblast (MEF) cultures consist of highly heterogeneous populations of distinct cell types. Singhal, P. K., et al., 2016. MEF cells have a limited growth capacity and on prolonged passaging spontaneously immortalize at a low frequency. In contrast to transformation of primary MEF cells that requires the presence of two cooperating oncogenes, immortalised MEF cells can be transformed by a single oncogene (Ras) resulting in a loss of contact inhibition, anchorage-independent growth, and tumour formation in nude mice. Studies of MEF cells have played an important role in the elucidation of the molecular mechanisms underlying cellular immortalization, transformation, and tumorigenesis. Additionally, utilization of MEF cells disrupted for specific genes has provided a powerful tool to analyse the genetic regulation of these cellular processes. Sun and Taneja, 2007.

For these experiments, HT-29 or MEF cells were diluted on αMEM media (supplemented with 10% FBS+1% penicillin-streptomycin) to give a 250 cells/μL suspension of which 200 μL were aliquoted into each well of a 96 well plate followed by incubation for 24 hours. Next, 2 μL of 100 μM stock solutions in DMSO from the conjugates coupled to BODIPY were diluted with 998 μL of αMEM media to give a 200 nM solution. After 24 hour incubation, media was removed and 200 μL of 138 and pantothenic acid/pantothenic acid derivatives coupled to BODIPY 200 nM solutions were added and cells were incubated for 5 minutes at 37 °C. After 5 minutes, the compounds and DMSO solutions were removed and cells were washed with PBS. Then, 100 μL of TrypLE trypsin were added to each well and incubated for 5 minutes at 37 °C. After this time, 100 μL of DMEM media were added to the wells and samples were sorted through FACS biosort. The studies were completed in duplicated wells.

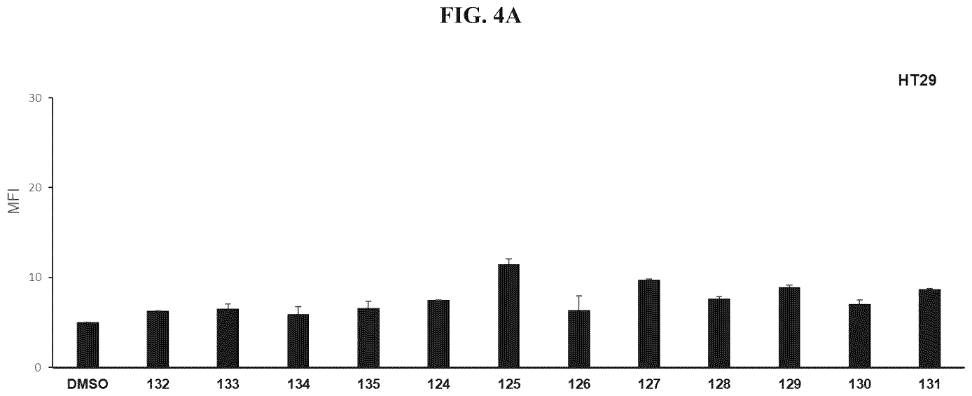

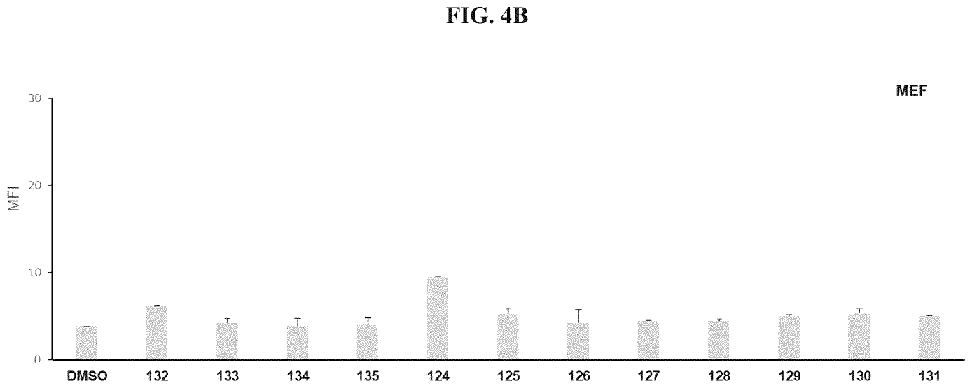

Results: The results are shown in FIGS. 4A-4B. The 125 conjugate, and its enantiomer 127, showed moderately higher fluorescent intensities in HT29 cells, as compared to other conjugates (see FIG. 4A). These compounds represent the ketal protected version of 138 on the pantoyl core. As well these trans derivatives, the cis derivatives (129 and 131) -bearing the ketal group-showed higher uptakes than their diol counterparts. It is possible that lower polarity derivates are preferred, with no significant impact on steric effect from the ketal group. The 124 and 132 conjugates exhibited the highest uptake in mouse embryonic cells (see FIG. 4B). These two derivates represent the 138 group and pantothenic acid group (the natural substrate), respectively. Interestingly, their S enantiomers (126 and 134) showed lower intensities—around 50% less than the R derivatives—revealing potential selectivity.

Conclusion: The conjugates coupled to BODIPY FL reveal uptake in cancer cell line HT29, particularly 127. 124 and 132 showed uptake in mouse embryonic cells, which will have certain pre-cancerous traits. Extended incubation time could be useful in clarifying patterns, especially in MEF cells. In addition to this, it will be helpful to screen additional cell lines.

### Example 5: Time Course for Fluorescent Conjugates Targeting Melanoma Cell Line

Overview: Experiments were performed to evaluate the uptake at different periods of time for conjugates 124, 125, 131, 132 and 3-azido BODIPY FL in relation to melanoma cell line NZM11 (BRAF V600E).

Materials and methods: NZM11 cells were suspended at 500 cells per mL in MEMα media and 100 mL dispensed into 96 well plates and left to settle for 48 hours. 10 μL of 10 mM stock solutions in DMSO from the conjugates coupled to BODIPY were diluted with HBSS to afford 400 nM solution. Then, 400 nM compounds and DMSO solutions were diluted 1:1 with MEMα media to afford 200 nM solutions. After 48 hours the media was removed from the 96 well plates and 200 μL of 200 nM conjugates solutions dispensed to each well (in duplicate) and incubated for 5 minutes, 30 minutes and 60 minutes at 37 °C (for the first test) or 60 minutes and 3 hours (for the second test). After indicated incubation time, the media was removed and cells washed with PBS. 100 μL of TrypLE trypsin (ThermoFisher Scientific) were added to each well and incubated for 6.5 minutes at 37 °C after which, 100 μL of MEMα media were added and samples were sorted through FACS biosort. FACS results output represented the mean of the fluorescence intensities (in arbitrary units) measured for cells sorted in every sample. Uptake rate was calculated by the equation: Uptake rate=(MFI₂−MFI₁)/(T₂−T₁)

whereby: MFI=mean fluorescent intensities; T=time (at which time the sample was taken); Uptake rate units: fluorescence intensities units per minute.

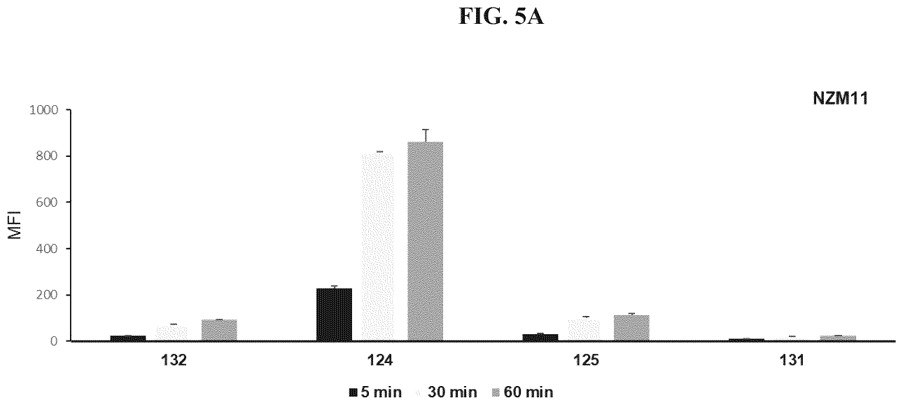

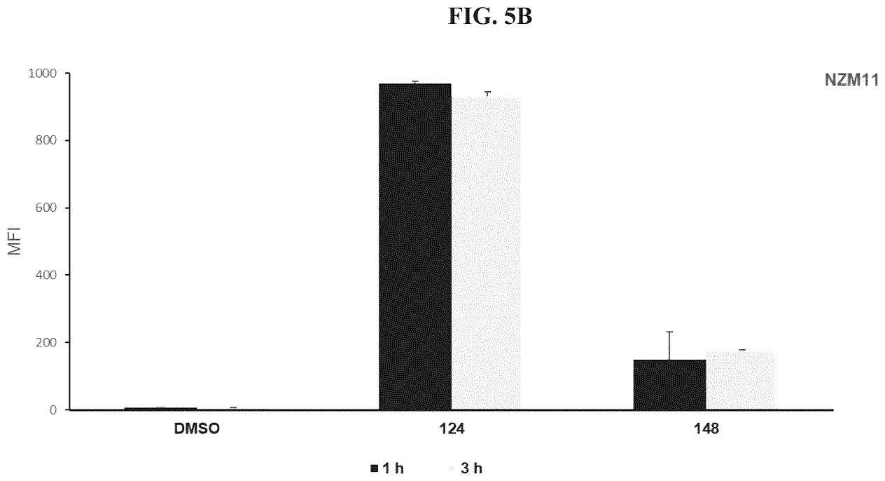

Results: The results are shown in FIGS. 5A and 5B. The time course studies reveal an outstanding uptake of 124 compared to the other conjugates tested in relation to NZM11. 124 showed the highest uptake rate after five minutes (45 units per minute). This rate started to reduce in samples taken at 30 minutes (23 units per minute). The highest intensities for 124 were reached at 60 minutes. At this point, the uptake rate had been reduced (1 unit per minute) (see FIG. 5A). After a 60 minutes peak, cells showed 4% lower intensities at 3 hour incubation than 60 minute incubation (see FIG. 5B). From these studies, it appeared that cells could rapidly take up the 124 conjugate before a saturation point was reached.

The 132 and 125 conjugates showed 10 times lower and 7 time lower uptake rates, as compared to 124. 132 and 125 reached highest intensities at 60 minutes. Without wishing to be bound to theory, it was possible that the absence of the enamide on 132 conjugate and the steric effect of the ketal group on 125 conjugate could reduce uptake levels. Cells incubated with 131 displayed non-significant intensities throughout. This was noted as an S enantiomer, making it possible that the cis geometry and the steric effect of a ketal group on the pantoyl core could reduce uptake for certain cells. Finally, cells incubated with 3-azido BODIPY FL exhibited a low uptake rate and 6 times lower intensities than 124 at 1 hour and 3 hours (see FIG. 5B).

Conclusion: Uptake of 124 conjugate into NZM11 cells is rapid at the first minutes of incubation, and presents saturation after 60 minutes. The spatial arrangement (R enantiomer and trans geometry), the reduction of steric effect (diol) and the presence of enamide on the vector appear to be favourable qualities to increase the uptake in these cells.

### Example 6: Time Course for Fluorescent Conjugates Targeting Leukaemia Cell Line

Overview: These experiments were used to determine the quantitative uptake of three conjugates, including 124, 126, and 132, each of which are coupled to BODIPY FL, at various time points after incubation with Jurkat cells, a human leukaemia derived cell line.

Materials and methods: Jurkat cells were diluted on RPMI media (supplemented with 10% FBS+1% penicillin-streptomycin) to 400 cells/μL. 5 μL of 10 mM stock solutions in DMSO from 124, 126 and 132 were diluted with 1245 μL of RPMI media (supplemented with 10% FBS+1% penicillin-streptomycin) to give a 400 nM solution. 250 μL of cell suspensions were then added to each well in a 96 well plate and 250 μL of conjugate solutions (giving a 200 nM final concentration). 250 μL of media only was added to the DMSO control wells. Incubation was carried out for 5, 30 and 60 minutes at 37 °C in triplicate wells for each compound and time point.

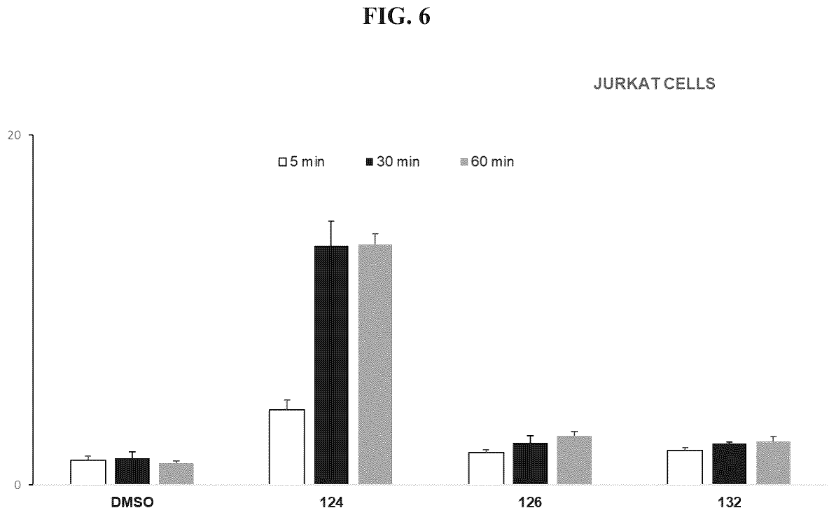

Results: This experiment was designed as a follow up to Example 3. In Example 3, a significant uptake of 124 was observed at 200 nM after 5 minute incubation on Jurkat cells. In this study, the 124 conjugate showed double the fluorescent intensities after 5 minutes incubation compared to its enantiomer 126 and 132 (pantothenic acid conjugate), confirming previous findings. Cells incubated for 30 minutes with 124 showed a six times greater fluorescent intensity than cells incubated with 126 and 132. At 60 minutes, the fluorescent intensity pattern did not change significantly. Cells incubated with the 124 conjugate displayed a 6 times greater fluorescence than the 126 and 132 treated samples. From the results, it appeared that cells incubated with the 124 conjugate reached a saturation point at or before 30 minute incubation (FIG. 6). Uptake was clearly increased for the 124 conjugate as compared to its enantiomer 126. The uptake of 124 increased between 5 and 30 minutes. Previously, a lower uptake on HPBC (specifically on lymphocytes) was observed (Example 3).

Conclusion: Human leukaemia T-lymphocytes (Jurkat cells) reveal a selective uptake of the 124 conjugate.

### Example 7: Lack of Toxicity of Fluorescent Conjugates in Melanoma Cells

Overview: Experiments were performed to evaluate the effect of 124, which is conjugated to BODIPY FL, on proliferation of NZM40 (NRAS Q16H) melanoma cell line. A sulforhodamine B assay (SRB assay) was utilised.

Materials and methods: NZM40 cell line is described above. 1×10⁶ NZM40 cells diluted in MEMα media were added to 2×10 cm plates and left to settle for 24 hours. After 24 hour settle period, media was removed from cells and 9900 μL fresh MEMα media added to each plate. 100 μL of DMSO were added to control plate and 100 μL [100 μM 124 in DMSO] were added to treatment plate to afford a final concentration of 1 μM 124. Cells were incubated at 37 °C and 5% 02 for 72 hours and cell images were taken every 24 hours. After 72 hour incubation, sulforhodamine B (SRB) assay was performed to compare final cell density. For this, 2.5 mL of cold 50% (w/v) TCA were added to DMSO (control) and 124 plates. Cells were incubated for 1 hour in TCA at 4 °C. Then, supernatant was removed from the cells and applied milliQ H₂O washes (5×). The plates left to air dry and 5 mL SRB solution (0.4% (w/v) SRB in 1% acetic acid) were added to the plates. The cells were incubated for 10 min at room temperature and then, excess of SRB solution was removed from cells and were washed with milliQ H₂O washes (5×). 5 mL of 10 mM Tris base were added to cells and remaining SRB solubilized. 100 μL were transferred to a 96 well plate. Further 4900 μL of 10 mM tris base were added to cells (1:2 dilution) and 100 μL transferred to 96 well plate. Last step was repeated to afford 1:4 and 1:8 dilutions. The 96 well plate was analysed on plate reader, at wavelength of 515 nm. Results represent the absorbance of cells without dilution, 1:2 dilution, 1:4 dilution and 1:8 dilution.

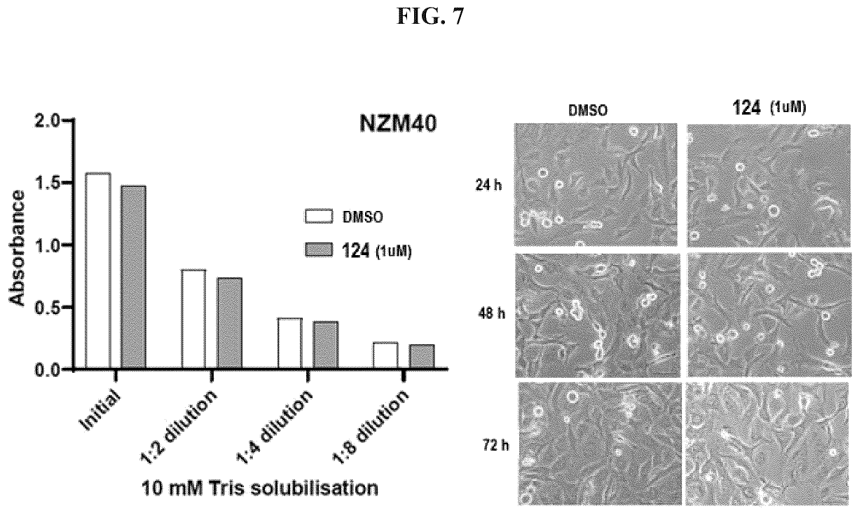

Results: The SRB assay relies on the properties of sulforhodamine binding stoichiometrically to proteins under mild acidic conditions. This can then be extracted using basic conditions. Thus, the amount of bound dye can be used as a proxy for cell mass, which can then be extrapolated to measure cell proliferation. NZM40 cells treated with the 124 conjugate (1 μM final concentration) showed comparable proliferation to DMSO control cells in the graph (FIG. 7). Absorbances from SRB assay were analysed using student T-test, and statistical test shows no significant difference on proliferation between control cells and cells incubated with 124. Cells treated with the 124 conjugate do not exhibit significant changes on morphology under microscope (FIG. 7). It was concluded that the 124 conjugate produced little to no effect on proliferation of NZM40 cell line at 1 μM after 72 h incubation. In order to calculate IC₅₀ of 124 conjugate, it was decided that the SRB assay should be done at different concentrations (more than 5 points) in triplicate.

Conclusion: NZM40 cells incubated with the 124 conjugate showed little to no effect on proliferation at 1 μM final concentration and 72 h incubation.

### Example 8: Selective Targeting and Inhibition of Melanoma Cells by Auranofin Conjugates

Overview: There are several small-molecule drugs approved by the FDA to treat melanoma. These include MEK and BRAF kinase inhibitors, and also non-specific DNA alkylators and chelators. All can lead to intolerable toxicities and long term injury (see, e.g., Domingues et al., 2022; Zhao et al., 2022). The present tests were carried out to determine the IC₅₀ for 136, 137 and auranofin itself (as control) on NZM11 (BRAF V600E mutant metastatic melanoma). A sulforhodamine B assay (SRB assay) was employed. Initial tests on proliferation were also carried out.

### Compounds Utilised:

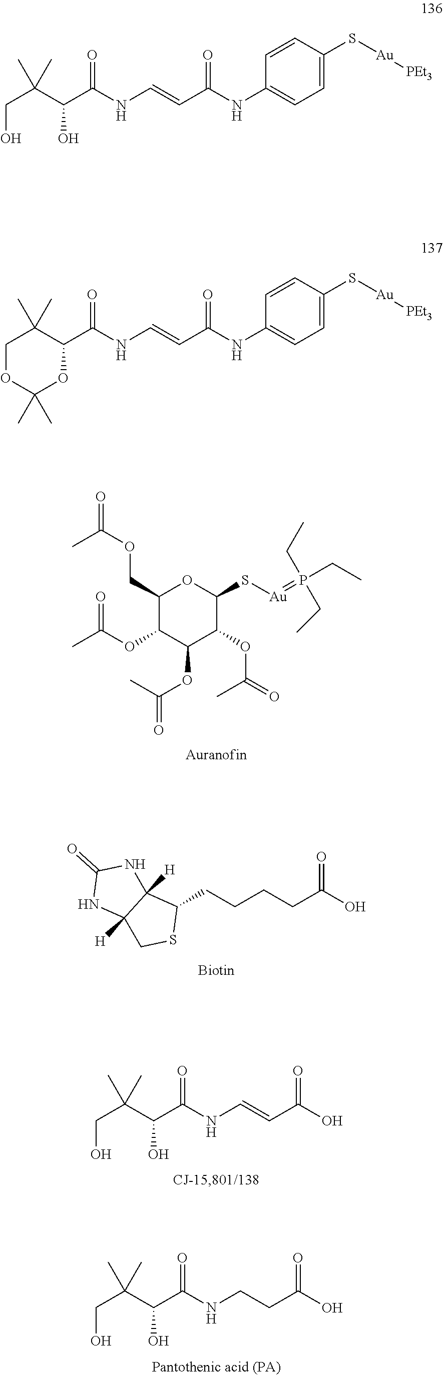

Materials and methods: NZM11 cells were cultured in MEMα media with an initial count of 1020 cells/μL. The cells were then diluted to get 25 cells per μL and 200 μL of this suspension (5000 cells per well), dispensed into 96 well plates and left to settle for 24 hours. 10 mM stock solutions of 136, 137, and auranofin were diluted with 1% FBS MEMα media to give 10 nM, 30 nM, 100 nM, 300 nM, 1 μM, 3 μM, 10 μM, 30 μM and 100 μM solutions. After 24 hours the media was removed from the 96 well plates. To individual wells 200 μL of each dilution were dispensed in triplicate for three groups (136, 137, and auranofin): 1% FBS MEMα media only (DMSO control), 10 nM, 30 nM, 100 nM, 300 nM, 1 μM, 3 μM, 10 μM, 30 μM and 100 μM. The cells were incubated at 37 °C, 5% 02 and 5% CO₂ for 48 hours.

In order to test the potency of 136 and 137, the IC₅₀ values were calculated for these auranofin conjugates. For analysis of the treatment plate (Tz) a SRB assay was utilised. For this assay, 50 μL of 50% (w/v) ice-cold trichloroacetic acid (TCA) was added to each well. To achieve this, 5 g of TCA was made up to 10 mL with milliQ H₂O. This was cooled on ice. Incubation was carried out for 1 hour at 4 °C to fix cells. TCA and media were removed from cells. Five milliQ washes were carried out and the plate was left to air dry. Next, 50 μL of 0.4% SRB solution (prepared in 1% acetic acid) was added to each well and incubated for 10 minutes at room temperature. The SRB solution was removed and five milliQ washes were carried out. Then, 100 μL of 10 mM Tris solution was added to each well, and the dye was solubilised. Absorbance at 515 nm was read on plate reader. Data was normalized to Tz plate, as per NCI60 screening method (https://dtp.cancer.gov/discovery_development/nci-60/methodology.htm).

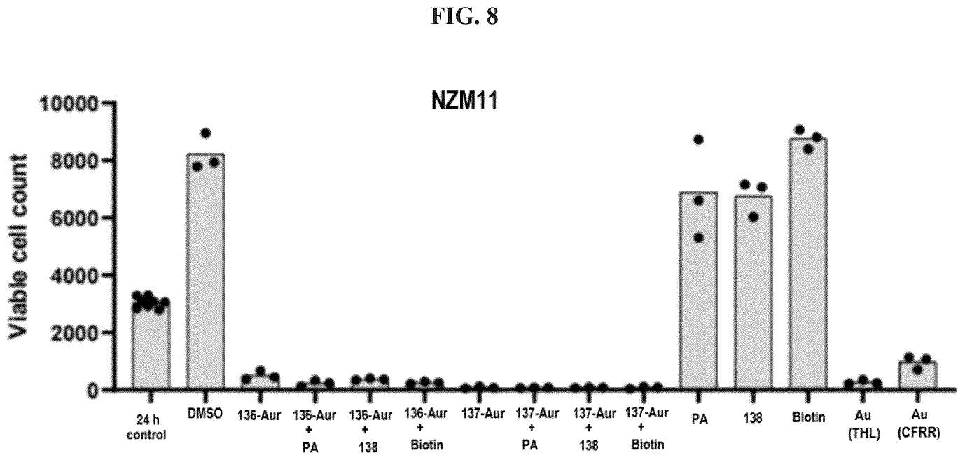

Results: The effects on proliferation are shown in FIG. 8. The cells treated with 136 and 137 exhibited more than 95% reduction on viability, as compared to auranofin itself. Activity of 136 and 137 was not affected by biotin and pantothenic acid (vitamins were 20 times more concentrated in these tests). Activity of 136 and 137 is not affected by CJ-15,801/138 (20 times more concentrated in these tests). Remarkably, the cells treated with 200 μM 138 itself, i.e., the vector in these analogues, showed similar viability as cells treated with pantothenic acid. These finding indicate that the 138 group has a little to no effect on cell proliferation in this cell line at these test conditions.

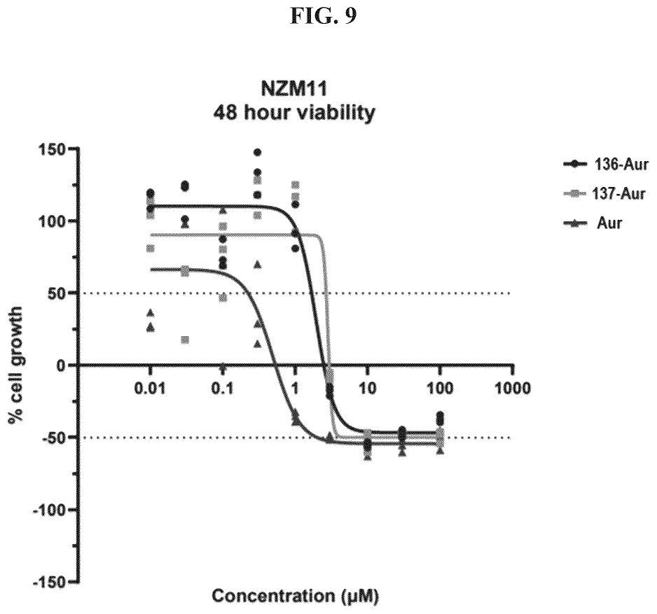

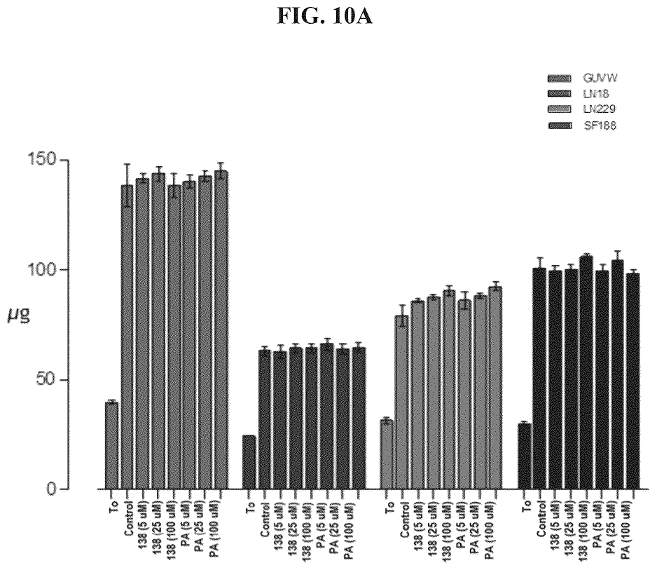

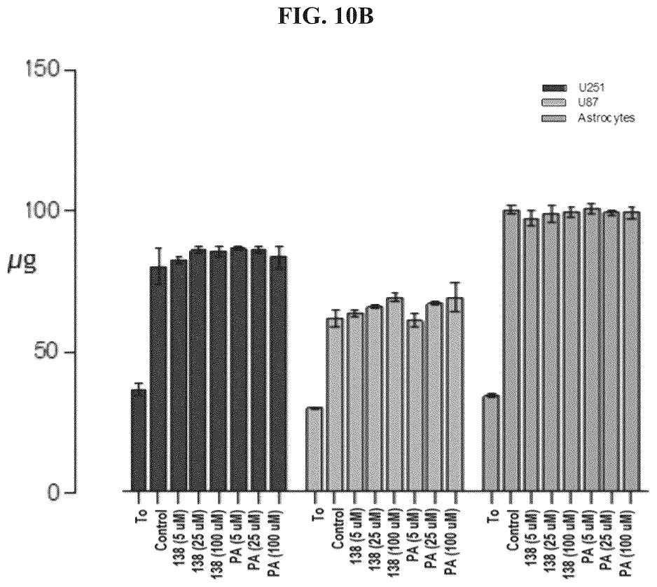

The results for IC₅₀ assessments are shown in FIG. 9. After 48 hours of incubation, the IC₅₀ was calculated for cells treated with auranofin. This was determined to be approximately 0.5 μM, which correlates with previous results (Sachweh et al., 2015). In these previous finding, auranofin showed inhibition of 50% of the population in tested melanoma cell lines at 0.5 μM concentration or less. The IC₅₀ calculated for 137 was five times higher than auranofin, this being determined as approximately 2.8 μM. In addition, 136 showed even greater potency, with the IC₅₀ calculated as approximately 2.9 μM. It is possible that the differences between 136 and 137 may be related to the chemical qualities and correlates with the fluorescent derivatives counterparts (124 and 125). For example, 136 bears the diol group in the pantoyl core, which is more polar than the ketal group on 137. It is possible that the increased polarity and a less hindered pantoyl core could improve the uptake of 136 into the NZM11 cells. Without wishing to be bound by theory, this may help to increase potency.

Summary: These experiments provide unexpected findings that the disclosed chemical conjugates demonstrate a surprising and unexpected level of discrimination when administered to different cell types. The inventors' experiments provides the first indication that the compounds of this disclosure exhibit specificity and selectivity for malignant cells. Thus, these chemical conjugates are particularly advantageous for targeting malignant cells, including melanoma cells, leukaemia cells, and colorectal cancer cells. The conjugates show higher uptake in malignant cells than in non-malignant cells, and can be readily used for detection methods for malignancies, as well as methods of treatment and prevention for malignancies. High contrast detection of the chemical conjugates is specifically noted.

Accordingly, the methods of this disclosure find utility in screening for presence of malignant cells, in detecting malignant cells, and/or in detecting the location of malignant cells. Thus, the disclosed methods may be used to screen, detect, or otherwise diagnose malignancies, and may also be used to localise malignant cells, for example, to tumour sites. This is particularly useful for targeting therapeutic interventions, for improving the prognosis of malignant disease, and for reducing the costs of malignant disease management. Furthermore, the methods of this disclosure can be used in therapeutic and preventative modalities, for example, by specifically targeting curative agents to malignant cells. Such methods improve the prognosis of malignant disease, prevent particular aspects of malignant disease (e.g., metastasis), and reduce the costs of malignant disease management.

### Example 9: Lack of Toxicity of Unconjugated Compounds in Glioblastoma Cells

Overview: These studies provided quantitative determination for proteins in human glioblastoma and astrocytes cells treated with different concentrations of compound 138 (CJ-15,801) and pantothenic acid (PA).

Materials and methods: Glioblastomas are the most aggressive and lethal primary astrocytic tumours of the central nervous system. These account for 60% to 70% of all gliomas and the majority are diagnosed in Caucasian male patients at advanced age. Based on the originating cell type or histological features, gliomas are divided into the subgroups of ependymomas, astrocytomas, and oligodendrogliomas. Of these, astrocytomas are the most common type of glial tumours (see, e.g., Jovčevska et al., 2019). Glioma cell lines are currently utilised in research due to their notable advantages in terms of availability, lower expense, ease of use, ease of propagation, and ethical considerations (for example, in avoiding animal and/or human studies) (see, e.g., Paolillo et al., 2021).

Glioblastoma cells were used to determine viability and growth by measurement of protein levels. Protein content of glioblastoma cells was determined after 48 hour incubation with compound 138 or PA at 5, 25 and 100 μM final concentration. For To, protein levels were determined at the start of the test (before addition of test compounds). For the control, protein levels were determined for cells incubated for 48 hours in the absence of the test compounds. Astrocytes were used as a non-cancer cell line control. U87: this cell line has epithelial morphology that was isolated from malignant gliomas from a male patient, likely with glioblastoma. SF188: this glioblastoma cell line was derived from the left temporal lobe tumour of an 8-year-old male patient. U251: this cell line expresses epidermal growth factor receptor (EGFR) receptors, for inhibition studies. LN229: This cell line has a mutation on TP53; Simple; p.Pro98Leu. LN18: This cell line is negative for glial fibrillary acidic proteins and S100 (S-100) protein. The cells exhibit mutated p53 (TP53) and possible homozygous deletions in the p16 and p14ARF tumour suppressor genes. GUVW cells were also utilised.

The glioblastoma cells and astrocytes showed comparable content of proteins with the control when were incubated with pantothenic acid. Pantothenic acid is the vitamin B5, that is involved in the synthesis of CoA, and it is involved in key processes for the growth and survival of cells. Interestingly, the cells exposed to compound 138, even at 100 μM, exhibited similar content of proteins than control cells. It seems that the cell's growth and viability are not affected by 138. This finding reveals the opportunity to test a set of fluorescent compounds as noted herein (e.g., 124 to 135). Flow cytometry could be applied in order to evaluate the fluorescent intensities in glioma cells and astrocytes.

Conclusions: Compound 138 and pantothenic acid showed little to no effect on protein levels in the cells at the tested conditions. This indicated lack of toxicity for the unconjugated compounds. As further studies, testing of the fluorescent conjugates can be carried out on glioblastoma cells and astrocytes.

### Example 10: Selective Targeting by Fluorescent Conjugates in an Animal Model for Melanoma

Overview: These studies have been designed to assess the uptake for compound 124 (also called CJA-BODIPY) in vivo. The binding to melanoma tumours is being determined in a athymic mouse model of melanoma. These studies are also being used to determine the degree of background fluorescence in non-tumour tissues.

Materials and methods: Compound 124 was supplied by Transfection Holdings Limited as a 10 mM stock solution in DMSO. The NZM40 cell line was used for this study. NZM40 cells are a melanoma cell line expressing mutant NRAS Q16H. Studies are being performed in the 6-8 week old NIH-111 strain of athymic mice at Ichor Biosciences (LaFayette, USA).

NZM40 cells were injected into the dorsal area at 5×10⁶ cells per site of six mice. Tumour size was measured twice weekly with a Vernier calliper until clearly established. When the tumours were established mice were dosed intravenously (tail vein) with a solution of 124 in phosphate buffered saline at a dose of 0.7 mg/kg. After up to two hours mice were sacrificed. These are being processed, with two for imaging, plus the un-treated mouse and the three remaining for additional analysis. See Table, below. Processing of euthanized mice and image analysis will be undertaken using the cryo-imaging protocols developed by BioInvision (Cleveland, USA). Frozen whole mouse sections will be imaged electronically under fluorescent light and the imaged sections integrated by computer.

| Mouse 1 | Mouse 2 | Mouse 3 | Mouse 4 | Mouse 5 | Mouse 6 |
|---|---|---|---|---|---|
| NZM40 | NZM40 | NZM40 | NZM40 | NZM40 | X |
| Cells | Cells | Cells | Cells | Cells | |
| Injected | Injected | Injected | Injected | Injected | |
| 124 | 124 | 124 | 124 | 124 | X |
| Injected | Injected | Injected | Injected | Injected | |
| Processed | Processed | Additional | Additional | Additional | Processed |
| for Imaging | for Imaging | analysis | analysis | analysis | for Imaging |

### Example 11: Assessing Uptake of Fluorescent Conjugates into Melanoma Cancer Cell Lines

Overview: This study was performed to evaluate of the level and specificity of uptake for compound 124, its enantiomer 126, and pantothenic acid conjugate compound 132 into NZM011 (BRAF V600E), NZM009 (BRAF and NRAS wildtype), NZM012 (BRAF V600E), NZM022 (BRAF G466A) and NZM077 (BRAF V600E) metastatic melanoma cells.

Cell lines: NZM011 is an adherent metastatic melanoma cell derived from a 47-year-old male patient with a subcutaneous lesion on his mid back. This human metastatic melanoma is identified with code CBA-1398 from Cell bank Australia. These cancer cells possess a BRAF mutation V600E. BRAF is a gene that makes a protein called B-Raf. The gene is involved in sending signals within cells that direct the cell's growth. Around 50% of melanomas bear activating BRAF mutations. In this family of mutations, V600E is the most common, which is a single mutation on codon 600. BRAF mutation is also most common in patients whose tumours arise on skin without chronic sun-induced damage (Ascierto et al., 2012). Additionally, NZM011 harbours a homozygous deletion CDKN2A. The cyclin-dependent kinase inhibitor 2A (CDKN2A), or p16INK4a, gene on 9p21 is important in the genesis of both familial and sporadic melanoma. Homozygous deletions and intragenic mutations of this gene have been identified in both melanoma cell lines and uncultured tumours (Walter et al., 1998).

NZM009 is an adherent cell line derived from a metastatic melanoma from an 80 year old male patient (from axillary lymph node, source: Cellosaurus database). These cancer cells are BRAF and NRAS wild type. NZM009 has a 179C/T mutation in the TP53 gene, which is the most frequent in human cancers. This gene encodes the protein p53, which acts as a tumour suppressor (Tran et al., 2021; Levine et al., 2009; Duffy et al., 2021). NZM9 also bears a homozygous silencing CDKN2A. Aberrant gene silencing is highly associated with altered cell cycle regulation during carcinogenesis. In particular, silencing of the CDKN2A tumour suppressor gene, which encodes the p16INK4a protein, has a causal link with several different types of cancers (Zhao et al., 2016).

NZM012 is an adherent cell line derived from a male, 19 year old patient with melanoma metastatic to small intestine. This cell line bears BRAF mutation V600E and partial deletion CDKN2A/B. It is wild-type for PIK3CA, PTEN, NRAS and temozolomide resistant. Low invasive cell line (Eccles) (Jeffs et al., 2009). Sourced from Cell Bank Australia. NZM022 bears BRAF G466A mutation, P53 241S/T mutant, NF1 non-frameshift insertion and PTEN epigenetic silencing. NZM077 harbours BRAF V600E mutation and missense NF1. The NF1 gene is located on chromosome 17. This gene produces a protein called neurofibromin that helps regulate cell growth.

Methods: For screening, NZM011, NZM009, NZM012, NZM022 and NZM077 cells were suspended at 250 cells per μL in MEMα media and 200 μL was dispensed into 96 well plates. The cells were left to settle for 24 hours for NZM011, NZM012 and NZM077 and 48 hours for NZM009 and NZM022 at 37 °C, 5% O₂, 5% CO₂. Before addition to the cells, 10 μL of 10 mM stock solutions in DMSO of compounds 124, 126, and 132 were diluted with 1% FBS MEMα media to give a 200 nM solution.

After the appropriate settle time, the media was removed from the 96 well plates and 200 μL of 200 nM conjugate solutions and DMSO control were dispensed to each well (in triplicate) and incubated for 30 minutes at 37 °C, 5% O₂, 5% CO₂. After 30 minutes, the media was removed and cells washed with PBS. Next, 100 μL of TrypLE trypsin (ThermoFisher Scientific) was added to each well and incubated for 5 minutes at 37 °C. After this, 150 μL of MEMα media (plus 1% FBS for NZM011, NZM012 and NZM077; plus 5% FBS for NZM009 and NZM022) were added. Cells were analysed by flow cytometry using a FACS biosort and FITC filter. Flow cytometry results represent the mean of the fluorescence intensities (in arbitrary units) measured for cells sorted in every sample.

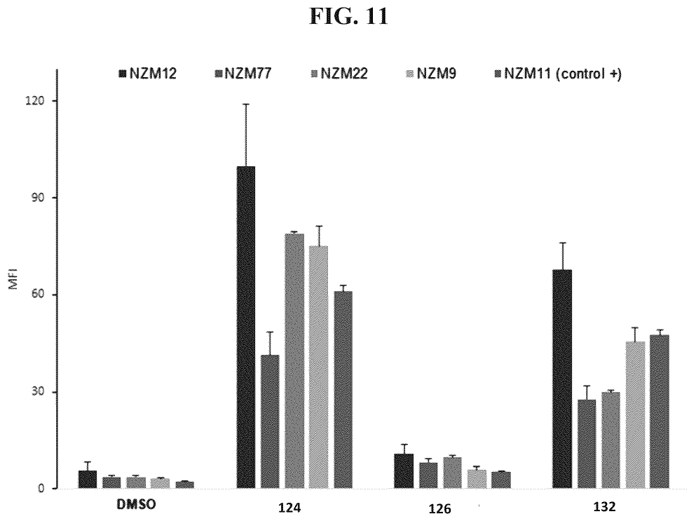

Results: Compound 124 showed the highest uptake levels in the five melanoma cell lines as compared to compounds 126 and 132 (FIG. 11). Interestingly, the enantiomer 126, exhibited 5-12 times less uptake. These results confirm the selectivity of uptake into the cells. A significant difference between the cell lines was demonstrated using Student's t-test analysis of compound 124 intensities of every cell line versus NZM011 (the first cell line tested), and using excel tool to calculate P value (with 2 tails, two sample equal variance). Without wishing to be bound by theory, this difference could represent the intrinsic properties of the cells, such as the level of expression of transporter in each cell line and/or transporter ability to differentiate a specific spatial arrangement on the pantoyl core in CJ-15,801 (compound 138) analogues.

Compound 124 showed 1.5-3 times greater uptake than pantothenic acid fluorescent conjugate 132 (FIG. 11). The cells treated with compound 132 also displayed significantly different uptake between cell lines. It is possible that this could mean a difference on the level of expression of transporter (SMVT) between cell lines. The uptake of compounds 124 and 132 in the same cell line (t-test), was significantly different. This finding could represent various possibilities, for example, that compounds 124 and 132 are transported by two different transporters or that they use the same transporter (not necessarily SMVT) but with different affinities. Future studies can be utilised to further elucidate a mechanism of uptake for compound 124.

Conclusions: Compound 124 shows a consistent, selective and high level of uptake to the four different melanoma cell lines having distinct genotypes. The differential uptake of compound 124 between cell lines could be related to intrinsic characteristics of the cells. This could include, for example, the types of transporters they express, post-translational modifications for the transporters, and the level of transporter expression.

### Example 12: Assessing Uptake of Fluorescent Conjugates into Leukaemia Cell Line

Overview: This study was designed to determine the quantitative uptake of three conjugates of pantothenic acid, compounds 124, 126 and 132, at various time points after incubation with Jurkat cells, a human leukaemia derived cell line.

Materials and methods: For this study, 7.7×105 Jurkat cells were centrifuged for 5 minutes at 2500 RPM. The supernatant was removed and the cells were diluted in RPMI media (supplemented with 1% FBS+1% penicillin-streptomycin) to 400 cells/μL. Next, 28 μL of 100 μM stock solutions from compound 124, 126, or 132 were diluted with 6972 μL of RPMI media (supplemented with 1% FBS+1% penicillin-streptomycin) to give a 400 nM solution. Following this, 250 μL of conjugate solutions (giving a 200 nM final concentration) and 250 μL of cell suspensions were then added to each well in a 96 well plate. In addition, 250 μL of media only was added to the DMSO control wells. Incubation was carried out for 5, 30 and 60 minutes at 37 °C in triplicate wells for each compound and time point.

After incubation, cells were centrifuged for 1.5 minutes at 5000 RCF. Supernatant was removed and the cell pellet re-suspended in 200 μL fresh RPMI media, transferred to 96 well-plate and each cell suspension analysed by flow cytometry. Flow cytometry results represent the mean of the fluorescence intensities (in arbitrary units) measured for cells sorted in every sample.

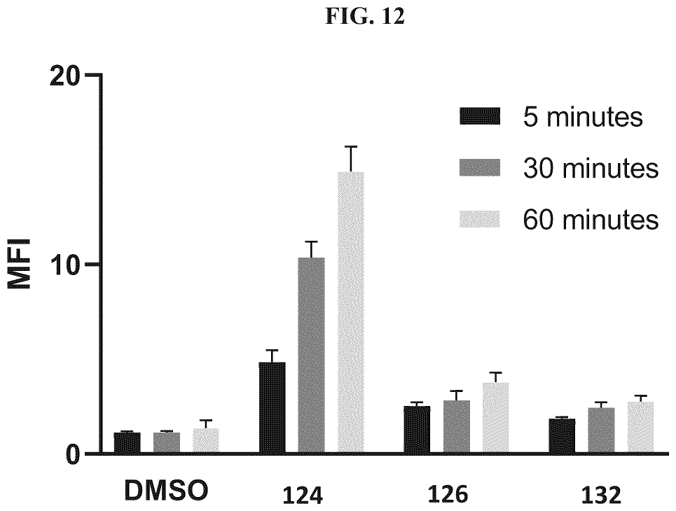

Results: Jurkat cells incubated with the conjugates showed a similar pattern to that observed previously. Cells incubated with compound 124 displayed the highest uptake at every timepoint. At 5 minutes, uptake was 4 times higher than the DMSO control. The uptake increased ten and fourteen times in cells incubated for 30 minutes and 60 minutes compared to DMSO controls, respectively (FIG. 12).

Jurkat cells incubated with compound 126 (the enantiomer of compound 124) exhibited highest uptake at 60 minutes, although only three times higher than the DMSO control and 4 times lower than 124 uptake (FIG. 12). This result reveals a preference for compound 124 over compound 126 indicating that the cells may be exhibiting spatial arrangement selectivity. The pantothenic acid conjugate (compound 132) showed the lowest uptake, close to the DMSO control.

Conclusions: In order to gain further insights into the uptake of the conjugates into Jurkat cells, this time course study was performed. Jurkat cells showed selective uptake of compound 124 which confirms the previous experiments provided herein. The data points to the use of the disclosed compounds for the selective targeting of T-cells for use in leukaemia and similar disorders.

### Example 13: Use of Fluorescent Conjugate for Whole Body Fluorescent Cryo-Imaging

Overview: This study was performed to determine the binding of fluorescent conjugate to melanoma tumours in a recognised mouse model of melanoma.

Materials and methods: Compound 124 was supplied as a 10 mM stock solution in DMSO. NZM011 cells were grown in AMEM, 5% FBS, 5 μg/mL insulin, 5 μg/mL transferrin, 5 ng/mL sodium selenite at 37 °C with 5% O₂, 90% N₂ 5% CO₂. The study was performed in 6-week-old male NIH-III athymic mice (strain code 201). Work was carried out by Ichor Biosciences La Fayette NY, USA. Mice were injected on one flank with 5×10⁶ NZM11 cells in a 50:50 matrigel:media suspension with a 22G needle. When tumours reach over 500 mm3 the mice were administered intravenously (tail vein) with a solution of compound 124 in phosphate buffered saline at a dose of 0.7 mg/kg. The mice were euthanized at 1 hour or 6 hours post dosing and frozen for cryoimaging. A control mouse received no tumour cells or compound 124 and was euthanized and frozen for cryoimaging at the same time as the treated mice.

Processed animals were cryoimaged imaged at BioInvision in Cleveland Ohio. Whole animal was sectioned at 40 microns and imaged using brightfield at fluorescent modes (503 nm). In-plane pixel size 10 microns. Mouse #1 with tumour on one flank at 500 mm3, treated with compound 124 (0.7 mg/kg) and euthanised 1 hour post treatment. Mouse #6 no tumour or compound 124 treatment euthanised on same day.

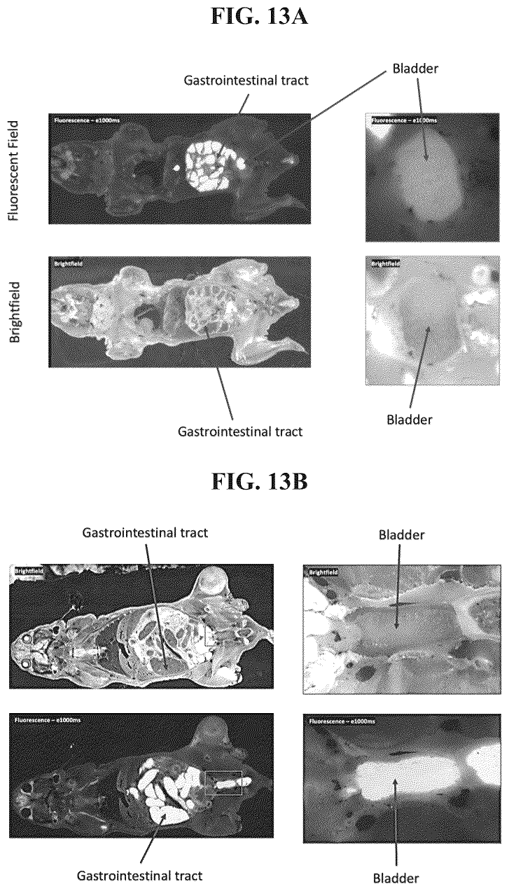

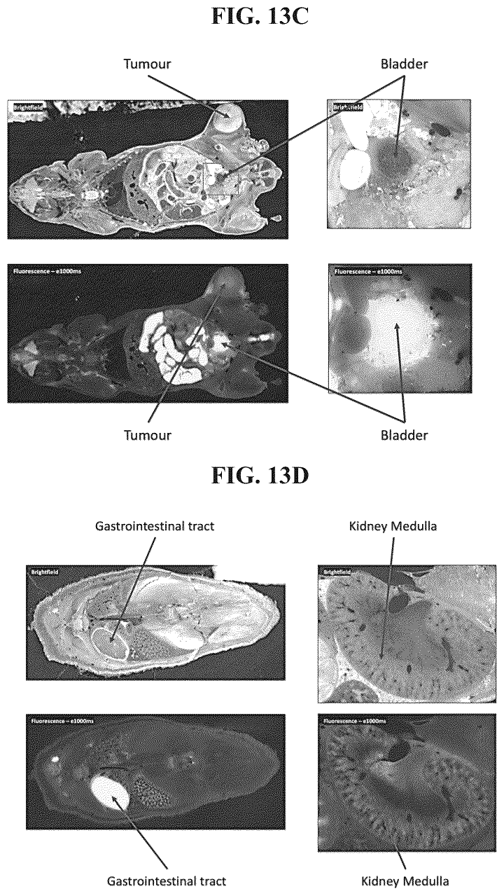

Results: In the control mouse (mouse #6), as expected no specific green fluorescence was seen. Characteristic faint autofluorescence was observed in the gastrointestinal tract, the bladder and the genitourinary tract as expected (FIG. 13A). In the compound 124-treated mouse (mouse #1), specific green fluorescence from compound 124 was seen in the proximity of the tumour. Fluorescence specific for compound 124 was seen in the proximity of one of the tumours as well as kidneys (mainly renal medulla) and bladder (FIGS. 13B-13D). No fluorescence associated compound 124 was seen in any other tissues.

Conclusion: Compound 124 specifically targeted and bound to tumour in vivo. The bladder also showed specific fluorescence indicating that the compound is being excreted by the renal route.

One of ordinary skill in the art will readily appreciate from the present disclosure that later modifications, substitutions, and/or variations performing substantially the same function or achieving substantially the same result as embodiments described herein may be utilised according to such related embodiments. Thus, the present disclosure is intended to encompass, within its scope, the modifications, substitutions, and variations to processes, manufactures, compositions of matter, compounds, means, methods, and/or steps set out herein.

In this specification, where reference has been made to external sources of information, including patent specifications and other documents, this is generally for the purpose of providing a context for discussing the aspects of the present disclosure. Unless stated otherwise, reference to such sources of information is not to be construed, in any jurisdiction, as an admission that such sources of information are prior art or form part of the common general knowledge in the art.

The description herein may contain subject matter that falls outside of the scope of the claimed invention. This subject matter is included to aid understanding of the invention.

### REFERENCES

Abraham, R. T, and A. Weiss, Jurkat T cells and development of the T-cell receptor signalling paradigm. Nature Reviews. Immunology, 2004, 4(4): p. 301-8.
Ascierto, P. A., et al., The role of BRAF V600 mutation in melanoma. Journal of translational medicine, 2012, 10: p. 85-85.
Bertoli, E., et al., Neuroblastoma rat sarcoma mutated melanoma: That's what we got so far. Pigment Cell & Melanoma Research, 2019, 32(6): p. 744-752.
Chirapu, S. R., et al., High specificity in response of the sodium-dependent multivitamin transporter to derivatives of pantothenic acid. Current Topics in Medicinal Chemistry, 2013. 13(7): p. 837-842.
Cho, Y., et al., Crystal structure of a p53 tumor suppressor-DNA complex: understanding tumorigenic mutations. Science, 1994, 265(5170): p. 346-355.
Conn, P. M., Animal models for the study of human disease. 2013, London; Waltham, MA; Elsevier.
Domingues, B, et al., Melanoma treatment in review. ImmunoTargets and Therapy 2018, 7, 35-49.
Duffy, M. J. and J. Crown, Drugging “undruggable” genes for cancer treatment: Are we making progress?International Journal of Cancer, 2021, 148(1): p. 8-17.
Garcia-Alvarez, A., C. Ortiz, and E. Muñoz-Couselo, current perspectives and novel strategies of nRas-mutant melanoma. OncoTargets & Therapy, 2021, 14: p. 3709-3719.
Gaudelli, N. M. and C. A. Townsend, Stereocontrolled syntheses of peptide thioesters containing modified seryl residues as probes of antibiotic biosynthesis. Journal of Organic Chemistry, 2013. 78(21): p. 11115-11115.
Hansen, A. M., et al., Tunable BODIPY derivatives amenable to ‘click’ and peptide chemistry. Tetrahedron, 2013. 69(39): p. 8527-8533.
Jeffs, A. R., et al., A gene expression signature of invasive potential in metastatic melanoma cells. PLOS ONE, 2009. 4(12): p. e8461.
Jovčevska, I., Genetic secrets of long-term glioblastoma survivors. Bosnian Journal of Basic Medical Sciences, 2019. 19(2): p. 116-124.
Levine, A. J. and M. Oren, The first 30 years of p53: growing ever more complex. Nat Rev Cancer, 2009, 9(10): p. 749-58.
Lin, Z., et al., Comparative microarray analysis of gene expression during activation of human peripheral blood T cells and leukemic Jurkat T cells. Laboratory Investigation, 2003, 83(6): p. 765-776.
Momodu J. I, and Vangu M. D. T., 2022, F-18 fluoro-2-deoxyglucose positron emission tomography (PET)/computed tomography (CT) imaging in melanoma: normal variants, pitfalls, and artifacts. Front. Nucl. Med. 2:835404. doi: 10.3389/fnume.2022.835404.
Paolillo, M., S. Comincini, and S. Schinelli, In vitro glioblastoma models: A journey into the third dimension. Cancers, 2021. 13(10): p. 2449.
Plonka, P. M., et al., What are melanocytes really doing all day long . . . ?Experimental dermatology, 2009, 18(9): p. 799-819.
Sachweh, M. C., et al., Redox effects and cytotoxic profiles of MJ25 and auranofin towards malignant melanoma cells. Oncotarget, 2015. 6(18): p. 16488-506.
Sewell A. L., Villa M. V. J., Matheson M., Whittingham W. G., Marquez R., Fast and flexible synthesis of pantothenic acid and CJ-15,801. Organic Letters. 2011; 13(4): 800-3.
Singhal, P. K., et al., Mouse embryonic fibroblasts exhibit extensive developmental and phenotypic diversity. Proceedings of the National Academy of Sciences, 2016, 113(1): p. 122-127.
Silva, J. M., et al., PIK3CA-mutated melanoma cells rely on cooperative signaling through mTORC1/2 for sustained proliferation. Pigment cell & melanoma research, 2017, 30(3): p. 353-367.
Sun, H, and R. Taneja, Analysis of transformation and tumorigenicity using mouse embryonic fibroblast cells. Methods Mol Biol, 2007, 383: p. 303-10.
Tran, K. B., et al., Genomic and signalling pathway characterization of the NZM panel of melanoma cell lines: A valuable model for studying the impact of genetic diversity in melanoma. Pigment Cell Melanoma Res, 2021, 34(1): p. 136-143.
Verhoeckx, K., et al., in The Impact of Food Bioactives on Health: in vitro and ex vivo models, 2015, Springer. doi: 10.1007/978-3-319-16104-4.
Walker, G. J., et al., Virtually 100% of melanoma cell lines harbor alterations at the DNA level within CDKN2A, CDKN2B, or one of their downstream targets. Genes,
Chromosomes and Cancer, 1998. 22(2): p. 157-163. Wu, B., X. Yang, and M. Yan, Synthesis and structure-activity relationship study of antimicrobial auranofin against ESKAPE pathogens. Journal of Medicinal Chemistry, 2019. 62(17): p. 7751-7768.
Yamaguchi, Y, and V. J. Hearing, Melanocytes and their diseases. Cold Spring Harbor perspectives in medicine, 2014, 4(5): p. a017046.
Zhao, R., et al., Implications of genetic and epigenetic alterations of CDKN2A (p16(INK4a)) in cancer. EBioMedicine, 2016. 8: p. 30-39.
Zhao, Q. et al., Recent advances in metabolism and toxicity of tyrosine kinase inhibitors. Pharmacol Therapy, 2022, 237. doi.org/10.1016/j.pharmthera.2022.108256.

What is claimed is:

1. A compound for use in:
(i) targeting at least one chemical agent to a malignant cell;
(ii) detecting a malignant cell;
(iii) treating a malignant cell;
(iv) preventing activity of a malignant cell; or
(v) modifying a malignant cell;
wherein:
for (i), the compound is a chemical conjugate comprising a pantothenic acid group, a pantothenic acid derivative group, or a pantothenic acid analogue, and at least one chemical agent;
for (ii), the compound is a chemical conjugate comprising a pantothenic acid group, a pantothenic acid derivative group, or a pantothenic acid analogue, and at least one detection agent;
for (iii) or (iv), the compound is a chemical conjugate comprising a pantothenic acid group, a pantothenic acid derivative group, or a pantothenic acid analogue, and at least one therapeutic agent; or
for (v), the compound is a chemical conjugate comprising a pantothenic acid group, a pantothenic acid derivative group, or a pantothenic acid analogue, and at least one cell modifying agent.

2. The compound of claim 1, which is of formula (I): 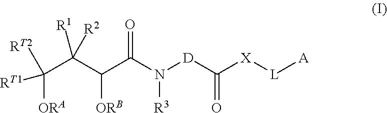
wherein:
—R^{A} and —R, are each independently selected from hydrogen, alkyl, alkenyl, alkynyl, aralkyl, cycloalkylalkyl, alkanoyl and aralkanoyl;
or —R^{A} and —R^{B} are together —C(R^{C1})(R^{C2})—, forming a 6-membered ring, where —R^{C1} is independently selected from hydrogen, alkyl, alkenyl, alkynyl, aralkyl and cycloalkylalkyl, and —R^{C2} is independently selected from hydrogen, alkyl, alkenyl, alkynyl, aralkyl, cycloalkylalkyl, alkoxy, alkenoxy, alkynoxy, aralkoxy and cycloalkylalkoxy, or —R^{C1} and —R^{C2} are together oxo (═O);
—R^{T1} and —R^{T2} are each independently hydrogen or alkyl;
—R¹ and —R² are each independently selected from hydrogen, alkyl, alkenyl, alkynyl, aralkyl and cycloalkylalkyl;
—R³ is hydrogen or alkyl;
-D- is C₂₋₄ alkenylene or Ca-4 alkylene, where the alkenylene or alkylene is optionally substituted with alkyl or halo;
—X— is a covalent bond, —N(R⁴)—, —O—, —S—, or —Se—, where —R⁴ is hydrogen or alkyl;
-L- is a linker or a covalent bond; and
-A comprises at least one chemical agent;
and salts, solvates and protected forms thereof.

3. The compound of claim 2, wherein the linker is selected from the group consisting of bivalent linkers; multivalent linkers; cleavable linkers; enzyme cleavable linkers; protease cleavable linkers; esterase cleavable linkers.

4. The compound of claim 2, wherein the linker is selected from the group consisting of triazole linkers; glycol linkers; polyethylene glycol (PEG) linkers; alkyl linkers; heteroalkyl linkers; and alkylene linkers.

5. The compound of any one of claims 1 to 4, wherein the at least one detection agent is selected from the group consisting of: dyes; fluorophores; fluorescent proteins; unlabelled chelating agents; labelled chelating agents; optical contrast agents; X-ray contrast agents; magnetic resonance imaging contrast agents; positron emission tomography contrast agents; and any combination thereof.

6. The compound of any one of claims 1 to 4, wherein the at least one detection agent is selected from the group consisting of BODIPY; BODIPY derivatives; DOTA chelator; indocyanine green; fluorine-18 [¹⁸F] radioisotopes; and any combination thereof.

7. The compound of any one of claims 1 to 4, wherein the at least one therapeutic agent is selected from the group consisting of: antibodies; antimetabolites; biologic response modifiers; chain terminators; growth factor inhibitors; hormonal agents; nucleic acid intercalators; pathway inhibitors; proteasome inhibitors; enzyme inhibitors; radionuclides; radionucleotides; and any combination thereof.

8. The compound of any one of claims 1 to 4, wherein the at least one therapeutic agent is selected from the group consisting of auranofin; vemurafenib, dabrafenib; trametinib; and any combination thereof.

9. The compound of any one of claims 1 to 4, wherein the at least one cell modifying agent is selected from the group consisting of: polypeptides; peptides; enzymes; enzyme fragments; antibodies; antibody fragments; antibody epitopes; polynucleotides; RNAs; DNAs; genes; gene fragments; and any combination thereof.

10. The compound of any one of claims 1 to 9, wherein the malignant cell is a THL positive cell.

11. The compound of any one of claims 1 to 9, wherein the malignant cell is selected from the group consisting of: bladder cancer cells, bone cancer cells, breast cancer cells, brain cancer cells, colorectal cancer cells, endometrial cancer cells, head and neck cancer cells, kidney cancer cells, leukaemia cells, liver cancer cells, lung cancer cells, lymphoma cells, melanoma cells, ovarian cancer cells, pancreatic cancer cells, prostate cancer cells, and thyroid cancer cells.

12. The compound of any one of claims 1 to 11, wherein the malignant cell is selected from the group consisting of: melanoma cells; colon cancer cells; rectal cancer cells; and leukaemia cells.

13. The compound of any one of claims 1 to 11, wherein the malignant cell is selected from the group consisting of:
(a) malignant melanocytes; melanoma tissue cells, melanoma lesion cells; and melanoma tumour cells;
(b) melanoma metastasis cells; cutaneous melanoma cells; ocular melanoma cells; superficial spreading melanoma cells; nodular melanoma cells; lentigo maligna melanoma cells; amelanotic melanoma cells; acral lentiginous melanoma cells; mucosal melanoma cells; desmoplastic melanoma cells; and uveal melanoma cells;
(c) melanoma cells having mutation of one or more of CDK2NA, MDm2, CDK4EGF, RB1, MC1R, TYR, TYRP1, and ASIP genes; and
(d) melanoma cells having mutation of one or more of BRAF, EGF, Fas, and PTEN genes.

14. The compound of any one of claims 1 to 11, wherein the malignant cell is selected from the group consisting of: colorectal adenocarcinoma cells; colorectal mucinous adenocarcinoma cells; colorectal signet ring adenocarcinoma cells; colorectal lymphoma cells; gastrointestinal stromal tumour cells; colorectal leiomyosarcoma cells; colorectal melanoma cells; colorectal squamous cell carcinoma cells; hereditary nonpolyposis colorectal cancer cells; metastatic colorectal cancer cells; familial adenomatous polyposis cells; juvenile polyposis coli cells; Turcot Syndrome cancer cells; and Peutz-Jeghers Syndrome cancer cells.

15. The compound of any one of claims 1 to 11, wherein the malignant cell is selected from the group consisting of: lymphocytic leukaemia cells; acute lymphocytic leukaemia cells; chronic lymphocytic leukaemia cells; myeloid leukaemia cells; acute myeloid leukaemia cells; chronic myeloid leukaemia cells; prolymphocytic leukaemia cells; large granular lymphocytic (LGL) leukaemia cells, hairy cell leukaemia cells, and myelodysplastic syndrome cells.

16. The compound of any one of claims 1 to 11, wherein the malignant cell is selected from the group consisting of: tumour node and metastasis (TNM) staged cancer cells; Stage 0 cancer cells; Stage I cancer cells: Stage II cancer cells; Stage III cancer cells; Stage IIIA cancer cells; Stage IIIB cancer cells; Stage IIIC cancer cells, Stage IIID cancer cells; Stage IV cancer cells.

17. The compound of any one of claims 1 to 16, wherein:
(a) the targeting of the malignant cell is performed in vitro or in vivo;
(b) the detecting of the malignant cell is performed in vitro or in vivo;
(c) the treating of the malignant cell is performed in vitro or in vivo;
(d) the preventing of the activity of the malignant cell is performed in vitro or in vivo; or
(e) the modifying of the malignant cell is performed in vitro or in vivo.

18. The compound of any one of claims 1 to 17, wherein the detecting includes one or more of: illumination; visualisation; imaging; optical imaging; fluorescence imaging; infra-red imaging; radio imaging; microscopy imaging; machine imaging; extended depth of field imaging; image recording; image capturing; image analysis; image filtering; image reconstruction; computer-aided image analysis; image analysis using artificial intelligence (AI); deep learning; image storage; image storage by cloud computing; image retrieval; image transmission; image recognition; image reconstruction, tomography; computed tomography (CT or CAT); positron emission tomography (PET) or PET-CT; and magnetic resonance imaging (MRI).

19. The compound of any one of claims 1 to 17, wherein the treating includes one or more of:
(a) reducing viability, replication, and/or tumour formation of the malignant cell; or
(b) reducing vascularity, the growth, and/or the size of a tumour which comprises the malignant cell.

20. The compound of any one of claims 1 to 17, wherein the preventing the activity includes one or more of:
(a) impeding growth or spread of the malignant cell;
(b) impeding growth or spread of a tumour which comprises the malignant cell; or
(c) halting, decreasing, or delaying metastasis by the malignant cell; or
(d) halting, decreasing, or delaying metastasis of a tumour which comprises the malignant cell.

21. The compound of any one of claims 1 to 20, wherein the compound is selected from the group consisting of formulae: 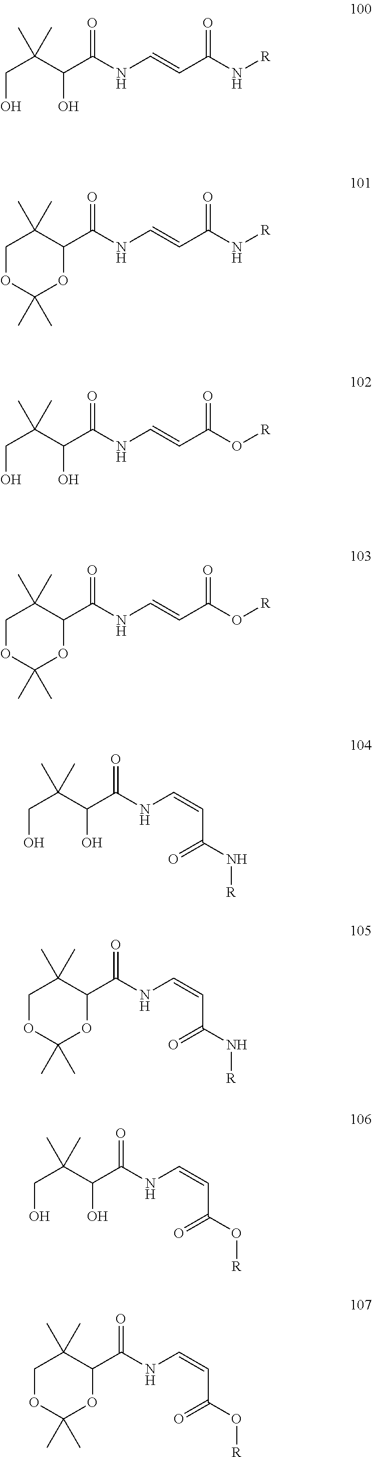 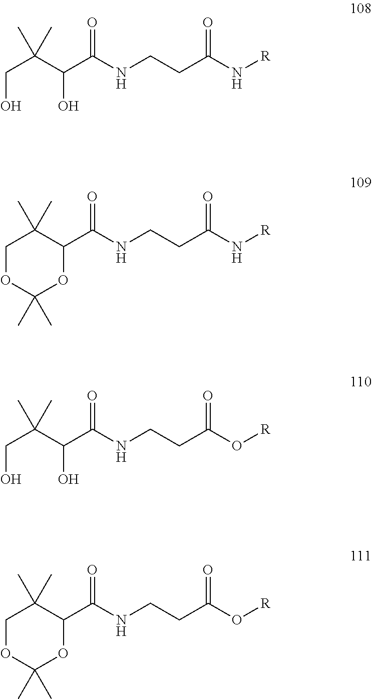
wherein —R is -L-A;
wherein -L- is a linker or a covalent bond; and -A is at least one chemical agent,
and salts, solvates, and protected forms thereof.

22. The compound of any one of claims 1 to 20, wherein the compound is selected from the group consisting of formulae: 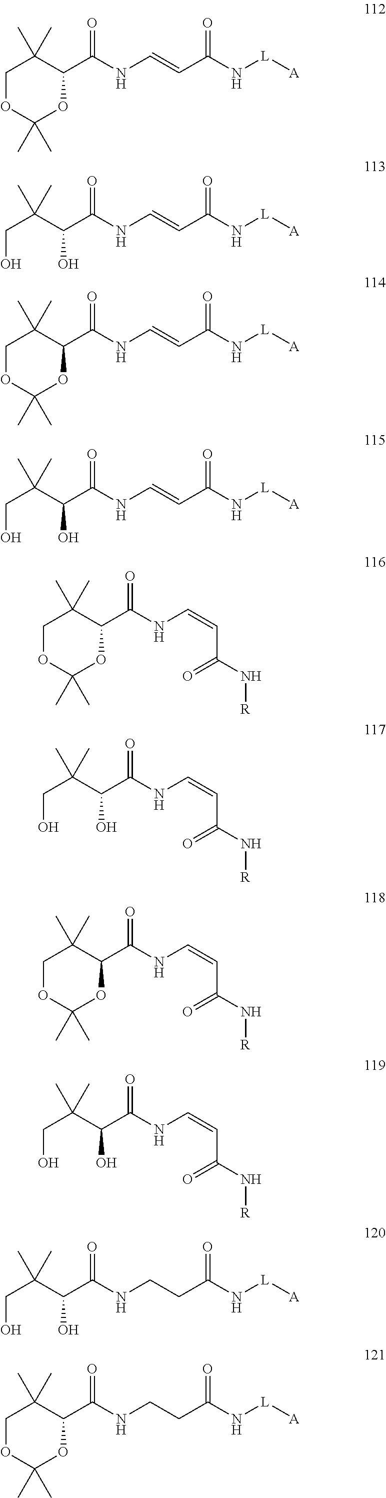 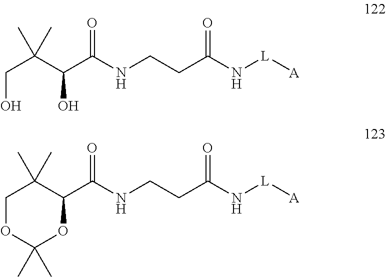
wherein -L- is a linker or a covalent bond; and -A is at least one chemical agent, and salts, solvates, and protected forms thereof.

23. The compound of any one of claims 1 to 20, wherein the compound is selected from the group consisting of: 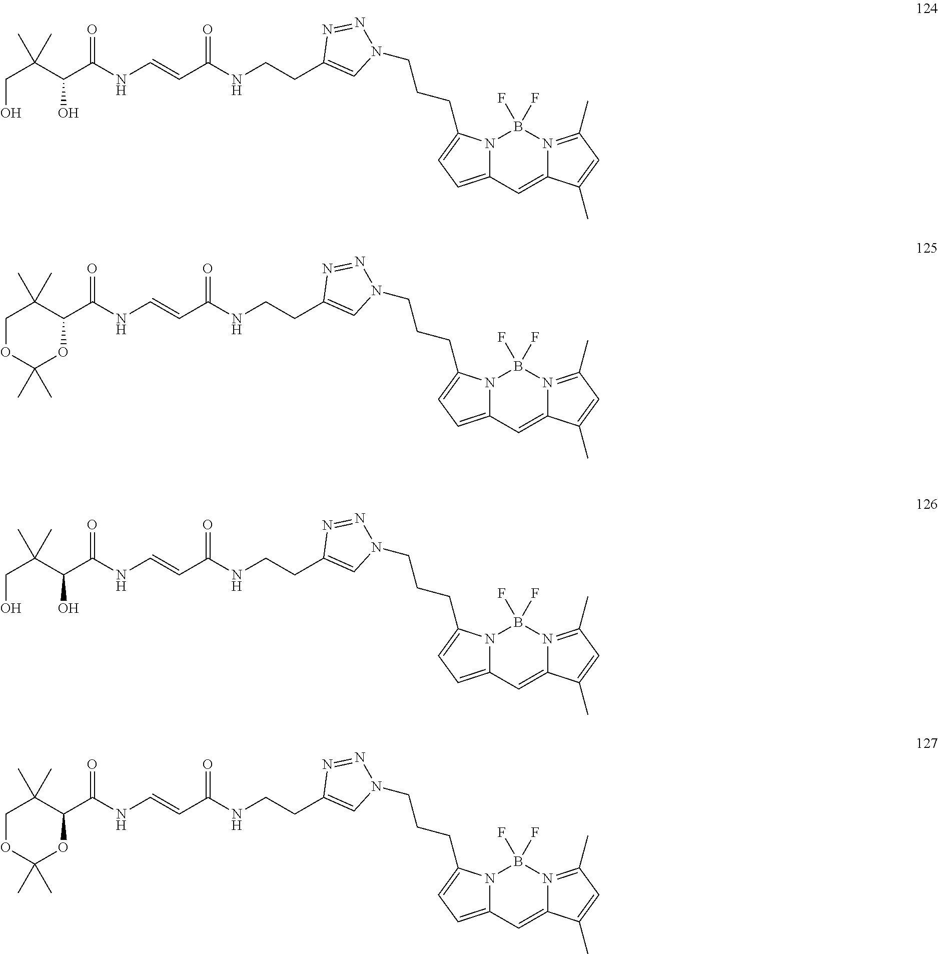 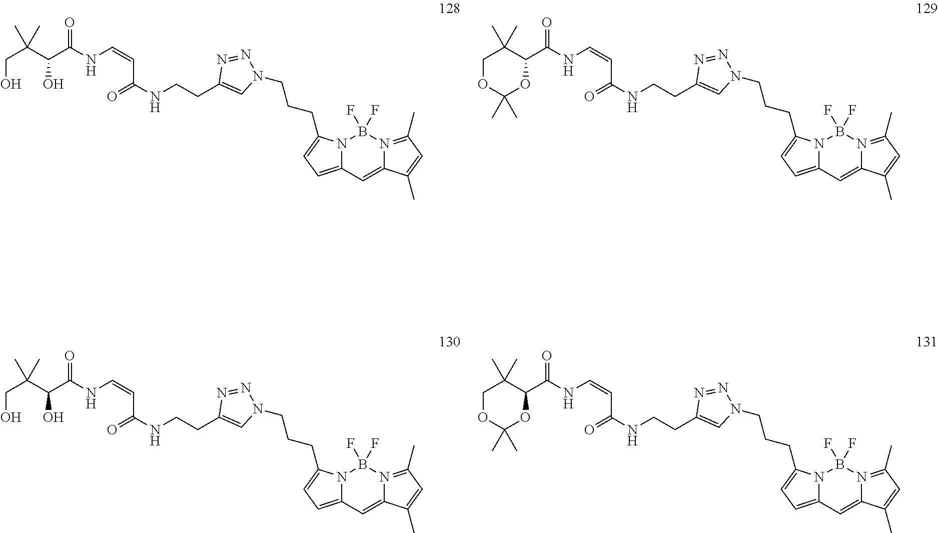 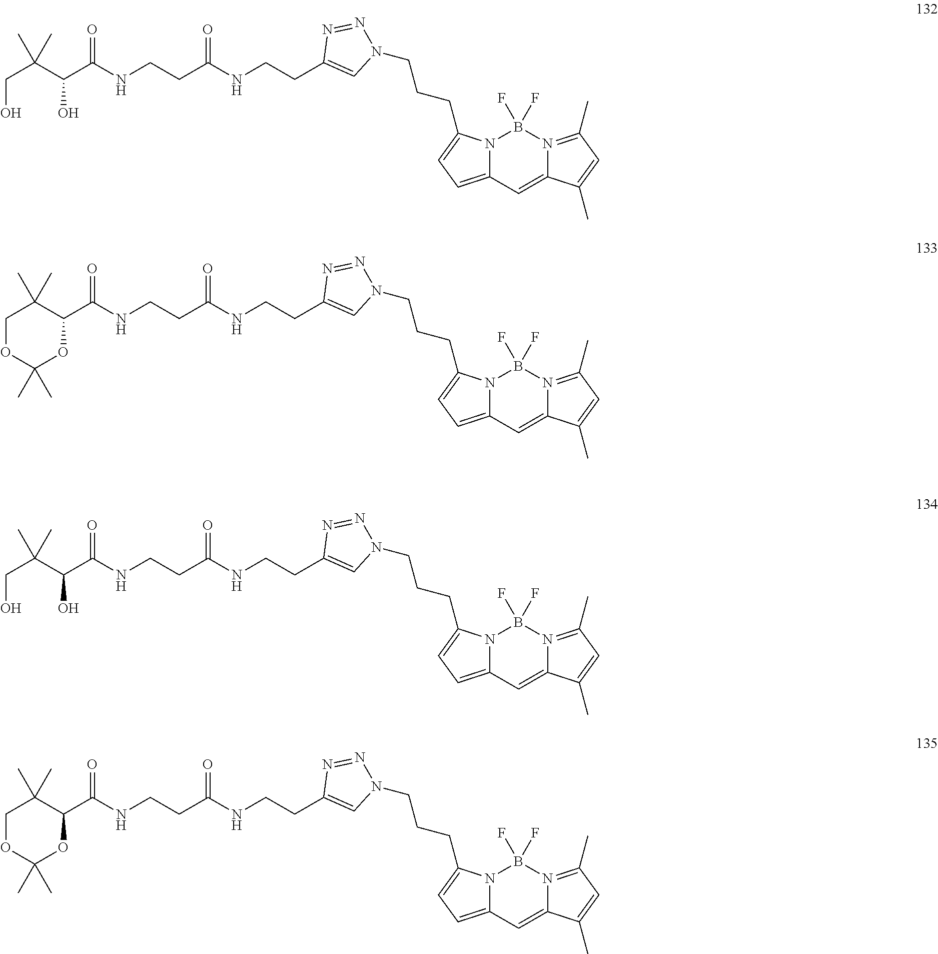 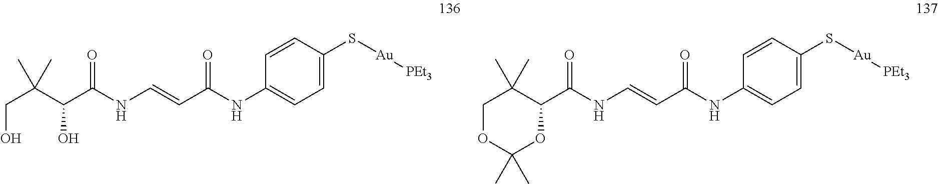
and salts, solvates, and protected forms thereof.

24. The compound of any one of claims 1 to 20, which is selected from the group consisting of: 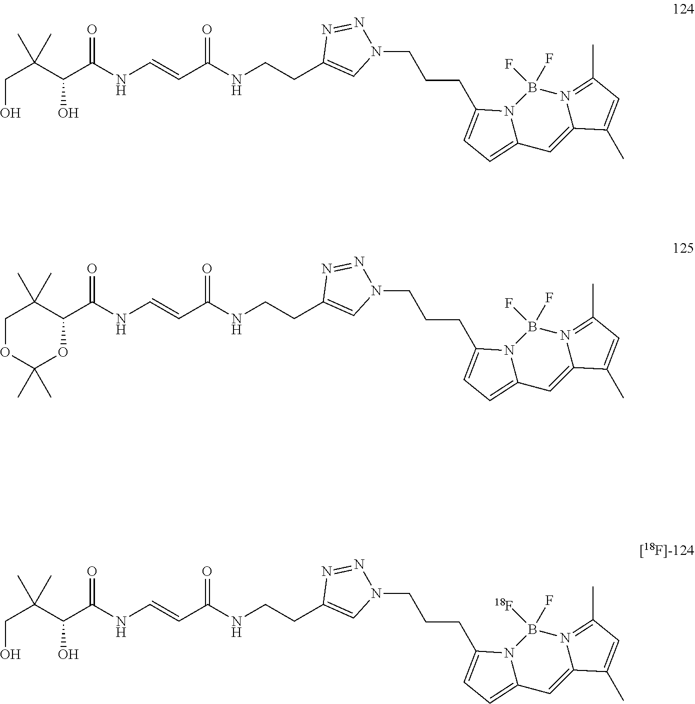
and salts, solvates, or protected forms thereof.

25. The compound of any one of claims 1 to 24, wherein:
(a) the compound is formulated as a pharmaceutical composition;
(b) the compound is formulated for administration by means selected from the group consisting of oral administration; buccal administration; sublingual administration; transdermal administration; transmucosal administration; intranasal administration; ocular administration; pulmonary administration; rectal administration; vaginal administration; parenteral administration; and administration via implant; and/or
(c) the compound is formulated for administration by means selected from the group consisting of: subcutaneous injection, intradermal injection, intramuscular injection, intravenous injection, intraarterial injection, intracardiac injection, intrathecal injection, intraspinal injection, intracapsular injection, subcapsular injection, intraorbital injection, intraperitoneal injection, intratracheal injection, subcuticular injection, intraarticular injection, subarachnoid injection, and intrasternal injection.

26. A method of:
(i) treating a malignant cell;
(ii) preventing activity of a malignant cell;
(iii) modifying a malignant cell;
(iv) treating a malignant cell in a subject; or
(v) preventing activity of a malignant cell in a subject;
the method comprising:
for (i), contacting the malignant cell with a compound, the compound being a chemical conjugate comprising a pantothenic acid group, a pantothenic acid derivative group, or a pantothenic acid analogue, and at least one therapeutic agent, thereby treating the malignant cell;
for (ii), contacting the malignant cell with a compound, the compound being a chemical conjugate comprising a pantothenic acid group, a pantothenic acid derivative group, or a pantothenic acid analogue, and at least one therapeutic agent, thereby preventing the activity of the malignant cell;
for (iii), contacting the malignant cell with a compound, the compound being a chemical conjugate comprising a pantothenic acid group, a pantothenic acid derivative group, or a pantothenic acid analogue, and at least one cell modifying agent, thereby modifying the malignant cell;
for (iv), administering to the subject a compound, the compound being a chemical conjugate comprising a pantothenic acid group, a pantothenic acid derivative group, or a pantothenic acid analogue, and at least one therapeutic agent, thereby treating the malignant cell in the subject; or
for (v), administering to the subject a compound, the compound being a chemical conjugate comprising a pantothenic acid group, a pantothenic acid derivative group, or a pantothenic acid analogue, and at least one therapeutic agent, thereby preventing the activity of the malignant cell in the subject.

27. The method of claim 26, wherein the compound is of formula (I): 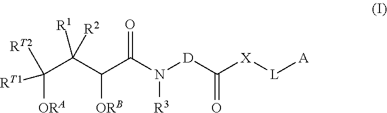
wherein:
—R^{A} and —R^{B} are each independently selected from hydrogen, alkyl, alkenyl, alkynyl, aralkyl, cycloalkylalkyl, alkanoyl and aralkanoyl;
or —R^{A} and —R^{B} are together —C(R^{C1})(R^{C2})—, forming a 6-membered ring, where —R^{C1} is independently selected from hydrogen, alkyl, alkenyl, alkynyl, aralkyl and cycloalkylalkyl, and —R^{C2} is independently selected from hydrogen, alkyl, alkenyl, alkynyl, aralkyl, cycloalkylalkyl, alkoxy, alkenoxy, alkynoxy, aralkoxy and cycloalkylalkoxy, or —R^{C1} and —R^{C2} are together oxo (═O);
—R^{T1} and —R^{T2} are each independently hydrogen or alkyl;
—R¹ and —R² are each independently selected from hydrogen, alkyl, alkenyl, alkynyl, aralkyl and cycloalkylalkyl;
—R³ is hydrogen or alkyl;
-D- is C₂₋₄ alkenylene or C₁₋₄ alkylene, where the alkenylene or alkylene is optionally substituted with alkyl or halo;
—X— is a covalent bond, —N(R⁴)—, —O—, —S—, or —Se—, where —R⁴ is hydrogen or alkyl;
-L- is a linker or a covalent bond; and
-A comprises at least one chemical agent;
and salts, solvates and protected forms thereof.

28. The method of claim 27, wherein the linker is selected from the group consisting of bivalent linkers; multivalent linkers; cleavable linkers; enzyme cleavable linkers; protease cleavable linkers; esterase cleavable linkers.

29. The method of claim 27, wherein the linker is selected from the group consisting of triazole linkers; glycol linkers; polyethylene glycol (PEG) linkers; alkyl linkers; heteroalkyl linkers; and alkylene linkers.

30. The method of any one of claims 26 to 29, wherein the at least one therapeutic agent is selected from the group consisting of: antibodies; antimetabolites; biologic response modifiers; chain terminators; growth factor inhibitors; hormonal agents; nucleic acid intercalators; pathway inhibitors; proteasome inhibitors; enzyme inhibitors; radionuclides; radionucleotides; and any combination thereof.

31. The method of any one of claims 26 to 29, wherein the at least one therapeutic agent is selected from the group consisting of auranofin; vemurafenib; dabrafenib; trametinib; and any combination thereof.

32. The method of any one of claims 26 to 29, wherein the at least one cell modifying agent is selected from the group consisting of: polypeptides; peptides; enzymes; enzyme fragments; antibodies; antibody fragments; antibody epitopes; polynucleotides; RNAs; DNAs; genes; gene fragments; and any combination thereof.

33. The method of any one of claims 26 to 34, wherein the malignant cell is a THL positive cell.

34. The method of any one of claims 26 to 34, wherein the malignant cell is selected from the group consisting of: bladder cancer cells, bone cancer cells, breast cancer cells, brain cancer cells, colorectal cancer cells, endometrial cancer cells, head and neck cancer cells, kidney cancer cells, leukaemia cells, liver cancer cells, lung cancer cells, lymphoma cells, melanoma cells, ovarian cancer cells, pancreatic cancer cells, prostate cancer cells, and thyroid cancer cells.

35. The method of any one of claims 26 to 34, wherein the malignant cell is selected from the group consisting of: melanoma cells; colon cancer cells; rectal cancer cells; and leukaemia cells.

36. The method of any one of claims 26 to 34, wherein the malignant cell is selected from the group consisting of: malignant melanocytes; melanoma tissue cells; melanoma lesion cells; and melanoma tumour cells.

37. The method of any one of claims 26 to 34, wherein the malignant cell is selected from the group consisting of: melanoma metastasis cells; cutaneous melanoma cells; ocular melanoma cells; superficial spreading melanoma cells; nodular melanoma cells; lentigo maligna melanoma cells; amelanotic melanoma cells; acral lentiginous melanoma cells; mucosal melanoma cells; desmoplastic melanoma cells; and uveal melanoma cells.

38. The method of any one of claims 26 to 34, wherein the malignant cell is selected from the group consisting of: (a) melanoma cells having mutation of one or more of CDK2NA, MDm2, CDK4EGF, RB1, MC1R, TYR, TYRP1, and ASIP genes; and (b) melanoma cells having mutation of one or more of BRAF, EGF, Fas, and PTEN genes.

39. The method of any one of claims 26 to 34, wherein the malignant cell is selected from the group consisting of: colorectal adenocarcinoma cells; colorectal mucinous adenocarcinoma cells; colorectal signet ring adenocarcinoma cells; colorectal lymphoma cells; gastrointestinal stromal tumour cells; colorectal leiomyosarcoma cells; colorectal melanoma cells; colorectal squamous cell carcinoma cells; hereditary nonpolyposis colorectal cancer cells; metastatic colorectal cancer cells; familial adenomatous polyposis cells; juvenile polyposis coli cells; Turcot Syndrome cancer cells; and Peutz-Jeghers Syndrome cancer cells.

40. The method of any one of claims 26 to 34, wherein the malignant cell is selected from the group consisting of: lymphocytic leukaemia cells; acute lymphocytic leukaemia cells; chronic lymphocytic leukaemia cells; myeloid leukaemia cells; acute myeloid leukaemia cells; chronic myeloid leukaemia cells; prolymphocytic leukaemia cells; large granular lymphocytic (LGL) leukaemia cells; hairy cell leukaemia cells; and myelodysplastic syndrome cells.

41. The method of any one of claims 26 to 34, wherein the malignant cell is selected from the group consisting of: tumour node and metastasis (TNM) staged cancer cells; Stage 0 cancer cells; Stage I cancer cells; Stage I cancer cells; Stage ITT cancer cells; Stage IIIA cancer cells; Stage IIIB cancer cells: Stage IIIC cancer cells; Stage IIID cancer cells; Stage IV cancer cells.

42. The method of any one of claims 26 to 41, wherein:
(a) the contacting of the malignant cell is carried out in vitro or in vivo;
(b) the administrating to the subject is by one or more of: oral administration; buccal administration; sublingual administration; transdermal administration; transmucosal administration; intranasal administration; ocular administration; pulmonary administration; rectal administration; vaginal administration; parenteral administration; and administration via implant;
(c) the administrating to the subject is by one or more of: subcutaneous injection, intradermal injection, intramuscular injection, intravenous injection, intraarterial injection, intracardiac injection, intrathecal injection, intraspinal injection, intracapsular injection, subcapsular injection, intraorbital injection, intraperitoneal injection, intratracheal injection, subcuticular injection, intraarticular injection, subarachnoid injection, and intrasternal injection; and/or
(d) the administrating is to a human subject.

43. The method of any one of claims 26 to 42, wherein the treating includes one or more of:
(a) reducing viability, replication, and/or tumour forming ability of the malignant cell; or
(b) reducing vascularity, the growth, and/or the size of a tumour which comprises the malignant cell;
(c) reducing at least one symptom in the subject caused by a presence of the malignant cell;
(d) reducing at least one symptom in the subject caused by a presence of a tumour which comprises the malignant cell;
(e) eliminating the malignant cell in the subject; or
(f) eliminating a tumour in the subject which comprises the malignant cell.

44. The method of any one of claims 26 to 42, wherein the preventing the activity includes one or more of:
(a) impeding growth or spread of the malignant cell;
(b) impeding growth or spread of a tumour which comprises the malignant cell; or
(c) halting, decreasing, or delaying metastasis by the malignant cell;
(d) halting, decreasing, or delaying metastasis of a tumour which comprises the malignant cell;
(e) delaying onset of at least one symptom in the subject caused by a presence of the malignant cell in the subject; or
(f) delaying onset of at least one symptom in the subject caused by a presence of a tumour which comprises the malignant cell.

45. The method of any one of claims 26 to 44, wherein the compound is selected from the group consisting of formulae: 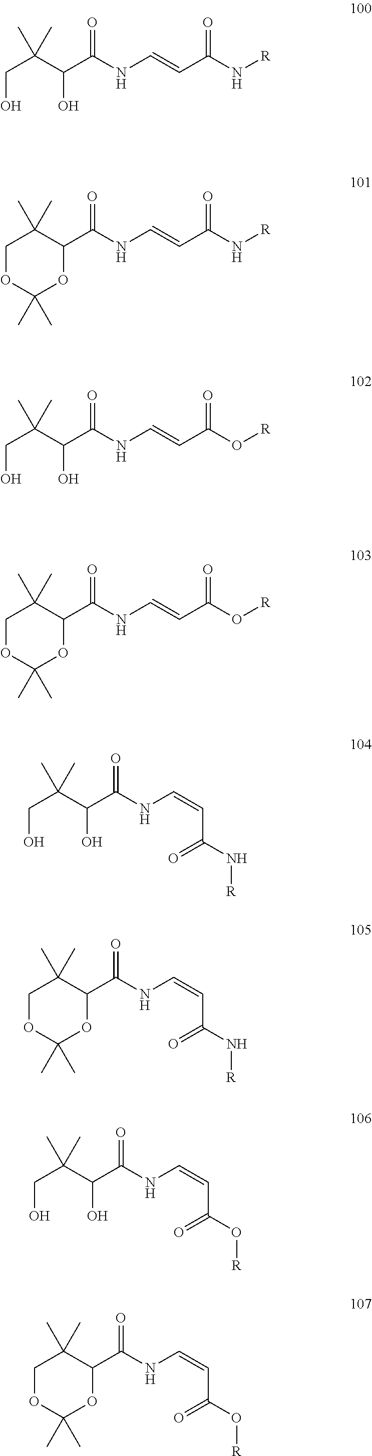 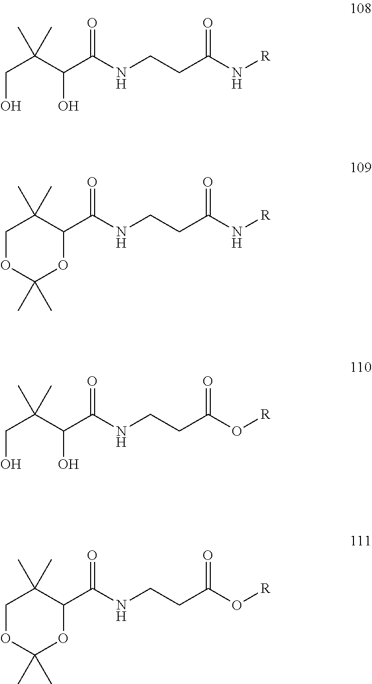
wherein —R is -L-A;
wherein -L- is a linker or a covalent bond; and -A is at least one chemical agent,
and salts, solvates, and protected forms thereof.

46. The method of any one of claims 26 to 44, wherein the compound is selected from the group consisting of formulae: 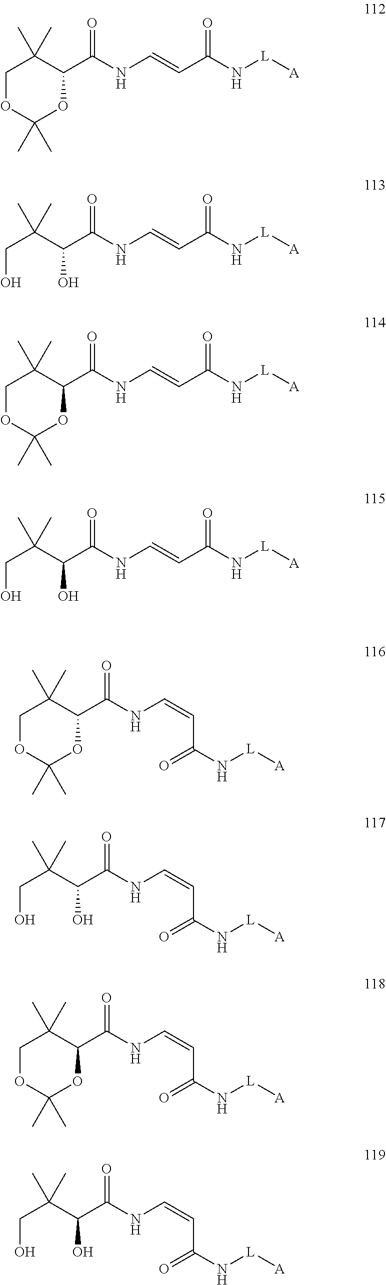 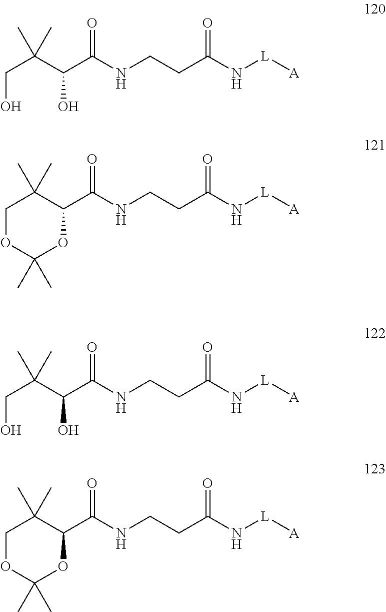
wherein -L- is a linker or a covalent bond; and -A is at least one chemical agent,
and salts, solvates, and protected forms thereof.

47. The method of any one of claims 26 to 44 wherein the compound is selected from the group consisting of: 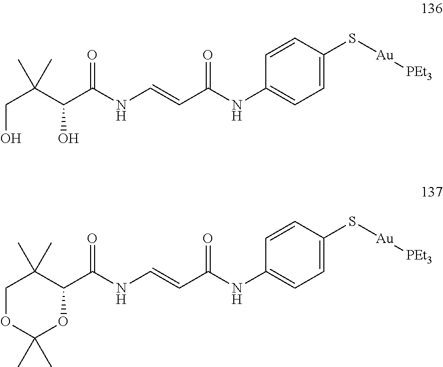
and salts, solvates, and protected forms thereof.

48. The method of any one of claims 26 to 47, wherein:
(a) the compound is formulated as a pharmaceutical composition;
(b) the compound is formulated for administration by means selected from the group consisting of: oral administration; buccal administration; sublingual administration; transdermal administration; transmucosal administration; intranasal administration; ocular administration; pulmonary administration; rectal administration; vaginal administration; parenteral administration; and administration via implant; and/or
(c) the compound is formulated for administration by means selected from the group consisting of: subcutaneous injection, intradermal injection, intramuscular injection, intravenous injection, intraarterial injection, intracardiac injection, intrathecal injection, intraspinal injection, intracapsular injection, subcapsular injection, intraorbital injection, intraperitoneal injection, intratracheal injection, subcuticular injection, intraarticular injection, subarachnoid injection, and intrasternal injection.

49. A kit for use in:
(i) targeting at least one chemical agent to a malignant cell;
(ii) detecting a malignant cell;
(iii) treating a malignant cell;
(iv) preventing activity of a malignant cell; or
(v) modifying a malignant cell;
wherein:
for (i), the kit comprises a compound which is a chemical conjugate comprising a pantothenic acid group, a pantothenic acid derivative group, or a pantothenic acid analogue, and at least one chemical agent;
for (ii), the kit comprises a compound which is a chemical conjugate comprising a pantothenic acid group, a pantothenic acid derivative group, or a pantothenic acid analogue, and at least one detection agent;
for (iii) or (iv), the kit comprises a compound which is a chemical conjugate comprising a pantothenic acid group, a pantothenic acid derivative group, or a pantothenic acid analogue, and at least one therapeutic agent; or
for (v), the kit comprises a compound which is a chemical conjugate comprising a pantothenic acid group, a pantothenic acid derivative group, or a pantothenic acid analogue, and at least one cell modifying agent.

50. The kit of claim 49, wherein the compound is of formula (I): 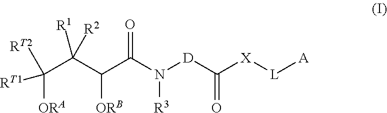
wherein:
—R^{A} and —R^{B} are each independently selected from hydrogen, alkyl, alkenyl, alkynyl, aralkyl, cycloalkylalkyl, alkanoyl and aralkanoyl;
or —R^{A} and —R^{B} are together —C(R^{C1})(R^{C2})— forming a 6-membered ring, where —R^{C1} is independently selected from hydrogen, alkyl, alkenyl, alkynyl, aralkyl and cycloalkylalkyl, and —R^{C2} is independently selected from hydrogen, alkyl, alkenyl, alkynyl, aralkyl, cycloalkylalkyl, alkoxy, alkenoxy, alkynoxy, aralkoxy and cycloalkylalkoxy, or —R^{C1} and —R^{C2} are together oxo (═O);
—R^{T1} and —R^{T2} are each independently hydrogen or alkyl;
—R¹ and —R² are each independently selected from hydrogen, alkyl, alkenyl, alkynyl, aralkyl and cycloalkylalkyl;
—R³ is hydrogen or alkyl;
-D- is C₂₋₄ alkenylene or C₁₋₄ alkylene, where the alkenylene or alkylene is optionally substituted with alkyl or halo;
—X— is a covalent bond, —N(R⁴)—, —O—, —S—, or —Se—, where —R⁴ is hydrogen or alkyl;
-L- is a linker or a covalent bond; and
-A comprises at least one chemical agent;
and salts, solvates and protected forms thereof.

51. The kit of claim 50, wherein the linker is selected from the group consisting of bivalent linkers; multivalent linkers; cleavable linkers; enzyme cleavable linkers; protease cleavable linkers; esterase cleavable linkers.

52. The kit of claim 50, wherein the linker is selected from the group consisting of triazole linkers; glycol linkers; polyethylene glycol (PEG) linkers; alkyl linkers; heteroalkyl linkers; and alkylene linkers.

53. The kit of any one of claims 49 to 52, wherein the at least one detection agent is selected from the group consisting of: dyes; fluorophores; fluorescent proteins; unlabelled chelating agents; labelled chelating agents; optical contrast agents; X-ray contrast agents; magnetic resonance imaging contrast agents; positron emission tomography contrast agents; and any combination thereof.

54. The kit any one of claims 49 to 52, wherein the at least one detection agent is selected from the group consisting of BODIPY; BODIPY derivatives; DOTA chelator; indocyanine green; fluorine-18 [¹⁸F] radioisotopes; and any combination thereof.

55. The kit of any one of claims 49 to 52, wherein the at least one therapeutic agent is selected from the group consisting of: antibodies; antimetabolites; biologic response modifiers; chain terminators; growth factor inhibitors; hormonal agents; nucleic acid intercalators; pathway inhibitors; proteasome inhibitors; enzyme inhibitors; radionuclides; radionucleotides; and any combination thereof.

56. The kit of any one of claims 49 to 52, wherein the at least one therapeutic agent is selected from the group consisting of auranofin; vemurafenib; dabrafenib; trametinib; and any combination thereof.

57. The kit of any one of claims 49 to 52, wherein the at least one cell modifying agent is selected from the group consisting of: polypeptides; peptides; enzymes; enzyme fragments; antibodies; antibody fragments; antibody epitopes; polynucleotides; RNAs; DNAs; genes; gene fragments; and any combination thereof.

58. The kit of any one of claims 49 to 57, wherein the malignant cell is a THL positive cell.

59. The kit of any one of claims 49 to 57, wherein the malignant cell is selected from the group consisting of: bladder cancer cells, bone cancer cells, breast cancer cells, brain cancer cells, colorectal cancer cells, endometrial cancer cells, head and neck cancer cells, kidney cancer cells, leukaemia cells, liver cancer cells, lung cancer cells, lymphoma cells, melanoma cells, ovarian cancer cells, pancreatic cancer cells, prostate cancer cells, and thyroid cancer cells.

60. The kit of any one of claims 49 to 59, wherein the malignant cell is selected from the group consisting of: melanoma cells; colon cancer cells; rectal cancer cells; and leukaemia cells.

61. The kit of any one of claims 49 to 59, wherein the malignant cell is selected from the group consisting of:
(a) malignant melanocytes; melanoma tissue cells; melanoma lesion cells; and melanoma tumour cells;
(b) melanoma metastasis cells; cutaneous melanoma cells; ocular melanoma cells; superficial spreading melanoma cells; nodular melanoma cells; lentigo maligna melanoma cells; amelanotic melanoma cells; acral lentiginous melanoma cells; mucosal melanoma cells; desmoplastic melanoma cells; and uveal melanoma cells;
(c) melanoma cells having mutation of one or more of CDK2NA, MDm2, CDK4EGF, RB1, MC1R, TYR, TYRP1, and ASIP genes; and
(d) melanoma cells having mutation of one or more of BRAF, EGF, Fas, and PTEN genes.

62. The kit of any one of claims 49 to 59, wherein the malignant cells selected from the group consisting of: colorectal adenocarcinoma cells; colorectal mucinous adenocarcinoma cells; colorectal signet ring adenocarcinoma cells; colorectal lymphoma cells; gastrointestinal stromal tumour cells; colorectal leiomyosarcoma cells; colorectal melanoma cells; colorectal squamous cell carcinoma cells; hereditary nonpolyposis colorectal cancer cells; metastatic colorectal cancer cells; familial adenomatous polyposis cells; juvenile polyposis coli cells; Turcot Syndrome cancer cells; and Peutz-Jeghers Syndrome cancer cells.

63. The kit of any one of claims 49 to 59, wherein the malignant cells selected from the group consisting of: lymphocytic leukaemia cells; acute lymphocytic leukaemia cells; chronic lymphocytic leukaemia cells; myeloid leukaemia cells; acute myeloid leukaemia cells; chronic myeloid leukaemia cells; prolymphocytic leukaemia cells; large granular lymphocytic (LGL) leukaemia cells; hairy cell leukaemia cells; and myelodysplastic syndrome cells.

64. The kit of any one of claims 49 to 59, wherein the malignant cell is selected from the group consisting of: tumour node and metastasis (TNM) staged cancer cells; Stage 0 cancer cells; Stage I cancer cells; Stage II cancer cells; Stage III cancer cells; Stage IIIA cancer cells; Stage IIIB cancer cells; Stage IIIC cancer cells; Stage IIID cancer cells; Stage IV cancer cells.

65. The kit of any one of claims 49 to 64, wherein:
(a) the targeting of the malignant cell is performed in vitro or in vivo;
(b) the detecting of the malignant cell is performed in vitro or in vivo;
(c) the treating of the malignant cell is performed in vitro or in vivo;
(d) the preventing of the activity of the malignant cell is performed in vitro or in vivo; or
(e) the modifying of the malignant cell is performed in vitro or in vivo.

66. The kit of any one of claims 49 to 65, wherein the detecting includes one or more of: illumination; visualisation; imaging; optical imaging; fluorescence imaging; infra-red imaging; radio imaging; microscopy imaging; machine imaging; extended depth of field imaging; image recording; image capturing; image analysis; image filtering; image reconstruction; computer-aided image analysis; image analysis using artificial intelligence (AI); deep learning; image storage; image storage by cloud computing; image retrieval; image transmission; image recognition; image reconstruction; tomography; computed tomography (CT or CAT); positron emission tomography (PET) or PET-CT; and magnetic resonance imaging (MRI).

67. The kit of any one of claims 49 to 66, wherein the compound is selected from the group consisting of formulae: 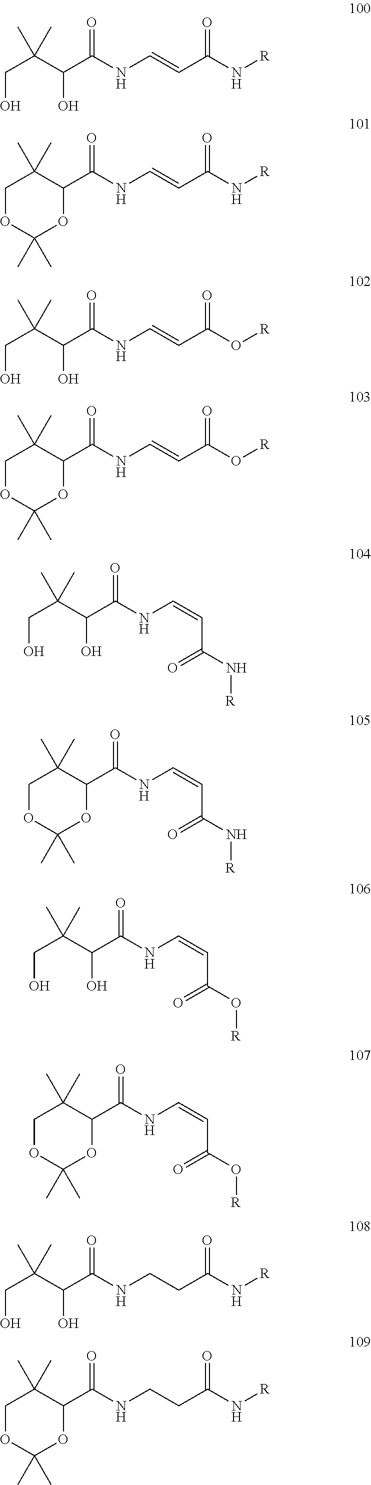 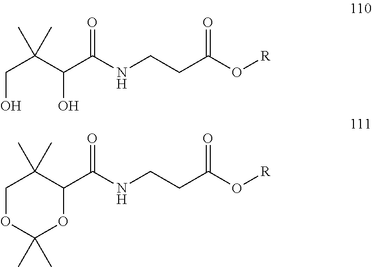
wherein —R is -L-A;
wherein -L- is a linker or a covalent bond; and -A is at least one chemical agent,
and salts, solvates, and protected forms thereof.

68. The kit of any one of claims 49 to 66, wherein the compound is selected from the group consisting of formulae: 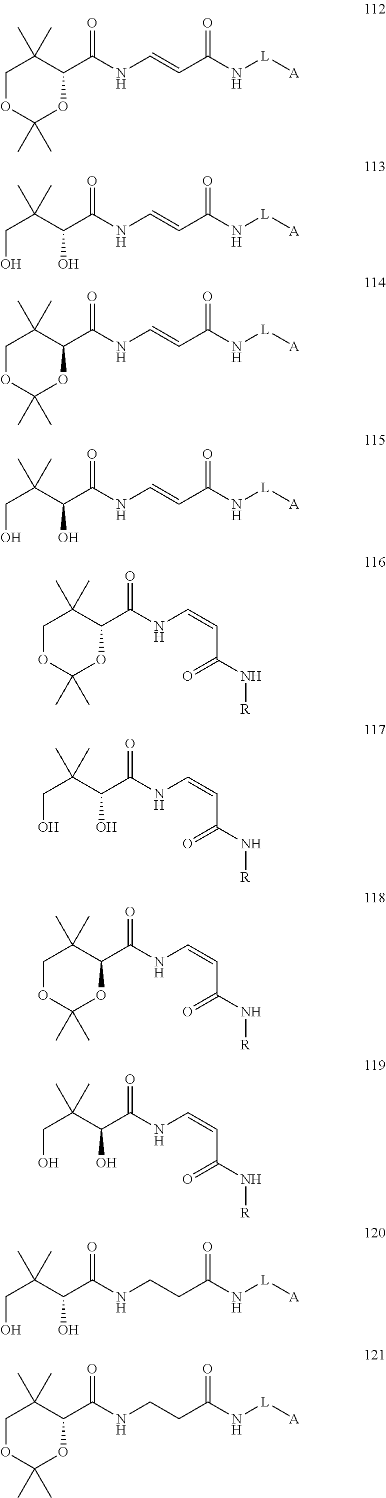 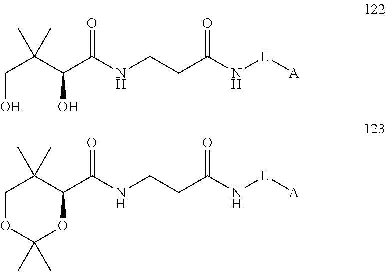
wherein -L- is a linker or a covalent bond; and -A is at least one chemical agent,
and salts, solvates, and protected forms thereof.

69. The kit of any one of claims 49 to 66, wherein the compound is selected from the group consisting of: 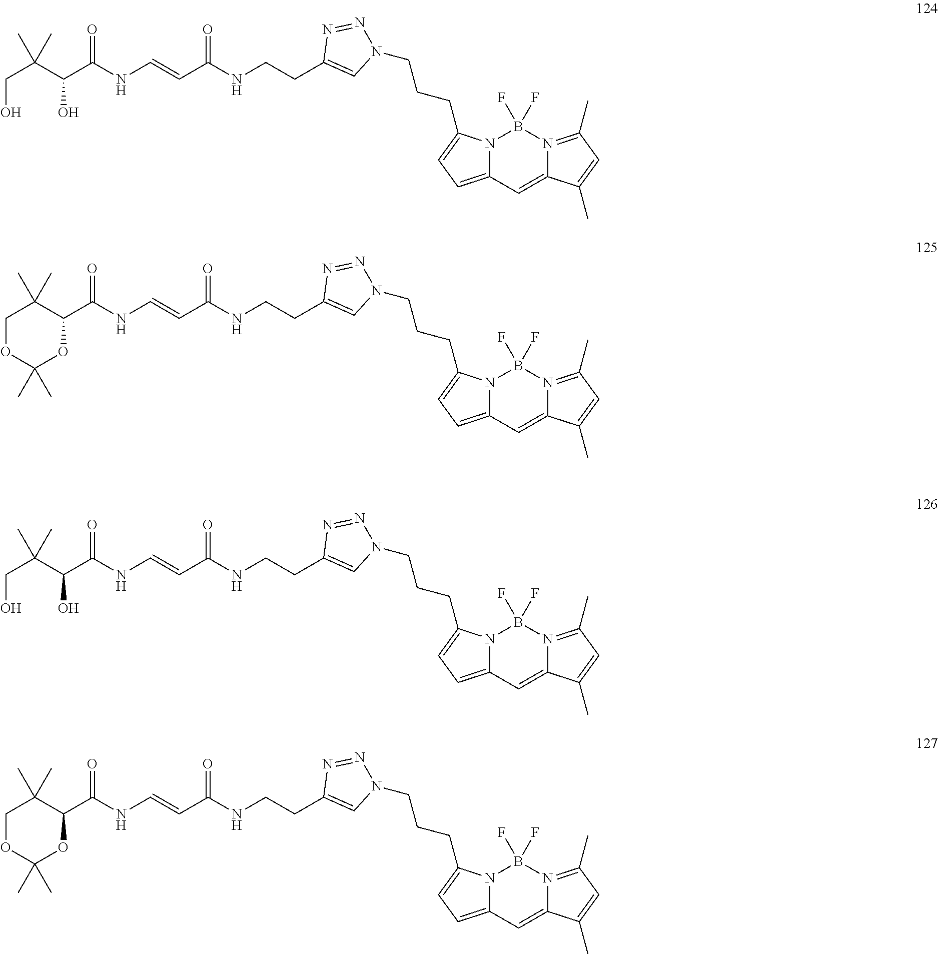 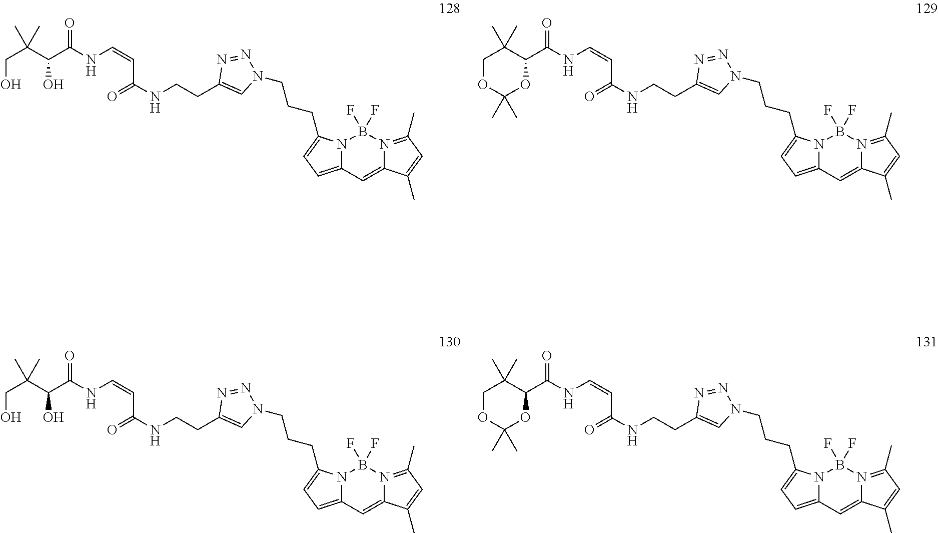 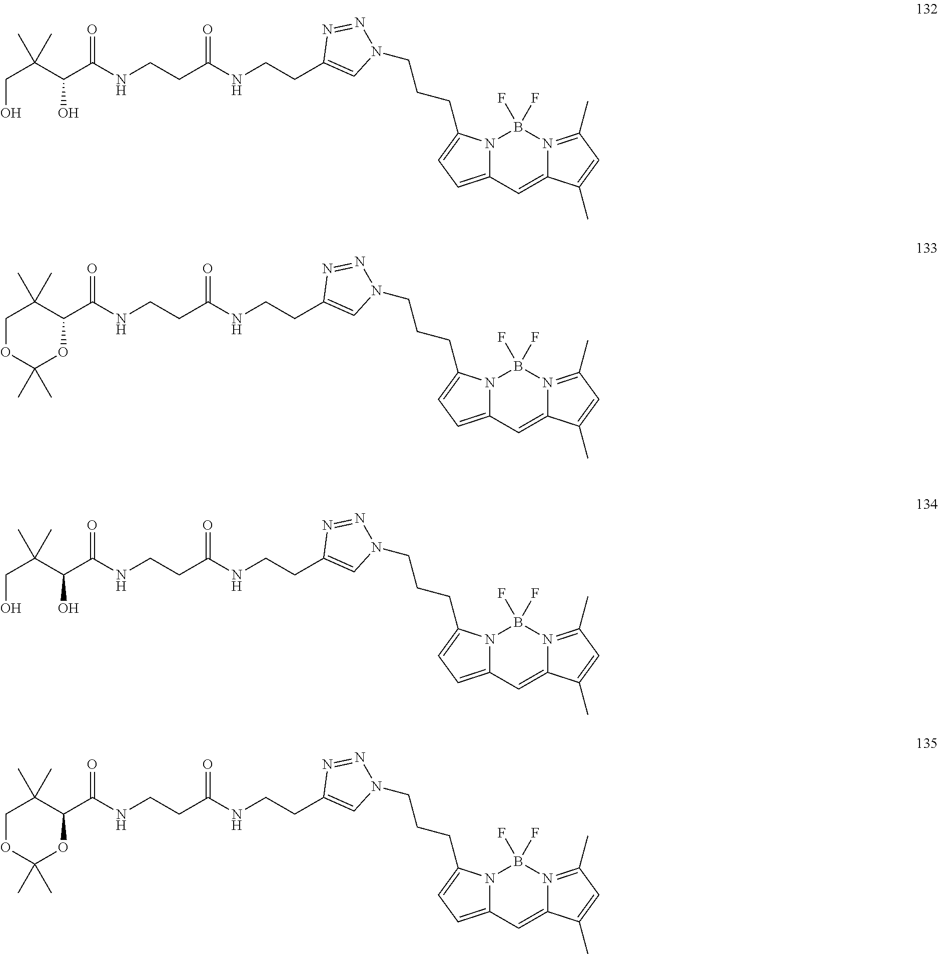 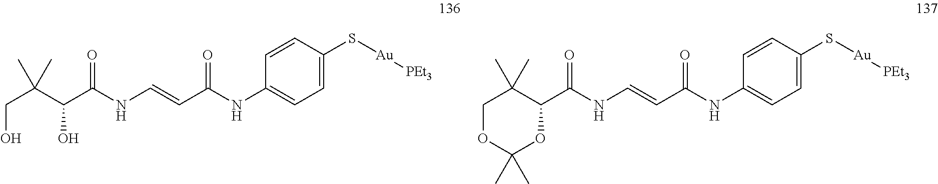
and salts, solvates, and protected forms thereof.

70. The kit of any one of claims 491 to 66, wherein the compound is selected from the group consisting of: 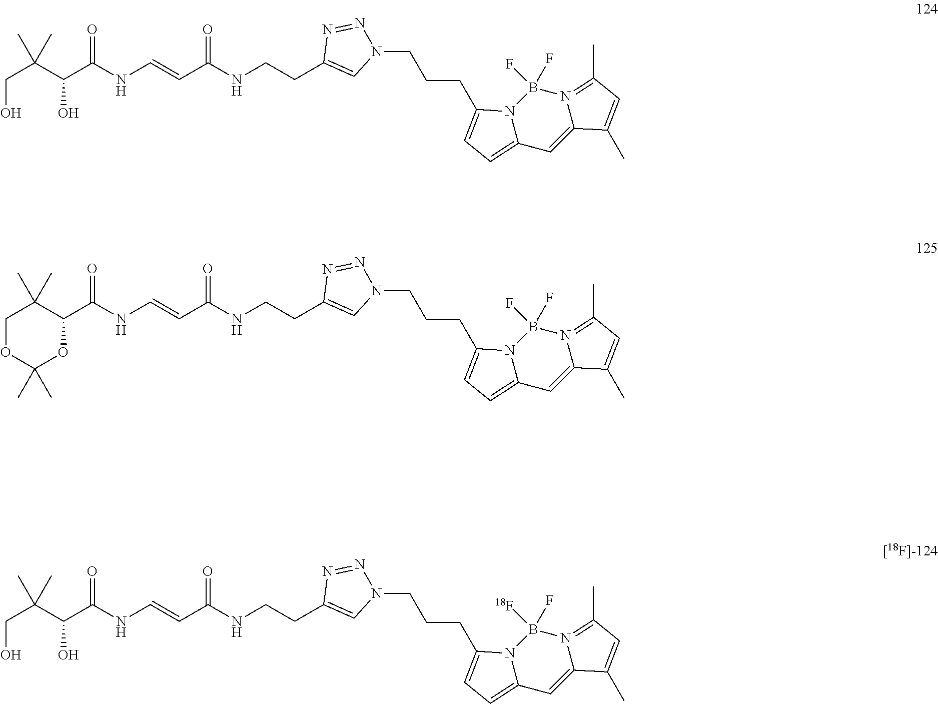
and salts, solvates, or protected forms thereof.

71. The kit of any one of claims 49 to 70, wherein:
(a) the compound is formulated as a pharmaceutical composition;
(b) the compound is formulated for administration by means selected from the group consisting of oral administration; buccal administration; sublingual administration; transdermal administration; transmucosal administration; intranasal administration; ocular administration; pulmonary administration; rectal administration; vaginal administration; parenteral administration; and administration via implant;
(c) the compound is formulated for administration by means selected from the group consisting of: subcutaneous injection, intradermal injection, intramuscular injection, intravenous injection, intraarterial injection, intracardiac injection, intrathecal injection, intraspinal injection, intracapsular injection, subcapsular injection, intraorbital injection, intraperitoneal injection, intratracheal injection, subcuticular injection, intraarticular injection, subarachnoid injection, and intrasternal injection; and/or
(d) the compound is formulated for administration to a human subject.

72. Use of a compound for preparing a medicament for:
(i) targeting at least one chemical agent to a malignant cell in a subject;
(ii) detecting a malignant cell in a subject;
(iii) treating a malignant cell in a subject; or
(iv) preventing activity of a malignant cell in a subject; or
(v) modifying a malignant cell in a subject;
wherein:
for (i), the compound is a chemical conjugate comprising a pantothenic acid group, a pantothenic acid derivative group, or a pantothenic acid analogue, and at least one chemical agent;
for (ii), the compound is a chemical conjugate comprising a pantothenic acid group, a pantothenic acid derivative group, or a pantothenic acid analogue, and at least one detection agent;
for (iii) or (iv), the compound is a chemical conjugate comprising a pantothenic acid group, a pantothenic acid derivative group, or a pantothenic acid analogue, and at least one therapeutic agent; or
for (v), the compound is a chemical conjugate comprising a pantothenic acid group, a pantothenic acid derivative group, or a pantothenic acid analogue, and at least one cell modifying agent.

73. The use of claim 72, wherein the compound is of formula (T): 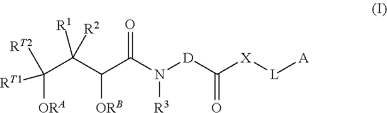
wherein:
—R^{A} and —R^{B} are each independently selected from hydrogen, alkyl, alkenyl, alkynyl, aralkyl, cycloalkylalkyl, alkanoyl and aralkanoyl;
or —R^{A} and —R^{B} are together —C(R^{C1})(R^{C2})—, forming a 6-membered ring, where —R^{C1} is independently selected from hydrogen, alkyl, alkenyl, alkynyl, aralkyl and cycloalkylalkyl, and —R^{C2} is independently selected from hydrogen, alkyl, alkenyl, alkynyl, aralkyl, cycloalkylalkyl, alkoxy, alkenoxy, alkynoxy, aralkoxy and cycloalkylalkoxy, or —R^{C1} and —R^{C2} are together oxo (═O);
—R^{T1} and —R^{T2} are each independently hydrogen or alkyl;
—R¹ and —R² are each independently selected from hydrogen, alkyl, alkenyl, alkynyl, aralkyl and cycloalkylalkyl;
—R³ is hydrogen or alkyl;
-D- is C₂₋₄ alkenylene or C₁₋₄ alkylene, where the alkenylene or alkylene is optionally substituted with alkyl or halo;
—X— is a covalent bond, —N(R⁴)—, —O—, —S—, or —Se—, where —R⁴ is hydrogen or alkyl;
-L- is a linker or a covalent bond; and
-A comprises at least one chemical agent;
and salts, solvates and protected forms thereof.

74. The use of claim 73, wherein the linker is selected from the group consisting of bivalent linkers; multivalent linkers; cleavable linkers; enzyme cleavable linkers; protease cleavable linkers; esterase cleavable linkers.

75. The use of claim 73, wherein the linker is selected from the group consisting of triazole linkers; glycol linkers; polyethylene glycol (PEG) linkers; alkyl linkers; heteroalkyl linkers; and alkylene linkers.

76. The use of any one of claims 72 to 75, wherein the at least one detection agent is selected from the group consisting of: dyes; fluorophores; fluorescent proteins; unlabelled chelating agents; labelled chelating agents; optical contrast agents; X-ray contrast agents; magnetic resonance imaging contrast agents; positron emission tomography contrast agents; and any combination thereof.

77. The use of any one of claims 72 to 75, wherein the at least one detection agent is selected from the group consisting of BODIPY; BODIPY derivatives; DOTA chelator; indocyanine green; fluorine-18 [¹⁸F] radioisotopes; and any combination thereof.

78. The use of any one of claims 72 to 75, wherein the at least one therapeutic agent is selected from the group consisting of: antibodies; antimetabolites; biologic response modifiers; chain terminators; growth factor inhibitors; hormonal agents; nucleic acid intercalators; pathway inhibitors; proteasome inhibitors; enzyme inhibitors; radionuclides; radionucleotides; and any combination thereof.

79. The use of any one of claims 72 to 75, wherein the at least one therapeutic agent is selected from the group consisting of auranofin; vemurafenib; dabrafenib; trametinib; and any combination thereof.

80. The use of any one of claims 72 to 75, wherein the at least one cell modifying agent is selected from the group consisting of: polypeptides; peptides; enzymes; enzyme fragments; antibodies; antibody fragments; antibody epitopes; polynucleotides; RNAs; DNAs; genes; gene fragments; and any combination thereof.

81. The use of any one of claims 72 to 80, wherein the malignant cell is a THL positive cell.

82. The use of any one of claims 72 to 80, wherein the malignant cell is selected from the group consisting of: bladder cancer cells, bone cancer cells, breast cancer cells, brain cancer cells, colorectal cancer cells, endometrial cancer cells, head and neck cancer cells, kidney cancer cells, leukaemia cells, liver cancer cells, lung cancer cells, lymphoma cells, melanoma cells, ovarian cancer cells, pancreatic cancer cells, prostate cancer cells, and thyroid cancer cells.

83. The use of any one of claims 72 to 82, wherein the malignant cell is selected from the group consisting of: melanoma cells; colon cancer cells; rectal cancer cells; and leukaemia cells.

84. The use of any one of claims 72 to 82, wherein the malignant cell is selected from the group consisting of:
(a) malignant melanocytes; melanoma tissue cells; melanoma lesion cells; and melanoma tumour cells;
(b) melanoma metastasis cells; cutaneous melanoma cells; ocular melanoma cells; superficial spreading melanoma cells; nodular melanoma cells; lentigo maligna melanoma cells;
amelanotic melanoma cells; acral lentiginous melanoma cells; mucosal melanoma cells;
desmoplastic melanoma cells; and uveal melanoma cells;
(c) melanoma cells having mutation of one or more of CDK2NA, MDm2, CDK4EGF, RBL MC1R, TYR, TYRP1, and ASIP genes; and
(d) melanoma cells having mutation of one or more of BRAF, OGF, Fas, and PTEN genes.

85. The use of any one of claims 72 to 82, wherein the malignant cell is selected from the group consisting of: colorectal adenocarcinoma cells; colorectal mucinous adenocarcinoma cells; colorectal signet ring adenocarcinoma cells; colorectal lymphoma cells; gastrointestinal stromal tumour cells; colorectal leiomyosarcoma cells; colorectal melanoma cells; colorectal squamous cell carcinoma cells; hereditary nonpolyposis colorectal cancer cells; metastatic colorectal cancer cells; familial adenomatous polyposis cells; juvenile polyposis coli cells; Turcot Syndrome cancer cells; and Peutz-Jeghers Syndrome cancer cells.

86. The use of any one of claims 72 to 82, wherein the malignant cell is selected from the group consisting of: lymphocytic leukaemia cells; acute lymphocytic leukaemia cells; chronic lymphocytic leukaemia cells; myeloid leukaemia cells; acute myeloid leukaemia cells; chronic myeloid leukaemia cells; prolymphocytic leukaemia cells; large granular lymphocytic (LGL) leukaemia cells; hairy cell leukaemia cells; and myelodysplastic syndrome cells.

87. The use of any one of claims 72 to 82, wherein the malignant cell is selected from the group consisting of: tumour node and metastasis (TNM) staged cancer cells; Stage 0 cancer cells; Stage I cancer cells; Stage II cancer cells; Stage III cancer cells; Stage IIIA cancer cells; Stage IIIB cancer cells; Stage IIIC cancer cells; Stage IIID cancer cells; Stage IV cancer cells.

88. The use of any one of claims 72 to 87, wherein the detecting includes one or more of: illumination; visualisation; imaging; optical imaging; fluorescence imaging; infra-red imaging; radio imaging; microscopy imaging; machine imaging; extended depth of field imaging; image recording; image capturing; image analysis; image filtering; image reconstruction; computer-aided image analysis; image analysis using artificial intelligence (AI); deep learning; image storage; image storage by cloud computing: image retrieval; image transmission; image recognition; image reconstruction; tomography; computed tomography (CT or CAT); positron emission tomography (PET) or PET-CT; and magnetic resonance imaging (MRI).

89. The use of any one of claims 72 to 87, wherein the treating includes one or more of:
(a) reducing viability, replication, and/or tumour forming ability of the malignant cell in the subject; or
(b) reducing vascularity, the growth, and/or the size of a tumour in the subject which comprises the malignant cell;
(c) reducing at least one symptom in the subject caused by a presence of the malignant cell;
(d) reducing at least one symptom in the subject caused by a presence of a tumour which comprises the malignant cell;
(e) eliminating the malignant cell in the subject; or
(f) eliminating a tumour in the subject which comprises the malignant cell.

90. The use of any one of claims 72 to 87, wherein the preventing the activity includes one or more of:
(a) impeding growth or spread of the malignant cell in the subject;
(b) impeding growth or spread of a tumour in the subject which comprises the malignant cell; or
(c) halting, decreasing, or delaying metastasis by the malignant cell in the subject;
(d) halting, decreasing, or delaying metastasis of a tumour in the subject which comprises the malignant cell;
(e) delaying onset of at least one symptom in the subject caused by a presence of the malignant cell in the subject: or
(f) delaying onset of at least one symptom in the subject caused by a presence of a tumour which comprises the malignant cell.

91. The use of any one of claims 72 to 90, wherein the compound is selected from the group consisting of formulae: 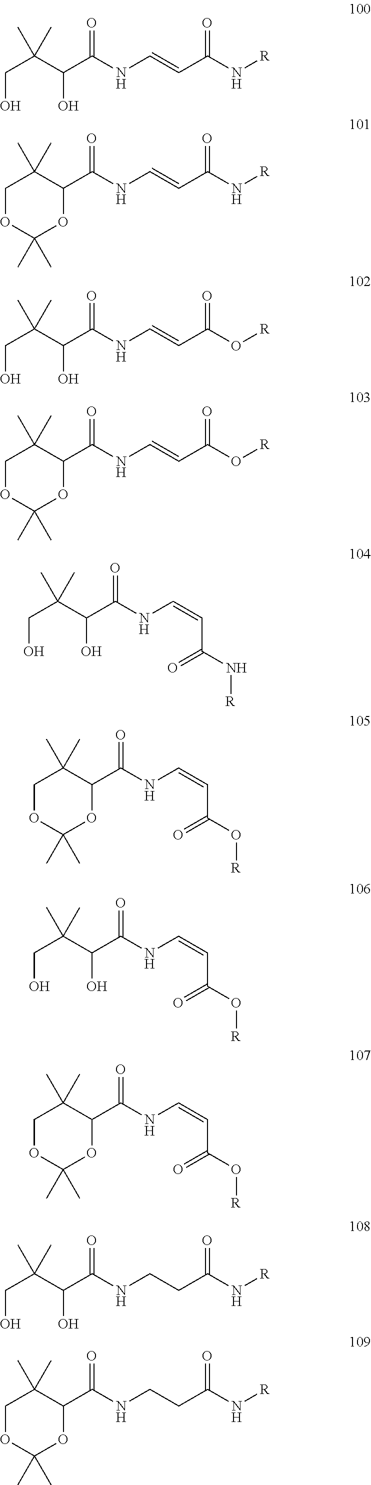 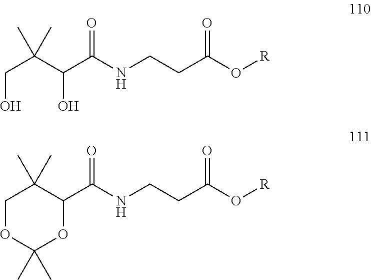
wherein —R is -L-A;
wherein -L- is a linker or a covalent bond; and -A is at least one chemical agent,
and salts, solvates, and protected forms thereof.

92. The use ofany one of claims 72 to 90, wherein the compound is selected from the group consisting of formulae: 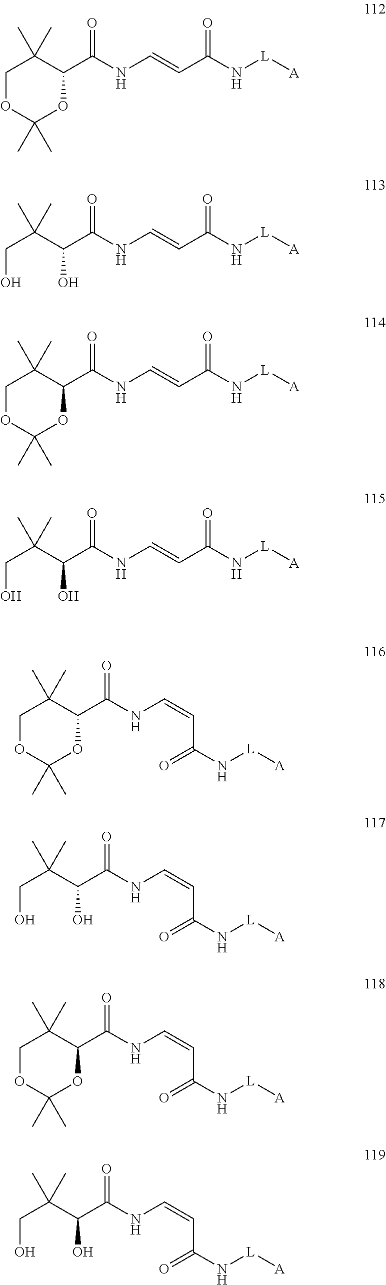 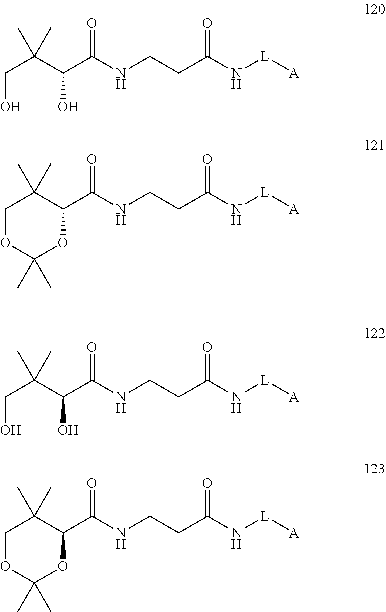
wherein -L- is a linker or a covalent bond; and -A is at least one chemical agent,
and salts, solvates, and protected forms thereof.

93. The use of any one of claims 72 to 90, wherein the compound is selected from the group consisting of 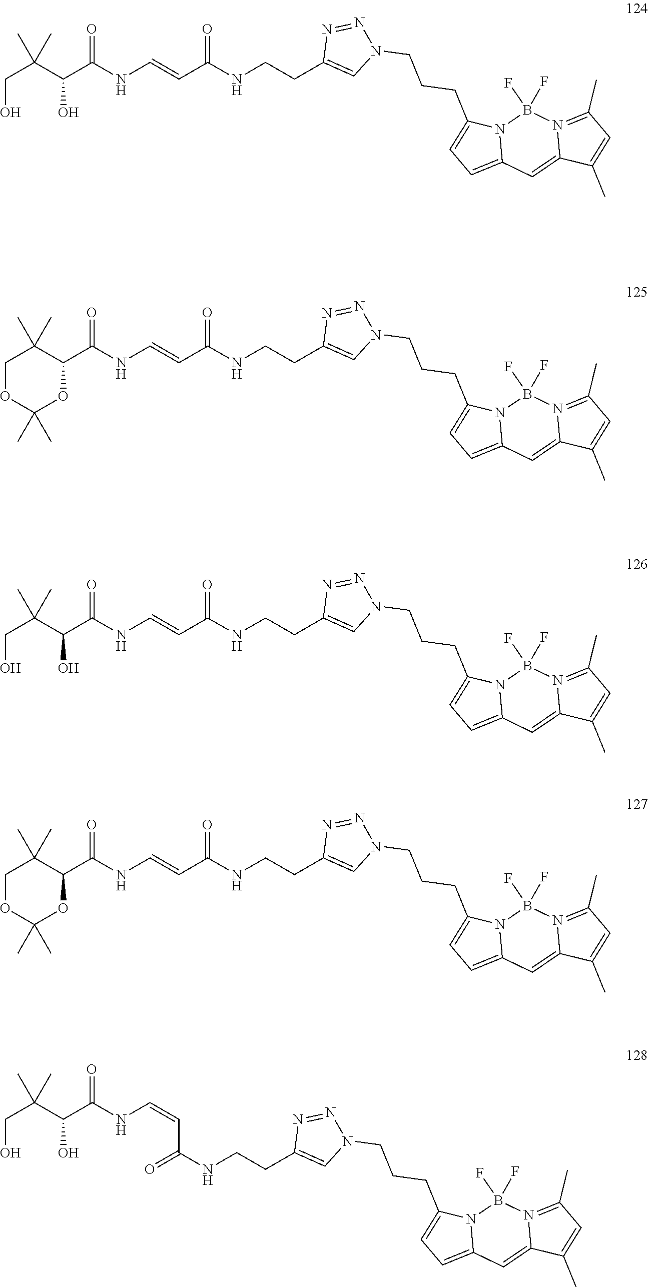 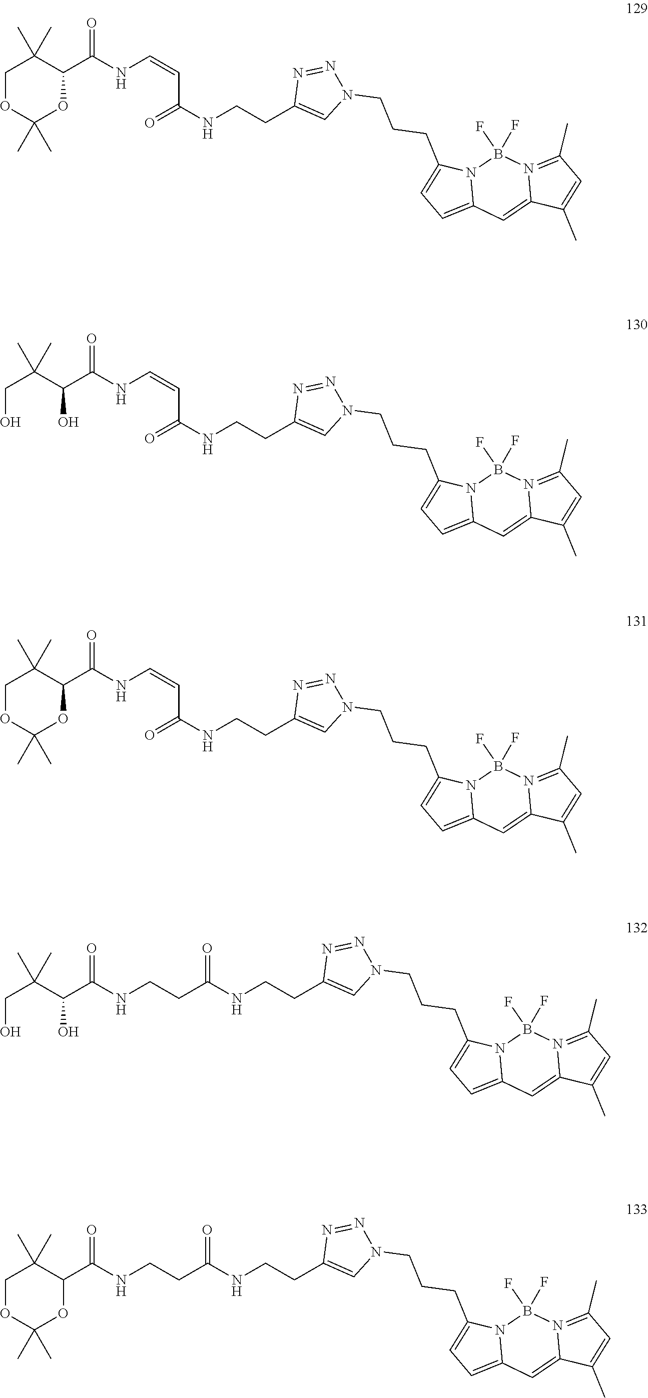 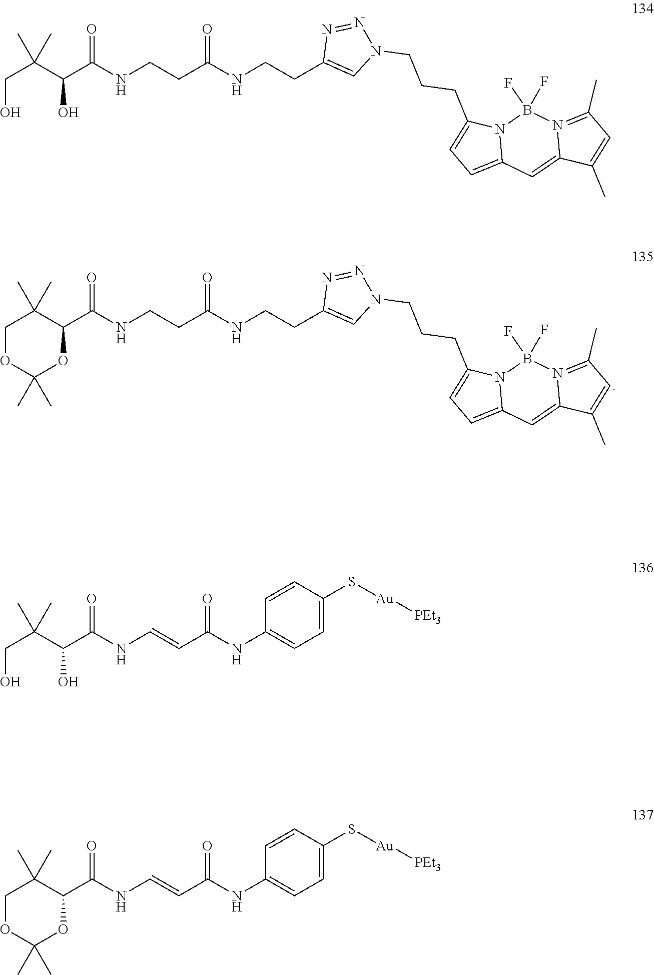
and salts, solvates, and protected forms thereof.

94. The use of any one of claims 72 to 90, wherein the compound is selected from the group consisting of: 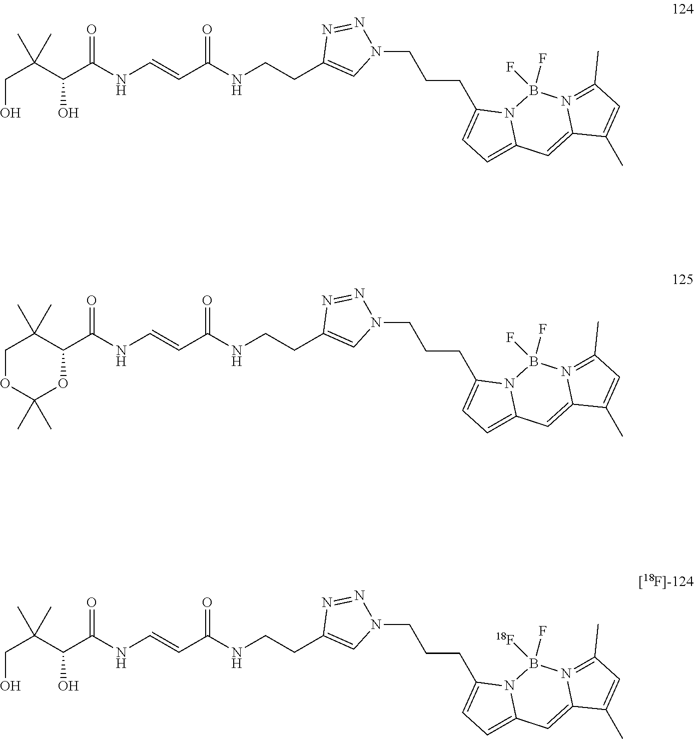
and salts, solvates, or protected forms thereof.

95. The use of any one of claims 72 to 94, wherein:
(a) the medicament is formulated to include one or more pharmaceutically acceptable excipients;
(b) the medicament is formulated for administration by means selected from the group consisting of: oral administration; buccal administration; sublingual administration; transdermal administration; transmucosal administration; intranasal administration; ocular administration; pulmonary administration; rectal administration; vaginal administration; parenteral administration; and administration via implant; and/or
(c) the medicament is formulated for administration by means selected from the group consisting of subcutaneous injection, intradermal injection, intramuscular injection, intravenous injection, intraarterial injection, intracardiac injection, intrathecal injection, intraspinal injection, intracapsular injection, subcapsular injection, intraorbital injection, intraperitoneal injection, intratracheal injection, subcuticular injection, intraarticular injection, subarachnoid injection, and intrasternal injection;
(d) the medicament is formulated for administration to a human subject.

96. A method of detecting at least one malignant cell, the method comprising:
(i) contacting a plurality of cells with a compound, the compound being a chemical conjugate comprising a pantothenic acid group, a pantothenic acid derivative group, or a pantothenic acid analogue, and at least one detection agent, and determining targeting of the compound to at least one of the plurality of cells or an absence of such targeting, wherein the targeting indicates presence of at least one malignant cell in the plurality of cells, while the absence of targeting indicates absence of at least one malignant cell in the plurality of cells; or
(ii) administering a compound to a subject, the compound being a chemical conjugate comprising a pantothenic acid group, a pantothenic acid derivative group, or a pantothenic acid analogue, and at least one detection agent, and determining targeting of the compound to at least one cell in the subject or an absence of such targeting, wherein the targeting indicates presence of at least one malignant cell in the subject, while the absence of targeting indicates absence of at least one malignant cell in the subject; or
(iii) contacting a biological sample with a compound, the compound being a chemical conjugate comprising a pantothenic acid group or pantothenic acid derivative group and at least one detection agent, and (ii) determining targeting of the compound to at least one cell in the biological sample or an absence of such targeting, wherein the targeting indicates presence of at least one malignant cell in the subject, while the absence of targeting indicates absence of at least one malignant cell in the subject.

97. The method of claim 96, wherein the compound is of formula (I): 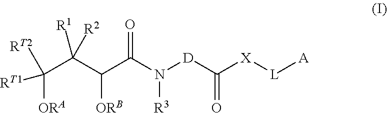
wherein:
—R^{A} and —R^{B} are each independently selected from hydrogen, alkyl, alkenyl, alkynyl, aralkyl, cycloalkylalkyl, alkanoyl and aralkanoyl;
or —R^{A} and —R^{B} are together —C(R^{C1})(R^{C2})—, forming a 6-membered ring, where —R^{C1} is independently selected from hydrogen, alkyl, alkenyl, alkynyl, aralkyl and cycloalkylalkyl, and —R^{C2} is independently selected from hydrogen, alkyl, alkenyl, alkynyl, aralkyl, cycloalkylalkyl, alkoxy, alkenoxy, alkynoxy, aralkoxy and cycloalkylalkoxy, or —R^{C1} and —R^{C2} are together oxo (═O);
—R^{T1} and —R^{T2} are each independently hydrogen or alkyl;
—R¹ and —R² are each independently selected from hydrogen, alkyl, alkenyl, alkynyl, aralkyl and cycloalkylalkyl;
—R³ is hydrogen or alkyl;
-D- is C₂₋₄ alkenylene or C₁₋₄ alkylene, where the alkenylene or alkylene is optionally substituted with alkyl or halo;
—X— is a covalent bond, —N(R⁴)—, —O—, —S—, or —Se—, where —R⁴ is hydrogen or alkyl;
-L- is a linker or a covalent bond; and
-A comprises at least one chemical agent;
and salts, solvates and protected forms thereof.

98. The method of claim 97, wherein the linker is selected from the group consisting of bivalent linkers; multivalent linkers; cleavable linkers; enzyme cleavable linkers; protease cleavable linkers; esterase cleavable linkers.

99. The method of claim 97, wherein the linker is selected from the group consisting of triazole linkers; glycol linkers; polyethylene glycol (PEG) linkers; alkyl linkers; heteroalkyl linkers; and alkylene linkers.

100. The method of any one of claims 96 to 99, wherein the at least one detection agent is selected from the group consisting of: dyes; fluorophores; fluorescent proteins; optical contrast agents; X-ray contrast agents; magnetic resonance imaging contrast agents; positron emission tomography contrast agents; and any combination thereof.

101. The method of any one of claims 96 to 99, wherein the at least one detection agent is selected from the group consisting of BODIPY; BODIPY derivatives; indocyanine green; fluorine-18 [¹⁸F] radioisotopes; and any combination thereof.

102. The method of any one of claims 96 to 101, wherein:
(a) the determining includes measuring a level of the compound in the at least one malignant cell; and/or
(b) the determining includes localising the compound to the at least one malignant cell.

103. The method of claim 102, wherein:
(a) the determining includes comparing the level of the compound to one or more control levels;
(b) the determining includes comparing the level of the compound to one or more threshold levels;
(c) the determining includes comparing the localisation of the compound to one or more controls for localisation;
(d) the determining includes comparing the localisation of the compound to one or more thresholds for localisation.

104. The method of any one of claims 96 to 103, wherein the malignant cell is a THL positive cell.

105. The method of anyone of claims 96 to 103, wherein the malignant cell is selected from the group consisting of: bladder cancer cells, bone cancer cells, breast cancer cells, brain cancer cells, colorectal cancer cells, endometrial cancer cells, head and neck cancer cells, kidney cancer cells, leukaemia cells, liver cancer cells, lung cancer cells, lymphoma cells, melanoma cells, ovarian cancer cells, pancreatic cancer cells, prostate cancer cells, and thyroid cancer cells.

106. The method of any one of claims 96 to 105, wherein the at least one malignant cell is selected from the group consisting of: melanoma cells; colon cancer cells; rectal cancer cells; and leukaemia cells.

107. The method of any one of claims 96 to 105, wherein the at least one malignant cell is selected from the group consisting of:
(a) malignant melanocytes; melanoma tissue cells; melanoma lesion cells; and melanoma tumour cells;
(b) melanoma metastasis cells; cutaneous melanoma cells; ocular melanoma cells; superficial spreading melanoma cells; nodular melanoma cells; lentigo maligna melanoma cells; amelanotic melanoma cells; acral lentiginous melanoma cells; mucosal melanoma cells; desmoplastic melanoma cells; and uveal melanoma cells;
(c) melanoma cells having mutation of one or more of CDK2NA, MDm2, CDK4EGF, RB1, MC1R, TYR, TYRP1, and ASIP genes; and
(d) melanoma cells having mutation of one or more of BRAF, EGF, Fas, and PTEN genes.

108. The method of any one of claims 96 to 105, wherein the malignant cell is selected from the group consisting of: colorectal adenocarcinoma cells; colorectal mucinous adenocarcinoma cells; colorectal signet ring adenocarcinoma cells; colorectal lymphoma cells; gastrointestinal stromal tumour cells; colorectal leiomyosarcoma cells; colorectal melanoma cells; colorectal squamous cell carcinoma cells, hereditary nonpolyposis colorectal cancer cells; metastatic colorectal cancer cells; familial adenomatous polyposis cells; juvenile polyposis coli cells; Turcot Syndrome cancer cells; and Peutz-Jeghers Syndrome cancer cells.

109. The method of any one of claims 96 to 105, wherein the malignant cell is selected from the group consisting of: lymphocytic leukaemia cells; acute lymphocytic leukaemia cells; chronic lymphocytic leukaemia cells; myeloid leukaemia cells; acute myeloid leukaemia cells; chronic myeloid leukaemia cells; prolymphocytic leukaemia cells; large granular lymphocytic (LGL) leukaemia cells; hairy cell leukaemia cells; and myelodysplastic syndrome cells.

110. The method of any one of claims 96 to 105, wherein the malignant cell is selected from the group consisting of: tumour node and metastasis (TNM) staged cancer cells; Stage 0 cancer cells; Stage I cancer cells; Stage II cancer cells; Stage III cancer cells; Stage IIIA cancer cells; Stage IIIB cancer cells; Stage IIIC cancer cells; Stage IIID cancer cells; Stage IV cancer cells.

111. The method of any one of claims 96 to 110, wherein:
(a) the biological sample is selected from the group consisting of: at least one cell; at least one skin cell; at least one organ cell; at least one colon cell; at least one rectal cell; at least one small intestine cell; at least one gastric cell; at least one immune cell; tissue; skin tissue; organ tissue; mucosal tissue; colon tissue; rectal tissue; small intestine tissue; gastric tissue; eye tissue; bone; brain tissue; liver tissue; lung tissue; lymph tissue; anal tissue; vaginal tissue; penis tissue; vulval tissue; and mouth tissue;
(b) the biological sample is selected from the group consisting of blood, plasma, serum, cell culture cell, cell culture cells, cell culture media, cellular matter, extracellular matter, urine, faeces, faecal occult, ascites fluid, bile, secretions, and saliva; and/or
(c) the biological sample is selected from the group consisting of: at least one cell obtained by biopsy; at least one cell obtained by surgery; tissue obtained by biopsy; and tissue obtained by surgery.

112. The method of any one of claims 96 to 110, wherein:
(a) the contacting is performed in vitro or in vivo;
(b) the administrating to the subject is by one or more of: oral administration; buccal administration; sublingual administration; transdermal administration; transmucosal administration; intranasal administration; ocular administration; pulmonary administration; rectal administration; vaginal administration; parenteral administration; and administration via implant;
(c) the administrating to the subject is by one or more of subcutaneous injection, intradermal injection, intramuscular injection, intravenous injection, intraarterial injection, intracardiac injection, intrathecal injection, intraspinal injection, intracapsular injection, subcapsular injection, intraorbital injection, intraperitoneal injection, intratracheal injection, subcuticular injection, intraarticular injection, subarachnoid injection, and intrasternal injection; and/or
(c) the administrating is to a human subject.

113. The method of any one of claims 96 to 112, wherein the detecting includes one or more of; illumination; visualisation; imaging; optical imaging; fluorescence imaging; infra-red imaging; radio imaging; microscopy imaging; machine imaging; extended depth of field imaging; image recording; image capturing; image analysis; image filtering; image reconstruction; computer-aided image analysis; image analysis using artificial intelligence (AI); deep learning; image storage; image storage by cloud computing; image retrieval; image transmission; image recognition: image reconstruction; tomography; computed tomography (CT or CAT): positron emission tomography (PET) or PET-CT; and magnetic resonance imaging (MRI).

114. The method of any one of claims 96 to 113, wherein the compound is selected from the group consisting of formulae: 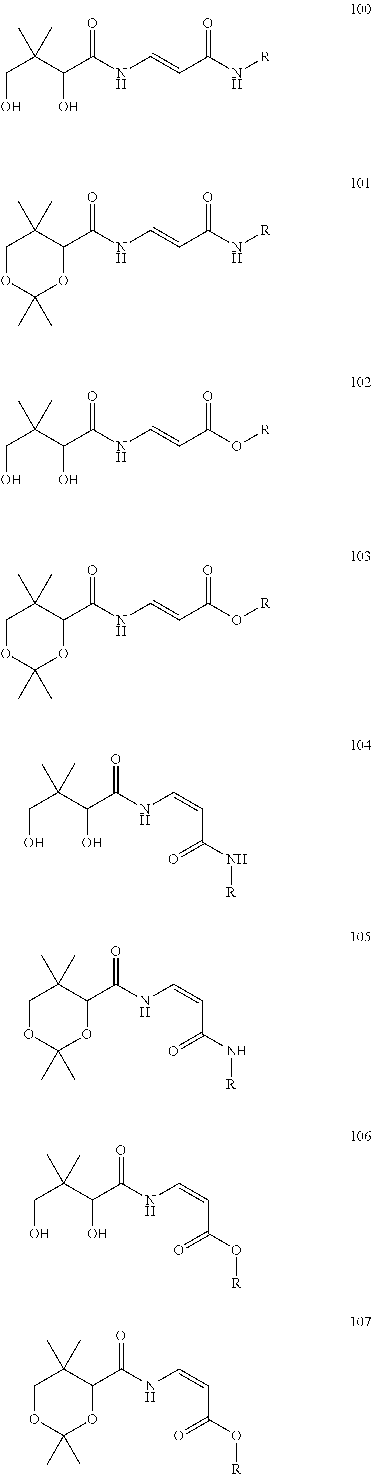 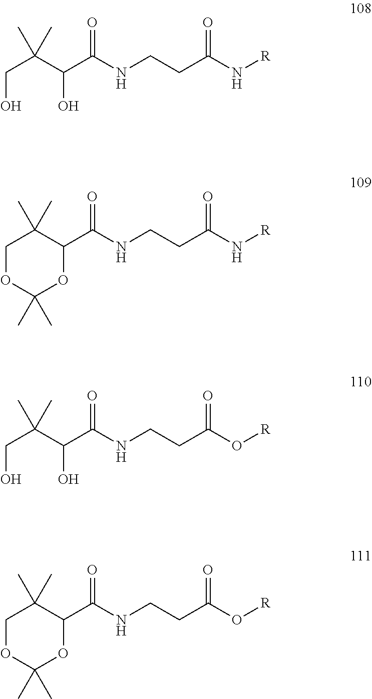
wherein —R is -L-A;
wherein -L- is a linker or a covalent bond; and -A is at least one chemical agent,
and salts, solvates, and protected forms thereof.

115. The method of any one of claims 96 to 113, wherein the compound is selected from the group consisting of formulae: 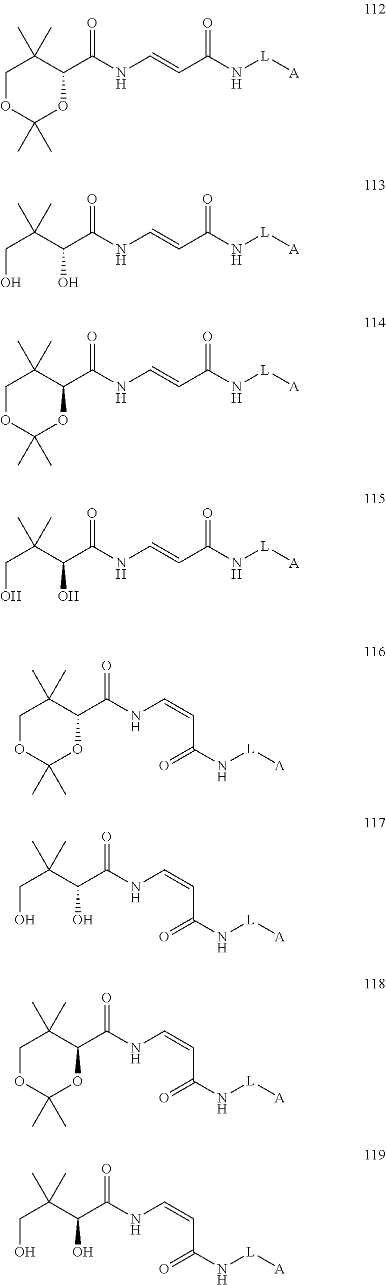 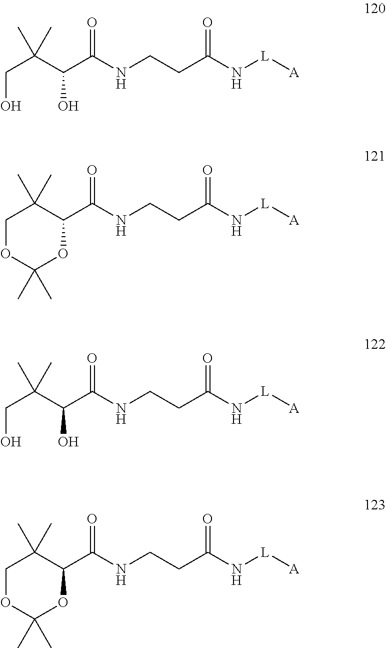
wherein -L- is a linker or a covalent bond; and -A is at least one chemical agent,
and salts, solvates, and protected forms thereof.

116. The method of any one of claims 96 to 113, wherein the compound is selected from the group consisting of: 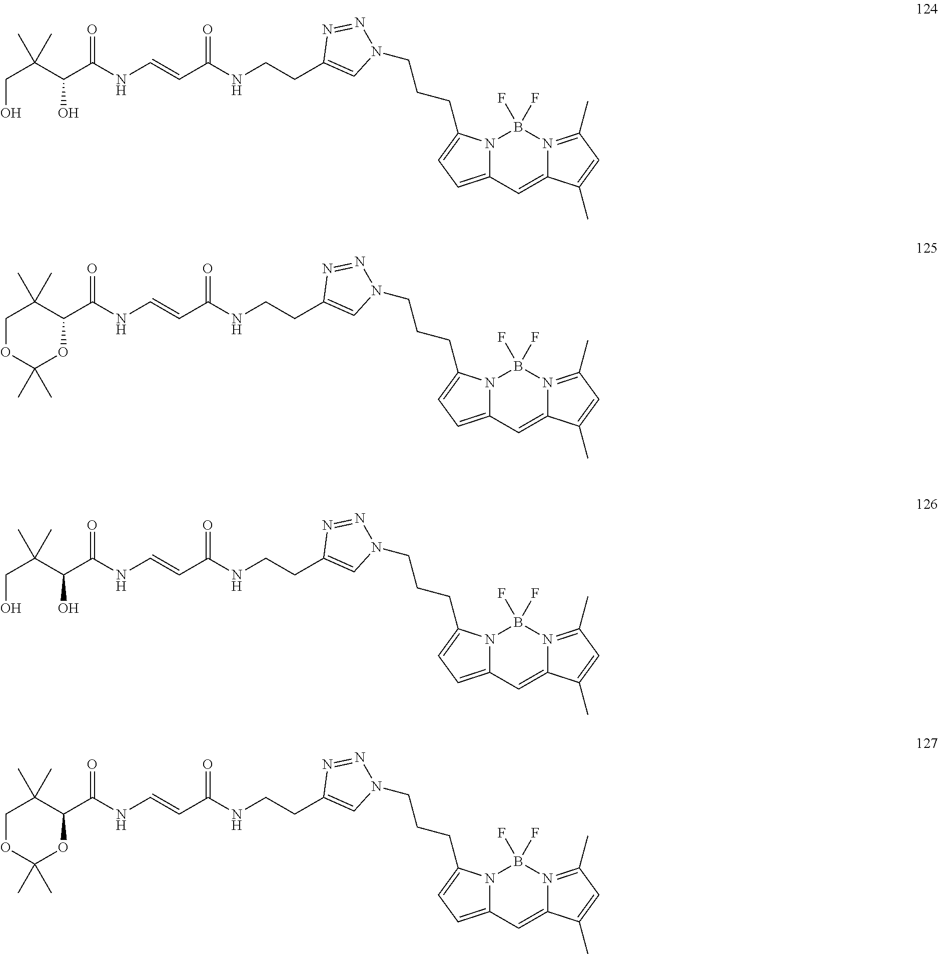 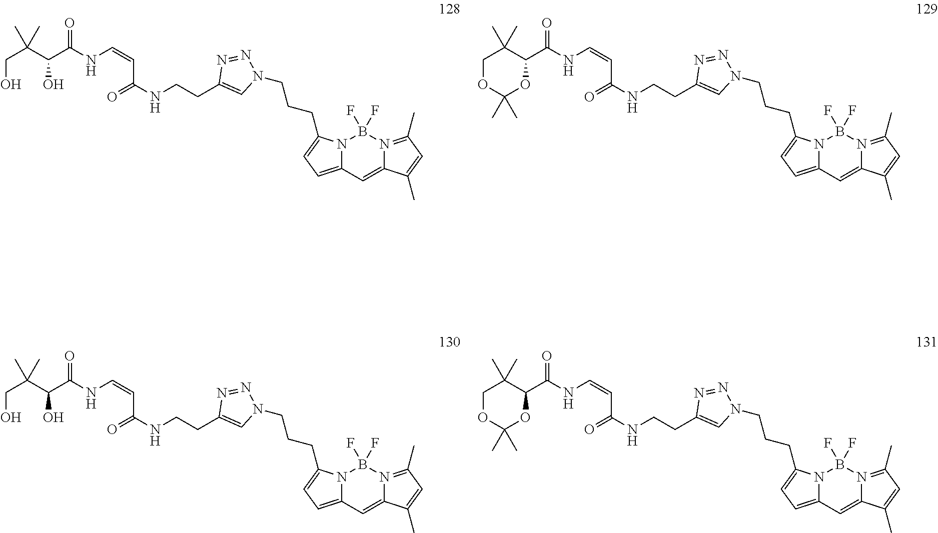 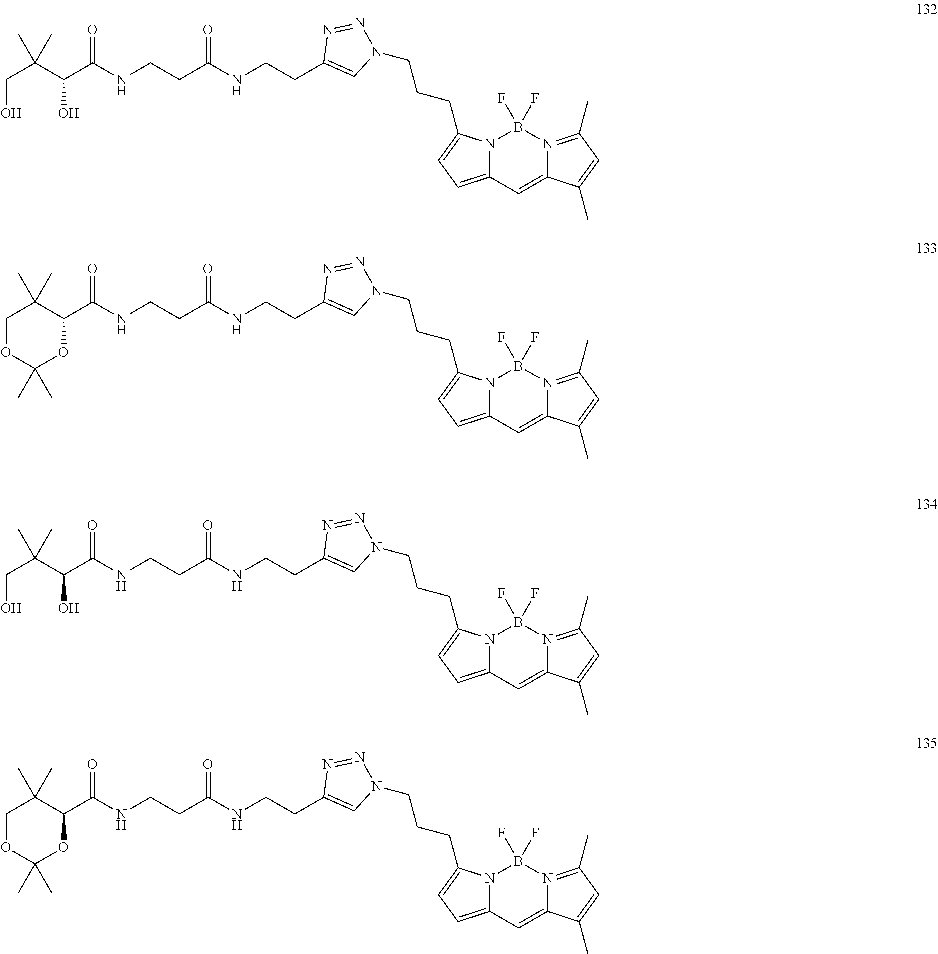
and salts, solvates, and protected forms thereof.

117. The method of any one of claims 96 to 113, wherein the compound is selected from the group consisting of: 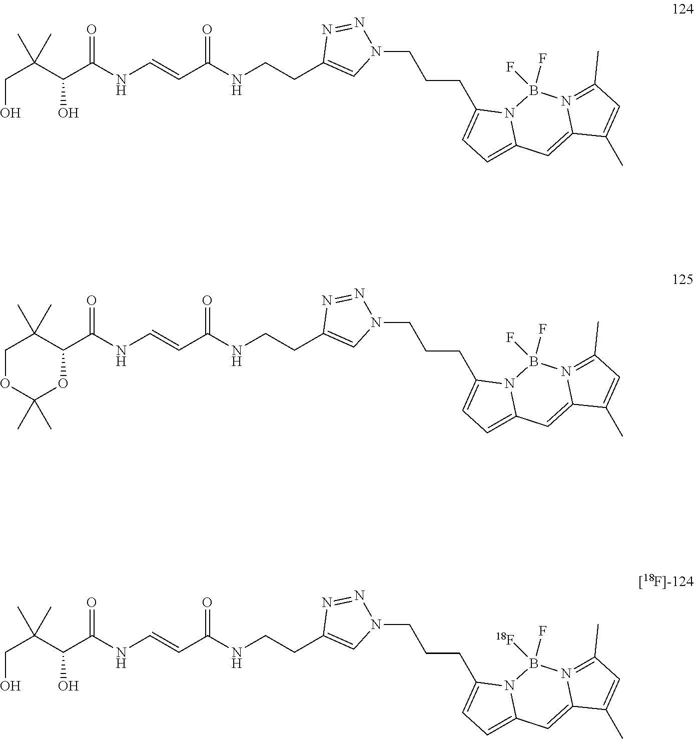
and salts, solvates, or protected forms thereof.

118. The method of any one of claims 96 to 117, wherein:
(a) the compound is formulated as a pharmaceutical composition;
(b) the compound is formulated for administration by means selected from the group consisting of: oral administration; buccal administration; sublingual administration; transdermal administration transmucosal administration; intranasal administration; ocular administration; pulmonary administration; rectal administration; vaginal administration; parenteral administration; and administration via implant; and/or
(c) the compound is formulated for administration by means selected from the group consisting of: subcutaneous injection, intradermal injection, intramuscular injection, intravenous injection, intraarterial injection, intracardiac injection, intrathecal injection, intraspinal injection, intracapsular injection, subcapsular injection, intraorbital injection, intraperitoneal injection, intratracheal injection, subcuticular injection, intraarticular injection, subarachnoid injection, and intrasternal injection.
